(19) United States
(12) Patent Application Publication
(10) Pub. No.: US 2013/0090263 A1
(43) Pub. Date: Apr. 11, 2013

(54) FLUORESCENT PROTEINS, NUCLEIC ACIDS ENCODING THEM AND METHODS FOR MAKING AND USING THEM

(71) Applicant: BP CORPORATION NORTH AMERICA INC., Houston, TX (US)

(72) Inventors: Eileen Tozer, San Diego, CA (US); Feiyu Zhang, Del Mar, CA (US); Carl Abulencia, San Diego, CA (US); Gerhard Frey, San Diego, CA (US); Lilian Parra-Gessert, Redwood City, CA (US)

(73) Assignee: BP CORPORATION NORTH AMERICA INC., Houston, TX (US)

(21) Appl. No.: 13/690,805

(22) Filed: Nov. 30, 2012

Related U.S. Application Data
(63) Continuation of application No. 10/624,909, filed on Jul. 21, 2003, now abandoned.
(60) Provisional application No. 60/397,684, filed on Jul. 19, 2002.

(51) Int. Cl. C07K 14/00; G01N 21/64

(52) U.S. Cl. CPC C07K 14/00; G01N 21/6486
USPC 506/18; 536/23.1; 536/24.3; 536/24.33; 435/91.2; 435/320.1; 435/252.3; 435/325; 435/254.2; 435/254.11; 435/348; 435/419; 536/24.5; 435/69.1; 435/6.11; 436/501; 530/350; 530/387.9; 530/395; 250/459.1

(57) ABSTRACT

The invention is directed to polypeptides having a fluorescent activity, e.g., an auto-fluorescent activity, polynucleotides encoding the polypeptides, and methods for making and using these polynucleotides and polypeptides. The polypeptides of the invention can be used as noninvasive fluorescent markers in living cells and intact organs and animals. The polypeptides of the invention can be used as, e.g., in vivo markers/tracers of gene expression and protein localization, activity indicators, fluorescent resonance energy transfer (FRET) markers, cell lineage markers/tracers, reporters of gene expression and as markers/tracers in protein-protein interactions.

### CROSS-REFERENCE TO RELATED APPLICATIONS

The present application is a continuation of U.S. application Ser. No. 10/624,909, filed Jul. 21, 2003, which claims the benefit of priority to U.S. Provisional Application No. 60/397,684, filed Jul. 19, 2002. The aforementioned applications are explicitly incorporated herein by reference in their entirety and for all purposes.

### TECHNICAL FIELD

This invention relates to molecular and cellular biology and biochemistry. In particular, the invention provides isolated or recombinant nucleic acids and polypeptides originally derived from environmental samples, including nucleic acids from marine samples, such as tide pool samples and reef samples. The invention is directed to polypeptides having fluorescent activity, e.g., auto-fluorescent activity, polynucleotides encoding the polypeptides, and methods for making and using these polynucleotides and polypeptides. The polypeptides of the invention can be used as noninvasive fluorescent markers in living cells and intact organs and animals. The polypeptides of the invention can be used as, e.g., in vivo markers/tracers of gene expression and protein localization, activity indicators, fluorescent resonance energy transfer (FRET) markers, cell lineage markers/tracers, reporters of gene expression and as markers/tracers in protein-protein interactions.

### BACKGROUND

Green fluorescent protein, GFP, is a spontaneously fluorescent protein (i.e., an auto-fluorescent protein). GFP has been isolated from coelenterates, such as the Pacific jellyfish, Aequoria victoria, or from the sea pansy, Renilla reniformis. Its role in coelenterates is to transduce, by energy transfer, the blue chemiluminescence of another protein, aequorin, into green fluorescent light. The family of proteins homologous to GFP from Aequorea victoria exhibits several different types of autocatalytically synthesized chromophores. Phylogenetic analysis has shown that GFP-like proteins from representatives of subclass Zoantharia fall into at least four distinct clades, each Glade containing proteins of more than one emission color (see, e.g., Labas (2002) Proc. Natl. Acad. Sci. USA 99:4256-4261).

Auto-fluorescent proteins, e.g., the green fluorescent protein (GFP) of Aequorea victoria, have become popular research tools. The advantage of these proteins is that the chromophore is autocatalytically formed and does not require addition of a substrate to induce fluorescence. They are used as, e.g., in vivo markers of gene expression (see, e.g., Oshima (2002) Exp. Eye Res. 74:191-198), protein localization (see, e.g., Toyoshima (2002) J. Neurosci. Res. 68:442-448), activity indicators (e.g., pH, Ca2+ levels), and for fluorescent resonance energy transfer (FRET) applications (see, e.g., Ruiz-Velasco (2001) J. Physiol. 537(Pt 3):679-692). GFP can function as a protein tag, as it tolerates N- and C-terminal fusion to a broad variety of proteins many of which have been shown to retain native function.

Fluorescent GFP has been expressed in bacteria, yeast, slime mold, plants, Drosophila, zebrafish, and in mammalian cells. When expressed in mammalian cells, fluorescence from wild type GFP is typically distributed throughout the cytoplasm and nucleus, but excluded from the nucleolus and vesicular organelles. Highly specific intracellular localization including the nucleus, mitochondria, secretory pathway, plasma membrane and cytoskeleton can be achieved via fusions of GFP both to whole proteins and individual targeting sequences. The enormous flexibility as a noninvasive marker in living cells allows for numerous other applications such as a cell lineage tracer, reporter of gene expression and as a potential measure of protein-protein interactions.

Aequorea victoria GFP is 238 amino acids long and has a wild-type absorbance/excitation peak at 395 nm with a minor peak at 475 nm with extinction coefficients of roughly 30,000 and 7,000 M-1 cm-1, respectively. The emission peak is at 508 nm. Interestingly, excitation at 395 nm leads to decrease over time of the 395 nm excitation peak and a reciprocal increase in the 475 nm excitation band. This presumed photoisomerization effect is especially evident with irradiation of GFP by UV light. Analysis of a hexapeptide derived by proteolysis of purified GFP led to the prediction that the fluorophore originates from an internal Ser-Tyr-Gly sequence which is post-translationally modified to a 4-(p-hydroxybenzylidene)-imidazolidin-5-one structure. While no known co-factors or enzymatic components are required for this apparently auto-catalytic process, it is rather thermosensitive with the yield of fluorescently active to total GFP protein decreasing at temperatures greater than 30° C. However, once produced GFP is quite thermostable. The GFP from the sea pansy, Renilla reniformis, exhibits a single major excitation peak at 498 nm, apparently utilizes an identical core fluorophore to that of A. victoria GFP.

Physical and chemical studies of purified GFP have identified several important characteristics. It is very resistant to denaturation requiring treatment with 6 M guanidine hydrochloride at 90° C. or pH of <4.0 or >12.0. Partial to near total renaturation occurs within minutes following reversal of denaturing conditions by dialysis or neutralization. Over a nondenaturing range of pH, increasing pH leads to a reduction in fluorescence by 395 nm excitation and an increased sensitivity to 475 nm excitation.

### SUMMARY

The invention is directed to polypeptides having a fluorescent activity, e.g., auto-fluorescent activity, polynucleotides encoding the polypeptides, and methods for making and using these polynucleotides and polypeptides. In one aspect, the invention provides isolated or recombinant nucleic acids and polypeptides originally derived from environmental samples, including nucleic acids from marine samples, such as tide pool samples and reef samples.

The invention provides isolated or recombinant nucleic acids having at least 85%, 90%, 95%, 96%, 97%, 98%, 99%, or more, sequence identity to SEQ ID NO:1 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection. The invention provides isolated or recombinant nucleic acid comprising a nucleic acid sequence having at least 85%, 90%, 95%, 96%, 97%, 98%, 99%, or more, sequence identity to SEQ ID NO:3 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection. The invention provides isolated or recombinant nucleic acid comprising a nucleic acid sequence having at least 85%, 90%, 95%, 96%, 97%, 98%, 99%, or more, sequence identity to SEQ ID NO:5 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection. The invention provides isolated or recombinant nucleic acid comprising a nucleic acid sequence having at least 85%, 90%, 95%, 96%, 97%, 98%, 99%, or more, sequence identity to SEQ ID NO:7 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection. The invention provides isolated or recombinant nucleic acid comprising a nucleic acid sequence having at least 75%, 80%, 85%, 90%, 95%, 96%, 97%, 98%, 99%, or more, sequence identity to SEQ ID NO:9 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection. The invention provides isolated or recombinant nucleic acid comprising a nucleic acid sequence having at least 75%, 80%, 85%, 90%, 95%, 96%, 97%, 98%, 99%, or more, sequence identity to SEQ ID NO:11 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection. The invention provides isolated or recombinant nucleic acid comprising a nucleic acid sequence having at least 75%, 80%, 85%, 90%, 95%, 96%, 97%, 98%, 99%, or more, sequence identity to SEQ ID NO:13 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection. The invention provides isolated or recombinant nucleic acid comprising a nucleic acid sequence having at least 70%, 75%, 80%, 85%, 90%, 95%, 96%, 97%, 98%, 99%, or more, sequence identity to SEQ ID NO:15 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection. The invention provides isolated or recombinant nucleic acid comprising a nucleic acid sequence having at least 75%, 80%, 85%, 90%, 95%, 96%, 97%, 98%, 99%, or more, sequence identity to SEQ ID NO:17 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection. The invention provides isolated or recombinant nucleic acid comprising a nucleic acid sequence having at least 70%, 75%, 80%, 85%, 90%, 95%, 96%, 97%, 98%, 99%, or more, sequence identity to SEQ ID NO:19 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection. The invention provides isolated or recombinant nucleic acid comprising a nucleic acid sequence having at least 85%, 90%, 95%, 96%, 97%, 98%, 99%, or more, sequence identity to SEQ ID NO:21 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection. The invention provides isolated or recombinant nucleic acid comprising a nucleic acid sequence having at least 85%, 90%, 95%, 96%, 97%, 98%, 99%, or more, sequence identity to SEQ ID NO:23 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection. The invention provides isolated or recombinant nucleic acid comprising a nucleic acid sequence having at least 85%, 90%, 95%, 96%, 97%, 98%, 99%, or more, sequence identity to SEQ ID NO:25 over a region of at least about 100, 150, 200, 250, 300, 350, 400, 450, 500, 550, 600, 650, or more, residues, wherein the nucleic acid encodes a fluorescent polypeptide and the sequence identities are determined by analysis with a sequence comparison algorithm or by a visual inspection.

In one aspect, the invention provides isolated or recombinant nucleic acid, wherein the nucleic acid comprises a nucleic acid having a sequence as set forth in SEQ ID NO:1, sequence as set forth in SEQ ID NO:3, sequence as set forth in SEQ ID NO:5, sequence as set forth in SEQ ID NO:7, sequence as set forth in SEQ ID NO:9, sequence as set forth in SEQ ID NO:11, sequence as set forth in SEQ ID NO:13, sequence as set forth in SEQ ID NO:15, sequence as set forth in SEQ ID NO:17, sequence as set forth in SEQ ID NO:19, sequence as set forth in SEQ ID NO:21, sequence as set forth in SEQ ID NO:23, or sequence as set forth in SEQ ID NO:25. In one aspect, the invention provides isolated or recombinant nucleic acid encoding a polypeptide having a sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:26.

In one aspect, the sequence comparison algorithm is a BLAST version 2.2.2 algorithm where a filtering setting is set to blastall -p blastp -d “nr pataa”-F F, and all other options are set to default.

In one aspect, the isolated or recombinant nucleic acid encodes a green fluorescent protein. In another aspect, the isolated or recombinant nucleic acid encodes a cyan fluorescent protein. The fluorescent activity of the polypeptide can comprise an emission max at 507 (green) and 491 (cyan), an excitation at 487 (green) and 448 (major), 463 (secondary peak). In another aspect, the fluorescent activity of the polypeptide can comprise emission at 500 nm (green). Alternatively, the fluorescent activity can comprise emission at 490 nm (cyan). In one aspect, the polypeptide encoded by the isolated or recombinant nucleic acid can comprise fluorescent activity after excitation at 485 nm (for green). In another aspect, the polypeptide can comprise fluorescent activity after excitation at 460 nm (for cyan).

In one aspect, the isolated or recombinant nucleic acid encodes a polypeptide that retains a fluorescent activity under conditions comprising about pH 3.0, 3.5, 4.0, 4.5, 5.0, 5.5, 6.0 or more. In one aspect, the isolated or recombinant nucleic acid encodes a polypeptide that retains a fluorescent activity under conditions comprising about pH 8.0, 8.5, 9.0, 9.5, 10.0, 10.5, 11.0 or more.

In one aspect, the isolated or recombinant nucleic acid encodes a polypeptide having a fluorescent activity that is thermostable. The polypeptide can retain a fluorescent activity under conditions comprising a temperature in the range of between about 30° C. to about 90° C., or between about 0° C. and 30° C. In one aspect, the isolated or recombinant nucleic acid encodes a polypeptide having a fluorescent activity that is thermotolerant. The polypeptide can retain a fluorescent activity after being exposed to conditions comprising a temperature in the range of between about 30° C. to about 100° C., or, between about 0° C. and 30° C.

In one aspect, the isolated or recombinant nucleic acid encodes a polypeptide having a fluorescent activity under conditions comprising treatment with a chaotropic agent, e.g., conditions comprising a period up to about 50 hours with 6M guanidine HCL, 8M urea or 1% SDS. The polypeptide can retain a fluorescent activity under conditions comprising treatment with a protease, e.g., a protease, such as trypsin, chymotrypsin, papain, subtilisin, thermolisin, or pancreatin, for a period up to about 50 hours, and, in one aspect, under conditions comprising a concentration range of up to about 1 mg/ml.

In one aspect, the isolated or recombinant nucleic acid comprises a sequence that hybridizes under stringent conditions to a nucleic acid sequence as set forth in SEQ ID NO:1, a sequence as set forth in SEQ ID NO:3, a sequence as set forth in SEQ ID NO:5, a sequence as set forth in SEQ ID NO:7, a sequence as set forth in SEQ ID NO:9, a sequence as set forth in SEQ ID NO:11, a sequence as set forth in SEQ ID NO:13, a sequence as set forth in SEQ ID NO:15, a sequence as set forth in SEQ ID NO:17, a sequence as set forth in SEQ ID NO:19, a sequence as set forth in SEQ ID NO:21, a sequence as set forth in SEQ ID NO:23, or a sequence as set forth in SEQ ID NO:25, wherein the nucleic acid encodes a fluorescent polypeptide. The nucleic acid can be at least about 10, 20, 30, 40, 50, 60, 70, 80, 90, 100, 150, 200, 250, 300, 350, 400, 450, 500, 550 or 600 or more residues in length or the full length of the gene or transcript. The stringent conditions can include a wash step comprising a wash in 0.2×SSC at a temperature of about 65° C. for about 15 minutes.

The invention provides a nucleic acid probe for identifying a nucleic acid encoding a fluorescent polypeptide, wherein the probe comprises at least 10, 20, 30, 40, 50, 60, 70, 80, 90, 100, 150, 200, 250, 300, 350, 400, 450, 500, 550 or 600 or more consecutive bases of a sequence comprising a sequence as set forth in SEQ ID NO:1, a sequence as set forth in SEQ ID NO:3, a sequence as set forth in SEQ ID NO:5, a sequence as set forth in SEQ ID NO:7, a sequence as set forth in SEQ ID NO:9, a sequence as set forth in SEQ ID NO:11, a sequence as set forth in SEQ ID NO:13, a sequence as set forth in SEQ ID NO:15, a sequence as set forth in SEQ ID NO:17, a sequence as set forth in SEQ ID NO:19, a sequence as set forth in SEQ ID NO:21, a sequence as set forth in SEQ ID NO:23, or a sequence as set forth in SEQ ID NO:25, wherein the probe identifies the nucleic acid by binding or hybridization. The probe can comprise at least about 10 to 50, about 20 to 60, about 30 to 70, about 40 to 80, or about 60 to 100 consecutive bases of a sequence as set forth in SEQ ID NO:1, a sequence as set forth in SEQ ID NO:3, a sequence as set forth in SEQ ID NO:5, a sequence as set forth in SEQ ID NO:7, a sequence as set forth in SEQ ID NO:9, a sequence as set forth in SEQ ID NO:11, a sequence as set forth in SEQ ID NO:13, a sequence as set forth in SEQ ID NO:15, a sequence as set forth in SEQ ID NO:17, a sequence as set forth in SEQ ID NO:19, a sequence as set forth in SEQ ID NO:21, a sequence as set forth in SEQ ID NO:23, or a sequence as set forth in SEQ ID NO:25.

The invention provides a nucleic acid probe for identifying a nucleic acid encoding a fluorescent polypeptide, wherein the probe comprises a nucleic acid sequence having at least 85%, 90%, 95%, 96%, 97%, 98%, 99%, or more, sequence identity to SEQ ID NO:1, or a subsequence thereof, over a region of at least about 100 residues. The invention provides a nucleic acid probe for identifying a nucleic acid encoding a fluorescent polypeptide, wherein the probe comprises a nucleic acid sequence having at least 85%, 90%, 95%, 96%, 97%, 98%, 99%, or more, sequence identity to SEQ ID NO:3, or a subsequence thereof, over a region of at least about 100 residues. The invention provides a nucleic acid probe for identifying a nucleic acid encoding a fluorescent polypeptide, wherein the probe comprises a nucleic acid sequence having at least 85%, 90%, 95%, 96%, 97%, 98%, 99%, or more, sequence identity to SEQ ID NO:5, or a subsequence thereof, over a region of at least about 100 residues. The invention provides a nucleic acid probe for identifying a nucleic acid encoding a fluorescent polypeptide, wherein the probe comprises a nucleic acid sequence having at least 85%, 90%, 95%, 96%, 97%, 98%, 99%, or more, sequence identity to SEQ ID NO:7, or a subsequence thereof, over a region of at least about 100 residues. The invention provides a nucleic acid probe for identifying a nucleic acid encoding a fluorescent polypeptide, wherein the probe comprises a nucleic acid sequence having at least 75%, 80%, 85%, 90%, 95%, 96%, 97%, 98%, 99%, or more, sequence identity to SEQ ID NO:9, or a subsequence thereof, over a region of at least about 100 residues. The invention provides a nucleic acid probe for identifying a nucleic acid encoding a fluorescent polypeptide, wherein the probe comprises a nucleic acid sequence having at least 75%, 80%, 85%, 90%, 95%, 96%, 97%, 98%, 99%, or more, sequence identity to SEQ ID NO:11, or a subsequence thereof, over a region of at least about 100 residues. The invention provides a nucleic acid probe for identifying a nucleic acid encoding a fluorescent polypeptide, wherein the probe comprises a nucleic acid sequence having at least 75%, 80%, 85%, 90%, 95%, 96%, 97%, 98%, 99%, or more, sequence identity to SEQ ID NO:13, or a subsequence thereof, over a region of at least about 100 residues. The invention provides a nucleic acid probe for identifying a nucleic acid encoding a fluorescent polypeptide, wherein the probe comprises a nucleic acid sequence having at least 70%, 75%, 80%, 85%, 90%, 95%, 96%, 97%, 98%, 99%, or more, sequence identity to SEQ ID NO:15, or a subsequence thereof, over a region of at least about 100 residues. The invention provides a nucleic acid probe for identifying a nucleic acid encoding a fluorescent polypeptide, wherein the probe comprises a nucleic acid sequence having at least 75%, 80%, 85%, 90%, 95%, 96%, 97%, 98%, 99%, or more, sequence identity to SEQ ID NO:17, or a subsequence thereof, over a region of at least about 100 residues. The invention provides a nucleic acid probe for identifying a nucleic acid encoding a fluorescent polypeptide, wherein the probe comprises a nucleic acid sequence having at least 70%, 75%, 80%, 85%, 90%, 95%, 96%, 97%, 98%, 99%, or more, sequence identity to SEQ ID NO:19, or a subsequence thereof, over a region of at least about 100 residues. The invention provides a nucleic acid probe for identifying a nucleic acid encoding a fluorescent polypeptide, wherein the probe comprises a nucleic acid sequence having at least 85%, 90%, 95%, 96%, 97%, 98%, 99%, or more, sequence identity to SEQ ID NO:21, or a subsequence thereof, over a region of at least about 100 residues. The invention provides a nucleic acid probe for identifying a nucleic acid encoding a fluorescent polypeptide, wherein the probe comprises a nucleic acid sequence having at least 85%, 90%, 95%, 96%, 97%, 98%, 99%, or more, sequence identity to SEQ ID NO:23, or a subsequence thereof, over a region of at least about 100 residues. The invention provides a nucleic acid probe for identifying a nucleic acid encoding a fluorescent polypeptide, wherein the probe comprises a nucleic acid sequence having at least 85%, 90%, 95%, 96%, 97%, 98%, 99%, or more, sequence identity to SEQ ID NO:25, or a subsequence thereof, over a region of at least about 100 residues. The sequence identities can be determined by analysis with a sequence comparison algorithm or by visual inspection. The probe can comprise an oligonucleotide comprising at least about 10 to 50, about 20 to 60, about 30 to 70, about 40 to 80, or about 60 to 100 consecutive bases of a nucleic acid sequence comprising a sequence as set forth in SEQ ID NO:1, or a subsequence thereof; a sequence as set forth in SEQ ID NO:3, or a subsequence thereof; a sequence as set forth in SEQ ID NO:5, or a subsequence thereof; a sequence as set forth in SEQ ID NO:7, or a subsequence thereof; a sequence as set forth in SEQ ID NO:9, or a subsequence thereof; a sequence as set forth in SEQ ID NO:11, or a subsequence thereof; a sequence as set forth in SEQ ID NO:13, or a subsequence thereof; a sequence as set forth in SEQ ID NO:15, or a subsequence thereof; a sequence as set forth in SEQ ID NO:17, or a subsequence thereof, a sequence as set forth in SEQ ID NO:19, or a subsequence thereof, a sequence as set forth in SEQ ID NO:21, or a subsequence thereof, a sequence as set forth in SEQ ID NO:23, or a subsequence thereof; or, a sequence as set forth in SEQ ID NO:25, or a subsequence thereof. The probes can comprise at least 10, 20, 30, 40, 50, 60, 70, 80, 90, 100, 150, 200, 250, 300, 350, 400, 450, 500, 550 or 600 or more consecutive bases of a sequence of the invention.

The invention provides an amplification primer sequence pair for amplifying a nucleic acid encoding a polypeptide with a fluorescent activity, wherein the primer pair is capable of amplifying a nucleic acid comprising a sequence as set forth in SEQ ID NO:1, or a subsequence thereof; a sequence as set forth in SEQ ID NO:3, or a subsequence thereof; a sequence as set forth in SEQ ID NO:5, or a subsequence thereof; a sequence as set forth in SEQ ID NO:7, or a subsequence thereof; a sequence as set forth in SEQ ID NO:9, or a subsequence thereof; a sequence as set forth in SEQ ID NO:11, or a subsequence thereof; a sequence as set forth in SEQ ID NO:13, or a subsequence thereof; and, a sequence as set forth in SEQ ID NO:15, or a subsequence thereof, a sequence as set forth in SEQ ID NO:17, or a subsequence thereof, a sequence as set forth in SEQ ID NO:19, or a subsequence thereof, a sequence as set forth in SEQ ID NO:21, or a subsequence thereof, a sequence as set forth in SEQ ID NO:23, or a subsequence thereof; or, a sequence as set forth in SEQ ID NO:25, or a subsequence thereof. One or each member of the amplification primer sequence pair can comprise an oligonucleotide comprising at least about 10 to 50 consecutive bases of the sequence.

The invention provides methods of amplifying a nucleic acid encoding a fluorescent polypeptide comprising amplification of a template nucleic acid with an amplification primer sequence pair capable of amplifying a nucleic acid sequence comprising a sequence as set forth in SEQ ID NO:1, or a subsequence thereof; a sequence as set forth in SEQ ID NO:3, or a subsequence thereof; a sequence as set forth in SEQ ID NO:5, or a subsequence thereof; a sequence as set forth in SEQ ID NO:7, or a subsequence thereof; a sequence as set forth in SEQ ID NO:9, or a subsequence thereof; a sequence as set forth in SEQ ID NO:11, or a subsequence thereof; a sequence as set forth in SEQ ID NO:13, or a subsequence thereof; and, a sequence as set forth in SEQ ID NO:15, or a subsequence thereof, a sequence as set forth in SEQ ID NO:17, or a subsequence thereof, a sequence as set forth in SEQ ID NO:19, or a subsequence thereof, a sequence as set forth in SEQ ID NO:21, or a subsequence thereof, a sequence as set forth in SEQ ID NO:23, or a subsequence thereof; or, a sequence as set forth in SEQ ID NO:25, or a subsequence thereof.

The invention provides expression cassettes comprising a nucleic acid of the invention, e.g., comprising (i) a nucleic acid sequence having at least 85% sequence identity to SEQ ID NO:1 over a region of at least about 100 residues, at least 85% sequence identity to SEQ ID NO:3 over a region of at least about 100 residues, at least 85% sequence identity to SEQ ID NO:5 over a region of at least about 100 residues, at least 85% sequence identity to SEQ ID NO:7 over a region of at least about 100 residues, at least 75% sequence identity to SEQ ID NO:9 over a region of at least about 100 residues, at least 75% sequence identity to SEQ ID NO:11 over a region of at least about 100 residues, at least 75% sequence identity to SEQ ID NO:13 over a region of at least about 100 residues, at least 70% sequence identity to SEQ ID NO:15 over a region of at least about 100 residues, at least 75% sequence identity to SEQ ID NO:17 over a region of at least about 100 residues, at least 70% sequence identity to SEQ ID NO:19 over a region of at least about 100 residues, at least 85% sequence identity to SEQ ID NO:21 over a region of at least about 100 residues, at least 85% sequence identity to SEQ ID NO:23 over a region of at least about 100 residues, or at least 85% sequence identity to SEQ ID NO:25 over a region of at least about 100 residues, wherein the sequence identities are determined by analysis with a sequence comparison algorithm or by visual inspection; or, (ii) a nucleic acid that hybridizes under stringent conditions to a nucleic acid comprising a sequence as set forth in SEQ ID NO:1, or a subsequence thereof; a sequence as set forth in SEQ ID NO:3, or a subsequence thereof; a sequence as set forth in SEQ ID NO:5, or a subsequence thereof; and, a sequence as set forth in SEQ ID NO:7, or a subsequence thereof; a sequence as set forth in SEQ ID NO:9, or a subsequence thereof; a sequence as set forth in SEQ ID NO:11, or a subsequence thereof; a sequence as set forth in SEQ ID NO:13, or a subsequence thereof; and, a sequence as set forth in SEQ ID NO:15, or a subsequence thereof, a sequence as set forth in SEQ ID NO:17, or a subsequence thereof, a sequence as set forth in SEQ ID NO:19, or a subsequence thereof, a sequence as set forth in SEQ ID NO:21, or a subsequence thereof, a sequence as set forth in SEQ ID NO:23, or a subsequence thereof; or, a sequence as set forth in SEQ ID NO:25, or a subsequence thereof.

The invention provides vectors comprising a nucleic acid of the invention, e.g., (i) a nucleic acid sequence having at least 85% sequence identity to SEQ ID NO:1 over a region of at least about 100 residues, a nucleic acid sequence having at least 85% sequence identity to SEQ ID NO:3 over a region of at least about 100 residues, a nucleic acid sequence having at least 85% sequence identity to SEQ ID NO:5 over a region of at least about 100 residues, a nucleic acid sequence having at least 85% sequence identity to SEQ ID NO:7 over a region of at least about 100 residues, a nucleic acid sequence having at least 75% sequence identity to SEQ ID NO:9 over a region of at least about 100 residues, a nucleic acid sequence having at least 75% sequence identity to SEQ ID NO:11 over a region of at least about 100 residues, a nucleic acid sequence having at least 75% sequence identity to SEQ ID NO:13 over a region of at least about 100 residues, a nucleic acid sequence having at least 70% sequence identity to SEQ ID NO:15 over a region of at least about 100 residues, a nucleic acid sequence having at least 75% sequence identity to SEQ ID NO:17 over a region of at least about 100 residues, a nucleic acid sequence having at least 70% sequence identity to SEQ ID NO:19 over a region of at least about 100 residues, a nucleic acid sequence having at least 85% sequence identity to SEQ ID NO:21 over a region of at least about 100 residues, a nucleic acid sequence having at least 85% sequence identity to SEQ ID NO:23 over a region of at least about 100 residues, or a nucleic acid sequence having at least 85% sequence identity to SEQ ID NO:25 over a region of at least about 100 residues, wherein the sequence identities are determined by analysis with a sequence comparison algorithm or by visual inspection; or, (ii) a nucleic acid that hybridizes under stringent conditions to a nucleic acid comprising a sequence as set forth in SEQ ID NO:1, or a subsequence thereof; a sequence as set forth in SEQ ID NO:3, or a subsequence thereof; a sequence as set forth in SEQ ID NO:5, or a subsequence thereof; and, a sequence as set forth in SEQ ID NO:7, or a subsequence thereof; a sequence as set forth in SEQ ID NO:9, or a subsequence thereof a sequence as set forth in SEQ ID NO:11, or a subsequence thereof; a sequence as set forth in SEQ ID NO:13, or a subsequence thereof; and, a sequence as set forth in SEQ ID NO:15, or a subsequence thereof, a sequence as set forth in SEQ ID NO:17, or a subsequence thereof, a sequence as set forth in SEQ ID NO:19, or a subsequence thereof, a sequence as set forth in SEQ ID NO:21, or a subsequence thereof, a sequence as set forth in SEQ ID NO:23, or a subsequence thereof; or, a sequence as set forth in SEQ ID NO:25, or a subsequence thereof.

The invention provides cloning vehicles comprising a nucleic acid of the invention or a vector of the invention. The cloning vehicle can be a viral vector, a plasmid, a phage, a phagemid, a cosmid, a fosmid, a bacteriophage or an artificial chromosome. The viral vector can comprise an adenovirus vector, a retroviral vectors or an adeno-associated viral vector. The cloning vehicle can comprise a bacterial artificial chromosome (BAC), a plasmid, a bacteriophage P1-derived vector (PAC), a yeast artificial chromosome (YAC), a mammalian artificial chromosome (MAC).

The invention provides transformed cells comprising a nucleic acid of the invention or a vector of the invention or a cloning vehicle of the invention. The vector can comprise a nucleic acid of the invention or a nucleic acid that hybridizes under stringent conditions to a nucleic acid comprising a sequence as set forth in SEQ ID NO:1, or a subsequence thereof; a sequence as set forth in SEQ ID NO:3, or a subsequence thereof; a sequence as set forth in SEQ ID NO:5, or a subsequence thereof; and, a sequence as set forth in SEQ ID NO:7, or a subsequence thereof; a sequence as set forth in SEQ ID NO:9, or a subsequence thereof; a sequence as set forth in SEQ ID NO:11, or a subsequence thereof; a sequence as set forth in SEQ ID NO:13, or a subsequence thereof; and, a sequence as set forth in SEQ ID NO:15, or a subsequence thereof, a sequence as set forth in SEQ ID NO:17, or a subsequence thereof, a sequence as set forth in SEQ ID NO:19, or a subsequence thereof, a sequence as set forth in SEQ ID NO:21, or a subsequence thereof, a sequence as set forth in SEQ ID NO:23, or a subsequence thereof; or, a sequence as set forth in SEQ ID NO:25, or a subsequence thereof.

The invention provides transformed cells comprising a nucleic acid of the invention, e.g., a nucleic acid comprising (i) a nucleic acid sequence having at least 85% sequence identity to SEQ ID NO:1 over a region of at least about 100 residues, a nucleic acid sequence having at least 85% sequence identity to SEQ ID NO:3 over a region of at least about 100 residues, a nucleic acid sequence having at least 85% sequence identity to SEQ ID NO:5 over a region of at least about 100 residues, a nucleic acid sequence having at least 85% sequence identity to SEQ ID NO:7 over a region of at least about 100 residues, a nucleic acid sequence having at least 75% sequence identity to SEQ ID NO:9 over a region of at least about 100 residues, a nucleic acid sequence having at least 75% sequence identity to SEQ ID NO:11 over a region of at least about 100 residues, a nucleic acid sequence having at least 75% sequence identity to SEQ ID NO:13 over a region of at least about 100 residues, a nucleic acid sequence having at least 70% sequence identity to SEQ ID NO:15 over a region of at least about 100 residues, a nucleic acid sequence having at least 75% sequence identity to SEQ ID NO:17 over a region of at least about 100 residues, a nucleic acid sequence having at least 70% sequence identity to SEQ ID NO:19 over a region of at least about 100 residues, a nucleic acid sequence having at least 85% sequence identity to SEQ ID NO:21 over a region of at least about 100 residues, a nucleic acid sequence having at least 85% sequence identity to SEQ ID NO:23 over a region of at least about 100 residues, or a nucleic acid sequence having at least 85% sequence identity to SEQ ID NO:25 over a region of at least about 100 residues, wherein the sequence identities are determined by analysis with a sequence comparison algorithm or by visual inspection; or, (ii) a nucleic acid that hybridizes under stringent conditions to a nucleic acid comprising a sequence as set forth in SEQ ID NO:1, or a subsequence thereof; a sequence as set forth in SEQ ID NO:3, or a subsequence thereof; a sequence as set forth in SEQ ID NO:5, or a subsequence thereof; and, a sequence as set forth in SEQ ID NO:7, or a subsequence thereof; a sequence as set forth in SEQ ID NO:9, or a subsequence thereof; a sequence as set forth in SEQ ID NO:11, or a subsequence thereof; a sequence as set forth in SEQ ID NO:13, or a subsequence thereof; and, a sequence as set forth in SEQ ID NO:15, or a subsequence thereof, a sequence as set forth in SEQ ID NO:17, or a subsequence thereof, a sequence as set forth in SEQ ID NO:19, or a subsequence thereof, a sequence as set forth in SEQ ID NO:21, or a subsequence thereof, a sequence as set forth in SEQ ID NO:23, or a subsequence thereof; or, a sequence as set forth in SEQ ID NO:25, or a subsequence thereof. In one aspect, the transformed cell is a bacterial cell, a mammalian cell, a fungal cell, a yeast cell, an insect cell or a plant cell.

The invention provides transgenic non-human animals comprising a nucleic acid of the invention or a vector of the invention. In one aspect, the transgenic animal is a mouse. In another aspect, the animal is a rabbit.

The invention provides transgenic plants comprising a nucleic acid of the invention or a vector of the invention. The transgenic plant can be an oilseed plant, a rapeseed plant, a soybean plant, a palm, a canola plant, a sunflower plant, a sesame plant, a peanut plant or a tobacco plant.

The invention provides transgenic seeds comprising a nucleic acid of the invention or a vector of the invention. The transgenic seed can be an oilseed, a rapeseed, a soybean seed, a palm kernel, a canola plant seed, a sunflower seed, a sesame seed, a peanut or a tobacco plant seed.

The invention provides an antisense oligonucleotide comprising a nucleic acid sequence complementary to or capable of hybridizing under stringent conditions to a nucleic acid of the invention, e.g., (i) a nucleic acid comprising a nucleic acid sequence having at least 85% sequence identity to SEQ ID NO:1 over a region of at least about 100 residues, a nucleic acid sequence having at least 85% sequence identity to SEQ ID NO:3 over a region of at least about 100 residues, a nucleic acid sequence having at least 85% sequence identity to SEQ ID NO:5 over a region of at least about 100 residues, a nucleic acid sequence having at least 85% sequence identity to SEQ ID NO:7 over a region of at least about 100 residues, a nucleic acid sequence having at least 75% sequence identity to SEQ ID NO:9 over a region of at least about 100 residues, a nucleic acid sequence having at least 75% sequence identity to SEQ ID NO:11 over a region of at least about 100 residues, a nucleic acid sequence having at least 75% sequence identity to SEQ ID NO:13 over a region of at least about 100 residues, a nucleic acid sequence having at least 70% sequence identity to SEQ ID NO:15 over a region of at least about 100 residues, a nucleic acid sequence having at least 75% sequence identity to SEQ ID NO:17 over a region of at least about 100 residues, a nucleic acid sequence having at least 70% sequence identity to SEQ ID NO:19 over a region of at least about 100 residues, a nucleic acid sequence having at least 85% sequence identity to SEQ ID NO:21 over a region of at least about 100 residues, a nucleic acid sequence having at least 85% sequence identity to SEQ ID NO:23 over a region of at least about 100 residues, or a nucleic acid sequence having at least 85% sequence identity to SEQ ID NO:25 over a region of at least about 100 residues, wherein the sequence identities are determined by analysis with a sequence comparison algorithm or by visual inspection; or, (ii) a nucleic acid that hybridizes under stringent conditions to a nucleic acid comprising a sequence as set forth in SEQ ID NO:1, or a subsequence thereof a sequence as set forth in SEQ ID NO:3, or a subsequence thereof a sequence as set forth in SEQ ID NO:5, or a subsequence thereof and, a sequence as set forth in SEQ ID NO:7, or a subsequence thereof; a sequence as set forth in SEQ ID NO:9, or a subsequence thereof a sequence as set forth in SEQ ID NO:11, or a subsequence thereof a sequence as set forth in SEQ ID NO:13, or a subsequence thereof and, a sequence as set forth in SEQ ID NO:15, or a subsequence thereof, a sequence as set forth in SEQ ID NO:17, or a subsequence thereof, a sequence as set forth in SEQ ID NO:19, or a subsequence thereof, a sequence as set forth in SEQ ID NO:21, or a subsequence thereof, a sequence as set forth in SEQ ID NO:23, or a subsequence thereof; or, a sequence as set forth in SEQ ID NO:25, or a subsequence thereof. The antisense oligonucleotide can be between about 10 to 50, about 20 to 60, about 30 to 70, about 40 to 80, or about 60 to 100 bases in length.

The invention provides an isolated or recombinant polypeptide comprising an amino acid sequence of the invention, e.g., a sequence having at least about 70%, 75%, 80%, 85%, 90%, 95%, 98%, 99%, or more, sequence identity to SEQ ID NO:2 over a region of at least about 10, 20, 30, 40, 50, 60, 70, 80, 90, 100, 150, 200, or more, residues, an amino acid sequence having at least 70%, 75%, 80%, 85%, 90%, 95%, 98%, 99%, or more, sequence identity to SEQ ID NO:4 over a region of at least about 10, 20, 30, 40, 50, 60, 70, 80, 90, 100, 150, 200, or more, residues, an amino acid sequence having at least 70%, 75%, 80%, 85%, 90%, 95%, 98%, 99%, or more, sequence identity to SEQ ID NO:6 over a region of at least about 10, 20, 30, 40, 50, 60, 70, 80, 90, 100, 150, 200, or more, residues, an amino acid sequence having at least 70%, 75%, 80%, 85%, 90%, 95%, 98%, 99%, or more, sequence identity to SEQ ID NO:8 over a region of at least about 100 residues, an amino acid sequence having at least 65% sequence identity to SEQ ID NO:10 over a region of at least about 10, 20, 30, 40, 50, 60, 70, 80, 90, 100, 150, 200, or more, residues, an amino acid sequence having at least 65%, 70%, 75%, 80%, 85%, 90%, 95%, 98%, 99%, or more, sequence identity to SEQ ID NO:12 over a region of at least about 10, 20, 30, 40, 50, 60, 70, 80, 90, 100, 150, 200, or more, residues, an amino acid sequence having at least 65%, 70%, 75%, 80%, 85%, 90%, 95%, 98%, 99%, or more, sequence identity to SEQ ID NO:14 over a region of at least about 10, 20, 30, 40, 50, 60, 70, 80, 90, 100, 150, 200, or more, residues, an amino acid sequence having at least 60%, 65%, 70%, 75%, 80%, 85%, 90%, 95%, 98%, 99%, or more, sequence identity to SEQ ID NO:16 over a region of at least about 10, 20, 30, 40, 50, 60, 70, 80, 90, 100, 150, 200, or more, residues, an amino acid sequence having at least 65%, 70%, 75%, 80%, 85%, 90%, 95%, 98%, 99%, or more, sequence identity to SEQ ID NO:18 over a region of at least about 10, 20, 30, 40, 50, 60, 70, 80, 90, 100, 150, 200, or more, residues, an amino acid sequence having at least 60%, 65%, 70%, 75%, 80%, 85%, 90%, 95%, 98%, 99%, or more, sequence identity to SEQ ID NO:20 over a region of at least about 10, 20, 30, 40, 50, 60, 70, 80, 90, 100, 150, 200, or more, residues, an amino acid sequence having at least 85% sequence identity to SEQ ID NO:22 over a region of at least about 10, 20, 30, 40, 50, 60, 70, 80, 90, 100, 150, 200, or more, residues, an amino acid sequence having at least 85%, 90%, 95%, 98%, 99%, or more, sequence identity to SEQ ID NO:24 over a region of at least about 10, 20, 30, 40, 50, 60, 70, 80, 90, 100, 150, 200, or more, residues, an amino acid sequence having at least 85%, 90%, 95%, 98%, 99%, or more, sequence identity to SEQ ID NO:26 over a region of at least about 10, 20, 30, 40, 50, 60, 70, 80, 90, 100, 150, 200, or more, residues, wherein the sequence identities are determined by analysis with a sequence comparison algorithm or by visual inspection; or, a polypeptide encoded by a nucleic acid comprising (i) a nucleic acid sequence having at least 85% sequence identity to SEQ ID NO:1 over a region of at least about 100 residues, a nucleic acid sequence having at least 85% sequence identity to SEQ ID NO:3 over a region of at least about 100 residues, a nucleic acid sequence having at least 85% sequence identity to SEQ ID NO:5 over a region of at least about 100 residues, a nucleic acid sequence having at least 85% sequence identity to SEQ ID NO:7 over a region of at least about 100 residues, a nucleic acid sequence having at least 75% sequence identity to SEQ ID NO:9 over a region of at least about 100 residues, a nucleic acid sequence having at least 75% sequence identity to SEQ ID NO:11 over a region of at least about 100 residues, a nucleic acid sequence having at least 75% sequence identity to SEQ ID NO:13 over a region of at least about 100 residues, a nucleic acid sequence having at least 70% sequence identity to SEQ ID NO:15 over a region of at least about 100 residues, a nucleic acid sequence having at least 75% sequence identity to SEQ ID NO:17 over a region of at least about 100 residues, a nucleic acid sequence having at least 70% sequence identity to SEQ ID NO:19 over a region of at least about 100 residues, a nucleic acid sequence having at least 85% sequence identity to SEQ ID NO:21 over a region of at least about 100 residues, a nucleic acid sequence having at least 85% sequence identity to SEQ ID NO:23 over a region of at least about 100 residues, or a nucleic acid sequence having at least 85% sequence identity to SEQ ID NO:25 over a region of at least about 100 residues, wherein the sequence identities are determined by analysis with a sequence comparison algorithm or by visual inspection; or, (ii) a nucleic acid that hybridizes under stringent conditions to a nucleic acid comprising a sequence as set forth in SEQ ID NO:1, or a subsequence thereof; a sequence as set forth in SEQ ID NO:3, or a subsequence thereof; a sequence as set forth in SEQ ID NO:5, or a subsequence thereof; and, a sequence as set forth in SEQ ID NO:7, or a subsequence thereof; a sequence as set forth in SEQ ID NO:9, or a subsequence thereof; a sequence as set forth in SEQ ID NO:11, or a subsequence thereof; a sequence as set forth in SEQ ID NO:13, or a subsequence thereof; and, a sequence as set forth in SEQ ID NO:15, or a subsequence thereof, a sequence as set forth in SEQ ID NO:17, or a subsequence thereof, a sequence as set forth in SEQ ID NO:19, or a subsequence thereof, a sequence as set forth in SEQ ID NO:21, or a subsequence thereof, a sequence as set forth in SEQ ID NO:23, or a subsequence thereof; or, a sequence as set forth in SEQ ID NO:25, or a subsequence thereof. In one aspect, the polypeptide can have a fluorescent activity.

In one aspect, the invention provides isolated or recombinant polypeptide comprising an amino acid sequence as set forth in SEQ ID NO:2, an amino acid sequence as set forth in SEQ ID NO:4, an amino acid sequence as set forth in SEQ ID NO:6, an amino acid sequence as set forth in SEQ ID NO:8, an amino acid sequence as set forth in SEQ ID NO:10, an amino acid sequence as set forth in SEQ ID NO:12, an amino acid sequence as set forth in SEQ ID NO:14, an amino acid sequence as set forth in SEQ ID NO:16, a sequence as set forth in SEQ ID NO:18, or a subsequence thereof, a sequence as set forth in SEQ ID NO:20, or a subsequence thereof, a sequence as set forth in SEQ ID NO:22, or a subsequence thereof, a sequence as set forth in SEQ ID NO:24, or a subsequence thereof; or, a sequence as set forth in SEQ ID NO:26, or a subsequence thereof.

In one aspect, the isolated or recombinant polypeptide can comprise the polypeptide of the invention and a heterologous signal sequence. In one aspect, the fluorescent activity of the polypeptide can comprise an emission max at 507 (green) and 491 (cyan), an excitation at 487 (green) and 448 (major), 463 (secondary peak). In one aspect, the fluorescent activity can comprise emission at 500 nm (green). Alternatively, the fluorescent activity can comprise emission at 490 nm (cyan). In one aspect, the polypeptide can comprise fluorescent activity after excitation at 485 nm (for green). In another aspect, the polypeptide comprises fluorescent activity after excitation at 460 nm (for cyan).

The invention provides protein preparations comprising a polypeptide of the invention, wherein the protein preparation comprises a liquid, a solid or a gel.

The invention provides homodimers comprising a polypeptide of the invention. In one aspect, the invention provides heterodimers comprising a polypeptide of the invention and a second domain. The second domain can be a polypeptide and the heterodimer can be a fusion protein. Alternatively, the second domain can be an epitope, a tag, or a signal sequence. In one aspect, the fusion protein of the invention comprises a signal sequence capable of localizing the fusion protein to a predetermined cellular locale, e.g., a subcellular location such as the Golgi, endoplasmic reticulum, nucleus, nucleoli, nuclear membrane, mitochondria, chloroplast, secretory vesicles, lysosome, and cellular membrane; or an extracellular location, e.g., by secretion from the cell.

The invention provides immobilized polypeptides having a fluorescent activity, wherein the polypeptide is a polypeptide of the invention, or is a polypeptide encoded by a nucleic acid of the invention, or a polypeptide comprising a polypeptide of the invention and a second domain. The polypeptide can be immobilized on a cell, a metal, a resin, a polymer, a ceramic, a glass, a microelectrode, a graphitic particle, a bead, a gel, a plate, an array or a capillary tube.

The invention provides arrays comprising an immobilized polypeptide, wherein the polypeptide is a polypeptide of the invention, or is a polypeptide encoded by a nucleic acid of the invention, or a polypeptide comprising a polypeptide of the invention and a second domain. The invention provides an array comprising an immobilized nucleic acid of the invention. The invention provides an array comprising an antibody of the invention.

The invention provides isolated or recombinant antibodies that specifically bind to a polypeptide of the invention or to a polypeptide encoded by a nucleic acid of the invention. The antibody can be a monoclonal or a polyclonal antibody. The antibody can be single-stranded. The invention provides hybridomas comprising an antibody of the invention.

The invention provides methods of isolating or identifying a fluorescent polypeptide comprising the steps of: (a) providing an antibody of the invention; (b) providing a sample comprising polypeptides; and (c) contacting the sample of step (b) with the antibody of step (a) under conditions wherein the antibody can specifically bind to the polypeptide, thereby isolating or identifying a fluorescent protein. The invention provides methods of making an anti-fluorescent protein antibody comprising administering to a non-human animal a nucleic acid of the invention, or a polypeptide of the invention, in an amount sufficient to generate a humoral immune response, thereby making an anti-fluorescent protein antibody.

The invention provides methods of producing a recombinant polypeptide comprising the steps of: (a) providing a nucleic acid of the invention operably linked to a promoter; and (b) expressing the nucleic acid of step (a) under conditions that allow expression of the polypeptide, thereby producing a recombinant polypeptide. The method can further comprise transforming a host cell with the nucleic acid of step (a) followed by expressing the nucleic acid of step (a), thereby producing a recombinant polypeptide in a transformed cell.

The invention provides methods for identifying a polypeptide having a fluorescent activity comprising the following steps (a) providing a polypeptide of the invention or a polypeptide encoded by a nucleic acid of the invention; (b) providing an excitation source; and (c) subjecting the polypeptide or a fragment or variant thereof of step (a) to an excitation energy provided by the excitation source of step (b) and detecting an emitted light by the polypeptide of step (a) thereby identifying a polypeptide having a fluorescent activity. In one aspect, the excitation can occur at a wavelength comprising the range from about 380 nm to about 510 nm. In one aspect, the emission can occur at a wavelength comprising the range from about 490 nm to about 510 nm.

The invention provides methods for identifying an agent that changes a fluorescent polypeptide emission comprising the following steps: (a) providing a polypeptide of the invention or a polypeptide encoded by a nucleic acid of the invention; (b) providing a test agent; (c) contacting the polypeptide of step (a) with the agent of step (b) and measuring a fluorescent activity of the polypeptide of the invention, wherein a change in the fluorescent activity measured in the presence of the test agent compared to the activity in the absence of the test agent provides a determination that the test agent changes the fluorescent activity. In one aspect, the test agent can be a quencher of a fluorescent activity. In one aspect, a decrease in the amount of fluorescence with the test agent compared to the amount of fluorescence without the test agent identifies the test agent as a quencher of a fluorescent activity.

The invention provides computer systems comprising a processor and a data storage device wherein said data storage device has stored thereon a polypeptide sequence or a nucleic acid sequence, wherein the polypeptide can be a polypeptide of the invention or a subsequence thereof, and the nucleic acid can be a nucleic acid of the invention or a subsequence thereof. The computer system can further comprise a sequence comparison algorithm and a data storage device having at least one reference sequence stored thereon. The sequence comparison algorithm can comprise a computer program that indicates polymorphisms. The computer system can further comprise an identifier that identifies one or more features in the sequence.

The invention provides computer readable mediums having stored thereon a polypeptide sequence or a nucleic acid sequence, wherein the polypeptide can be a polypeptide of the invention, or subsequence thereof, the nucleic acid can be a nucleic acid of the invention, or subsequence thereof.

The invention provides methods for identifying a feature in a sequence comprising the steps of: (a) reading the sequence using a computer program which identifies one or more features in a sequence, wherein the sequence comprises a polypeptide sequence or a nucleic acid sequence, wherein the polypeptide comprises a polypeptide of the invention, and the nucleic acid sequence comprises a sequence of a nucleic acid of the invention; (b) identifying one or more features in the sequence with the computer program.

The invention provides methods for comparing a first sequence to a second sequence comprising the steps of: (a) reading the first sequence and the second sequence through use of a computer program which compares sequences, wherein the first sequence comprises a polypeptide sequence or a nucleic acid sequence, wherein the polypeptide comprises sequence of a polypeptide of the invention, or subsequence thereof, and the nucleic acid comprises a sequence of a nucleic acid of the invention or subsequence thereof; and (b) determining differences between the first sequence and the second sequence with the computer program. In one aspect, the step of determining differences between the first sequence and the second sequence further comprises the step of identifying polymorphisms. In one aspect, the method further comprises an identifier that identifies one or more features in a sequence. In one aspect, the method further comprises reading the first sequence using a computer program and identifying one or more features in the sequence.

The invention provides methods for isolating or recovering a nucleic acid encoding a polypeptide with a fluorescent activity from an environmental sample comprising the steps of: (a) providing an amplification primer sequence pair for amplifying a nucleic acid encoding a polypeptide with a fluorescent activity, wherein the primer pair is capable of amplifying SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 or a subsequence thereof; (b) isolating a nucleic acid from the environmental sample or treating the environmental sample such that nucleic acid in the sample is accessible for hybridization to the amplification primer pair; and, (c) combining the nucleic acid of step (b) with the amplification primer pair of step (a) and amplifying nucleic acid from the environmental sample, thereby isolating or recovering a nucleic acid encoding a fluorescent polypeptide from an environmental sample. In one aspect, each member of the amplification primer sequence pair comprises an oligonucleotide comprising at least about 10 to 50, or about 20 to 60, consecutive bases of a sequence as set forth in SEQ ID NO:1, SEQ ID NO:3, SEQ ID NO:5, SEQ ID NO:7, SEQ ID NO:9, SEQ ID NO:11, SEQ ID NO:13, or SEQ ID NO:15, SEQ ID NO:17, SEQ ID NO:19, SEQ ID NO:21, SEQ ID NO:23, SEQ ID NO:25, or a subsequence thereof.

The invention provides methods for isolating or recovering a nucleic acid encoding a polypeptide with a fluorescent activity from an environmental sample comprising the steps of: (a) providing a polynucleotide probe comprising a sequence or a subsequence comprising a nucleic acid of the invention; (b) isolating a nucleic acid from the environmental sample or treating the environmental sample such that nucleic acid in the sample is accessible for hybridization to a polynucleotide probe of step (a); (c) combining the isolated nucleic acid or the treated environmental sample of step (b) with the polynucleotide probe of step (a); and (d) isolating a nucleic acid that specifically hybridizes with the polynucleotide probe of step (a), thereby isolating or recovering a nucleic acid encoding a polypeptide with a fluorescent activity from an environmental sample. In alternative aspects, the environmental sample comprises a water sample, a liquid sample, a soil sample, an air sample or a biological sample. In one aspect, the biological sample is derived from a bacterial cell, a protozoan cell, an insect cell, a yeast cell, a plant cell, a fungal cell or a mammalian cell.

The invention provides methods of generating a variant of a nucleic acid encoding a fluorescent protein comprising the steps of: (a) providing a template nucleic acid comprising a nucleic acid of the invention; and (b) modifying, deleting or adding one or more nucleotides in the template sequence, or a combination thereof, to generate a variant of the template nucleic acid. The method can further comprise expressing the variant nucleic acid to generate a variant fluorescent polypeptide.

In alternative aspects, the modifications, additions or deletions are introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM™), synthetic ligation reassembly (SLR) and a combination thereof. In some aspects, the modifications, additions or deletions are introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

In one aspect, the method can be iteratively repeated until a fluorescent polypeptide having an altered or different activity or an altered or different stability from that of a fluorescent polypeptide encoded by the template nucleic acid is produced. In one aspect, the polypeptide of the invention retains a fluorescent activity under denaturing conditions, wherein the polypeptide encoded by the template nucleic acid is not fluorescent under the denaturing conditions. In another aspect, the method could be iteratively repeated until a polypeptide retains fluorescence under a high temperature, wherein the fluorescent polypeptide encoded by the template nucleic acid is not fluorescent under the high temperature. Alternatively, the method could be iteratively repeated until a fluorescent polypeptide coding sequence having an altered codon usage from that of the template nucleic acid is produced. The method can be iteratively repeated until a fluorescent polypeptide gene having higher or lower level of message expression or stability from that of the template nucleic acid is produced.

The invention provides methods for modifying codons in a nucleic acid encoding a fluorescent polypeptide to increase its expression in a host cell, the method comprising the following steps: (a) providing a nucleic acid encoding a fluorescent polypeptide comprising a nucleic acid of the invention; and (b) modifying, deleting or adding one or more nucleotides in the template sequence, or a combination thereof, to generate a variant of the template nucleic acid (b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

The invention provides methods for modifying codons in a nucleic acid encoding a fluorescent polypeptide, the method comprising (a) providing a nucleic acid of the invention encoding a fluorescent polypeptide; and (b) identifying a codon in the nucleic acid of step (a) and replacing it with a different codon encoding the same amino acid as the replaced codon, thereby modifying codons in a nucleic acid encoding a fluorescent polypeptide.

The invention provides methods for modifying codons in a nucleic acid encoding a fluorescent polypeptide to increase its expression in a host cell, the method comprising (a) providing a nucleic acid of the invention encoding a fluorescent polypeptide; and (b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon underrepresented in coding sequences in genes in the host cell, thereby modifying the nucleic acid to increase its expression in a host cell.

The invention provides methods for modifying a codon in a nucleic acid encoding a fluorescent polypeptide to decrease its expression in a host cell, the method comprising (a) providing a nucleic acid of the invention encoding a fluorescent polypeptide; and (b) identifying at least one preferred codon in the nucleic acid of step (a) and replacing it with a non-preferred or less preferred codon encoding the same amino acid as the replaced codon, wherein a preferred codon is a codon over-represented in coding sequences in genes in a host cell and a non-preferred or less preferred codon is a codon under-represented in coding sequences in genes in the host cell, thereby modifying the nucleic acid to decrease its expression in a host cell. In one aspect, the host cell is a bacterial cell, a fungal cell, an insect cell, a yeast cell, a plant cell or a mammalian cell.

The invention provides methods for producing a library of nucleic acids encoding a plurality of modified fluorescent polypeptide active sites, wherein the modified active sites are derived from a first nucleic acid comprising a sequence encoding a first active site, the method comprising: (a) providing a first nucleic acid encoding a first active site, wherein the first nucleic acid sequence comprises a sequence that hybridizes under stringent conditions to a sequence comprising a sequence as set forth in SEQ ID NO:1, a sequence as set forth in SEQ ID NO:3; a sequence as set forth in SEQ ID NO:5, a sequence as set forth in SEQ ID NO:7, a sequence as set forth in SEQ ID NO:9, a sequence as set forth in SEQ ID NO:11, a sequence as set forth in SEQ ID NO:13, and a sequence as set forth in SEQ ID NO:15 or a subsequence thereof, a sequence as set forth in SEQ ID NO:17, or a subsequence thereof, a sequence as set forth in SEQ ID NO:19, or a subsequence thereof, a sequence as set forth in SEQ ID NO:21, or a subsequence thereof, a sequence as set forth in SEQ ID NO:23, or a subsequence thereof; or, a sequence as set forth in SEQ ID NO:25, or a subsequence thereof, and the nucleic acid encodes a fluorescent polypeptide active site; (b) providing a set of mutagenic oligonucleotides that encode naturally-occurring amino acid variants at a plurality of targeted codons in the first nucleic acid; and, (c) using the set of mutagenic oligonucleotides to generate a set of active site-encoding variant nucleic acids encoding a range of amino acid variations at each amino acid codon that was mutagenized, thereby producing a library of nucleic acids encoding a plurality of modified fluorescent polypeptide active sites.

In one aspect, the method can comprise mutagenizing the first nucleic acid of step (a) by a method comprising an optimized directed evolution system, gene site-saturation mutagenesis (GSSM™), synthetic ligation reassembly (SLR), error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

The invention provides methods for determining a functional fragment of a fluorescent polypeptide comprising the steps of: (a) providing a fluorescent polypeptide wherein the polypeptide comprises an amino acid sequence of a polypeptide of the invention, or, is encoded by a nucleic acid of the invention; and (b) deleting a plurality of amino acid residues from the sequence of step (a) and testing the remaining subsequence for a fluorescent activity, thereby determining a functional fragment of a fluorescent polypeptide. In one aspect, the fluorescence is measured by providing an excitation source set at the absorption wavelength of a fluorescent polypeptide and detecting an emission at the wavelength of the emission of a fluorescent polypeptide. In another aspect, a decrease in the amount of the fluorescence activity with the test agent as compared to the amount of fluorescence without the test agent identifies the test agent as a fluorescence quencher of the fluorescent activity.

The invention provides methods for producing a chimeric polypeptide comprising the following steps: (a) providing a fluorescent polypeptide, wherein the polypeptide comprises an amino acid sequence of a polypeptide of the invention, or, is encoded by a nucleic acid of the invention; (b) providing a second polypeptide; and (c) contacting the polypeptide of step (a) and the second polypeptide of step (b) under conditions wherein the fluorescent polypeptide can be fused with the second polypeptide, thereby producing a chimeric polypeptide. In one aspect, the chimeric polypeptide retains a fluorescent activity. In one aspect, the conditions under which the fluorescent polypeptide is fused with the second polypeptide comprise N-terminal fusion. In another aspect, the conditions under which the fluorescent polypeptide is fused with the second polypeptide comprise C-terminal fusion. In one aspect, the second polypeptide is capable of recognizing specific molecular structures. Particularly, the second polypeptide can be a polyclonal or monoclonal antibody.

The invention provides methods for producing a chimeric compound comprising the following steps: (a) providing a first fluorescent polypeptide, wherein the polypeptide comprises an amino acid sequence of a polypeptide of the invention, or, is encoded by a nucleic acid of the invention; (b) providing a second compound; and (c) contacting the polypeptide of step (a) and the second compound of step (b) under conditions wherein the fluorescent polypeptide can be fused with the second compound, thereby producing a chimeric compound. In one aspect, the resulting chimeric compound retains a fluorescent activity. In one aspect, the fusion can be N-terminal fusion. In another aspect, the fusion is C-terminal fusion.

The invention provides methods for producing a nucleic acid with a fluorescent tag comprising of following steps: (a) providing a first fluorescent polypeptide, wherein the polypeptide comprises an amino acid sequence of a polypeptide of the invention, or, is encoded by a nucleic acid of the invention; (b) providing a nucleic acid; and (c) contacting the polypeptide of step (a) and the nucleic acid of step (b) under conditions wherein the fluorescent polypeptide can covalently bind with the nucleic acid, thereby producing a nucleic acid with a fluorescent tag.

The invention provides methods for using a polypeptide as a fluorescent marker comprising the following steps: (a) providing a first fluorescent polypeptide, wherein the polypeptide comprises an amino acid sequence of a polypeptide of the invention, or, is encoded by a nucleic acid of the invention; or a chimeric polypeptide comprising a polypeptide of the invention, or a chimeric compound comprising a polypeptide of the invention, or a nucleic acid with a fluorescent tag comprising a polypeptide of the invention; (b) providing an excitation source emitting light at the absorption wavelength of the fluorescent polypeptide; and (c) detecting a fluorescent activity of the compound of step (a) at the emission wavelength of the fluorescent polypeptide. In one aspect, the use as a fluorescent marker can comprise receptor-ligand binding. In one aspect, the polypeptide can be used as a fluorescent marker in immunoassays, single-step homogenous assays, multiple-step heterogeneous assays, enzyme assays. In another aspect, the polypeptide can be used as a fluorescent marker to measure protein-protein interactions. In one aspect, the polypeptide can be used as a fluorescent marker in protein transport. In one aspect, the polypeptide is used as a fluorescent marker to monitor the subcellular targeting.

The invention provides methods for using a fluorescent polypeptide in gene therapy to identify a cell comprising a desired nucleic acid comprising the following steps: (a) obtaining from a subject a sample of cells; (b) inserting in the cells of step (a) a nucleic acid segment; (c) introducing in the cell of step (b) a nucleic acid of the invention; (d) identifying and isolating cells or cell lines that comprise the nucleic acid of step (b); (e) re-introducing the cells of step (d) into the subject; (f) removing from the subject an aliquot of cells; (g) determining whether the cells of step (f) express a fluorescent protein; thereby identifying a cell comprising the desired nucleic acid.

The invention provides methods of gene therapy comprising the following steps: (a) providing a plurality of cells; (b) providing a retroviral vector comprising a desired nucleic acid; (c) providing a vector of the invention, wherein the vector comprises a nucleic acid encoding a fluorescent polypeptide; and (d) contacting the cells of step (a) with the vector of step (b) and a vector of step (c) under conditions wherein the cells of step (a) are transfected with the vectors of steps (b) and (c) allowing co-expression of the fluorescent, thereby allowing assessment of proportion of transfected cells and levels of expression. The cells can comprise cancerous or diseased cells.

The invention provides methods for identifying an inducing agent for a promoter comprising the following steps: (a) providing a nucleic of the invention encoding a fluorescent polypeptide; (b) placing the nucleic acid of step (a) under control of a promoter; (c) providing a test compound to induce the promoter of step (b); and (d) contacting the agent of step (c) with the promoter of step (b) under conditions wherein the agent of step (c) induces the promoter of step (b), thereby causing the expression of a fluorescent polypeptide in a cell, a cell line or a tissue, wherein the cell, cell line or tissue will become fluorescent in the presence of an inducing agent.

The invention provides methods for assessing the effect of selected culture components and conditions on gene expression comprising the following steps: (a) providing a cell comprising a nucleic acid of the invention, that encodes a fluorescent polypeptide, operably linked to a regulatory sequence derived from a selected gene; (b) incubating the cell of step (a) under selected culture conditions or in the presence of the selected components, and (c) detecting the presence and subcellular localization of a fluorescent signal, thereby assessing the effect of selected cultures components or condition on expression of a selected gene. The selected culture conditions or components can comprise salt concentration, pH, temperature, transacting regulatory substance, hormones, cell-cell contacts, ligands of cell surface or internal receptors.

The invention provides methods for assessing a mutagenic potential of a test agent in a tissue culture or a transgenic non-human animal comprising the following steps: (a) providing the nucleic acid of the invention that encodes a fluorescent polypeptide, operably linked to a transcriptional control element, wherein the transcription control element can be negatively regulated by a repressor; (b) providing a repressor under control of a constitutively expressed gene; (c) providing a test compound capable of interacting with a promoter of the constitutively expressed gene, thereby turning it off; (d) contacting the test agent of step (c) with the repressor of step (b) under conditions wherein the test agent can inactivate or turn off the gene expressing the repressor, thereby causing the expression of the polypeptide of the invention; and (e) identifying whether the fluorescent polypeptide is expressed, thereby assessing the mutagenic potential of the test agent. In one aspect, the mutagenicity of a test agent can be assessed qualitatively by direct visualization of fluorescence in the cells. In another aspect, the mutagenicity of a test agent is assessed quantitatively comprising FACS analysis.

The invention provides methods for identifying a compound capable of changing expression of a target gene comprising of the following steps: (a) providing a first nucleic acid of the invention, wherein the nucleic acid is operably linked to a promoter of a target gene in a cell, and a nucleic acid encodes a first fluorescent polypeptide; (b) providing a second nucleic acid of the invention, wherein the second nucleic acid is operably linked to a promoter of a constitutively expressed gene in a cell and encodes a second fluorescent polypeptide, and the first polypeptide emits a light at a wavelength different than the wavelength of light emitted by the second fluorescent polypeptide; (c) providing a test compound affecting the expression of the target gene of step (a) by binding to the promoter of the target gene; (d) contacting the compound of step (c) with the cell of step (a); (e) expressing the first and second polypeptide, and (f) detecting fluorescence of the first and second polypeptides, wherein altered fluorescence of the first polypeptide and unchanged fluorescence of the second polypeptide demonstrates that the compound binds to the target gene promoter and has no non-specific or cytotoxic effects, thereby not altering expression of the second polypeptide; or wherein altered fluorescence of the first polypeptide and altered fluorescence of the second polypeptide demonstrates that the test drug has non-specific or cytotoxic effects thereby affecting the expression of the second polypeptide.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The following drawings are illustrative of aspects of the invention and are not meant to limit the scope of the invention as encompassed by the claims.

FIG. 1 is a block diagram of a computer system.

FIG. 2 is a flow diagram illustrating one aspect of a process for comparing a new nucleotide or protein sequence with a database of sequences in order to determine the homology levels between the new sequence and the sequences in the database.

FIG. 3 is a flow diagram illustrating one aspect of a process in a computer for determining whether two sequences are homologous.

FIG. 4 is a flow diagram illustrating one aspect of an identifier process 300 for detecting the presence of a feature in a sequence.

FIG. 5 is a summary of data comparing the properties of exemplary fluorescent polypeptides of the invention.

FIG. 6 is a graphic representation of data comparing excitation properties of an exemplary fluorescent polypeptide of the invention to other fluorescent polypeptides.

FIG. 7 is a graphic representation of data comparing emission properties of an exemplary fluorescent polypeptide of the invention to other fluorescent polypeptides.

FIG. 8 is a graphic representation of data comparing excitation properties of exemplary fluorescent polypeptides of the invention to other fluorescent polypeptides.

FIG. 9 is a graphic representation of data comparing emission properties of an exemplary fluorescent polypeptide of the invention to other fluorescent polypeptides.

FIG. 10 is a graphic representation of data comparing excitation and emission spectra of exemplary fluorescent polypeptides of the invention.

FIG. 11 is a summary of data comparing the properties of exemplary fluorescent polypeptides of the invention and other fluorescent polypeptides.

FIG. 12 is a graphic representation of data comparing excitation and emission spectra properties of exemplary fluorescent polypeptides of the invention, Cyan-FP and Green-FP.

FIG. 13 is a summary of data comparing selected properties of exemplary fluorescent polypeptides of the invention, SEQ ID NO:8 (DISCOVERYPOINT™ CYAN-FP) and SEQ ID NO:18 (DISCOVERYPOINT™ GREEN-FP) and other fluorescent polypeptides.

FIG. 14 is a summary of data comparing various properties of exemplary fluorescent polypeptides of the invention, SEQ ID NO:8 (DISCOVERYPOINT™ CYAN-FP) and SEQ ID NO:18 (DISCOVERYPOINT™ GREEN-FP).

FIG. 15 is a summary of the sequences of overhangs used to construct exemplary sequences of the invention.

Like reference symbols in the various drawings indicate like elements.

### DETAILED DESCRIPTION

The invention provides polypeptides having a fluorescent activity, e.g., an auto-fluorescent activity, polynucleotides encoding the polypeptides, and methods for making and using these polynucleotides and polypeptides. The polypeptides of the invention can be used as noninvasive fluorescent markers in living cells and intact organs and animals. The polypeptides of the invention can be used as, e.g., in vivo markers/tracers of gene expression and protein localization, activity indicators, fluorescent resonance energy transfer (FRET) markers, cell lineage markers/tracers, reporters of gene expression and as markers/tracers in protein-protein interactions.

The present invention provides novel fluorescent proteins, polynucleotides encoding them and methods for making and using them. The invention provides a number of fluorescent proteins that can be used research tools, e.g., as in vivo markers of gene expression, protein localization, activity indicators (i.e., pH, Ca²⁺ levels), and for FRET applications. In one aspect, the fluorescent proteins of the invention can be fused to peptides or to complete polypeptides to observe the location, movement and dynamics of the proteins. In one aspect, the fluorescent proteins of the invention can be fused to specific targeting peptides or polypeptides to observe the location, structure, and dynamics of intracellular organelles over extended periods of time. In other aspects, the fluorescent proteins of the invention can be used as an alternative to immunofluorescence microscopy. For example, the expression of fluorescent protein gene fusions of the invention can be used to probe the function of cellular components for DNA replication, translation, protein export, and signal transduction that have been difficult to study in living cells. The invention also encompasses compositions such as vectors and cells that comprise either the nucleic acids or the protein gene products.

In one aspect, the fluorescent proteins of the invention are used as noninvasive fluorescent markers in living cells. These fluorescent proteins allow for a wide range of applications where they may function as cell lineage tracers, reporters of gene expression, or as measures of protein-protein interactions. The fluorescent proteins of the invention can have a variety of brightness (e.g., decreased or increased brightness), altered excitation and emission maxima, altered stability and/or differential sensitivity to pH. They can be used for following the trafficking and function of proteins in living cells and for monitoring the intracellular environment.

In one aspect, the fluorescent polypeptides of the invention are active at a high and/or at a low temperature, or, over a wide range of temperature, e.g., they can be active in the temperatures ranging between 20° C. to 90° C., between 30° C. to 80° C., or between 40° C. to 70° C. The invention also provides fluorescent polypeptides of the invention that have activity at alkaline pHs or at acidic pHs, e.g., low water acidity. In alternative aspects, the fluorescent polypeptides of the invention can have activity in acidic pHs as low as pH 5.0, pH 4.5, pH 4.0, pH 3.5, pH 3.0, and pH 2.5. In alternative aspects, the fluorescent polypeptides of the invention can have activity in alkaline pHs as high as pH 7.5, pH 8.0, pH 8.5, pH 9.0, and pH 9.5. In one aspect, the fluorescent polypeptides of the invention are active in the temperature range of between about 40° C. to about 70° C. under conditions of low water activity (low water content).

The invention also provides methods for further modifying the exemplary fluorescent polypeptides of the invention to generate proteins with desirable properties. For example, fluorescent polypeptides generated by the methods of the invention can have altered emission and absorption patterns, thermal stability, pH/activity profile, pH/stability profile (such as increased stability at low, e.g. pH<6 or pH<5, or high, e.g. pH>9, pH values), stability towards oxidation, Ca²⁺ dependency, specific activity and the like. The invention provides for altering any property of interest. For instance, the alteration may result in a variant, which, as compared to a parent fluorescent polypeptide, has altered emission and absorption patterns, or, pH or temperature fluorescent profiles.

### Definitions

The term “fluorescent polypeptide” encompasses any protein having a fluorescent activity, e.g., an auto-fluorescent activity. Fluorescent activity includes emission of radiation, generally light, from a material during illumination by radiation of usually higher frequency or from the impact of electrons. For example, the fluorescent polypeptides of the invention can emit light of a characteristic wavelength when excited by light, which is generally of a characteristic and different wavelength than that used to generate the emission. The term fluorescent polypeptide also includes the proteins in which the chromophore autocatalytically formed and does not require addition of a substrate to induce fluorescence. The term “cellular fluorescence” denotes the fluorescence of a fluorescent protein of the present invention when expressed in a cell.

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The terms “array” or “microarray” or “biochip” or “chip” as used herein is a plurality of target elements, each target element comprising a defined amount of one or more polypeptides (including antibodies) or nucleic acids immobilized onto a defined area of a substrate surface, as discussed in further detail, below.

As used herein, the terms “computer,” “computer program” and “processor” are used in their broadest general contexts and incorporate all such devices, as described in detail, below.

A “coding sequence of” or a “sequence encodes” a particular polypeptide or protein, is a nucleic acid sequence which is transcribed and translated into a polypeptide or protein when placed under the control of appropriate regulatory sequences.

The term “expression cassette” as used herein refers to a nucleotide sequence which is capable of affecting expression of a structural gene (i.e., a protein coding sequence, such as a fluorescent polypeptide of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like. A “vector” comprises a nucleic acid that can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

“Plasmids” can be commercially available, publicly available on an unrestricted basis, or can be constructed from available plasmids in accord with published procedures. Equivalent plasmids to those described herein are known in the art and will be apparent to the ordinarily skilled artisan.

The term “gene” means a nucleic acid sequence comprising a segment of DNA involved in producing a transcription product (e.g., a message), which in turn is translated to produce a polypeptide chain, or regulates gene transcription, reproduction or stability. Genes can include, inter alia, regions preceding and following the coding region, such as leader and trailer, promoters and enhancers, as well as, where applicable, intervening sequences (introns) between individual coding segments (exons).

The phrases “nucleic acid” or “nucleic acid sequence” as used herein refer to an oligonucleotide, nucleotide, polynucleotide, or to a fragment of any of these, to DNA or RNA (e.g., mRNA, rRNA, tRNA) of genomic or synthetic origin which may be single-stranded or double-stranded and may represent a sense or antisense strand, to peptide nucleic acid (PNA), or to any DNA-like or RNA-like material, natural or synthetic in origin, including, e.g., iRNA, ribonucleoproteins (e.g., iRNPs). The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

“Amino acid” or “amino acid sequence” as used herein refer to an oligopeptide, peptide, polypeptide, or protein sequence, or to a fragment, portion, or subunit of any of these, and to naturally occurring or synthetic molecules.

The terms “polypeptide” and “protein” as used herein, refer to amino acids joined to each other by peptide bonds or modified peptide bonds, i.e., peptide isosteres, and may contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

As used herein, the term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids that have been purified from genomic DNA or from other sequences in a library or other environment by at least one, two, three, four, five or more orders of magnitude.

As used herein, the term “recombinant” means that the nucleic acid is adjacent to a “backbone” nucleic acid to which it is not adjacent in its natural environment. In one aspect, nucleic acids represent 5% or more of the number of nucleic acid inserts in a population of nucleic acid “backbone molecules.” “Backbone molecules” according to the invention include nucleic acids such as expression vectors, self-replicating nucleic acids, viruses, integrating nucleic acids, and other vectors or nucleic acids used to maintain or manipulate a nucleic acid insert of interest. In one aspect, the enriched nucleic acids represent 15%, 20%, 30%, 40%, 50%, 60%, 70%, 80%, 90% or more of the number of nucleic acid inserts in the population of recombinant backbone molecules. “Recombinant” polypeptides or proteins refer to polypeptides or proteins produced by recombinant DNA techniques; e.g., produced from cells transformed by an exogenous DNA construct encoding the desired polypeptide or protein. “Synthetic” polypeptides or protein are those prepared by chemical synthesis, as described in further detail, below.

A promoter sequence is “operably linked to” a coding sequence when RNA polymerase which initiates transcription at the promoter will transcribe the coding sequence into mRNA, as discussed further, below.

“Oligonucleotide” refers to either a single stranded polydeoxynucleotide or two complementary polydeoxynucleotide strands that may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

The phrase “substantially identical” in the context of two nucleic acids or polypeptides, can refer to two or more sequences that have, e.g., at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more nucleotide or amino acid residue (sequence) identity, when compared and aligned for maximum correspondence, as measured using one any known sequence comparison algorithm, as discussed in detail below, or by visual inspection. In alternative aspects, the invention provides nucleic acid and polypeptide sequences having substantial identity to an exemplary sequence of the invention, e.g., SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26 over a region of at least about 10, 20, 30, 40, 50, 100, 150, 200, 250, 300, 350, 400, 450, 500, 550, 600, 650, 700, 750, 800, 850, 900, 950, 1000, 1050, 1100, 1150, 1200 or more residues, or a region ranging from between about 50 residues to the full length of the nucleic acid or polypeptide. Nucleic acid sequences of the invention can be substantially identical over the entire length of a polypeptide coding region.

Additionally a “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a fluorescent polypeptide, resulting in modification of the structure of the polypeptide, without significantly altering its biological activity. For example, amino- or carboxyl-terminal amino acids that are not required for fluorescent activity can be removed.

“Hybridization” refers to the process by which a nucleic acid strand joins with a complementary strand through base pairing. Hybridization reactions can be sensitive and selective so that a particular sequence of interest can be identified even in samples in which it is present at low concentrations. Stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization, as described in detail, below. In alternative aspects, nucleic acids of the invention are defined by their ability to hybridize under various stringency conditions (e.g., high, medium, and low), as set forth herein.

The term “variant” refers to polynucleotides or polypeptides of the invention modified at one or more base pairs, codons, introns, exons, or amino acid residues (respectively) yet still retain the biological activity of a fluorescent polypeptide of the invention. Variants can be produced by any number of means included methods such as, for example, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, GSSM™ and any combination thereof. Techniques for producing variant fluorescent polypeptide having activity at a pH or temperature, for example, that is different from a wild-type GFP, are included herein.

The term “saturation mutagenesis” or “GSSM™” includes a method that uses degenerate oligonucleotide primers to introduce point mutations into a polynucleotide, as described in detail, below.

The term “optimized directed evolution system” or “optimized directed evolution” includes a method for reassembling fragments of related nucleic acid sequences, e.g., related genes, and explained in detail, below.

The term “synthetic ligation reassembly” or “SLR” includes a method of ligating oligonucleotide fragments in a non-stochastic fashion, and explained in detail, below.

### Generating and Manipulating Nucleic Acids

The invention provides nucleic acids, including expression cassettes such as expression vectors, encoding the polypeptides of the invention. Exemplary nucleic acids of the invention comprise sequences having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:199, SEQ ID NO:161, SEQ ID NO:163, SEQ ID NO:165, SEQ ID NO:167, SEQ ID NO:169, SEQ ID NO:171, SEQ ID NO:173, SEQ ID NO:175, SEQ ID NO:177, SEQ ID NO:179, SEQ ID NO:181, SEQ ID NO:183, SEQ ID NO:185, SEQ ID NO:187, SEQ ID NO:189, SEQ ID NO:191, SEQ ID NO:193, SEQ ID NO:195, SEQ ID NO:197.

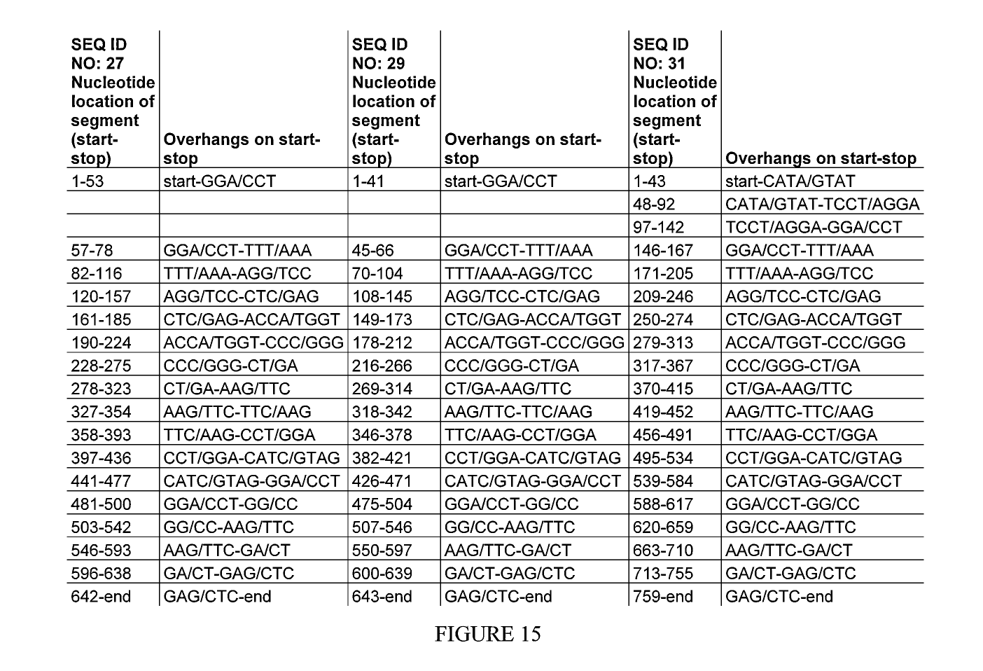

FIG. 15 describes nucleic acid segments of indicated SEQ ID NO:s used to synthesize exemplary fluorescent protein-encoding nucleic acids of the invention. The table indicates the sequence of the overhangs that are in addition to the SEQ ID residues of the protein coding sequences set forth in the table. SEQ ID NO:27, SEQ ID NO:29 and SEQ ID NO:31 are the parental sequences for the new SEQ ID NO:33 to SEQ ID NO:198. For the segment residues 1 to 53 of SEQ ID NO:27, 1 to 41 of SEQ NO:29 and 1 to 43 of SEQ ID NO:31, the term “start” represents ATG, which is part of the segment of residues 1 to 53. For example, in reading the table, for segment residues 1 to 53 of SEQ ID NO:27, the residues GGA are additional to the 3′ end of the sense strand, and the residues CCT are additional to the 5′ end of the non-coding strand, etc., carrying to all of the other segments listed in FIG. 15.

The parental sequences SEQ ID NO:27, SEQ ID NO:29 and SEQ ID NO:31 were codon optimized using SEQ ID NO:17 as a parental template.

In one aspect, the invention provides nucleic acids comprising all of the combination of segments as set forth in FIG. 15, or, alternatively, all combination of segments whose overhangs (described in FIG. 15) can anneal to each other.

Table 1 describes sources of selected exemplary sequences of the invention.

TABLE 1

| SEQ ID NO: | Source for application |
|---|---|
| 101, 102 | Artificial |
| 103, 104 | Artificial |
| 105, 106 | Artificial |
| 107, 108 | Artificial |
| 109, 110 | Artificial |
| 111, 112 | Artificial |
| 113, 114 | Artificial |
| 115, 116 | Artificial |
| 117, 118 | Artificial |
| 119, 120 | Artificial |
| 121, 122 | Artificial |
| 123, 124 | Artificial |
| 125, 126 | Artificial |
| 127, 128 | Artificial |
| 129, 130 | Artificial |
| 131, 132 | Artificial |
| 133, 134 | Artificial |
| 135, 136 | Artificial |
| 137, 138 | Artificial |
| 139, 140 | Artificial |
| 141, 142 | Artificial |
| 143, 144 | Artificial |
| 145, 146 | Artificial |
| 147, 148 | Artificial |
| 149, 150 | Artificial |
| 151, 152 | Artificial |
| 153, 154 | Artificial |
| 155, 156 | Artificial |
| 157, 158 | Artificial |
| 159, 160 | Artificial |
| 161, 162 | Artificial |
| 163, 164 | Artificial |
| 165, 166 | Artificial |
| 167, 168 | Artificial |
| 169, 170 | Artificial |
| 171, 172 | Artificial |
| 173, 174 | Artificial |
| 175, 176 | Artificial |
| 177, 178 | Artificial |
| 179, 180 | Artificial |
| 181, 182 | Artificial |
| 183, 184 | Artificial |
| 185, 186 | Artificial |
| 187, 188 | Artificial |
| 189, 190 | Artificial |
| 191, 192 | Artificial |
| 193, 194 | Artificial |
| 195, 196 | Artificial |
| 197, 198 | Artificial |
| 27, 28 | Artificial |
| 29, 30 | Artificial |
| 31, 32 | Artificial |
| 33, 34 | Artificial |
| 35, 36 | Artificial |
| 37, 38 | Artificial |
| 39, 40 | Artificial |
| 41, 42 | Artificial |
| 43, 44 | Artificial |
| 45, 46 | Artificial |
| 47, 48 | Artificial |
| 49, 50 | Artificial |
| 51, 52 | Artificial |
| 53, 54 | Artificial |
| 55, 56 | Artificial |
| 57, 58 | Artificial |
| 59, 60 | Artificial |
| 61, 62 | Artificial |
| 63, 64 | Artificial |
| 65, 66 | Artificial |
| 67, 68 | Artificial |
| 69, 70 | Artificial |
| 71, 72 | Artificial |
| 73, 74 | Artificial |
| 75, 76 | Artificial |
| 77, 78 | Artificial |
| 79, 80 | Artificial |
| 81, 82 | Artificial |
| 83, 84 | Artificial |
| 85, 86 | Artificial |
| 87, 88 | Artificial |
| 89, 90 | Artificial |
| 91, 92 | Artificial |
| 93, 94 | Artificial |
| 95, 96 | Artificial |
| 97, 98 | Artificial |
| 99, 100 | Artificial |
| 1, 2 | Environmental |
| 11, 12 | Environmental |
| 13, 14 | Environmental |
| 15, 16 | Environmental |
| 17, 18 | Environmental |
| 19, 20 | Environmental |
| 21, 22 | Environmental |
| 23, 24 | Environmental |
| 25, 26 | Environmental |
| 3, 4 | Environmental |
| 5, 6 | Environmental |
| 7, 8 | Environmental |
| 9, 10 | Environmental |

The invention also includes methods for discovering new fluorescent polypeptide sequences using the nucleic acids of the invention. Also provided are methods for modifying the nucleic acids of the invention by, e.g., synthetic ligation reassembly, optimized directed evolution system and/or saturation mutagenesis.

The nucleic acids of the invention can be made, isolated and/or manipulated by, e.g., cloning and expression of cDNA libraries, amplification of message or genomic DNA by PCR, and the like. In practicing the methods of the invention, homologous genes can be modified by manipulating a template nucleic acid, as described herein. The invention can be practiced in conjunction with any method or protocol or device known in the art, which are well described in the scientific and patent literature.

General Techniques

The nucleic acids used to practice this invention, whether RNA, iRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In one aspect, a nucleic acid encoding a polypeptide of the invention is assembled in appropriate phase with a leader sequence capable of directing secretion of the translated polypeptide or fragment thereof.

The invention provides fusion proteins and nucleic acids encoding them. A polypeptide of the invention can be fused to a heterologous peptide or polypeptide, such as N-terminal identification peptides that impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll (1993) DNA Cell. Biol., 12:441-53.

Transcriptional and Translational Control Sequences

The invention provides nucleic acid (e.g., DNA) sequences of the invention operatively linked to expression (e.g., transcriptional or translational) control sequence(s), e.g., promoters or enhancers, to direct or modulate RNA synthesis/expression. The expression control sequence can be in an expression vector. Exemplary bacterial promoters include lad, lacZ, T3, T7, gpt, lambda PR, PL and trp. Exemplary eukaryotic promoters include CMV immediate early, HSV thymidine kinase, early and late SV40, LTRs from retrovirus, and mouse metallothionein I.

Promoters suitable for expressing a polypeptide in bacteria include the E. coli lac or trp promoters, the lad promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter. Eukaryotic promoters include the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used.

Tissue-Specific Plant Promoters

The invention provides expression cassettes that can be expressed in a tissue-specific manner, e.g., that can express a pectate lyase of the invention in a tissue-specific manner. The invention also provides plants or seeds that express a nucleic acid or polypeptide of the invention in a tissue-specific manner. The tissue-specificity can be seed specific, stem specific, leaf specific, root specific, fruit specific and the like.

In one aspect, a constitutive promoter such as the CaMV 35S promoter can be used for expression in specific parts of the plant or seed or throughout the plant. For example, for overexpression, a plant promoter fragment can be employed which will direct expression of a nucleic acid in some or all tissues of a plant, e.g., a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, and other transcription initiation regions from various plant genes known to those of skill. Such genes include, e.g., ACT11 from Arabidopsis (Huang (1996) Plant Mol. Biol. 33:125-139); Cat3 from Arabidopsis (GenBank No. U43147, Zhong (1996) Mol. Gen. Genet. 251:196-203); the gene encoding stearoyl-acyl carrier protein desaturase from Brassica napus (Genbank No. X74782, Solocombe (1994) Plant Physiol. 104:1167-1176); GPc1 from maize (GenBank No. X15596; Martinez (1989) J. Mol. Biol. 208:551-565); the Gpc2 from maize (GenBank No. U45855, Manjunath (1997) Plant Mol. Biol. 33:97-112); plant promoters described in U.S. Pat. Nos. 4,962,028; 5,633,440.

The invention uses tissue-specific or constitutive promoters derived from viruses which can include, e.g., the tobamovirus subgenomic promoter (Kumagai (1995) Proc. Natl. Acad. Sci. USA 92:1679-1683; the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer (1996) Plant Mol. Biol. 31:1129-1139).

Alternatively, the plant promoter may direct expression of a fluorescent protein-expressing nucleic acid in a specific tissue, organ or cell type (i.e. tissue-specific promoters) or may be otherwise under more precise environmental or developmental control or under the control of an inducible promoter. Examples of environmental conditions that may affect transcription include anaerobic conditions, elevated temperature, the presence of light, or sprayed with chemicals/hormones. For example, the invention incorporates the drought-inducible promoter of maize (Busk (1997) supra); the cold, drought, and high salt inducible promoter from potato (Kirch (1997) Plant Mol. Biol. 33:897 909).

Tissue-specific promoters can promote transcription only within a certain time frame of developmental stage within that tissue. See, e.g., Blazquez (1998) Plant Cell 10:791-800, characterizing the Arabidopsis LEAFY gene promoter. See also Cardon (1997) Plant J 12:367-77, describing the transcription factor SPL3, which recognizes a conserved sequence motif in the promoter region of the A. thaliana floral meristem identity gene AP1; and Mandel (1995) Plant Molecular Biology, Vol. 29, pp 995-1004, describing the meristem promoter eIF4. Tissue specific promoters which are active throughout the life cycle of a particular tissue can be used. In one aspect, the nucleic acids of the invention are operably linked to a promoter active primarily only in cotton fiber cells. In one aspect, the nucleic acids of the invention are operably linked to a promoter active primarily during the stages of cotton fiber cell elongation, e.g., as described by Rinehart (1996) supra. The nucleic acids can be operably linked to the Fb12A gene promoter to be preferentially expressed in cotton fiber cells (Ibid). See also, John (1997) Proc. Natl. Acad. Sci. USA 89:5769-5773; John, et al., U.S. Pat. Nos. 5,608,148 and 5,602,321, describing cotton fiber-specific promoters and methods for the construction of transgenic cotton plants. Root-specific promoters may also be used to express the nucleic acids of the invention. Examples of root-specific promoters include the promoter from the alcohol dehydrogenase gene (DeLisle (1990) Int. Rev. Cytol. 123:39-60). Other promoters that can be used to express the nucleic acids of the invention include, e.g., ovule-specific, embryo-specific, endosperm-specific, integument-specific, seed coat-specific promoters, or some combination thereof; a leaf-specific promoter (see, e.g., Busk (1997) Plant J. 11:1285 1295, describing a leaf-specific promoter in maize); the ORF13 promoter from Agrobacterium rhizogenes (which exhibits high activity in roots, see, e.g., Hansen (1997) supra); a maize pollen specific promoter (see, e.g., Guerrero (1990) Mol. Gen. Genet. 224:161 168); a tomato promoter active during fruit ripening, senescence and abscission of leaves and, to a lesser extent, of flowers can be used (see, e.g., Blume (1997) Plant J. 12:731 746); a pistil-specific promoter from the potato SK2 gene (see, e.g., Ficker (1997) Plant Mol. Biol. 35:425 431); the Blec4 gene from pea, which is active in epidermal tissue of vegetative and floral shoot apices of transgenic alfalfa making it a useful tool to target the expression of foreign genes to the epidermal layer of actively growing shoots or fibers; the ovule-specific BEL1 gene (see, e.g., Reiser (1995) Cell 83:735-742, GenBank No. U39944); and/or, the promoter in Klee, U.S. Pat. No. 5,589,583, describing a plant promoter region is capable of conferring high levels of transcription in meristematic tissue and/or rapidly dividing cells.

Alternatively, plant promoters which are inducible upon exposure to plant hormones, such as auxins, are used to express the nucleic acids of the invention. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant. Microbe Interact. 10:933-937); and, the promoter responsive to the stress hormone abscisic acid (Sheen (1996) Science 274:1900-1902).

The nucleic acids of the invention can also be operably linked to plant promoters which are inducible upon exposure to chemicals reagents which can be applied to the plant, such as herbicides or antibiotics. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. Coding sequence can be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324). Using chemically- (e.g., hormone- or pesticide-) induced promoters, i.e., promoter responsive to a chemical which can be applied to the transgenic plant in the field, expression of a polypeptide of the invention can be induced at a particular stage of development of the plant. Thus, the invention also provides for transgenic plants containing an inducible gene encoding for polypeptides of the invention whose host range is limited to target plant species, such as corn, rice, barley, wheat, potato or other crops, inducible at any stage of development of the crop.

One of skill will recognize that a tissue-specific plant promoter may drive expression of operably linked sequences in tissues other than the target tissue. Thus, a tissue-specific promoter is one that drives expression preferentially in the target tissue or cell type, but may also lead to some expression in other tissues as well.

The nucleic acids of the invention can also be operably linked to plant promoters which are inducible upon exposure to chemicals reagents. These reagents include, e.g., herbicides, synthetic auxins, or antibiotics which can be applied, e.g., sprayed, onto transgenic plants. Inducible expression of the pectate lyase-producing nucleic acids of the invention will allow the grower to select plants with the optimal pectate lyase expression and/or activity. The development of plant parts can thus controlled. In this way the invention provides the means to facilitate the harvesting of plants and plant parts. For example, in various embodiments, the maize In2-2 promoter, activated by benzenesulfonamide herbicide safeners, is used (De Veylder (1997) Plant Cell Physiol. 38:568-577); application of different herbicide safeners induces distinct gene expression patterns, including expression in the root, hydathodes, and the shoot apical meristem. Coding sequences of the invention are also under the control of a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324).

If proper polypeptide expression is desired, a polyadenylation region at the 3′-end of the coding region should be included. The polyadenylation region can be derived from the natural gene, from a variety of other plant genes, or from genes in the Agrobacterial T-DNA.

Expression Vectors and Cloning Vehicles

The invention provides expression vectors and cloning vehicles comprising nucleic acids of the invention, e.g., sequences encoding the fluorescent proteins of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available. Exemplary vectors are include: bacterial: pQE vectors (Qiagen), pBluescript plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used so long as they are replicable and viable in the host. Low copy number or high copy number vectors may be employed with the present invention.

The expression vector may comprise a promoter, a ribosome binding site for translation initiation and a transcription terminator. The vector may also include appropriate sequences for amplifying expression. Mammalian expression vectors can comprise an origin of replication, any necessary ribosome binding sites, a polyadenylation site, splice donor and acceptor sites, transcriptional termination sequences, and 5′ flanking non-transcribed sequences. In some aspects, DNA sequences derived from the SV40 splice and polyadenylation sites may be used to provide the required non-transcribed genetic elements.

In one aspect, the expression vectors contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in E. coli, and the S. cerevisiae TRP 1 gene. Promoter regions can be selected from any desired gene using chloramphenicol transferase (CAT) vectors or other vectors with selectable markers.

Vectors for expressing the polypeptide or fragment thereof in eukaryotic cells may also contain enhancers to increase expression levels. Enhancers are cis-acting elements of DNA, usually from about 10 to about 300 bp in length that act on a promoter to increase its transcription. Examples include the SV40 enhancer on the late side of the replication origin by 100 to 270, the cytomegalovirus early promoter enhancer, the polyoma enhancer on the late side of the replication origin, and the adenovirus enhancers.

A DNA sequence may be inserted into a vector by a variety of procedures. In general, the DNA sequence is ligated to the desired position in the vector following digestion of the insert and the vector with appropriate restriction endonucleases. Alternatively, blunt ends in both the insert and the vector may be ligated. A variety of cloning techniques are known in the art, e.g., as described in Ausubel and Sambrook. Such procedures and others are deemed to be within the scope of those skilled in the art.

The vector may be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook.

Particular bacterial vectors which may be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pD10, psiX174 pBluescript II KS, pNH8A, pNH16a, pNH18A, pNH46A (Stratagene), ptrc99a, pKK223-3, pKK233-3, DR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell.

Host Cells and Transformed Cells

The invention also provides a transformed cell comprising a nucleic acid sequence of the invention, e.g., a sequence encoding a fluorescent polypeptide of the invention, or a vector of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

The vector may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

Where appropriate, the engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Amplification of Nucleic Acids

In practicing the invention, nucleic acids encoding the polypeptides of the invention, or modified nucleic acids, can be reproduced by, e.g., amplification. The invention provides amplification primer sequence pairs for amplifying nucleic acids encoding fluorescent polypeptides, where the primer pairs are capable of amplifying nucleic acid sequences including the exemplary SEQ ID NO:1, or a subsequence thereof; a sequence as set forth in SEQ ID NO:3, or a subsequence thereof; a sequence as set forth in SEQ ID NO:5, or a subsequence thereof; and, a sequence as set forth in SEQ ID NO:7, or a subsequence thereof, a sequence as set forth in SEQ ID NO:9, or a subsequence thereof, a sequence as set forth in SEQ ID NO:11, or a subsequence thereof, a sequence as set forth in SEQ ID NO:13, or a subsequence thereof, a sequence as set forth in SEQ ID NO:15, or a subsequence thereof, a sequence as set forth in SEQ ID NO:17, or a subsequence thereof, a sequence as set forth in SEQ ID NO:19, or a subsequence thereof, a sequence as set forth in SEQ ID NO:21, or a subsequence thereof, a sequence as set forth in SEQ ID NO:23, or a subsequence thereof, a sequence as set forth in SEQ ID NO:25, or a subsequence thereof. One of skill in the art can design amplification primer sequence pairs for any part of or the full length of these sequences; for example:

The exemplary SEQ ID NO:1 is

atgagtcattccaagagtgtgatcaaggatgaaatgttcatcaagattcatctggaaggaacgttcaatgggcataagtttgaaatagaa
ggcgaaggacacgggaagccttatgcaggcaccaatttcgttaagcttgtggttaccaggggtggacctttgccatttggttggcacatt
ttgtcgccacaatttcagtatggaaacaagacgtttgtcagctaccctagagacatacccgattatataaagcagtcatttcctgagggctt
tacatgggaacggatcatgaccttcgaagacggtggcgtgtgttgtatcaccagtgatatcagtttgaaaagcaacaactgtttcttcaac
gacatcaagttcactggcatgaactttcctccaaatggatctgttgtgcagaagaagacgataggctgggaacccagcactgagcgttt
gtatctgcgtgacggggtgctgacaggagacattgataagacactgaagctcagcggaggtggtcattacacatgcgcctttaaaact
atttacaggtcgaagaagaacttgacgctgcctgattgcctttactatgttgacaccaaacttgatataaggaagttcgacgaaaattacat
caacgttgagcaggatgaaattgctactgcacgccaccatgggcttaaataa

Thus, an exemplary amplification primer sequence pair is residues 1 to 21 of SEQ ID NO:1 (i.e., atgagtcattccaagagtgtg) and the complementary strand of the last 21 residues of SEQ ID NO:1 (i.e., the complementary strand of cgccaccatgggcttaaataa).

The exemplary SEQ ID NO:3 is

atgagtcattccaagagtgtgatcaaggatgaaatgttcatcaagattcatctggaaggaacgttcaatgggcacaagtttgaaatagaa
ggcgaaggacacgggaagccttatgcaggcaccaatttcgttaagcttgtggttaccaagggtggacctttgccatttggttggcacatt
ttgtcgccacaatttcagtatggaaacaagacgtttgtcagctaccctagagacatacccgattatataaagcagtcatttcctgagggctt
tacatgggtacggatcatgacctttgaagacggtggcgtgtgttgtatcaccagtgatatcagtttgaaaagcaacaactgtttcttcaac
gacatcaagttcactggcatgaactttcctccaaatggacctgttgtgcagaagaagacgataggctgggaacccagcactgagcgttt
gtatctgcgtgacggggtgctgacaggagacattgataagacactgaagctcagcggaggtggtcattacacatgcgcctttaaaact
atttacaggtcgaagaagaacttgacgctgcctgattgcttttactatgttgacaccaaacttgatataaggaagttcgacgaaaattacat
caacgttgagcaggatgaaattgctactgcacgccaccatgggcttaaataa

Thus, an exemplary amplification primer sequence pair is residues 1 to 21 of SEQ ID NO:3 (i.e., atgagtcattccaagag) and the complementary strand of the last 21 residues of SEQ ID NO:3 (i.e., the complementary strand of cgccaccatgggcttaaataa).

The exemplary SEQ ID NO:5 is

atgagtcattctaagagtgtgatcaaggatgaaatgttcatcaagattcatctggaaggaacgttcaatgggcacaagtttgaaatagaa
ggcgaaggacacgggaagccttatgcaggcaccaatttcgttaagcttgtggttaccaagggtggacctttgccatttggttggcacatt
ttgtcgccacaatttcagtatggaaacaagacgtttgtcagctaccctagagacatacccgattatataaagcagtcatttcctgagggctt
tacatgggaacggatcatgacctttgaagacggtggcgtgtgttgtatcaccagtgatatcagtttgaaaagcaacaactgtttcttcaac
gacatcaagttcactggcatgaactttcctccaaatggacctgttgtgcagaagaagacgataggctgggaacccagcactgagcgttt
gtatctgcgtgacggggtgctgacaggagacattgataagacactgaagctcagcggaggtggtcattacacatgcgcctttaaaact
atttacaggtcgaagaagaacttgacgctgcctgattgcttttactatgttgacaccaaacttgatataaggaagttcgacgaaaattacat
caacgttgagcaggatgaaattgctactgcacgccaccatgggcttaaataa

Thus, an exemplary amplification primer sequence pair is residues 1 to 21 of SEQ ID NO:5 (i.e., atgagtcattctaagagtgtg) and the complementary strand of the last 21 residues of SEQ ID NO:5 (i.e., the complementary strand of cgccaccatgggcttaaataa).

The exemplary SEQ ID NO:7 is

atgagtcattccaagagtgtgatcaaggacgaaatgttcatcaagattcatctggaaggaacgttcaatgggcacaagtttgaaatagaa
ggcgagggaaacgggaagccttatgcaggcaccaatttcgttaagcttgtggttaccaagggtgggcctcttccatttggttggcacatt
ttgtcgccacaattacaatacggaaacaagtcgtttgtcagctaccctgcagacatacctgattatataaagctgtcatttcctgagggcttt
acatgggaaaggatcatgacctttgaagacggtggcgtgtgttgtatcaccagtgatatcagtatgaaaagcaacaactgtttcttctacg
acatcaagttcactggcatgaactttcctccaaatggacctgttgtgcagaagaagaccacaggctgggaacccagtactgagcgtttg
tatctgcgtgacggggtgctgacaggagacattcataagacactgaagctcagcggaggtggtcattacacatgcgtctttaaaactatt
tacaggtcgaagaagaacttgacgctgcctgattgcttttactatgttgacaccaaacttgatataaggaagttcgacgaaaattacatca
acgttgagcaggatgaaattgctactgcacgccaccatgggcttaaataa

Thus, an exemplary amplification primer sequence pair is residues 1 to 21 of SEQ ID NO:7 (i.e., atgagtcattccaagagtgtg) and the complementary strand of the last 21 residues of SEQ ID NO:7 (i.e., the complementary strand of cgccaccatgggcttaaataa).

The exemplary SEQ ID NO:9 is

atgaagggggtgaaggaagtaatgaagatcagtctggagatggactgcactgttaacggcgacaaatttaagatcactggggatgga
acaggagaaccttacgaaggaacacagactttacatcttacagagaaggaaggcaagcctctgacgttttctttcgatgtattgacacca
gcatttcagtatggaaaccgtacattcaccaaatacccaggcaatataccagactttttcaagcagaccgtttctggtggcgggtatacct
gggagcgaaaaatgacttatgaagacgggggcataagtaacgtccgaagcgacatcagtgtgaaaggtgactctttctactataagatt
cacttcactggcgagtttcctcctcatggtccagtgatgcagaggaagacagtaaaatgggagccatccactgaagtaatgtatgttgac
gacaagagtgacggtgtgctgaagggagatgtcaacatggctctgttgcttaaagatggccgccatttgagagttgactttaacacttctt
acatacccaagaagaaggtcgagaatatgcctgactaccattttatagaccaccgcattgagattctgggcaacccagaagacaagcc
ggtcaagctgtacgagtgtgctgtagctcgctattctctgctgcctgagaagaacaagtca

Thus, an exemplary amplification primer sequence pair is residues 1 to 21 of SEQ ID NO:9 (i.e., atgaagggggtgaaggaagta) and the complementary strand of the last 21 residues of SEQ ID NO:9 (i.e., the complementary strand of ctgcctgagaagaacaagtca).

The exemplary SEQ ID NO:11 is

atgaagggggtgaaggaagtcatgaagatcagtctggagatggactgcactgttaacggcgacaaatttaagatcactggggatgga
acaggagaaccttacgaaggaacacagactttacatcttacagagaaggaaggcaagcctctgacgttttctttcgatgtattgacacca
gcatttcagtatggaaaccgtacattcaccaaatacccaggcaatataccagactttttcaagcagaccgtttctggtggcgggtatacct
gggagcgaaaaatgacttatgaagacgggggcataagtaacgtccgaagcgacatcagtgtgaaaggtgactctttctactataagatt
cacttcactggcgagtttcctcctcatggtccagtgatgcagaggaagacagtaaaatgggagccatccactgaagtaatgtatgtgga
cgataagagtggtggtgagctgaagggagatgtcaacatggctctgttgcttaaagatggccgccatttgagagttgacttcaacacttc
ttacatacccaagaagaaggtcgagaatatgcctgactaccattttatagaccaccgcattgagattctgggcaacccagaagacaagc
cggtcaagctgtacgagtgtgctgtagctcgctattctctgctgcctgagaagaacaag

Thus, an exemplary amplification primer sequence pair is residues 1 to 21 of SEQ ID NO:11 (i.e., atgaagggggtgaaggaagtc) and the complementary strand of the last 21 residues of SEQ ID NO:11 (i.e., the complementary strand of ctgctgcctgagaagaacaag).

The exemplary SEQ ID NO:13 is

gtgaaggaagtaatgaagatcagtctggagatggactgcactgttaacggcgacaaatttaagatcactggggatggaacaggagaa
ccttacgaaggaacacagactttacatcttacagagaaggaaggcaagcctctgacgttttctttcgatgtattgacaccagcatttcagt
atggcaaccgtacattcaccaaatacccaggcaatataccagactttttcaagcagaccgtttctggtggcgggtatacctgggagcga
aaaatgacttatgaagacgggggcataagtaacgtccgaagcgacatcagtgtgaaaggtgactctttctactataagattcacttcact
ggcgaatttccttctcacggtccagtgatgcagaagaagacggtaaaatgggagccatccactgaagtaatgtatgtggacgataaga
gtgatggtgtgctgaagggagatgtcaacatggctctgttgcttaaagatggccgccatttgcgagtggacttcaacacttcttacatacc
caagaagaaggtcgagaatatgcctgactaccattttatagaccaccgcattgagattctgggcaacccagatgacaatccggtcaag
ctgtacgagtgtgctgtagctcgctgttctctgctgcctgagaagaacaag

Thus, an exemplary amplification primer sequence pair is residues 1 to 21 of SEQ ID NO:13 (i.e., gtgaaggaagtaatgaagatc) and the complementary strand of the last 21 residues of SEQ ID NO:13 (i.e., the complementary strand of ctgctgcctgagaagaacaag).

The exemplary SEQ ID NO:15 is

atgaagggggtgaaggaagtgatgaagatccaggtgaagatgaacatcactgttaacggcgacaaatttgtgatcactggggatgga
acaggcgaaccttacgacgggacacagattttaaatcttacagtggaaggaggcaagcctctgacattttctttcgatatattgacacca
gtatttatgtatggcaacagagcattcaccaaatacccagagagtatcccagactttttcaagcagaccgtttctggtggcgggtatactt
ggaaacgaaagatgatttatgatcacgaggctgagggcgtgagtaccgttgacggggacatcagtgtgaatggagactgtttcatctat
aagattacgtttgacggcacatttcgtgaagatggtgcagtgatgcagaagatgacggaaaaatgggaaccatccactgaagtgatgt
acaaggacgataaaaatgatgatgtgctgaagggagatgtcaaccatgctcttttgcttaaagatggccgccatgtgcgagttgatttca
atacctcttacaaagccaagtcaaagatcgagaatatgcctggttaccattttgtagaccaccgcattgagataatagggcgatcatcgc
aagacacgaaggtcaagctgttcgagaacgctgtcgctcgctgttctctgctgcctgagaagaaccag

Thus, an exemplary amplification primer sequence pair is residues 1 to 21 of SEQ ID NO:15 (i.e., atgaagggggtgaaggaagtg) and the complementary strand of the last 21 residues of SEQ ID NO:15 (i.e., the complementary strand of ctgctgcctgagaagaaccag).

The exemplary SEQ ID NO:17 is

atgaaggggg tgaaggaagt aatgaagatc agtctggaga tggactgcac tgttaacggc gacaaattta agatcactgg
ggatggaaca ggagaacctt acgaaggaac acagacttta catcttacag agaaggaagg caagcctctg acgttttctt
tcgatgtatt gacaccagca tttcagtatg gaaaccgtac attcaccaaa tacccaggca atataccaga ctttttcaag
cagaccgttt ctggtggcgg gtatacctgg gagcgaaaaa tgacttatga agacgggggc ataagtaacg tccgaagcga
catcagtgtg aaaggtgact ctttctacta taagattcac ttcactggcg agtttcctcc tcatggtcca gtgatgcaga
ggaagacagt aaaatgggag ccatccactg aagtaatgta tgttgacgac aagagtgacg gtgtgctgaa gggagatgtc
aacatggctc tgttgcttaa agatggccgc catttgagag ttgactttaa cacttcttac atacccaaga agaaggtcga
gaatatgcct gactaccatt ttatagacca ccgcattgag attctgggca acccagaaga caagccggtc aagctgtacg
agtgtgctgt agctcgctat tctctg ctgc ctgagaagaa caagtaa

Thus, an exemplary amplification primer sequence pair is residues 1 to 21 of SEQ ID NO:17 (i.e., atgaagggggtgaaggaagta) and the complementary strand of the last 21 residues of SEQ ID NO:17 (i.e., the complementary strand of ctgcctgagaagaacaagtaa).

The exemplary SEQ ID NO:19 is

atgaaggggg tgaaggaagt aatgaagatc agtctggaga tggactgcac tgttaacggc gacaaattta agatcactgg
ggatggaaca ggagaacctt acgaaggaac acagacttta catcttacag agaaggaagg caagcctctg acgttttctt
tcgatgtatt gacaccagca tttcagtatg gaaaccgtac attcaccaaa tacccaggca atataccaga ctttttcaag
cagaccgttt ctggtggcgg gtatacctgg gagcgaaaaa tgacttatga agacgggggc ataagtaacg tccgaagcga
catcagtgtg aaaggtgact ctttctacta taagattcac ttcactggcg agtttcctcc tcatggtcca gtgatgcaga
ggaagacagt aaaatgggag ccatccactg aagtaatgta tgttgacgac aagagtgacg gtgtgctgaa gggagatgtc
aacatggctc tgttgcttaa agatggccgc catttgagag ttgactttaa cacttcttac atacccaaga agaaggtcga
gaatatgcct gactaccatt ttatagacca ccgcattgag attctgggca acccagaaga caagccggtc aagctgtacg
agtgtgctgt agctcgctat tctctgctgc ctgagaagaa caagtcaaag ggcaattcga agcttgaagg taagcctatc
cctaaccctc tcctcggtct cgattctacg cgtaccggtt aa

Thus, an exemplary amplification primer sequence pair is residues 1 to 21 of SEQ ID NO:19 (i.e., atgaagggggtgaaggaagta) and the complementary strand of the last 21 residues of SEQ ID NO:19 (i.e., the complementary strand of gattctacgcgtaccggttaa).

The exemplary SEQ ID NO:21 is

gtgatggcga tttccgctct aaagaacgtc atcatcatcg taatcatata ctcctgcagc actagtgctg attcgtcgaa
ctcttactct ggatcctcct tcgcgaatgg gattgcagag gaaatgatga ctgacctgca tttagagggt gctgttaacg
ggcaccactt tacaattaaa ggcgaaggag gaggctaccc ttacgaggga gtgcagttta tgagcctcga ggtagtcaat
ggtgcccctc ttccgttctc ttttgatatc ttgacaccgg cattcatgta tggcaacaga gtgttcacca agtatccaaa
agagatacca cactatttca agcagacgtt tcctgaaggg tatcactggg aaagaagcat tccctttcaa gatcaggcct
cgtgcacggt aaccagccac ataaggatga aagaggaaga ggagcggcat tttcttctta acgtcaaatt ttactgtgtg
aattttcccc ccaatggtcc agtcatgcag aggaggatac ggggatggga gccatccact gagaacattt atccgcgtga
tgaatttcta gagggccatg atgacatgac tcttcgggtt gaaggaggtg gctattaccg agctgaattc agaagttctt
acaaaggaaa gcactcaatc aacatgccag actttcactt catagaccac cgcattgaga ttatggagca tgacgaagac
tacaaccatg ttaagctgcg tgaagtagcc catgctcgtt actct ccgct gccttctgtgcactaa

Thus, an exemplary amplification primer sequence pair is residues 1 to 21 of SEQ ID NO:21 (i.e., gtgatggcgatttccgctcta) and the complementary strand of the last 21 residues of SEQ ID NO:21 (i.e., the complementary strand of ccgctgccttctgtgcactaa).

The exemplary SEQ ID NO:23 is

gtgatggcga tttccgctct aaagaacgtc atcatcatcg taatcatata ctcctgcagc actagtgctg attcgtcgaa
ctcttactct ggatcctcct tcgcgaatgg gattgcagag gaaatgatga ctgacctgca tttagagggt gctgttaacg
ggcaccactt tacaattaaa ggcgaaggag gaggctaccc ttacgaggga gtgcagttta tgagcctcga ggtagtcaat
ggtgcccctc ttccgttctc ttttgatatc ttgacaccgg cattcatgta tggcaacaga gtgttcacca agtatccaaa
agagatacca gactatttca agcagacgtt tcctgaaggg tatcactggg aaagaagcat tccctttcaa gatcaggcct
cgtgcacggt aaccagccac ataaggatga aagaggaaga ggagcggcat tttcttctta acgtcaaatt ttactgtgtg
aattttcccc ccaatggtcc agtcatgcag aggaggatac ggggatggga gccatccact gagaacattt atccgcgtga
tgaatttcta gagggccatg atgacatgac tcttcgggtt gaaggaggtg gctattaccg agctgaattc agaagttctt
acaaaggaaa gcactcaatc aacatgccag actttcactt catagaccac cgcattgaga ttatggagca tgacgaagac
tacaaccatg ttaagctgcg tgaagtagcc catgctcgtt actctccgct gccttctgtgcactaa

Thus, an exemplary amplification primer sequence pair is residues 1 to 21 of SEQ ID NO:23 (i.e., gtgatggcgatttccgctcta) and the complementary strand of the last 21 residues of SEQ ID NO:23 (i.e., the complementary strand of ccgctgccttctgtgcactaa).

The exemplary SEQ ID NO:25 is

atggcgattt ccgctctaaa gaacgtcatc atcatcgtaa tcatatactc ccgcagcact agtgctgatt cgtcgaactc
ttactctgga tcctccttcg cgaatgggat tgcagaggaa atgatgactg acctgcattt agagggtgct gttaacgggc
accactttac aattaaaggc gaaggaggag gctaccctta cgagggagtg cagtttatga gcctcgaggt agtcaatggt
gcccctcttc cgttctcttt tgatatcttg acaccggcat tcatgtatgg caacagagtg ttcaccaagt atccaaaaga
gataccagac tatttcaagc agacgtttcc tgaagggtat cactgggaaa gaagcattcc ctttcaagat caggcctcgt
gcacggtaac cagccacata aggatgaaag aggaagagga gcggcatttt cttcttaacg tcaaatttta ctgtgtgaat
tttcccccca atggtccagt catgcagagg aggatacggg gatgggagcc atccactgag aacatttatc cgcgtgatga
atttctagag ggccatgatg acatgactct tcgggttgaa ggaggtggct attaccgagc tgaattcaga agttcttaca
aaggaaagca ctcaatcaac atgccagact ttcacttcat agaccaccgc attgagatta tggagcatga cgaagactac
aaccatgtta agctgcgtga agtagcctat gctcgttact ctccgctgcc ttctgtgcactaa

Thus, an exemplary amplification primer sequence pair is residues 1 to 21 of SEQ ID NO:25 (i.e., atggcgatttccgctctaaag) and the complementary strand of the last 21 residues of SEQ ID NO:25 (i.e., the complementary strand of ccgctgccttctgtgcactaa).

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified. The skilled artisan can select and design suitable oligonucleotide amplification primers. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

### Determining the Degree of Sequence Identity

The invention provides nucleic acids having least about 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to an exemplary nucleic acid of the invention. In one aspect, the invention provides nucleic acids having at least 85% sequence identity to SEQ ID NO:1, nucleic acids having at least 85% sequence identity to SEQ ID NO:3, nucleic acids having at least 85% sequence identity to SEQ ID NO:5, nucleic acids having at least 85% sequence identity to SEQ ID NO:7, nucleic acids having at least 75% sequence identity to SEQ ID NO:9, nucleic acids having at least 75% sequence identity to SEQ ID NO:11, nucleic acids having at least 75% sequence identity to SEQ ID NO:13, nucleic acids having at least 70% sequence identity to SEQ ID NO:15, nucleic acids having at least 70% sequence identity to SEQ ID NO:17, nucleic acids having at least 70% sequence identity to SEQ ID NO:19, nucleic acids having at least 85% sequence identity to SEQ ID NO:21, nucleic acids having at least 85% sequence identity to SEQ ID NO:23, and nucleic acids having at least 85% sequence identity to SEQ ID NO:25. In alternative embodiments, the invention provides nucleic acids and polypeptides having at least 99%, 98%, 97%, 96%, 95%, 90%, 85%, 80%, 75%, 70%, 65%, 60%, 55% or 50% sequence identity (homology) to SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, or SEQ ID NO:25. In alternative aspects, the sequence identify can be over a region of at least about 5, 10, 20, 30, 40, 50, 100, 150, 200, 250, 300, 350, 400, 450, 500, 550, 600, 650 consecutive residues, or the full length of the nucleic acid or polypeptide. The extent of sequence identity (homology) may be determined using any computer program and associated parameters, including those described herein, such as BLAST 2.2.2. or FASTA version 3.0t78, with the default parameters.

Homologous sequences also include RNA sequences in which uridines replace the thymines in the nucleic acid sequences. The homologous sequences may be obtained using any of the procedures described herein or may result from the correction of a sequencing error. It will be appreciated that the nucleic acid sequences as set forth herein can be represented in the traditional single character format (see, e.g., Stryer, Lubert. Biochemistry, 3rd Ed., W. H Freeman & Co., New York) or in any other format which records the identity of the nucleotides in a sequence.

Various sequence comparison programs identified herein are used in this aspect of the invention. Protein and/or nucleic acid sequence identities (homologies) may be evaluated using any of the variety of sequence comparison algorithms and programs known in the art. Such algorithms and programs include, but are not limited to, TBLASTN, BLASTP, FASTA, TFASTA, and CLUSTALW (Pearson and Lipman, Proc. Natl. Acad. Sci. USA 85(8):2444-2448, 1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

Homology or identity can be measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (an exemplary sequence SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, contiguous residues ranging anywhere from 20 to the full length of an exemplary polypeptide or nucleic acid sequence of the invention, e.g., SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23 and/or SEQ ID NO:25 are compared to a reference sequence of the same number of contiguous positions after the two sequences are optimally aligned. If the reference sequence has the requisite sequence identity to an exemplary polypeptide or nucleic acid sequence of the invention, e.g., 70%, 75%, 80%, 90% or 95% sequence identity to SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23 or SEQ ID NO:25, that sequence is within the scope of the invention. In alternative embodiments, subsequences ranging from about 20 to 600, about 50 to 200, and about 100 to 150 are compared to a reference sequence of the same number of contiguous positions after the two sequences are optimally aligned. Methods of alignment of sequence for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of person & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. Other algorithms for determining homology or identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF. Such alignment programs can also be used to screen genome databases to identify polynucleotide sequences having substantially identical sequences. A number of genome databases are available, for example, a substantial portion of the human genome is available as part of the Human Genome Sequencing Project (Gibbs, 1995). Several genomes have been sequenced, e.g., M. genitalium (Fraser et al., 1995), M. jannaschii (Bult et al., 1996), H. influenzae (Fleischmann et al., 1995), E. coli (Blattner et al., 1997), and yeast (S. cerevisiae) (Mewes et al., 1997), and D. melanogaster (Adams et al., 2000). Significant progress has also been made in sequencing the genomes of model organism, such as mouse, C. elegans, and Arabadopsis sp. Databases containing genomic information annotated with some functional information are maintained by different organization, and are accessible via the interne.

BLAST, BLAST 2.0 and BLAST 2.2.2 algorithms are also used to practice the invention. They are described, e.g., in Altschul (1977) Nuc. Acids Res. 25:3389-3402; Altschul (1990) J. Mol. Biol. 215:403-410. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectations (E) of 10, and the BLOSUM62 scoring matrix (see Henikoff & Henikoff (1989) Proc. Natl. Acad. Sci. USA 89:10915) alignments (B) of 50, expectation (E) of 10, M=5, N=−4, and a comparison of both strands. The BLAST algorithm also performs a statistical analysis of the similarity between two sequences (see, e.g., Karlin & Altschul (1993) Proc. Natl. Acad. Sci. USA 90:5873). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001. In one aspect, protein and nucleic acid sequence homologies are evaluated using the Basic Local Alignment Search Tool (“BLAST”). For example, five specific BLAST programs can be used to perform the following task: (1) BLASTP and BLAST3 compare an amino acid query sequence against a protein sequence database; (2) BLASTN compares a nucleotide query sequence against a nucleotide sequence database; (3) BLASTX compares the six-frame conceptual translation products of a query nucleotide sequence (both strands) against a protein sequence database; (4) TBLASTN compares a query protein sequence against a nucleotide sequence database translated in all six reading frames (both strands); and, (5) TBLASTX compares the six-frame translations of a nucleotide query sequence against the six-frame translations of a nucleotide sequence database. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., Science 256:1443-1445, 1992; Henikoff and Henikoff, Proteins 17:49-61, 1993). Less preferably, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation).

In one aspect of the invention, to determine if a nucleic acid has the requisite sequence identity to be within the scope of the invention, the NCBI BLAST 2.2.2 programs is used, default options to blastp. There are about 38 setting options in the BLAST 2.2.2 program. In this exemplary aspect of the invention, all default values are used except for the default filtering setting (i.e., all parameters set to default except filtering which is set to OFF); in its place a “—F F” setting is used, which disables filtering. Use of default filtering often results in Karlin-Altschul violations due to short length of sequence.
The default values used in this exemplary aspect of the invention include:
“Filter for low complexity: ON
Word Size: 3
Matrix: Blosum62
Gap Costs Existence:11
Extension:1”

Other default settings are: filter for low complexity OFF, word size of 3 for protein, BLOSUM62 matrix, gap existence penalty of −11 and a gap extension penalty of −1.

An exemplary NCBI BLAST 2.2.2 program setting is set forth in Example 1, below. Note that the “-W” option defaults to 0. This means that, if not set, the word size defaults to 3 for proteins and 11 for nucleotides.

Computer Systems and Computer Program Products

To determine and identify sequence identities, structural homologies, motifs and the like in silico, the sequence of the invention can be stored, recorded, and manipulated on any medium which can be read and accessed by a computer. Accordingly, the invention provides computers, computer systems, computer readable mediums, computer programs products and the like recorded or stored thereon the nucleic acid and polypeptide sequences of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

Another aspect of the invention is a computer readable medium having recorded thereon at least one nucleic acid and/or polypeptide sequence of the invention. Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

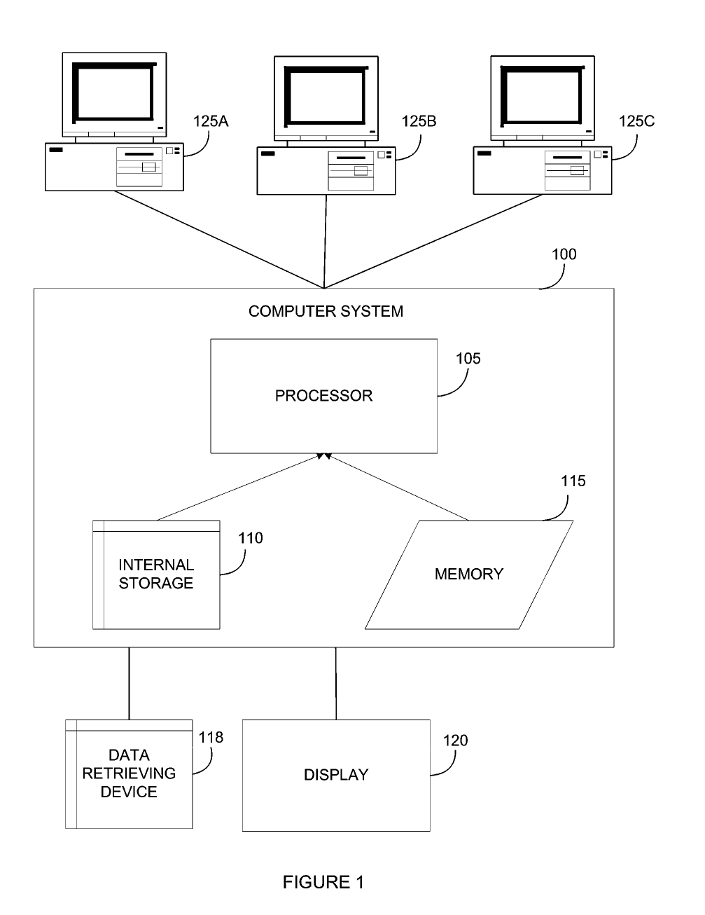

Aspects of the invention include systems (e.g., internet based systems), particularly computer systems, which store and manipulate the sequences and sequence information described herein. One example of a computer system 100 is illustrated in block diagram form in FIG. 1. As used herein, “a computer system” refers to the hardware components, software components, and data storage components used to analyze a nucleotide or polypeptide sequence of the invention. The computer system 100 can include a processor for processing, accessing and manipulating the sequence data. The processor 105 can be any well-known type of central processing unit, such as, for example, the Pentium III from Intel Corporation, or similar processor from Sun, Motorola, Compaq, AMD or International Business Machines. The computer system 100 is a general purpose system that comprises the processor 105 and one or more internal data storage components 110 for storing data, and one or more data retrieving devices for retrieving the data stored on the data storage components. A skilled artisan can readily appreciate that any one of the currently available computer systems are suitable.

In one aspect, the computer system 100 includes a processor 105 connected to a bus which is connected to a main memory 115 (preferably implemented as RAM) and one or more internal data storage devices 110, such as a hard drive and/or other computer readable media having data recorded thereon. The computer system 100 can further include one or more data retrieving device 118 for reading the data stored on the internal data storage devices 110. The data retrieving device 118 may represent, for example, a floppy disk drive, a compact disk drive, a magnetic tape drive, or a modem capable of connection to a remote data storage system (e.g., via the internet) etc. In some embodiments, the internal data storage device 110 is a removable computer readable medium such as a floppy disk, a compact disk, a magnetic tape, etc. containing control logic and/or data recorded thereon. The computer system 100 may advantageously include or be programmed by appropriate software for reading the control logic and/or the data from the data storage component once inserted in the data retrieving device. The computer system 100 includes a display 120 that is used to display output to a computer user. It should also be noted that the computer system 100 can be linked to other computer systems 125a-c in a network or wide area network to provide centralized access to the computer system 100. Software for accessing and processing the nucleotide or amino acid sequences of the invention can reside in main memory 115 during execution. In some aspects, the computer system 100 may further comprise a sequence comparison algorithm for comparing a nucleic acid sequence of the invention. The algorithm and sequence(s) can be stored on a computer readable medium. A “sequence comparison algorithm” refers to one or more programs that are implemented (locally or remotely) on the computer system 100 to compare a nucleotide sequence with other nucleotide sequences and/or compounds stored within a data storage means. For example, the sequence comparison algorithm may compare the nucleotide sequences of the invention stored on a computer readable medium to reference sequences stored on a computer readable medium to identify homologies or structural motifs.

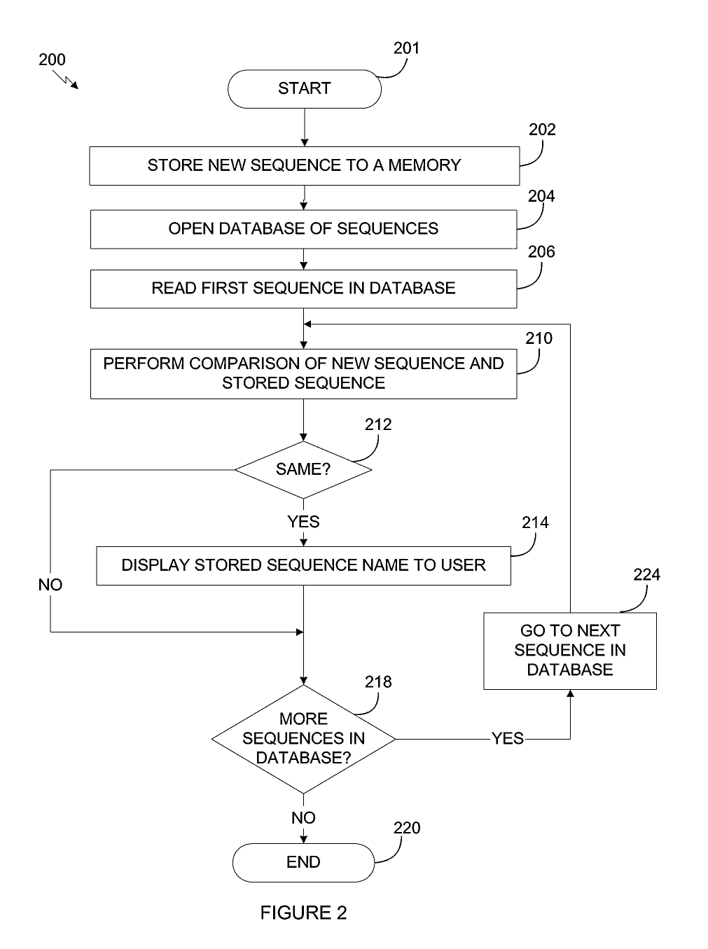

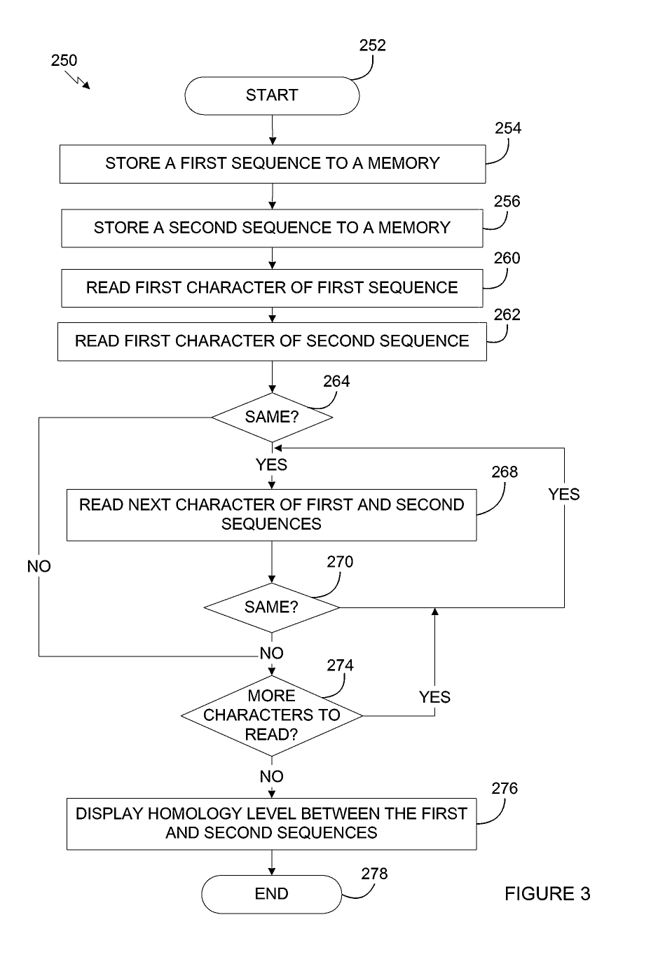

The parameters used with the above algorithms may be adapted depending on the sequence length and degree of homology studied. In some aspects, the parameters may be the default parameters used by the algorithms in the absence of instructions from the user. FIG. 2 is a flow diagram illustrating one aspect of a process 200 for comparing a new nucleotide or protein sequence with a database of sequences in order to determine the homology levels between the new sequence and the sequences in the database. The database of sequences can be a private database stored within the computer system 100, or a public database such as GENBANK that is available through the Internet. The process 200 begins at a start state 201 and then moves to a state 202 wherein the new sequence to be compared is stored to a memory in a computer system 100. As discussed above, the memory could be any type of memory, including RAM or an internal storage device. The process 200 then moves to a state 204 wherein a database of sequences is opened for analysis and comparison. The process 200 then moves to a state 206 wherein the first sequence stored in the database is read into a memory on the computer. A comparison is then performed at a state 210 to determine if the first sequence is the same as the second sequence. It is important to note that this step is not limited to performing an exact comparison between the new sequence and the first sequence in the database. Well-known methods are known to those of skill in the art for comparing two nucleotide or protein sequences, even if they are not identical. For example, gaps can be introduced into one sequence in order to raise the homology level between the two tested sequences. The parameters that control whether gaps or other features are introduced into a sequence during comparison are normally entered by the user of the computer system. Once a comparison of the two sequences has been performed at the state 210, a determination is made at a decision state 210 whether the two sequences are the same. Of course, the term “same” is not limited to sequences that are absolutely identical. Sequences that are within the homology parameters entered by the user will be marked as “same” in the process 200. If a determination is made that the two sequences are the same, the process 200 moves to a state 214 wherein the name of the sequence from the database is displayed to the user. This state notifies the user that the sequence with the displayed name fulfills the homology constraints that were entered. Once the name of the stored sequence is displayed to the user, the process 200 moves to a decision state 218 wherein a determination is made whether more sequences exist in the database. If no more sequences exist in the database, then the process 200 terminates at an end state 220. However, if more sequences do exist in the database, then the process 200 moves to a state 224 wherein a pointer is moved to the next sequence in the database so that it can be compared to the new sequence. In this manner, the new sequence is aligned and compared with every sequence in the database. It should be noted that if a determination had been made at the decision state 212 that the sequences were not homologous, then the process 200 would move immediately to the decision state 218 in order to determine if any other sequences were available in the database for comparison. Accordingly, one aspect of the invention is a computer system comprising a processor, a data storage device having stored thereon a nucleic acid sequence of the invention and a sequence comparer for conducting the comparison. The sequence comparer may indicate a homology level between the sequences compared or identify structural motifs, or it may identify structural motifs in sequences that are compared to these nucleic acid codes and polypeptide codes. FIG. 3 is a flow diagram illustrating one embodiment of a process 250 in a computer for determining whether two sequences are homologous. The process 250 begins at a start state 252 and then moves to a state 254 wherein a first sequence to be compared is stored to a memory. The second sequence to be compared is then stored to a memory at a state 256. The process 250 then moves to a state 260 wherein the first character in the first sequence is read and then to a state 262 wherein the first character of the second sequence is read. It should be understood that if the sequence is a nucleotide sequence, then the character would normally be either A, T, C, G or U. If the sequence is a protein sequence, then it can be a single letter amino acid code so that the first and sequence sequences can be easily compared. A determination is then made at a decision state 264 whether the two characters are the same. If they are the same, then the process 250 moves to a state 268 wherein the next characters in the first and second sequences are read. A determination is then made whether the next characters are the same. If they are, then the process 250 continues this loop until two characters are not the same. If a determination is made that the next two characters are not the same, the process 250 moves to a decision state 274 to determine whether there are any more characters either sequence to read. If there are not any more characters to read, then the process 250 moves to a state 276 wherein the level of homology between the first and second sequences is displayed to the user. The level of homology is determined by calculating the proportion of characters between the sequences that were the same out of the total number of sequences in the first sequence. Thus, if every character in a first 100 nucleotide sequence aligned with an every character in a second sequence, the homology level would be 100%.

Alternatively, the computer program can compare a reference sequence to a sequence of the invention to determine whether the sequences differ at one or more positions. The program can record the length and identity of inserted, deleted or substituted nucleotides or amino acid residues with respect to the sequence of either the reference or the invention. The computer program may be a program that determines whether a reference sequence contains a single nucleotide polymorphism (SNP) with respect to a sequence of the invention, or, whether a sequence of the invention comprises a SNP of a known sequence. Thus, in some aspects, the computer program is a program that identifies SNPs. The method may be implemented by the computer systems described above and the method illustrated in FIG. 3. The method can be performed by reading a sequence of the invention and the reference sequences through the use of the computer program and identifying differences with the computer program.

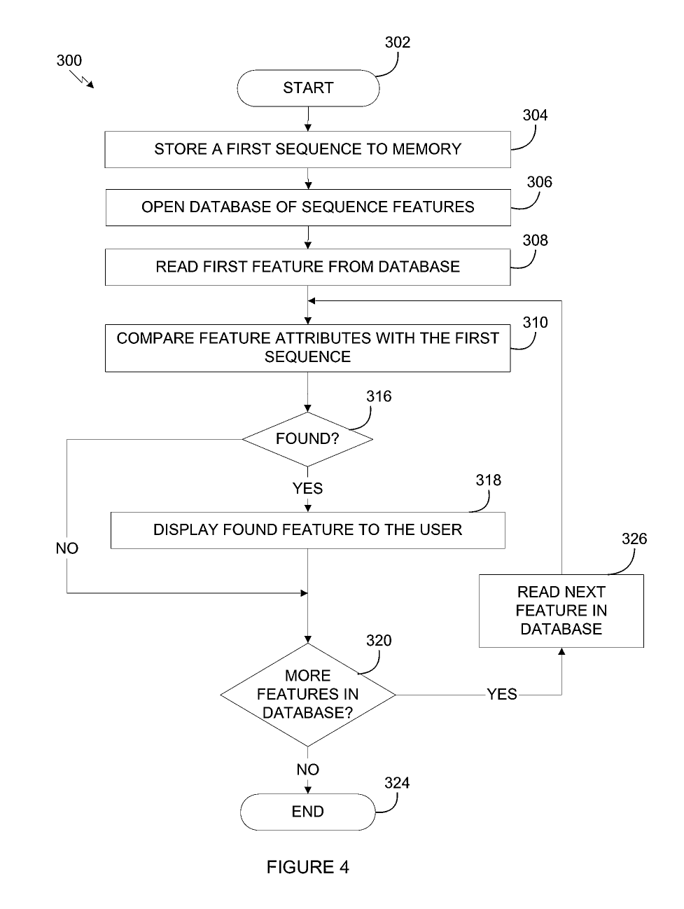

In other aspects the computer based system comprises an identifier for identifying features within a nucleic acid or polypeptide of the invention. An “identifier” refers to one or more programs that identifies certain features within a nucleic acid sequence. For example, an identifier may comprise a program that identifies an open reading frame (ORF) in a nucleic acid sequence. FIG. 4 is a flow diagram illustrating one aspect of an identifier process 300 for detecting the presence of a feature in a sequence. The process 300 begins at a start state 302 and then moves to a state 304 wherein a first sequence that is to be checked for features is stored to a memory 115 in the computer system 100. The process 300 then moves to a state 306 wherein a database of sequence features is opened. Such a database would include a list of each feature's attributes along with the name of the feature. For example, a feature name could be “Initiation Codon” and the attribute would be “ATG”. Another example would be the feature name “TAATAA Box” and the feature attribute would be “TAATAA”. An example of such a database is produced by the University of Wisconsin Genetics Computer Group. Alternatively, the features may be structural polypeptide motifs such as alpha helices, beta sheets, or functional polypeptide motifs such as enzymatic active sites, helix-turn-helix motifs or other motifs known to those skilled in the art. Once the database of features is opened at the state 306, the process 300 moves to a state 308 wherein the first feature is read from the database. A comparison of the attribute of the first feature with the first sequence is then made at a state 310. A determination is then made at a decision state 316 whether the attribute of the feature was found in the first sequence. If the attribute was found, then the process 300 moves to a state 318 wherein the name of the found feature is displayed to the user. The process 300 then moves to a decision state 320 wherein a determination is made whether move features exist in the database. If no more features do exist, then the process 300 terminates at an end state 324. However, if more features do exist in the database, then the process 300 reads the next sequence feature at a state 326 and loops back to the state 310 wherein the attribute of the next feature is compared against the first sequence. If the feature attribute is not found in the first sequence at the decision state 316, the process 300 moves directly to the decision state 320 in order to determine if any more features exist in the database. Thus, in one aspect, the invention provides a computer program that identifies open reading frames (ORFs).

A polypeptide or nucleic acid sequence of the invention may be stored and manipulated in a variety of data processor programs in a variety of formats. For example, a sequence can be stored as text in a word processing file, such as MicrosoftWORD or WORDPERFECT or as an ASCII file in a variety of database programs familiar to those of skill in the art, such as DB2, SYBASE, or ORACLE. In addition, many computer programs and databases may be used as sequence comparison algorithms, identifiers, or sources of reference nucleotide sequences or polypeptide sequences to be compared to a nucleic acid sequence of the invention. The programs and databases used to practice the invention include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, J. Mol. Biol. 215: 403, 1990), FASTA (Pearson and Lipman, Proc. Natl. Acad. Sci. USA, 85: 2444, 1988), FASTDB (Brutlag et al. Comp. App. Biosci. 6:237-245, 1990), Catalyst (Molecular Simulations Inc.), Catalyst/SHAPE (Molecular Simulations Inc.), Cerius2.DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the MDL Available Chemicals Directory database, the MDL Drug Data Report data base, the Comprehensive Medicinal Chemistry database, Derwent's World Drug Index database, the BioByteMasterFile database, the Genbank database, and the Genseqn database. Many other programs and data bases would be apparent to one of skill in the art given the present disclosure.

Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

### Hybridization of Nucleic Acids

The invention provides isolated or recombinant nucleic acids that hybridize under stringent conditions to an exemplary sequence of the invention, e.g., a sequence as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, or SEQ ID NO:25, or a nucleic acid that encodes a polypeptide of the invention. The stringent conditions can be highly stringent conditions, medium stringent conditions, low stringent conditions, including the high and reduced stringency conditions described herein.

In alternative embodiments, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650 residues in length. Nucleic acids shorter than full length are also included. These nucleic acids can be useful as, e.g., hybridization probes, labeling probes, PCR oligonucleotide probes, iRNA, antisense or sequences encoding antibody binding peptides (epitopes), motifs, active sites and the like.

In one aspect, nucleic acids of the invention are defined by their ability to hybridize under high stringency comprises conditions of about 50% formamide at about 37° C. to 42° C. In one aspect, nucleic acids of the invention are defined by their ability to hybridize under reduced stringency comprising conditions in about 35% to 25% formamide at about 30° C. to 35° C.

Alternatively, nucleic acids of the invention are defined by their ability to hybridize under high stringency comprising conditions at 42° C. in 50% formamide, 5×SSPE, 0.3% SDS, and a repetitive sequence blocking nucleic acid, such as cot-1 or salmon sperm DNA (e.g., 200 n/ml sheared and denatured salmon sperm DNA). In one aspect, nucleic acids of the invention are defined by their ability to hybridize under reduced stringency conditions comprising 35% formamide at a reduced temperature of 35° C.

Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

The temperature range corresponding to a particular level of stringency can be further narrowed by calculating the purine to pyrimidine ratio of the nucleic acid of interest and adjusting the temperature accordingly. Nucleic acids of the invention are also defined by their ability to hybridize under high, medium, and low stringency conditions as set forth in Ausubel and Sambrook. Variations on the above ranges and conditions are well known in the art. Hybridization conditions are discussed further, below.

The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na+ concentration of approximately 1M. Following hybridization, the filter may be washed with 2×SSC, 0.5% SDS at the temperature of hybridization. These conditions are considered to be “moderate” conditions above 50° C. and “low” conditions below 50° C. A specific example of “moderate” hybridization conditions is when the above hybridization is conducted at 55° C. A specific example of “low stringency” hybridization conditions is when the above hybridization is conducted at 45° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

However, the selection of a hybridization format is not critical—it is the stringency of the wash conditions that set forth the conditions that determine whether a nucleic acid is within the scope of the invention. Wash conditions used to identify nucleic acids within the scope of the invention include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

These methods may be used to isolate nucleic acids of the invention.

### Oligonucleotides Probes and Methods for Using Them

The invention also provides nucleic acid probes for identifying nucleic acids encoding a polypeptide with a fluorescent activity. In one aspect, the probe comprises at least 10 consecutive bases of a nucleic acid of the invention. Alternatively, a probe of the invention can be at least about 5, 6, 7, 8, 9, 10, 15, 20, 25, 30, 35, 40, 45, 50, 60, 70, 80, 90, 100, 110, 120, 130, 150 or about 10 to 50, about 20 to 60 about 30 to 70, consecutive bases of a sequence as set forth in a nucleic acid of the invention. The probes identify a nucleic acid by binding and/or hybridization. The probes can be used in arrays of the invention, see discussion below, including, e.g., capillary arrays. The probes of the invention can also be used to isolate other nucleic acids or polypeptides.

The probes of the invention can be used to determine whether a biological sample, such as a soil sample, contains an organism having a nucleic acid sequence of the invention or an organism from which the nucleic acid was obtained. In such procedures, a biological sample potentially harboring the organism from which the nucleic acid was isolated is obtained and nucleic acids are obtained from the sample. The nucleic acids are contacted with the probe under conditions that permit the probe to specifically hybridize to any complementary sequences present in the sample. Where necessary, conditions which permit the probe to specifically hybridize to complementary sequences may be determined by placing the probe in contact with complementary sequences from samples known to contain the complementary sequence, as well as control sequences which do not contain the complementary sequence. Hybridization conditions, such as the salt concentration of the hybridization buffer, the formamide concentration of the hybridization buffer, or the hybridization temperature, may be varied to identify conditions which allow the probe to hybridize specifically to complementary nucleic acids (see discussion on specific hybridization conditions).

If the sample contains the organism from which the nucleic acid was isolated, specific hybridization of the probe is then detected. Hybridization may be detected by labeling the probe with a detectable agent such as a radioactive isotope, a fluorescent dye or an enzyme capable of catalyzing the formation of a detectable product. Many methods for using the labeled probes to detect the presence of complementary nucleic acids in a sample are familiar to those skilled in the art. These include Southern Blots, Northern Blots, colony hybridization procedures, and dot blots. Protocols for each of these procedures are provided in Ausubel and Sambrook.

Alternatively, more than one probe (at least one of which is capable of specifically hybridizing to any complementary sequences which are present in the nucleic acid sample), may be used in an amplification reaction to determine whether the sample contains an organism containing a nucleic acid sequence of the invention (e.g., an organism from which the nucleic acid was isolated). In one aspect, the probes comprise oligonucleotides. In one aspect, the amplification reaction may comprise a PCR reaction. PCR protocols are described in Ausubel and Sambrook (see discussion on amplification reactions). In such procedures, the nucleic acids in the sample are contacted with the probes, the amplification reaction is performed, and any resulting amplification product is detected. The amplification product may be detected by performing gel electrophoresis on the reaction products and staining the gel with an intercalator such as ethidium bromide. Alternatively, one or more of the probes may be labeled with a radioactive isotope and the presence of a radioactive amplification product may be detected by autoradiography after gel electrophoresis.

Probes derived from sequences near the 3′ or 5′ ends of a nucleic acid sequence of the invention can also be used in chromosome walking procedures to identify clones containing additional, e.g., genomic sequences. Such methods allow the isolation of genes that encode additional proteins of interest from the host organism.

In one aspect, nucleic acid sequences of the invention are used as probes to identify and isolate related nucleic acids. In some aspects, the so-identified related nucleic acids may be cDNAs or genomic DNAs from organisms other than the one from which the nucleic acid of the invention was first isolated. In such procedures, a nucleic acid sample is contacted with the probe under conditions that permit the probe to specifically hybridize to related sequences. Hybridization of the probe to nucleic acids from the related organism is then detected using any of the methods described above.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter. Hybridization may be carried out under conditions of low stringency, moderate stringency or high stringency. As an example of nucleic acid hybridization, a polymer membrane containing immobilized denatured nucleic acids is first prehybridized for 30 minutes at 45° C. in a solution consisting of 0.9 M NaCl, 50 mM NaH₂PO₄, pH 7.0, 5.0 mM Na₂EDTA, 0.5% SDS, 10×Denhardt's, and 0.5 mg/ml polyriboadenylic acid. Approximately 2×10⁷ cpm (specific activity 4−9×108 cpm/ug) of ³²P end-labeled oligonucleotide probe are then added to the solution. After 12-16 hours of incubation, the membrane is washed for 30 minutes at room temperature (RT) in 1×SET (150 mM NaCl, 20 mM Tris hydrochloride, pH 7.8, 1 mM Na₂EDTA) containing 0.5% SDS, followed by a 30 minute wash in fresh 1×SET at Tm-10° C. for the oligonucleotide probe. The membrane is then exposed to auto-radiographic film for detection of hybridization signals.

By varying the stringency of the hybridization conditions used to identify nucleic acids, such as cDNAs or genomic DNAs, which hybridize to the detectable probe, nucleic acids having different levels of homology to the probe can be identified and isolated. Stringency may be varied by conducting the hybridization at varying temperatures below the melting temperatures of the probes. The melting temperature, Tm, is the temperature (under defined ionic strength and pH) at which 50% of the target sequence hybridizes to a perfectly complementary probe. Very stringent conditions are selected to be equal to or about 5° C. lower than the Tm for a particular probe. The melting temperature of the probe may be calculated using the following exemplary formulas. For probes between 14 and 70 nucleotides in length the melting temperature (Tm) is calculated using the formula: Tm=81.5+16.6(log [Na⁺])+0.41(fraction G+C)−(600/N) where N is the length of the probe. If the hybridization is carried out in a solution containing formamide, the melting temperature may be calculated using the equation: Tm=81.5+16.6(log [Na+])+0.41(fraction G+C)−(0.63% formamide)−(600/N) where N is the length of the probe. Prehybridization may be carried out in 6×SSC, 5×Denhardt's reagent, 0.5% SDS, 100 μg denatured fragmented salmon sperm DNA or 6×SSC, 5×Denhardt's reagent, 0.5% SDS, 100 μg denatured fragmented salmon sperm DNA, 50% formamide. Formulas for SSC and Denhardt's and other solutions are listed, e.g., in Sambrook.

Hybridization is conducted by adding the detectable probe to the prehybridization solutions listed above. Where the probe comprises double stranded DNA, it is denatured before addition to the hybridization solution. The filter is contacted with the hybridization solution for a sufficient period of time to allow the probe to hybridize to cDNAs or genomic DNAs containing sequences complementary thereto or homologous thereto. For probes over 200 nucleotides in length, the hybridization may be carried out at 15-25° C. below the Tm. For shorter probes, such as oligonucleotide probes, the hybridization may be conducted at 5-10° C. below the Tm. In one aspect, hybridizations in 6×SSC are conducted at approximately 68° C. In one aspect, hybridizations in 50% formamide containing solutions are conducted at approximately 42° C. All of the foregoing hybridizations would be considered to be under conditions of high stringency.

Following hybridization, the filter is washed to remove any non-specifically bound detectable probe. The stringency used to wash the filters can also be varied depending on the nature of the nucleic acids being hybridized, the length of the nucleic acids being hybridized, the degree of complementarity, the nucleotide sequence composition (e.g., GC v. AT content), and the nucleic acid type (e.g., RNA v. DNA). Examples of progressively higher stringency condition washes are as follows: 2×SSC, 0.1% SDS at room temperature for 15 minutes (low stringency); 0.1×SSC, 0.5% SDS at room temperature for 30 minutes to 1 hour (moderate stringency); 0.1×SSC, 0.5% SDS for 15 to 30 minutes at between the hybridization temperature and 68° C. (high stringency); and 0.15M NaCl for 15 minutes at 72° C. (very high stringency). A final low stringency wash can be conducted in 0.1×SSC at room temperature. The examples above are merely illustrative of one set of conditions that can be used to wash filters. One of skill in the art would know that there are numerous recipes for different stringency washes.

Nucleic acids that have hybridized to the probe can be identified by autoradiography or other conventional techniques. The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na⁺ concentration of approximately 1M. Following hybridization, the filter may be washed with 2×SSC, 0.5% SDS at the temperature of hybridization. These conditions are considered to be “moderate” conditions above 50° C. and “low” conditions below 50° C. An example of “moderate” hybridization conditions is when the above hybridization is conducted at 55° C. An example of “low stringency” hybridization conditions is when the above hybridization is conducted at 45° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

These probes and methods of the invention can be used to isolate nucleic acids having a sequence with at least about 99%, 98%, 97%, at least 95%, at least 90%, at least 85%, at least 80%, at least 75%, at least 70%, at least 65%, at least 60%, at least 55%, or at least 50% homology to a nucleic acid sequence of the invention comprising at least about 10, 15, 20, 25, 30, 35, 40, 50, 75, 100, 150, 200, 250, 300, 350, 400, or 500 consecutive bases thereof, and the sequences complementary thereto. Homology may be measured using an alignment algorithm, as discussed herein. For example, the homologous polynucleotides may have a coding sequence that is a naturally occurring allelic variant of one of the coding sequences described herein. Such allelic variants may have a substitution, deletion or addition of one or more nucleotides when compared to a nucleic acid of the invention.

Additionally, the probes and methods of the invention may be used to isolate nucleic acids which encode polypeptides having at least about 99%, at least 95%, at least 90%, at least 85%, at least 80%, at least 75%, at least 70%, at least 65%, at least 60%, at least 55%, or at least 50% sequence identity (homology) to a polypeptide of the invention comprising at least 5, 10, 15, 20, 25, 30, 35, 40, 50, 75, 100, or 150 consecutive amino acids thereof as determined using a sequence alignment algorithm (e.g., such as the FASTA version 3.0t78 algorithm with the default parameters, or a BLAST 2.2.2 program with exemplary settings as set forth herein).

Inhibiting Expression of Fluorescent Polypeptide

The invention further provides for nucleic acids complementary to (e.g., antisense sequences to) the nucleic acid sequences of the invention. Antisense sequences are capable of inhibiting the transport, splicing or transcription of fluorescent protein-encoding genes. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides that are able to either bind fluorescent protein gene or message, in either case preventing or inhibiting the production or function of fluorescent protein. The association can be through sequence specific hybridization. Another useful class of inhibitors includes oligonucleotides that cause inactivation or cleavage of fluorescent protein message. The oligonucleotide can have enzyme activity that causes such cleavage, such as ribozymes. The oligonucleotide can be chemically modified or conjugated to an enzyme or composition capable of cleaving the complementary nucleic acid. One may screen a pool of many different such oligonucleotides for those with the desired activity.

Antisense Oligonucleotides

The invention provides antisense oligonucleotides capable of binding fluorescent polypeptide message that can inhibit fluorescent activity by targeting mRNA. Strategies for designing antisense oligonucleotides are well described in the scientific and patent literature, and the skilled artisan can design such fluorescent oligonucleotides using the novel reagents of the invention. For example, gene walking/RNA mapping protocols to screen for effective antisense oligonucleotides are well known in the art, see, e.g., Ho (2000) Methods Enzymol. 314:168-183, describing an RNA mapping assay, which is based on standard molecular techniques to provide an easy and reliable method for potent antisense sequence selection. See also Smith (2000) Eur. J. Pharm. Sci. 11:191-198.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense fluorescent polypeptides sequences of the invention (see, e.g., Gold (1995) J. of Biol. Chem. 270:13581-13584).

Inhibitory Ribozymes

The invention provides for with ribozymes capable of binding fluorescent message that can inhibit fluorescent polypeptide activity by targeting mRNA. Strategies for designing ribozymes and selecting the fluorescent protein-specific antisense sequence for targeting are well described in the scientific and patent literature, and the skilled artisan can design such ribozymes using the novel reagents of the invention. Ribozymes act by binding to a target RNA through the target RNA binding portion of a ribozyme that is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but may also be formed in the motif of a hairpin, hepatitis delta virus, group I intron or RNaseP-like RNA (in association with an RNA guide sequence). Examples of such hammerhead motifs are described by Rossi (1992) Aids Research and Human Retroviruses 8:183; hairpin motifs by Hampel (1989) Biochemistry 28:4929, and Hampel (1990) Nuc. Acids Res. 18:299; the hepatitis delta virus motif by Perrotta (1992) Biochemistry 31:16; the RNaseP motif by Guerrier-Takada (1983) Cell 35:849; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

RNA interference (RNAi)

In one aspect, the invention provides an RNA inhibitory molecule, a so-called “RNAi” molecule, comprising a sequence of the invention. The RNAi molecule comprises a double-stranded RNA (dsRNA) molecule. The RNAi can inhibit expression of a sequence of the invention, e.g., a fluorescent protein gene, such as a green fluorescent protein gene. In one aspect, the RNAi is about 15, 16, 17, 18, 19, 20, 21, 22, 23, 24, 25 or more duplex nucleotides in length. While the invention is not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi is the breaking of a double-stranded RNA (dsRNA) matching a specific gene sequence into short pieces called short interfering RNA, which trigger the degradation of mRNA that matches its sequence. In one aspect, the RNAi's of the invention are used in gene-silencing therapeutics, see, e.g., Shuey (2002) Drug Discov. Today 7:1040-1046. In one aspect, the invention provides methods to selectively degrade RNA using the RNAi's of the invention. The process may be practiced in vitro, ex vivo or in vivo. In one aspect, the RNAi molecules of the invention can be used to generate a loss-of-function mutation in a cell, an organ or an animal. Methods for making and using RNAi molecules for selectively degrade RNA are well known in the art, see, e.g., U.S. Pat. Nos. 6,506,559; 6,511,824; 6,515,109; 6,489,127.

### Modification of Nucleic Acids

The invention provides methods of generating variants of the nucleic acids of the invention, e.g., those encoding a fluorescent polypeptide. These methods can be repeated or used in various combinations to generate fluorescent polypeptides having an altered or different activity or an altered or different stability from that of a fluorescent polypeptide encoded by the template nucleic acid. These methods also can be repeated or used in various combinations, e.g., to generate variations in gene/message expression, message translation or message stability. In another aspect, the genetic composition of a cell is altered by, e.g., modification of a homologous gene ex vivo, followed by its reinsertion into the cell.

A nucleic acid of the invention can be altered by any means. For example, random or stochastic methods, or, non-stochastic, or “directed evolution,” methods, see, e.g., U.S. Pat. No. 6,361,974. Methods for random mutation of genes are well known in the art, see, e.g., U.S. Pat. No. 5,830,696. For example, mutagens can be used to randomly mutate a gene. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used.

Any technique in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793. In alternative aspects, modifications, additions or deletions are introduced by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM™), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

The following publications describe a variety of recursive recombination procedures and/or methods which can be incorporated into the methods of the invention: Stemmer (1999) “Molecular breeding of viruses for targeting and other clinical properties” Tumor Targeting 4:1-4; Ness (1999) Nature Biotechnology 17:893-896; Chang (1999) “Evolution of a cytokine using DNA family shuffling” Nature Biotechnology 17:793-797; Minshull (1999) “Protein evolution by molecular breeding” Current Opinion in Chemical Biology 3:284-290; Christians (1999) “Directed evolution of thymidine kinase for AZT phosphorylation using DNA family shuffling” Nature Biotechnology 17:259-264; Crameri (1998) “DNA shuffling of a family of genes from diverse species accelerates directed evolution” Nature 391:288-291; Crameri (1997) “Molecular evolution of an arsenate detoxification pathway by DNA shuffling,” Nature Biotechnology 15:436-438; Zhang (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp. 447-45′7; Crameri and Stemmer (1995) “Combinatorial multiple cassette mutagenesis creates all the permutations of mutant and wildtype cassettes” BioTechniques 18:194-195; Stemmer et al. (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional protocols used in the methods of the invention include point mismatch repair (Kramer (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and 0 et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

See also U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Certain U.S. applications provide additional details regarding various diversity generating methods, including “SHUFFLING OF CODON ALTERED GENES” by Patten et al. filed Sep. 28, 1999, (U.S. Ser. No. 09/407,800); “EVOLUTION OF WHOLE CELLS AND ORGANISMS BY RECURSIVE SEQUENCE RECOMBINATION” by del Cardayre et al., filed Jul. 15, 1998 (U.S. Ser. No. 09/166,188), and Jul. 15, 1999 (U.S. Ser. No. 09/354,922); “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-VARIED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g. “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549).

Non-stochastic, or “directed evolution,” methods include, e.g., saturation mutagenesis (GSSM™), synthetic ligation reassembly (SLR), or a combination thereof are used to modify the nucleic acids of the invention to generate fluorescent polypeptides with new or altered properties (e.g., activity under highly acidic or alkaline conditions, high temperatures, and the like). Polypeptides encoded by the modified nucleic acids can be screened for an activity before testing for fluorescence or other activity. Any testing modality or protocol can be used, e.g., using a capillary array platform. See, e.g., U.S. Pat. Nos. 6,361,974; 6,280,926; 5,939,250.

Saturation Mutagenesis, or, GSSM™

In one aspect of the invention, non-stochastic gene modification, a “directed evolution process,” is used to generate fluorescent polypeptides with new or altered properties. Variations of this method have been termed “gene site-saturation mutagenesis,” “site-saturation mutagenesis,” “saturation mutagenesis” or simply “GSSM™.” It can be used in combination with other mutagenization processes. See, e.g., U.S. Pat. Nos. 6,171,820; 6,238,884. In one aspect, GSSM™ comprises providing a template polynucleotide and a plurality of oligonucleotides, wherein each oligonucleotide comprises a sequence homologous to the template polynucleotide, thereby targeting a specific sequence of the template polynucleotide, and a sequence that is a variant of the homologous gene; generating progeny polynucleotides comprising non-stochastic sequence variations by replicating the template polynucleotide with the oligonucleotides, thereby generating polynucleotides comprising homologous gene sequence variations.

In one aspect, codon primers containing a degenerate N,N,G/T sequence are used to introduce point mutations into a polynucleotide, so as to generate a set of progeny polypeptides in which a full range of single amino acid substitutions is represented at each amino acid position, e.g., an amino acid residue in an enzyme active site or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids. In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used—either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position×100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide (e.g., fluorescent polypeptides) molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual progeny polypeptide is identified by screening to display a favorable change in property (when compared to the parental polypeptide, such as increased fluorescent activity under alkaline or acidic conditions), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined—6 single point mutations (i.e. 2 at each of three positions) and no change at any position.

In another aspect, site-saturation mutagenesis can be used together with another stochastic or non-stochastic means to vary sequence, e.g., synthetic ligation reassembly (see below), shuffling, chimerization, recombination and other mutagenizing processes and mutagenizing agents. This invention provides for the use of any mutagenizing process(es), including saturation mutagenesis, in an iterative manner.

Synthetic Ligation Reassembly (SLR)

The invention provides a non-stochastic gene modification system termed “synthetic ligation reassembly,” or simply “SLR,” a “directed evolution process,” to generate fluorescent polypeptides with new or altered properties. SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. See, e.g., U.S. patent application Ser. No. 09/332,835 entitled “Synthetic Ligation Reassembly in Directed Evolution” and filed on Jun. 14, 1999 (“U.S. Ser. No. 09/332,835”). In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10¹⁰⁰ different chimeras. SLR can be used to generate libraries comprised of over 10¹⁰⁰⁰ different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus, the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g. T4 DNA ligase), to achieve covalent bonding of the building pieces.

In one aspect, the design of the oligonucleotide building blocks is obtained by analyzing a set of progenitor nucleic acid sequence templates that serve as a basis for producing a progeny set of finalized chimeric polynucleotides. These parental oligonucleotide templates thus serve as a source of sequence information that aids in the design of the nucleic acid building blocks that are to be mutagenized, e.g., chimerized or shuffled. In one aspect of this method, the sequences of a plurality of parental nucleic acid templates are aligned in order to select one or more demarcation points. The demarcation points can be located at an area of homology, and are comprised of one or more nucleotides. These demarcation points are preferably shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (comprised of at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Even more preferably a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

In one aspect, a ligation reassembly process is performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another aspect, the assembly order (i.e. the order of assembly of each building block in the 5′ to 3 sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g. one by one. In other words this invention provides that, through the selective and judicious use of specific nucleic acid building blocks, coupled with the selective and judicious use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups. Because of its ability to perform chimerizations in a manner that is highly flexible yet exhaustive and systematic as well, particularly when there is a low level of homology among the progenitor molecules, these methods provide for the generation of a library (or set) comprised of a large number of progeny molecules. Because of the non-stochastic nature of the instant ligation reassembly invention, the progeny molecules generated preferably comprise a library of finalized chimeric nucleic acid molecules having an overall assembly order that is chosen by design. The saturation mutagenesis and optimized directed evolution methods also can be used to generate different progeny molecular species. It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the operability of this invention. In fact, demarcation points can even be chosen in areas of little or no intermolecular homology. For example, because of codon wobble, i.e. the degeneracy of codons, nucleotide substitutions can be introduced into nucleic acid building blocks without altering the amino acid originally encoded in the corresponding progenitor template. Alternatively, a codon can be altered such that the coding for an originally amino acid is altered. This invention provides that such substitutions can be introduced into the nucleic acid building block in order to increase the incidence of intermolecularly homologous demarcation points and thus to allow an increased number of couplings to be achieved among the building blocks, which in turn allows a greater number of progeny chimeric molecules to be generated.

In another aspect, the synthetic nature of the step in which the building blocks are generated allows the design and introduction of nucleotides (e.g., one or more nucleotides, which may be, for example, codons or introns or regulatory sequences) that can later be optionally removed in an in vitro process (e.g. by mutagenesis) or in an in vivo process (e.g. by utilizing the gene splicing ability of a host organism). It is appreciated that in many instances the introduction of these nucleotides may also be desirable for many other reasons in addition to the potential benefit of creating a serviceable demarcation point.

In one aspect, a nucleic acid building block is used to introduce an intron. Thus, functional introns are introduced into a man-made gene manufactured according to the methods described herein. The artificially introduced intron(s) can be functional in a host cells for gene splicing much in the way that naturally-occurring introns serve functionally in gene splicing.

Optimized Directed Evolution System

The invention provides a non-stochastic gene modification system termed “optimized directed evolution system” to generate fluorescent polypeptides with new or altered properties. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events.

A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

In addition, this method provides a convenient means for exploring a tremendous amount of the possible protein variant space in comparison to other systems. Previously, if one generated, for example, 10¹³ chimeric molecules during a reaction, it would be extremely difficult to test such a high number of chimeric variants for a particular activity. Moreover, a significant portion of the progeny population would have a very high number of crossover events that resulted in proteins that were less likely to have increased levels of a particular activity. By using these methods, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

One method for creating a chimeric progeny polynucleotide sequence is to create oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. Additional information can also be found, e.g., in U.S. Ser. No. 09/332,835; U.S. Pat. No. 6,361,974. The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. By utilizing these methods, one can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events. These methods are directed to the use of repeated cycles of reductive reassortment, recombination and selection that allow for the directed molecular evolution of a nucleic acid encoding a polypeptide through recombination. This system allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. The method allows calculation of the correct concentrations of oligonucleotide sequences so that the final chimeric population of sequences is enriched for the chosen number of crossover events. This provides more control over choosing chimeric variants having a predetermined number of crossover events.

In addition, these methods provide a convenient means for exploring a tremendous amount of the possible protein variant space in comparison to other systems. By using the methods described herein, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

In one aspect, the method creates a chimeric progeny polynucleotide sequence by creating oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. See also U.S. Ser. No. 09/332,835.

The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. One can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events.

Determining Crossover Events

Aspects of the invention include a system and software that receive a desired crossover probability density function (PDF), the number of parent genes to be reassembled, and the number of fragments in the reassembly as inputs. The output of this program is a “fragment PDF” that can be used to determine a recipe for producing reassembled genes, and the estimated crossover PDF of those genes. The processing described herein is preferably performed in MATLAB® (The Mathworks, Natick, Mass.) a programming language and development environment for technical computing.

Iterative Processes

In practicing the invention, these processes can be iteratively repeated. For example a nucleic acid (or, the nucleic acid) responsible for an altered fluorescent polypeptide phenotype is identified, re-isolated, again modified, re-tested for activity. This process can be iteratively repeated until a desired phenotype is engineered. For example, an entire biochemical anabolic or catabolic pathway can be engineered into a cell, including fluorescent activity.

Similarly, if it is determined that a particular oligonucleotide has no affect at all on the desired trait (e.g., a new fluorescent phenotype), it can be removed as a variable by synthesizing larger parental oligonucleotides that include the sequence to be removed. Since incorporating the sequence within a larger sequence prevents any crossover events, there will no longer be any variation of this sequence in the progeny polynucleotides. This iterative practice of determining which oligonucleotides are most related to the desired trait, and which are unrelated, allows more efficient exploration all of the possible protein variants that might be provide a particular trait or activity.

In Vivo Shuffling

In vivo shuffling of molecules is use in methods of the invention that provide variants of polypeptides of the invention, e.g., antibodies, fluorescent polypeptides, and the like. In vivo shuffling can be performed utilizing the natural property of cells to recombine multimers. While recombination in vivo has provided the major natural route to molecular diversity, genetic recombination remains a relatively complex process that involves 1) the recognition of homologies; 2) strand cleavage, strand invasion, and metabolic steps leading to the production of recombinant chiasma; and finally 3) the resolution of chiasma into discrete recombined molecules. The formation of the chiasma requires the recognition of homologous sequences.

In one aspect, the invention provides a method for producing a hybrid polynucleotide from at least a first polynucleotide and a second polynucleotide. The invention can be used to produce a hybrid polynucleotide by introducing at least a first polynucleotide and a second polynucleotide that share at least one region of partial sequence homology into a suitable host cell. The regions of partial sequence homology promote processes that result in sequence reorganization producing a hybrid polynucleotide. The term “hybrid polynucleotide”, as used herein, is any nucleotide sequence that results from the method of the present invention and contains sequence from at least two original polynucleotide sequences. Such hybrid polynucleotides can result from intermolecular recombination events that promote sequence integration between DNA molecules. In addition, such hybrid polynucleotides can result from intramolecular reductive reassortment processes that utilize repeated sequences to alter a nucleotide sequence within a DNA molecule.

Producing Sequence Variants

The invention also provides methods of making sequence variants of the nucleic acid and fluorescent polypeptide sequences of the invention or isolating fluorescent polypeptides, e.g., green fluorescent protein sequence variants using the nucleic acids and polypeptides of the invention. In one aspect, the invention provides for variants of a fluorescent polypeptide gene of the invention, which can be altered by any means, including, e.g., random or stochastic methods, or, non-stochastic, or “directed evolution,” methods, as described above.

The isolated variants may be naturally occurring. Variant can also be created in vitro. Variants may be created using genetic engineering techniques such as site directed mutagenesis, random chemical mutagenesis, Exonuclease III deletion procedures, and standard cloning techniques. Alternatively, such variants, fragments, analogs, or derivatives may be created using chemical synthesis or modification procedures. Other methods of making variants are also familiar to those skilled in the art. These include procedures in which nucleic acid sequences obtained from natural isolates are modified to generate nucleic acids that encode polypeptides having characteristics that enhance their value in industrial or laboratory applications. In such procedures, a large number of variant sequences having one or more nucleotide differences with respect to the sequence obtained from the natural isolate are generated and characterized. These nucleotide differences can result in amino acid changes with respect to the polypeptides encoded by the nucleic acids from the natural isolates.

For example, variants may be created using error prone PCR. In error prone PCR, PCR is performed under conditions where the copying fidelity of the DNA polymerase is low, such that a high rate of point mutations is obtained along the entire length of the PCR product. Error prone PCR is described, e.g., in Leung, D. W., et al., Technique, 1:11-15, 1989) and Caldwell, R. C. & Joyce G. F., PCR Methods Applic., 2:28-33, 1992. Briefly, in such procedures, nucleic acids to be mutagenized are mixed with PCR primers, reaction buffer, MgCl₂, MnCl₂, Taq polymerase and an appropriate concentration of dNTPs for achieving a high rate of point mutation along the entire length of the PCR product. For example, the reaction may be performed using 20 fmoles of nucleic acid to be mutagenized, 30 pmole of each PCR primer, a reaction buffer comprising 50 mM KCl, 10 mM Tris HCl (pH 8.3) and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR may be performed for 30 cycles of 94° C. for 1 min, 45° C. for 1 min, and 72° C. for 1 min. However, it will be appreciated that these parameters may be varied as appropriate. The mutagenized nucleic acids are cloned into an appropriate vector and the activities of the polypeptides encoded by the mutagenized nucleic acids is evaluated.

Variants may also be created using oligonucleotide directed mutagenesis to generate site-specific mutations in any cloned DNA of interest. Oligonucleotide mutagenesis is described, e.g., in Reidhaar-Olson (1988) Science 241:53-57. Briefly, in such procedures a plurality of double stranded oligonucleotides bearing one or more mutations to be introduced into the cloned DNA are synthesized and inserted into the cloned DNA to be mutagenized. Clones containing the mutagenized DNA are recovered and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, e.g., U.S. Pat. No. 5,965,408.

Still another method of generating variants is sexual PCR mutagenesis. In sexual PCR mutagenesis, forced homologous recombination occurs between DNA molecules of different but highly related DNA sequence in vitro, as a result of random fragmentation of the DNA molecule based on sequence homology, followed by fixation of the crossover by primer extension in a PCR reaction. Sexual PCR mutagenesis is described, e.g., in Stemmer (1994) Proc. Natl. Acad. Sci. USA 91:10747-10751. Briefly, in such procedures a plurality of nucleic acids to be recombined are digested with DNase to generate fragments having an average size of 50-200 nucleotides. Fragments of the desired average size are purified and resuspended in a PCR mixture. PCR is conducted under conditions that facilitate recombination between the nucleic acid fragments. For example, PCR may be performed by resuspending the purified fragments at a concentration of 10-30 ng/:1 in a solution of 0.2 mM of each dNTP, 2.2 mM MgCl₂, 50 mM KCL, 10 mM Tris HCl, pH 9.0, and 0.1% Triton X-100. 2.5 units of Taq polymerase per 100:1 of reaction mixture is added and PCR is performed using the following regime: 94° C. for 60 seconds, 94° C. for 30 seconds, 50-55° C. for 30 seconds, 72° C. for 30 seconds (30-45 times) and 72° C. for 5 minutes. However, it will be appreciated that these parameters may be varied as appropriate. In some aspects, oligonucleotides may be included in the PCR reactions. In other aspects, the Klenow fragment of DNA polymerase I may be used in a first set of PCR reactions and Taq polymerase may be used in a subsequent set of PCR reactions. Recombinant sequences are isolated and the activities of the polypeptides they encode are assessed.

Variants may also be created by in vivo mutagenesis. In some aspects, random mutations in a sequence of interest are generated by propagating the sequence of interest in a bacterial strain, such as an E. coli strain, which carries mutations in one or more of the DNA repair pathways. Such “mutator” strains have a higher random mutation rate than that of a wild-type parent. Propagating the DNA in one of these strains will eventually generate random mutations within the DNA. Mutator strains suitable for use for in vivo mutagenesis are described, e.g., in PCT Publication No. WO 91/16427.

Variants may also be generated using cassette mutagenesis. In cassette mutagenesis a small region of a double stranded DNA molecule is replaced with a synthetic oligonucleotide “cassette” that differs from the native sequence. The oligonucleotide often contains completely and/or partially randomized native sequence.

Recursive ensemble mutagenesis may also be used to generate variants. Recursive ensemble mutagenesis is an algorithm for protein engineering (protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described, e.g., in Arkin (1992) Proc. Natl. Acad. Sci. USA 89:7811-7815.

In some aspects, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described, e.g., in Delegrave (1993) Biotechnology Res. 11:1548-1552. Random and site-directed mutagenesis are described, e.g., in Arnold (1993) Current Opinion in Biotechnology 4:450-455.

In some aspects, the variants are created using shuffling procedures wherein portions of a plurality of nucleic acids which encode distinct polypeptides are fused together to create chimeric nucleic acid sequences which encode chimeric polypeptides as described in, e.g., U.S. Pat. Nos. 5,965,408; 5,939,250.

The invention also provides variants of polypeptides of the invention comprising sequences in which one or more of the amino acid residues (e.g., of an exemplary polypeptide, such as SEQ ID NO:2) are substituted with a conserved or non-conserved amino acid residue (e.g., a conserved amino acid residue) and such substituted amino acid residue may or may not be one encoded by the genetic code. Conservative substitutions are those that substitute a given amino acid in a polypeptide by another amino acid of like characteristics. Thus, polypeptides of the invention include those with conservative substitutions of sequences of the invention, e.g., the exemplary SEQ ID NO:2, including but not limited to the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue. Other variants are those in which one or more of the amino acid residues of the polypeptides of the invention includes a substituent group.

Other variants within the scope of the invention are those in which the polypeptide is associated with another compound, such as a compound to increase the half-life of the polypeptide, for example, polyethylene glycol.

Additional variants within the scope of the invention are those in which additional amino acids are fused to the polypeptide, such as a leader sequence, a secretory sequence, a proprotein sequence or a sequence which facilitates purification, enrichment, or stabilization of the polypeptide.

In some aspects, the variants, fragments, derivatives and analogs of the polypeptides of the invention retain the same biological function or activity as the exemplary polypeptides, e.g., a fluorescent activity, as described herein. In other aspects, the variant, fragment, derivative, or analog includes a proprotein, such that the variant, fragment, derivative, or analog can be activated by cleavage of the proprotein portion to produce an active polypeptide.

### Optimizing Codons to Achieve High Levels of Protein Expression in Host Cells

The invention provides methods for modifying fluorescent protein-encoding nucleic acids to modify codon usage. In one aspect, the invention provides methods for modifying codons in a nucleic acid encoding a fluorescent polypeptide to increase or decrease its expression in a host cell. The invention also provides nucleic acids encoding a fluorescent polypeptide modified to increase its expression in a host cell, fluorescent polypeptides so modified, and methods of making the modified fluorescent polypeptides. The method comprises identifying a “non-preferred” or a “less preferred” codon in fluorescent protein-encoding nucleic acid and replacing one or more of these non-preferred or less preferred codons with a “preferred codon” encoding the same amino acid as the replaced codon and at least one non-preferred or less preferred codon in the nucleic acid has been replaced by a preferred codon encoding the same amino acid. A preferred codon is a codon over-represented in coding sequences in genes in the host cell and a non-preferred or less preferred codon is a codon under-represented in coding sequences in genes in the host cell.

Host cells for expressing the nucleic acids, expression cassettes and vectors of the invention include bacteria, yeast, fungi, plant cells, insect cells and mammalian cells. Thus, the invention provides methods for optimizing codon usage in all of these cells, codon-altered nucleic acids and polypeptides made by the codon-altered nucleic acids. Exemplary host cells include gram negative bacteria, such as Escherichia coli and Pseudomonas fluorescens; gram positive bacteria, such as Streptomyces diversa, Lactobacillus gasseri, Lactococcus lactis, Lactococcus cremoris, Bacillus subtilis. Exemplary host cells also include eukaryotic organisms, e.g., various yeast, such as Saccharomyces sp., including Saccharomyces cerevisiae, Schizosaccharomyces pombe, Pichia pastoris, and Kluyveromyces lactis, Hansenula polymorpha, Aspergillus niger, and mammalian cells and cell lines and insect cells and cell lines. Thus, the invention also includes nucleic acids and polypeptides optimized for expression in these organisms and species.

For example, the codons of a nucleic acid encoding a fluorescent polypeptide isolated from a bacterial cell are modified such that the nucleic acid is optimally expressed in a bacterial cell different from the bacteria from which the fluorescent polypeptide was derived, a yeast, a fungi, a plant cell, an insect cell or a mammalian cell. Methods for optimizing codons are well known in the art, see, e.g., U.S. Pat. No. 5,795,737; Baca (2000) Int. J. Parasitol. 30:113-118; Hale (1998) Protein Expr. Purif. 12:185-188; Narum (2001) Infect. Immun. 69:7250-7253. See also Narum (2001) Infect. Immun. 69:7250-7253, describing optimizing codons in mouse systems; Outchkourov (2002) Protein Expr. Purif. 24:18-24, describing optimizing codons in yeast; Feng (2000) Biochemistry 39:15399-15409, describing optimizing codons in E. coli; Humphreys (2000) Protein Expr. Purif. 20:252-264, describing optimizing codon usage that affects secretion in E. coli.

### Transgenic Non-Human Animals

The invention provides transgenic non-human animals comprising a nucleic acid, a polypeptide, an expression cassette or vector or a transfected or transformed cell of the invention. The transgenic non-human animals can be, e.g., fish, goats, rabbits, sheep, pigs, cows, rats and mice, comprising the nucleic acids of the invention. These animals can be used, e.g., as in vivo models to study fluorescent activity, or, as models to screen for agents that change the fluorescent activity in vivo. The coding sequences for the polypeptides to be expressed in the transgenic non-human animals can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, describes making and using transgenic non-human mammals that express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice whose cells express proteins related to the pathology of Alzheimer's disease. U.S. Pat. No. 6,187,992, describes making and using a transgenic mouse whose genome comprises a disruption of the gene encoding amyloid precursor protein (APP).

U.S. Pat. Nos. 5,998,697; 5,998,698; 6,015,713; 6,307,121 and 6,472,583, describe making transgenic fish. See also, Kinoshita (2003) Zoolog Sci. 2:869-875, that describes making a transgenic medaka (Oryzias latipes) containing a green fluorescent protein (GFP) gene controlled by a medaka beta-actin promoter; and, Long (1997) Development 124:4105-4111, that describes making a fluorescent protein-expressing transgenic fish.

“Knockout animals” can also be used to practice the methods of the invention. For example, in one aspect, the transgenic or modified animals of the invention comprise a “knockout animal,” e.g., a “knockout mouse,” engineered not to express an endogenous gene, which is replaced with a gene expressing a fluorescent polypeptide of the invention, or, a fusion protein comprising a fluorescent polypeptide of the invention.

Polypeptides and Peptides

The invention provides isolated or recombinant polypeptides having a sequence identity (e.g., at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity) to an exemplary sequence of the invention, e.g., SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132; SEQ ID NO:134; SEQ ID NO:136; SEQ ID NO:138; SEQ ID NO:140; SEQ ID NO:142; SEQ ID NO:144; NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, SEQ ID NO:172, SEQ ID NO:174, SEQ ID NO:176, SEQ ID NO:178, SEQ ID NO:180, SEQ ID NO:182, SEQ ID NO:184, SEQ ID NO:186, SEQ ID NO:188, SEQ ID NO:190, SEQ ID NO:192, SEQ ID NO:194, SEQ ID NO:196, SEQ ID NO:198. As discussed above, the identity can be over the full length of the polypeptide, or, the identity can be over a region of at least about 50, 60, 77, 80, 90, 100, 150, 200, 220 or more residues. Polypeptides of the invention can also be shorter than the full length of exemplary polypeptides (e.g., SEQ ID NO:2; SEQ ID NO:4; SEQ ID NO:6; SEQ ID NO:8, SEQ ID NO:10, SEQ ID NO:12, SEQ ID NO:14, SEQ ID NO:16; SEQ ID NO:18; SEQ ID NO:20; SEQ ID NO:22; SEQ ID NO:24; SEQ ID NO:26). In alternative aspects, the invention provides polypeptides (peptides, fragments) ranging in size between about 5 and the full length of a polypeptide, e.g., an enzyme, such as a fluorescent polypeptide, e.g., green fluorescent protein; exemplary sizes being of about 5, 10, 15, 20, 25, 30, 35, 40, 45, 50, 55, 60, 65, 70, 75, 80, 85, 90, 100, 125, 150, 175, 200, 220 or more residues, e.g., contiguous residues of an exemplary fluorescent polypeptide of the invention. Peptides of the invention can be useful as, e.g., labeling probes, antigens, toleragens, motifs, fluorescent active sites.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

The peptides and polypeptides of the invention can also be glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

The peptides and polypeptides of the invention, as defined above, include all “mimetic” and “peptidomimetic” forms. The terms “mimetic” and “peptidomimetic” refer to a synthetic chemical compound that has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, in one aspect, a mimetic composition is within the scope of the invention if it has a fluorescent activity.

Polypeptide mimetic compositions of the invention can contain any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH2- for —C(═O)—NH—), aminomethylene (CH2-NH), ethylene, olefin (CH═CH), ether (CH2-O), thioether (CH2-S), tetrazole (CN4-), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide of the invention can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L(3-pyridinyl)-alanine; D- or L-(2-pyrazinyl)-alanine; D- or L-(4-isopropyl)-phenylglycine; D(trifluoromethyl)-phenylglycine; D-(trifluoromethyl)-phenylalanine; D-p-fluorophenylalanine; D- or L-p-biphenyl-phenylalanine; K- or L-p-methoxybiphenylphenylalanine; D- or L-2-indole(alkyl)-alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A residue, e.g., an amino acid, of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but also can be referred to as the R- or S-form.

The invention also provides methods for modifying the polypeptides of the invention by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W.H. Freeman and Company, New York (1993); Posttranslational Covalent Modification of Proteins, B.C. Johnson, Ed., Academic Press, New York, pp. 1-12 (1983).

Solid-phase chemical peptide synthesis methods can also be used to synthesize the polypeptide or fragments of the invention. Such method have been known in the art since the early 1960's (Merrifield, R. B., J. Am. Chem. Soc., 85:2149-2154, 1963) (See also Stewart, J. M. and Young, J. D., Solid Phase Peptide Synthesis, 2nd Ed., Pierce Chemical Co., Rockford, Ill., pp. 11-12)) and have recently been employed in commercially available laboratory peptide design and synthesis kits (Cambridge Research Biochemicals). Such commercially available laboratory kits have generally utilized the teachings of H. M. Geysen et al, Proc. Natl. Acad. Sci., USA, 81:3998 (1984) and provide for synthesizing peptides upon the tips of a multitude of “rods” or “pins” all of which are connected to a single plate. When such a system is utilized, a plate of rods or pins is inverted and inserted into a second plate of corresponding wells or reservoirs, which contain solutions for attaching or anchoring an appropriate amino acid to the pin's or rod's tips. By repeating such a process step, i.e., inverting and inserting the rod's and pin's tips into appropriate solutions, amino acids are built into desired peptides. In addition, a number of available FMOC peptide synthesis systems are available. For example, assembly of a polypeptide or fragment can be carried out on a solid support using an Applied Biosystems, Inc. Model 431A™ automated peptide synthesizer. Such equipment provides ready access to the peptides of the invention, either by direct synthesis or by synthesis of a series of fragments that can be coupled using other known techniques.

Exemplary SEQ ID NO:2, obtained from an environmental sample, has the sequence

```
Met Ser His Ser Lys Ser Val Ile Lys Asp Glu Met Phe Ile Lys Ile
1 5 10 15
His Leu Glu Gly Thr Phe Asn Gly His Lys Phe Glu Ile Glu Gly Glu
              20 25 30
Gly His Gly Lys Pro Tyr Ala Gly Thr Asn Phe Val Lys Leu Val Val
        35 40 45
Thr Arg Gly Gly Pro Leu Pro Phe Gly Trp His Ile Leu Ser Pro Gln
    50 55 60
Phe Gln Tyr Gly Asn Lys Thr Phe Val Ser Tyr Pro Arg Asp Ile Pro
65 70 75 80
Asp Tyr Ile Lys Gln Ser Phe Pro Glu Gly Phe Thr Trp Glu Arg Ile
                  85 90 95
Met Thr Phe Glu Asp Gly Gly Val Cys Cys Ile Thr Ser Asp Ile Ser
            100 105 110
Leu Lys Ser Asn Asn Cys Phe Phe Asn Asp Ile Lys Phe Thr Gly Met
        115 120 125
Asn Phe Pro Pro Asn Gly Ser Val Val Gln Lys Lys Thr Ile Gly Trp
    130 135 140
Glu Pro Ser Thr Glu Arg Leu Tyr Leu Arg Asp Gly Val Leu Thr Gly
145 150 155 160
Asp Ile Asp Lys Thr Leu Lys Leu Ser Gly Gly Gly His Tyr Thr Cys
                    165 170 175
Ala Phe Lys Thr Ile Tyr Arg Ser Lys Lys Asn Leu Thr Leu Pro Asp
              180 185 190
Cys Leu Tyr Tyr Val Asp Thr Lys Leu Asp Ile Arg Lys Phe Asp Glu
        195 200 205
Asn Tyr Ile Asn Val Glu Gln Asp Glu Ile Ala Thr Ala Arg His His
  210 215 220
Gly Leu Lys
225
```

Exemplary SEQ ID NO:4, obtained from an environmental sample, has the sequence

```
Met Ser His Ser Lys Ser Val Ile Lys Asp Glu Met Phe Ile Lys Ile
1 5 10 15
His Leu Glu Gly Thr Phe Asn Gly His Lys Phe Glu Ile Glu Gly Glu
            20 25 30
Gly His Gly Lys Pro Tyr Ala Gly Thr Asn Phe Val Lys Leu Val Val
        35 40 45
Thr Lys Gly Gly Pro Leu Pro Phe Gly Trp His Ile Leu Ser Pro Gln
    50 55 60
Phe Gln Tyr Gly Asn Lys Thr Phe Val Ser Tyr Pro Arg Asp Ile Pro
65 70 75 80
Asp Tyr Ile Lys Gln Ser Phe Pro Glu Gly Phe Thr Trp Val Arg Ile
                  85 90 95
Met Thr Phe Glu Asp Gly Gly Val Cys Cys Ile Thr Ser Asp Ile Ser
            100 105 110
Leu Lys Ser Asn Asn Cys Phe Phe Asn Asp Ile Lys Phe Thr Gly Met
        115 120 125
Asn Phe Pro Pro Asn Gly Pro Val Val Gln Lys Lys Thr Ile Gly Trp
  130 135 140
Glu Pro Ser Thr Glu Arg Leu Tyr Leu Arg Asp Gly Val Leu Thr Gly
145 150 155 160
Asp Ile Asp Lys Thr Leu Lys Leu Ser Gly Gly Gly His Tyr Thr Cys
                  165 170 175
Ala Phe Lys Thr Ile Tyr Arg Ser Lys Lys Asn Leu Thr Leu Pro Asp
            180 185 190
Cys Phe Tyr Tyr Val Asp Thr Lys Leu Asp Ile Arg Lys Phe Asp Glu
        195 200 205
Asn Tyr Ile Asn Val Glu Gln Asp Glu Ile Ala Thr Ala Arg His His
  210 215 220
Gly Leu Lys
225
```

Exemplary SEQ ID NO:6, obtained from an environmental sample, has the sequence

```
Met Ser His Ser Lys Ser Val Ile Lys Asp Glu Met Phe Ile Lys Ile
1 5 10 15
His Leu Glu Gly Thr Phe Asn Gly His Lys Phe Glu Ile Glu Gly Glu
            20 25 30
Gly His Gly Lys Pro Tyr Ala Gly Thr Asn Phe Val Lys Leu Val Val
        35 40 45
Thr Lys Gly Gly Pro Leu Pro Phe Gly Trp His Ile Leu Ser Pro Gln
    50 55 60
Phe Gln Tyr Gly Asn Lys Thr Phe Val Ser Tyr Pro Arg Asp Ile Pro
65 70 75 80
Asp Tyr Ile Lys Gln Ser Phe Pro Glu Gly Phe Thr Trp Glu Arg Ile
                  85 90 95
Met Thr Phe Glu Asp Gly Gly Val Cys Cys Ile Thr Ser Asp Ile Ser
            100 105 110
Leu Lys Ser Asn Asn Cys Phe Phe Asn Asp Ile Lys Phe Thr Gly Met
        115 120 125
Asn Phe Pro Pro Asn Gly Pro Val Val Gln Lys Lys Thr Ile Gly Trp
  130 135 140
Glu Pro Ser Thr Glu Arg Leu Tyr Leu Arg Asp Gly Val Leu Thr Gly
145 150 155 160
Asp Ile Asp Lys Thr Leu Lys Leu Ser Gly Gly Gly His Tyr Thr Cys
                  165 170 175
Ala Phe Lys Thr Ile Tyr Arg Ser Lys Lys Asn Leu Thr Leu Pro Asp
              180 185 190
Cys Phe Tyr Tyr Val Asp Thr Lys Leu Asp Ile Arg Lys Phe Asp Glu
        195 200 205
Asn Tyr Ile Asn Val Glu Gln Asp Glu Ile Ala Thr Ala Arg His His
  210 215 220
Gly Leu Lys
225
```

Exemplary SEQ ID NO:8, obtained from an environmental sample, has the sequence

```
Met Ser His Ser Lys Ser Val Ile Lys Asp Glu Met Phe Ile Lys Ile
1 5 10 15
His Leu Glu Gly Thr Phe Asn Gly His Lys Phe Glu Ile Glu Gly Glu
              20 25 30
Gly Asn Gly Lys Pro Tyr Ala Gly Thr Asn Phe Val Lys Leu Val Val
        35 40 45
Thr Lys Gly Gly Pro Leu Pro Phe Gly Trp His Ile Leu Ser Pro Gln
    50 55 60
Leu Gln Tyr Gly Asn Lys Ser Phe Val Ser Tyr Pro Ala Asp Ile Pro
65 70 75 80
Asp Tyr Ile Lys Leu Ser Phe Pro Glu Gly Phe Thr Trp Glu Arg Ile
                  85 90 95
Met Thr Phe Glu Asp Gly Gly Val Cys Cys Ile Thr Ser Asp Ile Ser
            100 105 110
Met Lys Ser Asn Asn Cys Phe Phe Tyr Asp Ile Lys Phe Thr Gly Met
        115 120 125
Asn Phe Pro Pro Asn Gly Pro Val Val Gln Lys Lys Thr Thr Gly Trp
  130 135 140
Glu Pro Ser Thr Glu Arg Leu Tyr Leu Arg Asp Gly Val Leu Thr Gly
145 150 155 160
Asp Ile His Lys Thr Leu Lys Leu Ser Gly Gly Gly His Tyr Thr Cys
                    165 170 175
Val Phe Lys Thr Ile Tyr Arg Ser Lys Lys Asn Leu Thr Leu Pro Asp
            180 185 190
Cys Phe Tyr Tyr Val Asp Thr Lys Leu Asp Ile Arg Lys Phe Asp Glu
        195 200 205
Asn Tyr Ile Asn Val Glu Gln Asp Glu Ile Ala Thr Ala Arg His His
    210 215 220
Gly Leu Lys
225
```

Exemplary SEQ ID NO:10, obtained from an environmental sample, has the sequence

```
Met Lys Gly Val Lys Glu Val Met Lys Ile Ser Leu Glu Met Asp Cys
1 5 10 15
Thr Val Asn Gly Asp Lys Phe Lys Ile Thr Gly Asp Gly Thr Gly Glu
            20 25 30
Pro Tyr Glu Gly Thr Gln Thr Leu His Leu Thr Glu Lys Glu Gly Lys
        35 40 45
Pro Leu Thr Phe Ser Phe Asp Val Leu Thr Pro Ala Phe Gln Tyr Gly
    50 55 60
Asn Arg Thr Phe Thr Lys Tyr Pro Gly Asn Ile Pro Asp Phe Phe Lys
65 70 75 80
Gln Thr Val Ser Gly Gly Gly Tyr Thr Trp Glu Arg Lys Met Thr Tyr
                  85 90 95
Glu Asp Gly Gly Ile Ser Asn Val Arg Ser Asp Ile Ser Val Lys Gly
            100 105 110
Asp Ser Phe Tyr Tyr Lys Ile His Phe Thr Gly Glu Phe Pro Pro His
        115 120 125
Gly Pro Val Met Gln Arg Lys Thr Val Lys Trp Glu Pro Ser Thr Glu
    130 135 140
Val Met Tyr Val Asp Asp Lys Ser Asp Gly Val Leu Lys Gly Asp Val
145 150 155 160
Asn Met Ala Leu Leu Leu Lys Asp Gly Arg His Leu Arg Val Asp Phe
                165 170 175
Asn Thr Ser Tyr Ile Pro Lys Lys Lys Val Glu Asn Met Pro Asp Tyr
            180 185 190
His Phe Ile Asp His Arg Ile Glu Ile Leu Gly Asn Pro Glu Asp Lys
        195 200 205
Pro Val Lys Leu Tyr Glu Cys Ala Val Ala Arg Tyr Ser Leu Leu Pro
    210 215 220
Glu Lys Asn Lys Ser
225
```

Exemplary SEQ ID NO:12, obtained from an environmental sample, has the sequence

```
Met Lys Gly Val Lys Glu Val Met Lys Ile Ser Leu Glu Met Asp Cys
1 5 10 15
Thr Val Asn Gly Asp Lys Phe Lys Ile Thr Gly Asp Gly Thr Gly Glu
              20 25 30
Pro Tyr Glu Gly Thr Gln Thr Leu His Leu Thr Glu Lys Glu Gly Lys
        35 40 45
Pro Leu Thr Phe Ser Phe Asp Val Leu Thr Pro Ala Phe Gln Tyr Gly
    50 55 60
Asn Arg Thr Phe Thr Lys Tyr Pro Gly Asn Ile Pro Asp Phe Phe Lys
65 70 75 80
Gln Thr Val Ser Gly Gly Gly Tyr Thr Trp Glu Arg Lys Met Thr Tyr
                  85 90 95
Glu Asp Gly Gly Ile Ser Asn Val Arg Ser Asp Ile Ser Val Lys Gly
              100 105 110
Asp Ser Phe Tyr Tyr Lys Ile His Phe Thr Gly Glu Phe Pro Pro His
        115 120 125
Gly Pro Val Met Gln Arg Lys Thr Val Lys Trp Glu Pro Ser Thr Glu
    130 135 140
Val Met Tyr Val Asp Asp Lys Ser Gly Gly Glu Leu Lys Gly Asp Val
145 150 155 160
Asn Met Ala Leu Leu Leu Lys Asp Gly Arg His Leu Arg Val Asp Phe
                165 170 175
Asn Thr Ser Tyr Ile Pro Lys Lys Lys Val Glu Asn Met Pro Asp Tyr
            180 185 190
His Phe Ile Asp His Arg Ile Glu Ile Leu Gly Asn Pro Glu Asp Lys
        195 200 205
Pro Val Lys Leu Tyr Glu Cys Ala Val Ala Arg Tyr Ser Leu Leu Pro
    210 215 220
Glu Lys Asn Lys
225
```

Exemplary SEQ ID NO:14, obtained from an environmental sample, has the sequence

```
Met Lys Glu Val Met Lys Ile Ser Leu Glu Met Asp Cys Thr Val Asn
1 5 10 15
Gly Asp Lys Phe Lys Ile Thr Gly Asp Gly Thr Gly Glu Pro Tyr Glu
            20 25 30
Gly Thr Gln Thr Leu His Leu Thr Glu Lys Glu Gly Lys Pro Leu Thr
        35 40 45
Phe Ser Phe Asp Val Leu Thr Pro Ala Phe Gln Tyr Gly Asn Arg Thr
    50 55 60
Phe Thr Lys Tyr Pro Gly Asn Ile Pro Asp Phe Phe Lys Gln Thr Val
65 70 75 80
Ser Gly Gly Gly Tyr Thr Trp Glu Arg Lys Met Thr Tyr Glu Asp Gly
                  85 90 95
Gly Ile Ser Asn Val Arg Ser Asp Ile Ser Val Lys Gly Asp Ser Phe
                100 105 110
Tyr Tyr Lys Ile His Phe Thr Gly Glu Phe Pro Ser His Gly Pro Val
        115 120 125
Met Gln Lys Lys Thr Val Lys Trp Glu Pro Ser Thr Glu Val Met Tyr
  130 135 140
Val Asp Asp Lys Ser Asp Gly Val Leu Lys Gly Asp Val Asn Met Ala
145 150 155 160
Leu Leu Leu Lys Asp Gly Arg His Leu Arg Val Asp Phe Asn Thr Ser
                165 170 175
Tyr Ile Pro Lys Lys Lys Val Glu Asn Met Pro Asp Tyr His Phe Ile
                180 185 190
Asp His Arg Ile Glu Ile Leu Gly Asn Pro Asp Asp Asn Pro Val Lys
        195 200 205
Leu Tyr Glu Cys Ala Val Ala Arg Cys Ser Leu Leu Pro Glu Lys Asn
    210 215 220
Lys
225
```

Exemplary SEQ ID NO:16, obtained from an environmental sample, has the sequence

```
Met Lys Gly Val Lys Glu Val Met Lys Ile Gln Val Lys Met Asn Ile
1 5 10 15
Thr Val Asn Gly Asp Lys Phe Val Ile Thr Gly Asp Gly Thr Gly Glu
            20 25 30
Pro Tyr Asp Gly Thr Gln Ile Leu Asn Leu Thr Val Glu Gly Gly Lys
        35 40 45
Pro Leu Thr Phe Ser Phe Asp Ile Leu Thr Pro Val Phe Met Tyr Gly
    50 55 60
Asn Arg Ala Phe Thr Lys Tyr Pro Glu Ser Ile Pro Asp Phe Phe Lys
65 70 75 80
Gln Thr Val Ser Gly Gly Gly Tyr Thr Trp Lys Arg Lys Met Ile Tyr
                  85 90 95
Asp His Glu Ala Glu Gly Val Ser Thr Val Asp Gly Asp Ile Ser Val
            100 105 110
Asn Gly Asp Cys Phe Ile Tyr Lys Ile Thr Phe Asp Gly Thr Phe Arg
        115 120 125
Glu Asp Gly Ala Val Met Gln Lys Met Thr Glu Lys Trp Glu Pro Ser
  130 135 140
Thr Glu Val Met Tyr Lys Asp Asp Lys Asn Asp Asp Val Leu Lys Gly
145 150 155 160
Asp Val Asn His Ala Leu Leu Leu Lys Asp Gly Arg His Val Arg Val
                  165 170 175
Asp Phe Asn Thr Ser Tyr Lys Ala Lys Ser Lys Ile Glu Asn Met Pro
            180 185 190
Gly Tyr His Phe Val Asp His Arg Ile Glu Ile Ile Gly Arg Ser Ser
        195 200 205
Gln Asp Thr Lys Val Lys Leu Phe Glu Asn Ala Val Ala Arg Cys Ser
    210 215 220
Leu Leu Pro Glu Lys Asn Gln
225 230
```

Exemplary SEQ ID NO:18, obtained from an environmental sample, has the sequence

```
Met Lys Gly Val Lys Glu Val Met Lys Ile Ser Leu Glu Met Asp Cys
1 5 10 15
Thr Val Asn Gly Asp Lys Phe Lys Ile Thr Gly Asp Gly Thr Gly Glu
              20 25 30
Pro Tyr Glu Gly Thr Gln Thr Leu His Leu Thr Glu Lys Glu Gly Lys
          35 40 45
Pro Leu Thr Phe Ser Phe Asp Val Leu Thr Pro Ala Phe Gln Tyr Gly
    50 55 60
Asn Arg Thr Phe Thr Lys Tyr Pro Gly Asn Ile Pro Asp Phe Phe Lys
65 70 75 80
Gln Thr Val Ser Gly Gly Gly Tyr Thr Trp Glu Arg Lys Met Thr Tyr
                  85 90 95
Glu Asp Gly Gly Ile Ser Asn Val Arg Ser Asp Ile Ser Val Lys Gly
            100 105 110
Asp Ser Phe Tyr Tyr Lys Ile His Phe Thr Gly Glu Phe Pro Pro His
        115 120 125
Gly Pro Val Met Gln Arg Lys Thr Val Lys Trp Glu Pro Ser Thr Glu
    130 135 140
Val Met Tyr Val Asp Asp Lys Ser Asp Gly Val Leu Lys Gly Asp Val
145 150 155 160
Asn Met Ala Leu Leu Leu Lys Asp Gly Arg His Leu Arg Val Asp Phe
                  165 170 175
Asn Thr Ser Tyr Ile Pro Lys Lys Lys Val Glu Asn Met Pro Asp Tyr
            180 185 190
His Phe Ile Asp His Arg Ile Glu Ile Leu Gly Asn Pro Glu Asp Lys
          195 200 205
Pro Val Lys Leu Tyr Glu Cys Ala Val Ala Arg Tyr Ser Leu Leu Pro
    210 215 220
Glu Lys Asn Lys
225
```

Exemplary SEQ ID NO:20, obtained from an environmental sample, has the sequence

```
Met Lys Gly Val Lys Glu Val Met Lys Ile Ser Leu Glu Met Asp Cys
1 5 10 15
Thr Val Asn Gly Asp Lys Phe Lys Ile Thr Gly Asp Gly Thr Gly Glu
              20 25 30
Pro Tyr Glu Gly Thr Gln Thr Leu His Leu Thr Glu Lys Glu Gly Lys
          35 40 45
Pro Leu Thr Phe Ser Phe Asp Val Leu Thr Pro Ala Phe Gln Tyr Gly
    50 55 60
Asn Arg Thr Phe Thr Lys Tyr Pro Gly Asn Ile Pro Asp Phe Phe Lys
65 70 75 80
Gln Thr Val Ser Gly Gly Gly Tyr Thr Trp Glu Arg Lys Met Thr Tyr
                  85 90 95
Glu Asp Gly Gly Ile Ser Asn Val Arg Ser Asp Ile Ser Val Lys Gly
            100 105 110
Asp Ser Phe Tyr Tyr Lys Ile His Phe Thr Gly Glu Phe Pro Pro His
        115 120 125
Gly Pro Val Met Gln Arg Lys Thr Val Lys Trp Glu Pro Ser Thr Glu
    130 135 140
Val Met Tyr Val Asp Asp Lys Ser Asp Gly Val Leu Lys Gly Asp Val
145 150 155 160
Asn Met Ala Leu Leu Leu Lys Asp Gly Arg His Leu Arg Val Asp Phe
                165 170 175
Asn Thr Ser Tyr Ile Pro Lys Lys Lys Val Glu Asn Met Pro Asp Tyr
            180 185 190
His Phe Ile Asp His Arg Ile Glu Ile Leu Gly Asn Pro Glu Asp Lys
        195 200 205
Pro Val Lys Leu Tyr Glu Cys Ala Val Ala Arg Tyr Ser Leu Leu Pro
    210 215 220
Glu Lys Asn Lys Ser Lys Gly Asn Ser Lys Leu Glu Gly Lys Pro Ile
225 230 235 240
Pro Asn Pro Leu Leu Gly Leu Asp Ser Thr Arg Thr Gly
                  245 250
```

Exemplary SEQ ID NO:22, obtained from an environmental sample, has the sequence

```
Val Met Ala Ile Ser Ala Leu Lys Asn Val Ile Ile Ile Val Ile Ile
1 5 10 15
Tyr Ser Cys Ser Thr Ser Ala Asp Ser Ser Asn Ser Tyr Ser Gly Ser
              20 25 30
Ser Phe Ala Asn Gly Ile Ala Glu Glu Met Met Thr Asp Leu His Leu
          35 40 45
Glu Gly Ala Val Asn Gly His His Phe Thr Ile Lys Gly Glu Gly Gly
    50 55 60
Gly Tyr Pro Tyr Glu Gly Val Gln Phe Met Ser Leu Glu Val Val Asn
65 70 75 80
Gly Ala Pro Leu Pro Phe Ser Phe Asp Ile Leu Thr Pro Ala Phe Met
                  85 90 95
Tyr Gly Asn Arg Val Phe Thr Lys Tyr Pro Lys Glu Ile Pro His Tyr
              100 105 110
Phe Lys Gln Thr Phe Pro Glu Gly Tyr His Trp Glu Arg Ser Ile Pro
        115 120 125
Phe Gln Asp Gln Ala Ser Cys Thr Val Thr Ser His Ile Arg Met Lys
    130 135 140
Glu Glu Glu Glu Arg His Phe Leu Leu Asn Val Lys Phe Tyr Cys Val
145 150 155 160
Asn Phe Pro Pro Asn Gly Pro Val Met Gln Arg Arg Ile Arg Gly Trp
                  165 170 175
Glu Pro Ser Thr Glu Asn Ile Tyr Pro Arg Asp Glu Phe Leu Glu Gly
            180 185 190
His Asp Asp Met Thr Leu Arg Val Glu Gly Gly Gly Tyr Tyr Arg Ala
        195 200 205
Glu Phe Arg Ser Ser Tyr Lys Gly Lys His Ser Ile Asn Met Pro Asp
    210 215 220
Phe His Phe Ile Asp His Arg Ile Glu Ile Met Glu His Asp Glu Asp
225 230 235 240
Tyr Asn His Val Lys Leu Arg Glu Val Ala His Ala Arg Tyr Ser Pro
                245 250 255
Leu Pro Ser Val His
            260
```

Exemplary SEQ ID NO:24, obtained from an environmental sample, has the sequence

```
Val Met Ala Ile Ser Ala Leu Lys Asn Val Ile Ile Ile Val Ile Ile
1 5 10 15
Tyr Ser Cys Ser Thr Ser Ala Asp Ser Ser Asn Ser Tyr Ser Gly Ser
              20 25 30
Ser Phe Ala Asn Gly Ile Ala Glu Glu Met Met Thr Asp Leu His Leu
          35 40 45
Glu Gly Ala Val Asn Gly His His Phe Thr Ile Lys Gly Glu Gly Gly
    50 55 60
Gly Tyr Pro Tyr Glu Gly Val Gln Phe Met Ser Leu Glu Val Val Asn
65 70 75 80
Gly Ala Pro Leu Pro Phe Ser Phe Asp Ile Leu Thr Pro Ala Phe Met
                  85 90 95
Tyr Gly Asn Arg Val Phe Thr Lys Tyr Pro Lys Glu Ile Pro Asp Tyr
              100 105 110
Phe Lys Gln Thr Phe Pro Glu Gly Tyr His Trp Glu Arg Ser Ile Pro
        115 120 125
Phe Gln Asp Gln Ala Ser Cys Thr Val Thr Ser His Ile Arg Met Lys
    130 135 140
Glu Glu Glu Glu Arg His Phe Leu Leu Asn Val Lys Phe Tyr Cys Val
145 150 155 160
Asn Phe Pro Pro Asn Gly Pro Val Met Gln Arg Arg Ile Arg Gly Trp
                  165 170 175
Glu Pro Ser Thr Glu Asn Ile Tyr Pro Arg Asp Glu Phe Leu Glu Gly
              180 185 190
His Asp Asp Met Thr Leu Arg Val Glu Gly Gly Gly Tyr Tyr Arg Ala
        195 200 205
Glu Phe Arg Ser Ser Tyr Lys Gly Lys His Ser Ile Asn Met Pro Asp
    210 215 220
Phe His Phe Ile Asp His Arg Ile Glu Ile Met Glu His Asp Glu Asp
225 230 235 240
Tyr Asn His Val Lys Leu Arg Glu Val Ala His Ala Arg Tyr Ser Pro
                  245 250 255
Leu Pro Ser Val His
            260
```

Exemplary SEQ ID NO:26, obtained from an environmental sample, has the sequence

```
Met Ala Ile Ser Ala Leu Lys Asn Val Ile Ile Ile Val Ile Ile Tyr
1 5 10 15
Ser Arg Ser Thr Ser Ala Asp Ser Ser Asn Ser Tyr Ser Gly Ser Ser
              20 25 30
Phe Ala Asn Gly Ile Ala Glu Glu Met Met Thr Asp Leu His Leu Glu
        35 40 45
Gly Ala Val Asn Gly His His Phe Thr Ile Lys Gly Glu Gly Gly Gly
    50 55 60
Tyr Pro Tyr Glu Gly Val Gln Phe Met Ser Leu Glu Val Val Asn Gly
65 70 75 80
Ala Pro Leu Pro Phe Ser Phe Asp Ile Leu Thr Pro Ala Phe Met Tyr
                  85 90 95
Gly Asn Arg Val Phe Thr Lys Tyr Pro Lys Glu Ile Pro Asp Tyr Phe
            100 105 110
Lys Gln Thr Phe Pro Glu Gly Tyr His Trp Glu Arg Ser Ile Pro Phe
        115 120 125
Gln Asp Gln Ala Ser Cys Thr Val Thr Ser His Ile Arg Met Lys Glu
    130 135 140
Glu Glu Glu Arg His Phe Leu Leu Asn Val Lys Phe Tyr Cys Val Asn
145 150 155 160
Phe Pro Pro Asn Gly Pro Val Met Gln Arg Arg Ile Arg Gly Trp Glu
                  165 170 175
Pro Ser Thr Glu Asn Ile Tyr Pro Arg Asp Glu Phe Leu Glu Gly His
              180 185 190
Asp Asp Met Thr Leu Arg Val Glu Gly Gly Gly Tyr Tyr Arg Ala Glu
        195 200 205
Phe Arg Ser Ser Tyr Lys Gly Lys His Ser Ile Asn Met Pro Asp Phe
    210 215 220
His Phe Ile Asp His Arg Ile Glu Ile Met Glu His Asp Glu Asp Tyr
225 230 235 240
Asn His Val Lys Leu Arg Glu Val Ala Tyr Ala Arg Tyr Ser Pro Leu
                245 250 255
Pro Ser Val His
              260
```

Signal Sequence, Fluorescent Domains, Carbohydrate Binding Modules

The invention provides fluorescent protein signal sequences (e.g., signal peptides (SPs)) and nucleic acids encoding these signal sequences, e.g., a peptide having a sequence comprising/consisting of amino terminal residues of a polypeptide of the invention. In one aspect, the invention provides a signal sequence comprising a peptide comprising/consisting of a sequence as set forth in residues 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 28, 1 to 30, 1 to 31, 1 to 32, 1 to 33, 1 to 34, 1 to 35, 1 to 36, 1 to 37, 1 to 38, 1 to 39, 1 to 40, 1 to 41, 1 to 42, 1 to 43, 1 to 44 of a polypeptide of the invention, e.g., SEQ ID NO:2; SEQ ID NO:4; SEQ ID NO:6; SEQ ID NO:8, SEQ ID NO:10, SEQ ID NO:12, SEQ ID NO:14, SEQ ID NO:16, SEQ ID NO:18; SEQ ID NO:20; SEQ ID NO:22; SEQ ID NO:24; SEQ ID NO:26.

The fluorescent protein signal sequences of the invention can be isolated peptides, or, sequences joined to another fluorescent protein or a non-fluorescent protein polypeptide, e.g., as a fusion protein. In one aspect, the invention provides polypeptides comprising fluorescent protein signal sequences of the invention. In one aspect, polypeptides comprising fluorescent protein signal sequences of the invention comprise sequences heterologous to a fluorescent protein of the invention (e.g., a fusion protein comprising a fluorescent protein signal sequence of the invention and sequences from another fluorescent protein or a nonfluorescent protein). In one aspect, the invention provides fluorescent protein of the invention with heterologous signal sequences, e.g., sequences with a yeast signal sequence. A fluorescent protein of the invention can comprise a heterologous signal sequence in vectors, e.g., a pPIC series vector (Invitrogen, Carlsbad, Calif.).

In one aspect, the signal sequences of the invention are identified following identification of novel fluorescent protein polypeptides. The pathways by which proteins are sorted and transported to their proper cellular location are often referred to as protein targeting pathways. One of the most important elements in all of these targeting systems is a short amino acid sequence at the amino terminus of a newly synthesized polypeptide called the signal sequence. This signal sequence directs a protein to its appropriate location in the cell and is removed during transport or when the protein reaches its final destination. Most lysosomal, membrane, or secreted proteins have an amino-terminal signal sequence that marks them for translocation into the lumen of the endoplasmic reticulum. More than 100 signal sequences for proteins in this group have been determined. The signal sequences can vary in length from 13 to 36 amino acid residues. Various methods of recognition of signal sequences are known to those of skill in the art. For example, in one aspect, novel fluorescent protein signal peptides are identified by a method referred to as SignalP. SignalP uses a combined neural network that recognizes both signal peptides and their cleavage sites. (Nielsen, et al., “Identification of prokaryotic and eukaryotic signal peptides and prediction of their cleavage sites.” Protein Engineering, vol. 10, no. 1, p. 1-6 (1997).

It should be understood that in some aspects fluorescent proteins of the invention may not have signal sequences. In one aspect, the invention provides the fluorescent proteins of the invention lacking all or part of a signal sequence. In one aspect, the invention provides a nucleic acid sequence encoding a signal sequence from one fluorescent protein operably linked to a nucleic acid sequence of a different fluorescent protein or, optionally, a signal sequence from a non-fluorescent protein may be desired.

The invention also provides isolated or recombinant polypeptides comprising signal sequences (SPs) and fluorescent domains of the invention and heterologous sequences. The heterologous sequences are sequences not naturally associated with a signal sequences or fluorescent domains of the invention. The sequence to which the signal sequences or fluorescent domains are not naturally associated can be on the signal sequence's or fluorescent domain's amino terminal end, carboxy terminal end, and/or on both ends of the signal sequences or fluorescent domains. In one aspect, the invention provides an isolated or recombinant polypeptide comprising (or consisting of) a polypeptide comprising a signal sequence or fluorescent domain of the invention with the proviso that it is not associated with any sequence to which it is naturally associated (e.g., a fluorescent protein sequence). Similarly in one aspect, the invention provides isolated or recombinant nucleic acids encoding these polypeptides. Thus, in one aspect, the isolated or recombinant nucleic acid of the invention comprises coding sequence for a signal sequence or fluorescent domain of the invention and a heterologous sequence (i.e., a sequence not naturally associated with the a signal sequence or fluorescent domain of the invention). The heterologous sequence can be on the 3′ terminal end, 5′ terminal end, and/or on both ends of the signal sequence or fluorescent domain coding sequence.

Fusion Proteins with Signal Sequences

The invention provides fusion proteins comprising fluorescent proteins of the invention and signal sequences. Pathways by which proteins are sorted and transported to their proper cellular location are often referred to as protein targeting pathways. One of the most important elements in all of these targeting systems is a short amino acid sequence at the amino terminus of a newly synthesized polypeptide called the signal sequence. This signal sequence directs a protein to its appropriate location in the cell and is removed during transport or when the protein reaches its final destination. Most lysosomal, membrane, or secreted proteins have an amino-terminal signal sequence that marks them for translocation into the lumen of the endoplasmic reticulum. More than 100 signal sequences for proteins in this group have been determined. The sequences vary in length from 13 to 36 amino acid residues. Various methods of recognition of signal sequences are known to those of skill in the art. For example, in one aspect, novel signal peptides are identified by a method referred to as SIGNALP™. SignalP uses a combined neural network that recognizes both signal peptides and their cleavage sites. (see, e.g., Nielsen (1997) Protein Engineering 10:1-6).

A nucleic acid sequence encoding fluorescent proteins of the invention may be linked to a cleavable signal peptide sequence to promote secretion of the encoded protein by the transformed cell. Signal peptides can include signal peptides from tissue plasminogen activator, insulin, neuron growth factor or juvenile hormone esterase of Heliothis virescens. For example, in order to study intracellular protein function, a following construct can be used. In one aspect, a fusion protein can comprise the membrane-translocating peptide sequence (MTS), which facilitates entry of polypeptides and proteins into cells, a fluorescent polypeptide of the invention, and the protein to be studied. This construct can be administered to the cells as discussed above. Once administered to the extracellular environment, the MTS directs import of the chimeric protein into the interior of the cell and the molecular marker enables visualization of target protein localization. See, e.g., U.S. Pat. No. 6,248,558.

In one aspect, the targeting sequence comprises a fluorescent protein of the invention and a membrane anchoring signal sequence. Membrane-anchoring sequences are well known in the art and are based on the genetic geometry of mammalian transmembrane molecules. Peptides are inserted into the membrane based on a signal sequence and require a hydrophobic transmembrane domain. The transmembrane proteins are inserted into the membrane such that the regions encoded 5′ of the transmembrane domain are extracellular and the sequences 3′ become intracellular. If these transmembrane domains are placed 5′ of the variable region, they will serve to anchor it as an intracellular domain, which may be desirable in some aspects of the invention. Since many parasites and pathogens bind to the membrane, in addition to the fact that many intracellular events originate at the plasma membrane. Thus, the invention provides membrane-bound peptide libraries that are useful for both the identification of important elements in these processes as well as for the discovery of effective inhibitors. The invention provides methods for presenting the randomized expression product extracellularly or in the cytoplasmic space.

In one aspect, the targeting sequence comprises a fluorescent protein of the invention and a secretory signal sequence capable of effecting the secretion of the peptide. There is a large number of known secretory signal sequences which are placed 5′ to the variable peptide region, and are cleaved from the peptide region to effect secretion into the extracellular space. Secretory signal sequences and their transferability to unrelated proteins are well known, e.g., Silhavy, et al. (1985) Microbiol. Rev. 49, 398-418. This is particularly useful to generate a peptide capable of binding to the surface of, or affecting the physiology of, a target cell that is other than the host cell, e.g., the cell infected with the retrovirus.

### Fluorescent Polypeptides

The invention provides novel fluorescent polypeptides, nucleic acids encoding them, antibodies that bind them, and methods for making and using them. In one aspect, the polypeptides of the invention have a fluorescent activity, as described above (e.g., ability to emit radiation after absorbing it). In alternative aspects, the fluorescent polypeptides of the invention have activities that have been modified from those of the exemplary fluorescent polypeptides described herein. The invention includes fluorescent polypeptides with and without signal sequences and the signal sequences themselves. The invention includes immobilized fluorescent polypeptides, anti-fluorescent protein antibodies and fragments thereof. The invention includes heterocomplexes, e.g., fusion proteins, heterodimers, etc., comprising the fluorescent polypeptides of the invention.

The following Table 2 is a summary of selected properties of exemplary fluorescent polypeptides of the invention (Ex is excitation, Em is emission).

TABLE 2

| SEQ ID NOS: | Ex | Em | Phenotype |
|---|---|---|---|
| 7, 8 | 448 | 491 | Cyan |
| 17, 18 | 487 | 507 | Green |
| 155, 156 | 485 | 503 | Green |
| 99, 100 | 385 | 462 | Blue |
| 135, 136 | 385-395 | 499, 470 | Green and Blue (UV excitable) |
| 57, 58 | 385 | 496 | Green (UV excitable) |
| 97, 98 | 448 | 504 | Green |
| 183, 184 | 475 | 504 | Green |
| 153, 154 | 395 | 500 | Green (UV excitable) |
| 59, 60 | 380 | 502 | Green (UV excitable) |
| 41, 42 | 365-380 | 466 | Blue |
| 79, 80 | 475 | 502 | Green |
| 109, 110 | 390 | 500 | Green (UV excitable) |
| 139, 140 | 390 | 500 | Green (UV excitable) |
| 63, 64 | 355-380 | 466 | Blue |
| 69, 70 | 475 | 502 | Green |
| 167, 168 | 440 | 504 | Green |
| 141, 142 | 475 | 504 | Green |
| 81, 82 | 385, 475 | 500 | Green (UV excitable) |
| 163, 164 | 365-380 | 464, 470 | Blue |
| 165, 166 | 380 | 500 | Green (UV excitable) |
| 91, 92 | 385 | 460 | Blue |
| 39, 40 | 380 | 498 | Green (UV excitable) |
| 177, 178 | 490 | 504 | Green |
| 35, 36 | 380 | 498 | Green (UV excitable) |
| 55, 56 | 490 | 502 | Green |
| 121, 122 | 492 | 504 | Green |
| 77, 78 | 492 | 504 | Green |
| 159, 160 | 380 | 456 | Blue |
| 83, 84 | 380 | 458 | Blue |
| 149, 150 | 490 | 504 | Green |
| 113, 114 | 492 | 504 | Green |
| 191, 192 | 490 | 504 | Green |
| 131, 132 | 492 | 507 | Green |
| 175, 176 | 485 | 502 | Green |
| 89, 90 | 494 | 502 | Green |
| 173, 174 | 385 | 496 | Green (UV excitable) |

Fluorescent Labeling

The polypeptides of the invention are used in fluorescent labeling of compositions, e.g., polypeptides and nucleic acids, organelles, and cells. Fluorescent labeling can be used as a tool for labeling a protein, cell, or organism of interest. Alternatively, a protein of interest can be purified, then covalently conjugated to a fluorophore derivative, e.g., a polypeptide of the invention. For in vivo studies, the protein-dye complex can be inserted into cells of interest, e.g., using micropipetting or a method of reversible permeabilization.

However, the process of fluorophore attachment and insertion in the cells is laborious and difficult to control. An alternative method of labeling proteins of interest is to concatenate or fuse the gene expressing the protein of interest to a gene expressing a marker, e.g., a polypeptide of the invention, then express the fusion product.

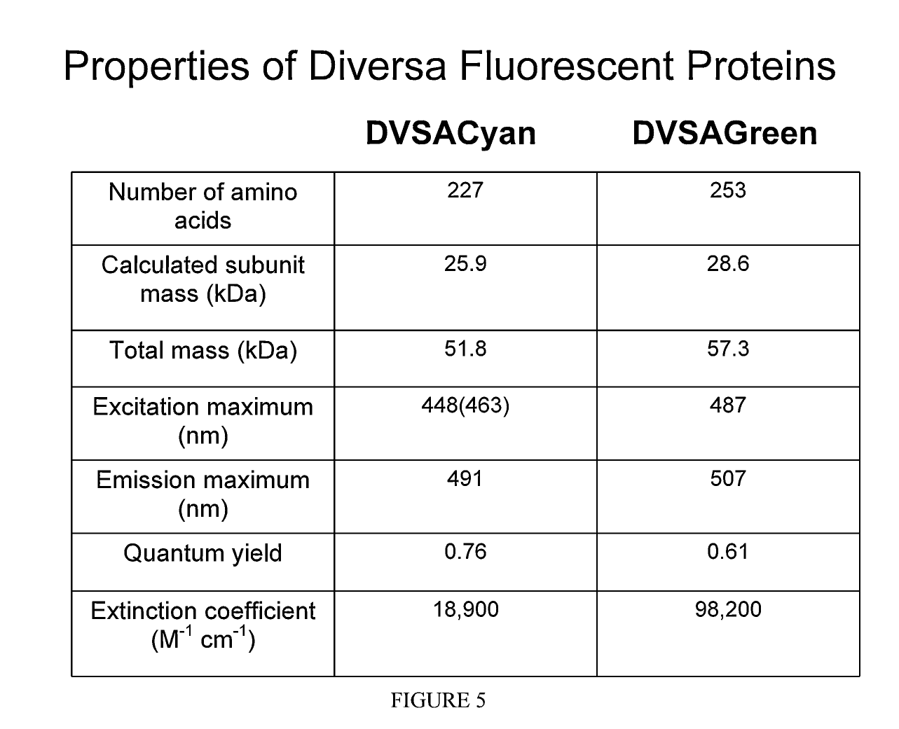

Selected properties of exemplary fluorescent polypeptides of the invention were determined and compared to other fluorescent proteins, as summarized below, and graphically represented in FIGS. 5 to 12. To determine maturation time, SEQ ID NO:18 (encoded by SEQ ID NO:17), designated DiscoveryPoint™ Green Fluorescent Protein, and SEQ ID NO:8 (encoded by SEQ ID NO:7), designated DiscoveryPoint™ Cyan Fluorescent Protein (SEQ ID NOS:7, 8), were expressed using host: BL21(DE3)pLysS (Stratagene, San Diego, Calif.) and vector: pCR®T7/CT-TOPO™ (Invitrogen, Carlsbad, Calif.), which were induced for one hour. An equal number of cells (1.250D) for each protein was aliquoted, sonicated, and centrifuged to obtain a clear lysate. The lysates were incubated at room temperature and the fluorescent intensity was monitor hourly by a TECAN SPECTRAFLOUR PLUS™ detection system. A maturation profile was generated for each protein. Proteins were incubated at 80° C. for 20 minutes to determine thermostability. Mass of the proteins were determined by size exclusion column chromatography (Sephacryl S200) with size standards: albumin, ovalbumin, chymotrypsinogen A, and ribonuclease A. Excitation and emission, along with quantum yield and extinction coefficients, were determined as described in Example 3, below. Stoke's shift is the difference between excitation and emission.

FIG. 5 is a summary of data comparing the properties of exemplary fluorescent polypeptides of the invention: DVSAGreen, which is SEQ ID NO:18, encoded by SEQ ID NO:17, and, DVSACyan, which is SEQ ID NO:8, encoded by SEQ ID NO:7. As noted in FIG. 5, SEQ ID NO:8 (DVSACyan) is 227 residues in length, has a calculated subunit mass of 25.9 kDa, a total mass of 51.8 kDa, an excitation maximum of 448 (463) nm, an emission maximum of 491 nm, a quantum yield of 0.76, and an extinction coefficient of 18,900 M⁻¹cm⁻¹. SEQ ID NO:18 (DVSAGreen) is 253 residues in length, has a calculated subunit mass of 28.6 kDa, a total mass of 57.3 kDa, an excitation maximum of 487 nm, an emission maximum of 507 nm, a quantum yield of 0.61, and an extinction coefficient of 98,200 M⁻¹cm⁻¹.

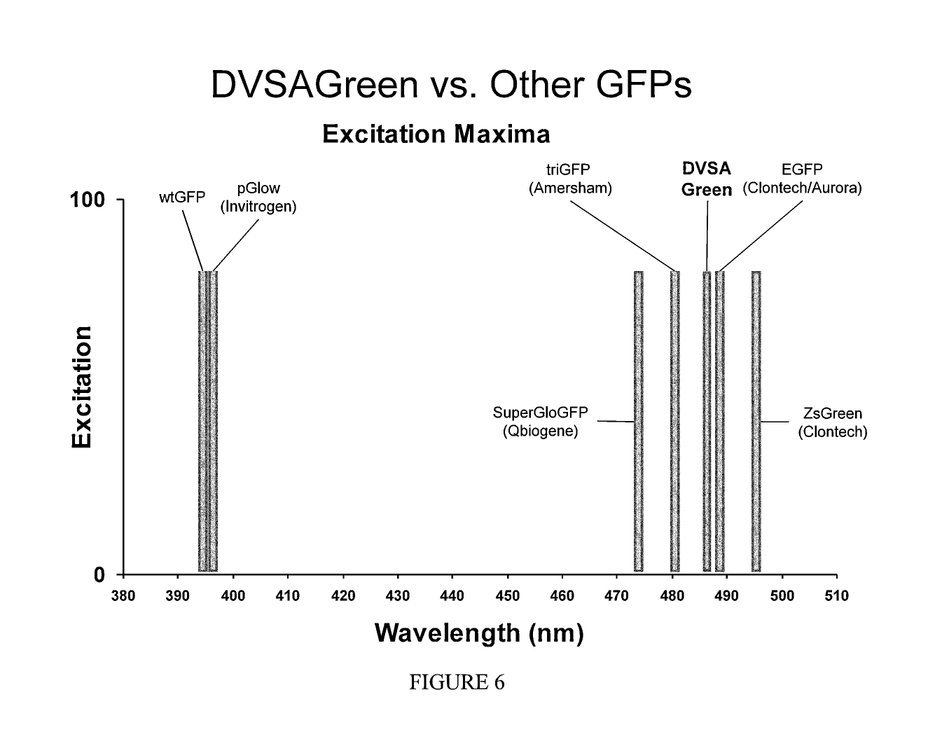

FIG. 6 is a graphic representation of data comparing excitation properties (excitation as a function of wavelength in nm), including excitation maxima, of an exemplary fluorescent polypeptide of the invention, SEQ ID NO:18 (DVSAGreen), to other fluorescent polypeptides.

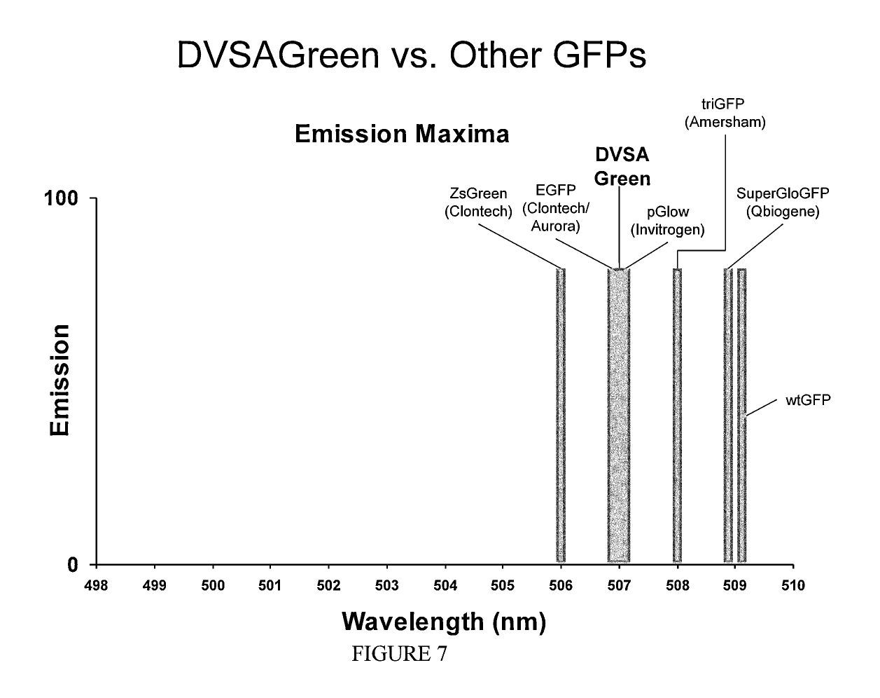

FIG. 7 is a graphic representation of data comparing emission properties (emission as a function of wavelength in nm), including emission maxima, of an exemplary fluorescent polypeptide of the invention, SEQ ID NO:18 (DVSAGreen), to other fluorescent polypeptides.

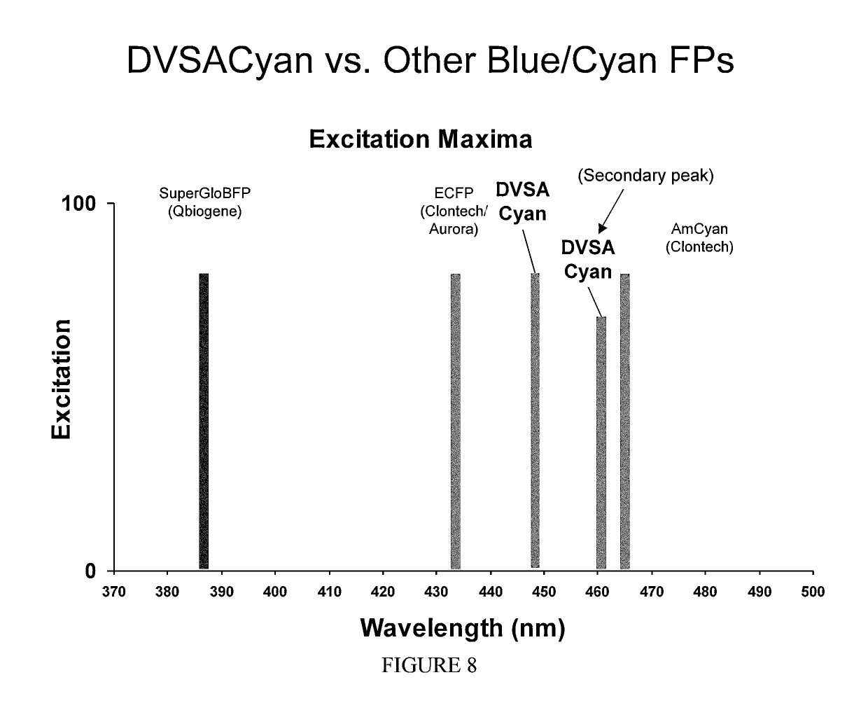

FIG. 8 is a graphic representation of data comparing excitation properties (excitation as a function of wavelength in nm), including excitation maxima, of an exemplary fluorescent polypeptide of the invention, SEQ ID NO:8 (DVSACyan), to other blue/cyan fluorescent polypeptides.

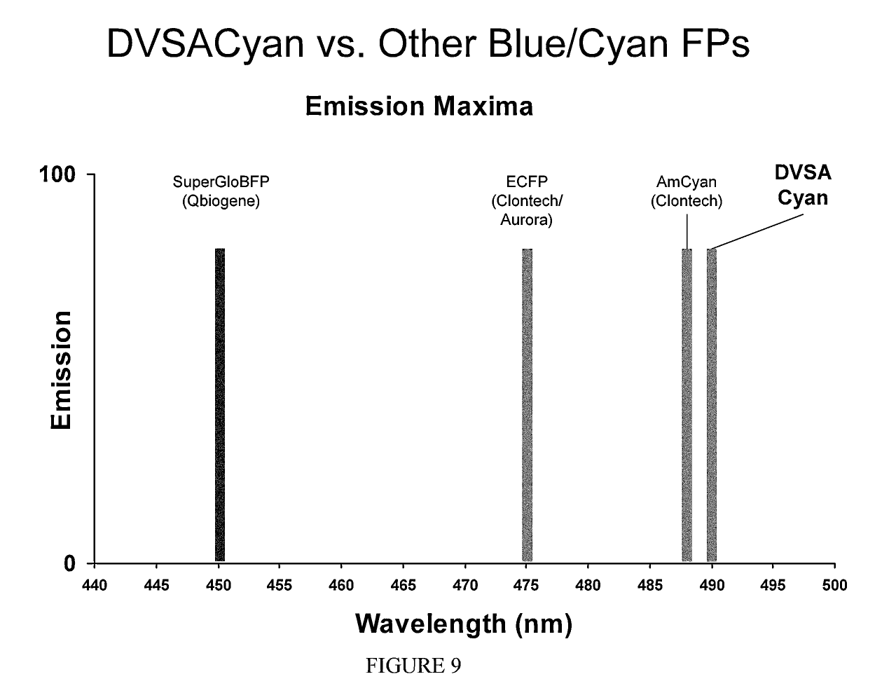

FIG. 9 is a graphic representation of data comparing emission properties (emission as a function of wavelength in nm), including emission maxima, of an exemplary fluorescent polypeptide of the invention, SEQ ID NO:8 (DVSACyan), to other blue/cyan fluorescent polypeptides.

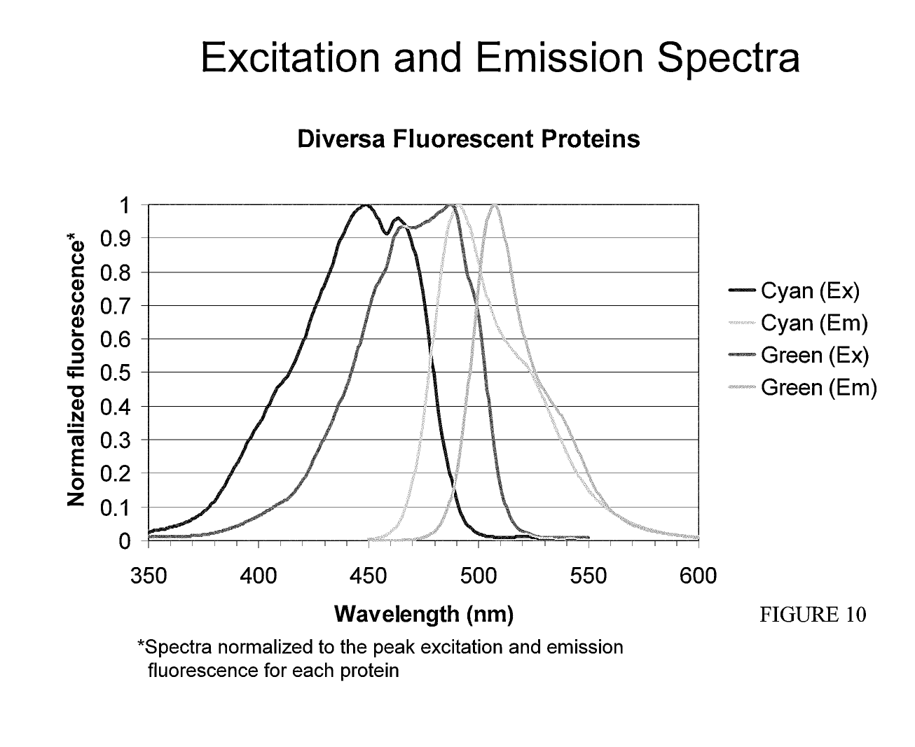

FIG. 10 is a graphic representation of data comparing excitation and emission spectra (normalized fluorescence as a function of wavelength in nm) of the exemplary fluorescent polypeptides of the invention SEQ ID NO:8 (DVSACyan, or “Cyan” in the graphic) and SEQ ID NO:18 (DVSAGreen, or “Green” in the graphic). Normalized fluorescence is spectra normalized to the peak excitation and emission fluorescence for each protein.

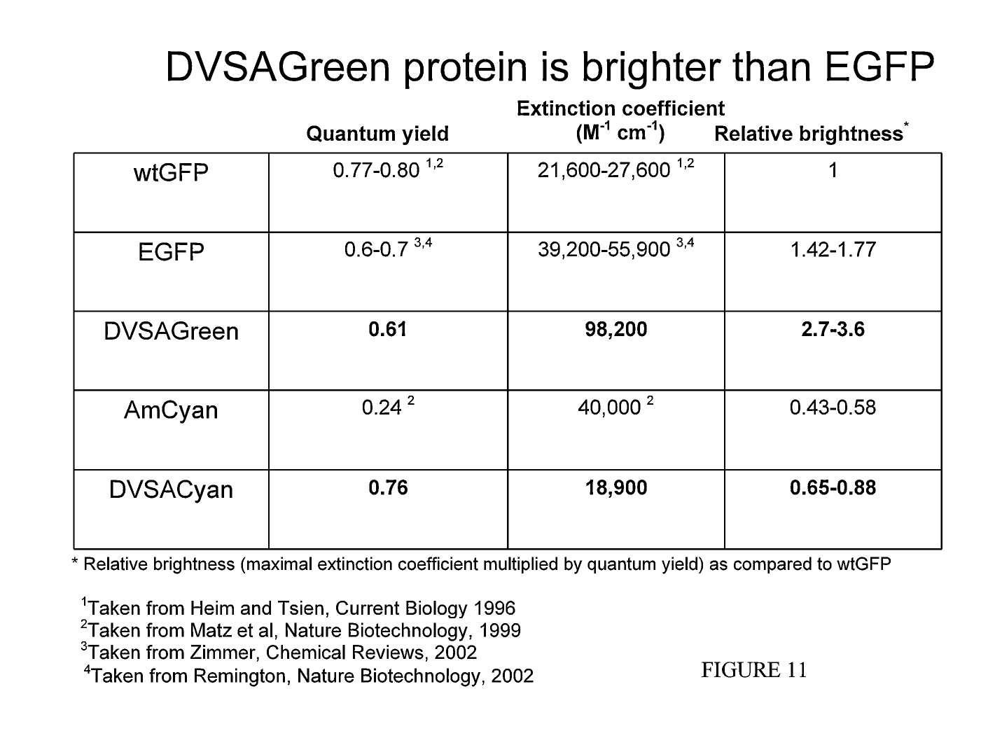

FIG. 11 is a summary of data comparing the properties (quantum yield, extinction coefficient, relative brightness) of exemplary fluorescent polypeptides of the invention, SEQ ID NO:8 (DVSACyan) and SEQ ID NO:18 (DVSAGreen) and other fluorescent polypeptides. Relative brightness is the maximal extinction coefficient multiplied by quantum yield.

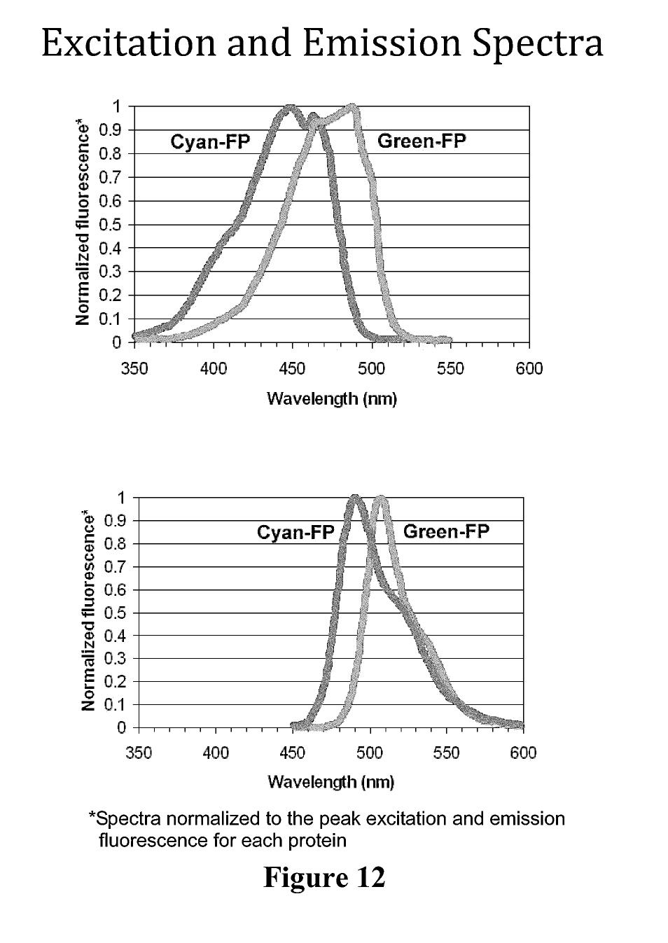

FIG. 12 is a graphic representation of data comparing excitation and emission spectra of the exemplary fluorescent polypeptides of the invention SEQ ID NO:8 (Cyan-FP in this graphic) and SEQ ID NO:18 (Green-FP in this graphic). Spectra normalized to the peak excitation and emission fluorescence for each protein.

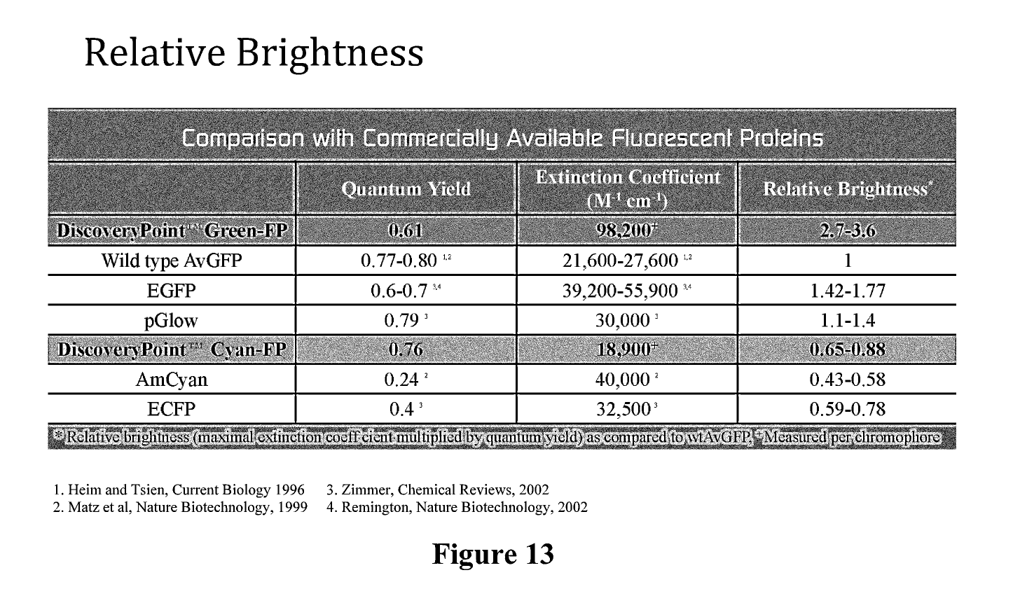

FIG. 13 is a summary of data comparing the properties (quantum yield, extinction coefficient, relative brightness) of exemplary fluorescent polypeptides of the invention, SEQ ID NO:8 (DISCOVERYPOINT™ CYAN-FP) and SEQ ID NO:18 (DISCOVERYPOINT™ GREEN-FP) and other fluorescent polypeptides. Relative brightness is the maximal extinction coefficient multiplied by quantum yield, as compared to wtAvGFP. Extinction coefficient was measured per chromophore.

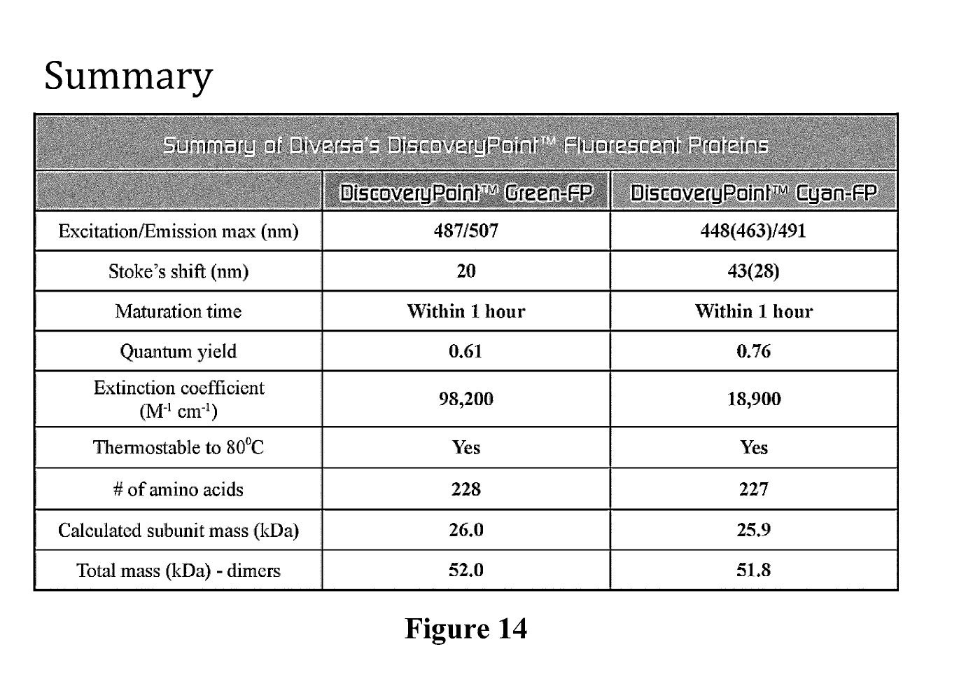

FIG. 14 is a summary of data comparing various properties (excitation/emission maxima, Stoke's shift in nm, maturation time, quantum yield, extinction coefficient, thermostability at 80° C., number of amino acid residues, calculated subunit mass in kDa, total mass in kDa for dimers) of exemplary fluorescent polypeptides of the invention, SEQ ID NO:8 (DISCOVERYPOINT™ CYAN-FP) and SEQ ID NO:18 (DISCOVERYPOINT™ GREEN-FP).

In addition to a polypeptide of the invention, other markers for protein labeling can also be used, e.g., galactosidase, firefly and bacterial luciferase. These other markers, however, require exogenous substrates and cofactors and therefore may be of limited use for in vivo studies.

The polypeptides of the invention marker do not require an exogenous cofactor or substrate. In one aspect, their absorbance/excitation peak is at 395 nm with a minor peak at 475 nm with extinction coefficients of roughly 30,000 and 7,000 M-1 cm-1, respectively. The emission peak can be at 508 nm. Excitation at 395 nm leads to decrease over time of the 395 nm excitation peak and a reciprocal increase in the 475 nm excitation band.

Fluorescence-based protein detection methods have recently surpassed conventional technologies, such as colloidal Coomassie blue and silver staining in terms of quantitative accuracy, detection sensitivity, and compatibility with modern downstream protein identification and characterization procedures, such as mass spectrometry. Additionally, specific detection methods suitable for revealing protein post-translational modifications have been devised over the years. Exemplary protocols for using polypeptides of the invention for the study of gene expression and protein localization are discussed in detail, e.g., in Chalfie et al. in Science 263 (1994), 802-8-5, and Heim et al. in Proc. Natl. Acad. Sci. 91 (1994), 12501-12504. Additionally, Rizzuto et al. in Curr. Biology 5 (19950, 635-642, discuss the use of fluorescent proteins as a tool for visualizing subcellular organelles in cells. Kaether and Gerdes in Febs. Letters 369 (1995), 267-271, describe the visualization of protein transport along the secretory pathway using fluorescent proteins. The expression of fluorescent proteins in plant cells is discussed by Hu and Cheng in Febs. Letters 360 (1995), 331-334, while fluorescent protein expression in Drosophila embryos is described by Davis et al. in Dev. Biology 170 (1995), 726-729. Use of the fluorescent proteins as an in vivo reporter has been reviewed by Hawes et al. in Protoplasma 215(1-4) (2001), 77-88. Magalhaes et al. in Luminescence16(2) (2001), 67-71, discuss how use fluorescent proteins to elucidate biological processes with fine spatio-temporal detail.

The fluorescent proteins of the invention (including fusion proteins comprising fluorescent proteins of the invention) are used to measure or probe cell signaling, physiological parameters or other activities (e.g., ion concentrations, protease activities, etc.). The demonstration that, using appropriate mutants and/or fusion proteins, fluorescent proteins can become sensitive to physiological parameters or activities (ion concentration, protease activity, etc.) has further expanded its applications and made fluorescent proteins the favorite probe of cell biologists. Exemplary applications of fluorescent proteins of the invention in the field of cell signaling include, e.g., those described by Chiesa et al. in Biochem J 355 (2001), 1-12. Condeelis et al. in Eur J. Cancer, 36(16) (2001), 2172-3, describe how the use of a fluorescent protein to fluorescently tag tumor cells has allowed to visualize the behavior of tumor cells in living tissues. Similarly, the fluorescent proteins of the invention are used to visualize the behavior of tumor cells, and other cells, pathological or normal, in living tissues, organs and whole animals.

The invention also provides crystals comprising the fluorescent proteins of the invention. Crystallographic structures of wild-type GFP and the mutant GFP S65T reveal that GFP tertiary structure resembles a barrel (Ormo et al., Science 273 (1996), 1392-1395; Yang et al., Nature Biotechnol. 14 (1996), 1246-1251). The barrel consists of beta sheets in a compact structure, where, in the center, an alpha helix containing the chromophore is shielded by the barrel, where it is almost completely protected from solvent access. The fluorescence of this protein is sensitive to a number of point mutations (Phillips, G. N., Curr. Opin. Struct. Biol. 7 (1997), 821-27). Similarly, the invention provides fluorescent proteins having similar point mutations.

The fluorescent proteins of the invention (including fusion proteins comprising fluorescent proteins of the invention) are used to investigate secondary, tertiary and quaternary structures of proteins, including the native structures of proteins. The fluorescence appears to be a sensitive indication of the preservation of the native structure of the protein, since any disruption of the structure allowing solvent access to the fluorophoric tripeptide will quench the fluorescence. The compact structure makes the proteins of the invention, e.g., GFP, very stable under diverse and/or harsh conditions such as protease treatment, making them extremely useful reporters in general.

In alternative aspects of the invention, proteins of the invention have fluorescent properties that are unaffected by prolonged treatment with bases, e.g., 6M guanidine HCl, chaotropic agents, e.g., 8M urea, detergents, e.g., 1% SDS, various proteases such as trypsin, chymotrypsin, papain, subtilisin, thermolysin or pancreatin. In alternative aspects of the invention, proteins of the invention have fluorescent properties that are unaffected by a broad range of pH stability, e.g., from about pH 3.5 to 12, or, about 5.5 to 11. For example, exemplary proteins can be very resistant to denaturation, requiring treatment with 6 M guanidine hydrochloride at 90° C. or pH of <4.0 or >12.0. In one aspect, partial to near total renaturation occurs within minutes following reversal of denaturing conditions by dialysis or neutralization. In one aspect, the fluorescent properties of the protein are unaffected by prolonged treatment with 6M guanidine HCl, 8M urea or 1% SDS, and two day treatment with various proteases such as trypsin, chymotrypsin, papain, subtilisin, thermolysin and pancreatin at concentrations up to 1 mg/ml fail to alter the intensity of GFP fluorescence. GFP is stable in neutral buffers up to 65° C., and displays a broad range of pH stability from 5.5 to 12.

The invention also provides a “humanized” fluorescent protein for use in mammalian cells (see, e.g., Haas et al., Current Biology 6 (1996), 315-324; Yang et al., Nucleic Acids Research 24 (1996), 4592-4593).

The present invention exploits the unique properties of novel fluorescent polypeptides to provide proteins that fluoresce in a variety of colors (wavelengths). The invention provides pH-dependent fluorescence proteins. Moreover, the fluorescent polypeptides of the invention are remarkably versatile. They can be tailored to function in organic solvents, operate at extreme pHs (for example, high pHs and low pHs), extreme temperatures (for example, high temperatures and low temperatures), and extreme salinity levels (for example, high salinity and low salinity).

Other benefits of the fluorescent proteins of the invention include fluorescence resonance energy transfer (FRET) possibilities based on new spectra and better suitability for larger excitation. One exemplary fluorescent polypeptide having a sequence as set forth in SEQ ID NO:8 has novel characteristics, e.g., excitation maximum at 448 nm, and the emission maximum at 491 nm.

The exemplary SEQ ID NO:8 is

Met Ser His Ser Lys Ser Val Ile Lys Asp Glu Met Phe Ile Lys Ile His Leu Glu Gly Thr
Phe Asn Gly His Lys Phe Glu Ile Glu Gly Glu Gly Asn Gly Lys Pro Tyr Ala Gly Thr Asn
Phe Val Lys Leu Val Val Thr Lys Gly Gly Pro Leu Pro Phe Gly Trp His Ile Leu Ser Pro
Gln Leu Gln Tyr Gly Asn Lys Ser Phe Val Ser Tyr Pro Ala Asp Ile Pro Asp Tyr Ile Lys
Leu Ser Phe Pro Glu Gly Phe Thr Trp Glu Arg Ile Met Thr Phe Glu Asp Gly Gly Val Cys
Cys Ile Thr Ser Asp Ile Ser Met Lys Ser Asn Asn Cys Phe Phe Tyr Asp Ile Lys Phe Thr
Gly Met Asn Phe Pro Pro Asn Gly Pro Val Val Gln Lys Lys Thr Thr Gly Trp Glu Pro Ser
Thr Glu Arg Leu Tyr Leu Arg Asp Gly Val Leu Thr Gly Asp Ile His Lys Thr Leu Lys Leu
Ser Gly Gly Gly His Tyr Thr Cys Val Phe Lys Thr Ile Tyr Arg Ser Lys Lys Asn Leu Thr
Leu Pro Asp Cys Phe Tyr Tyr Val Asp Thr Lys Leu Asp Ile Arg Lys Phe Asp Glu Asn Tyr
Ile Asn Val Glu Gln Asp Glu Ile Ala Thr Ala Arg His His Gly Leu Lys

The invention also provides methods of discovering new fluorescent polypeptides using the nucleic acids, polypeptides and antibodies of the invention. In one aspect, lambda phage libraries are screened for expression-based discovery of fluorescent polypeptides. In one aspect, the invention uses lambda phage libraries in screening to allow detection of toxic clones; improved access to substrate; reduced need for engineering a host, by-passing the potential for any bias resulting from mass excision of the library; and, faster growth at low clone densities. Screening of lambda phage libraries can be in liquid phase or in solid phase. In one aspect, the invention provides screening in liquid phase. This gives a greater flexibility in assay conditions; additional substrate flexibility; higher sensitivity for weak clones; and ease of automation over solid phase screening.

The invention provides screening methods using the proteins and nucleic acids of the invention and robotic automation to enable the execution of many thousands of biocatalytic reactions and screening assays in a short period of time, e.g., per day, as well as ensuring a high level of accuracy and reproducibility (see discussion of arrays, below). As a result, a library of derivative compounds can be produced in a matter of weeks. For further teachings on modification of molecules, including small molecules, see PCT/US94/09174.

### Hybrid Fluorescent Polypeptides and Peptide Libraries

In one aspect, the invention provides hybrid fluorescent polypeptides and fusion proteins, including peptide libraries, comprising sequences of the invention. The peptide libraries comprising sequences of the invention are used to isolate peptide inhibitors of targets (e.g., receptors, enzymes) and to identify formal binding partners of targets (e.g., ligands, such as cytokines, hormones and the like).

The field of biomolecule screening for biologically and therapeutically relevant compounds is rapidly growing. Relevant biomolecules that have been the focus of such screening include chemical libraries, nucleic acid libraries and peptide libraries, in search of molecules that either inhibit or augment the biological activity of identified target molecules. With particular regard to peptide libraries, the isolation of peptide inhibitors of targets and the identification of formal binding partners of targets has been a key focus. Screening of combinatorial libraries of potential drugs on therapeutically relevant target cells is a rapidly growing and important field. However, one particular problem with peptide libraries is the difficulty assessing whether any particular peptide has been expressed, and at what level, prior to determining whether the peptide has a biological effect. Thus, in order to express and subsequently screen functional peptides in cells, the peptides need to be expressed in sufficient quantities to overcome catabolic mechanisms such as proteolysis and transport out of the cytoplasm into endosomes.

In one aspect, the fusion proteins of the invention (e.g., the peptide moiety) are conformationally stabilized (relative to linear peptides) to allow a higher binding affinity for their cellular targets. The present invention provides fusions of fluorescent proteins of the invention and other peptides, including known and random peptides, that are fused in such a manner that the structure of the fluorescent polypeptides is not significantly perturbed and the peptide is metabolically or structurally conformationally stabilized. This allows the creation of a peptide library that is easily monitored, both for its presence within cells and its quantity.

The present invention provides fusions of fluorescent polypeptides of the invention, including green fluorescent protein (GFP) and cyan fluorescent protein (CFP) and random peptides. In one aspect, the fluorescent polypeptides of the invention are shorter or longer than a corresponding wild type sequence. Thus, in one aspect, included within the definition of fluorescent polypeptides are portions or fragments of the wild type sequence. For example, GFP and CFP deletion mutants are provided. It is known in the art that at the N-terminus, only the first amino acid of the protein may be deleted without loss of fluorescence. At the C-terminus, up to 7 residues can be deleted without loss of fluorescence, see, e.g., Phillips (1997) Current Opin. Structural Biol. 7:821.

In one aspect, the fluorescent polypeptides of the invention are derivatives or variants of GFP or CFP. For example, exemplary GFP or CFP may contain at least one amino acid substitution, deletion or insertion. The amino acid substitution, insertion or deletion may occur at any residue within the GFP or CFP. These variants can be prepared by site specific mutagenesis of nucleotides in the DNA encoding the GFP or CFP, using cassette or PCR mutagenesis or other techniques well known in the art, to produce DNA encoding the variant, and thereafter expressing the DNA in recombinant cell culture as outlined above. Also, variant GFP protein fragments having up to about 100-150 residues may be prepared by in vitro synthesis using established techniques.

Amino acid sequence variants of the invention can be characterized by the predetermined nature of the variation, a feature that sets them apart from naturally occurring allelic or interspecies variation of the GFP protein amino acid sequence. In one aspect, the variants of the invention exhibit the same qualitative biological activity as the naturally occurring analogue, although variants can also be selected which have modified characteristics. In one aspect, a derivative can have at least 0.65-0.88 or 2.7-3.6 relative brightness (maximum extinction coefficient multiplied by quantum field) as compared to wtGFP. In one aspect, a derivative has enough fluorescence to allow sorting and/or detection above background, for example, using a fluorescence-activated cell sorter (FACS) machine. In some aspects, it is possible to detect the fusion proteins non-fluorescently, using, for example, antibodies directed to either an epitope tag (i.e. purification sequence) or to the fluorescent polypeptide itself.

While the site or region for introducing an amino acid sequence variation is predetermined, the mutation per se need not be predetermined. For example, in order to optimize the performance of a mutation at a given site, random mutagenesis may be conducted at the target codon or region and the expressed fluorescent polypeptides variants screened for the optimal combination of desired activity. Techniques for making substitution mutations at predetermined sites in DNA having a known sequence are well known, for example, M13 primer mutagenesis and PCR mutagenesis. Screening of the mutants is done using assays of fluorescent protein activities, i.e. fluorescence. In alternative aspects, amino acid substitutions can be single residues; insertions can be on the order of from about 1 to 20 amino acids, although considerably larger insertions may be tolerated. Deletions can range from about 1 to about 20 residues, although in some cases deletions may be much larger. To obtain a final derivative with the optimal properties, substitutions, deletions, insertions or any combination thereof may be used. Generally, these changes are done on a few amino acids to minimize the alteration of the molecule. However, larger changes may be tolerated in certain circumstances.

The invention provides fluorescent polypeptides where the structure of the polypeptide backbone, the secondary or the tertiary structure, e.g., an alpha-helical or beta-sheet structure, has been modified. In one aspect, the charge or hydrophobicity has been modified. In one aspect, the bulk of a side chain has been modified. Substantial changes in function or immunological identity are made by selecting substitutions that are less conservative. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine. The variants can exhibit the same qualitative biological activity (i.e. fluorescence) although variants can be selected to modify the characteristics of the fluorescent proteins as needed.

In one aspect, fluorescent proteins of the invention comprise epitopes or purification tags, signal sequences or other fusion sequences, etc. In one aspect, the fluorescent proteins of the invention can be fused to a random peptide to form a fusion polypeptide. By “fused” or “operably linked” herein is meant that the random peptide and the fluorescent polypeptide are linked together, in such a manner as to minimize the disruption to the stability of the fluorescent polypeptide structure (i.e. it can retain fluorescence) or maintains a Tm of at least 42° C. The fusion polypeptide (or fusion polynucleotide encoding the fusion polypeptide) can comprise further components as well, including multiple peptides at multiple loops.

In one aspect, the peptides and nucleic acids encoding them are randomized, either fully randomized or they are biased in their randomization, e.g. in nucleotide/residue frequency generally or per position. “Randomized” means that each nucleic acid and peptide consists of essentially random nucleotides and amino acids, respectively. In one aspect, the nucleic acids that give rise to the peptides can be chemically synthesized, and thus may incorporate any nucleotide at any position. Thus, when the nucleic acids are expressed to form peptides, any amino acid residue may be incorporated at any position. The synthetic process can be designed to generate randomized nucleic acids, to allow the formation of all or most of the possible combinations over the length of the nucleic acid, thus forming a library of randomized nucleic acids. The library can provide a sufficiently structurally diverse population of randomized expression products to affect a probabilistically sufficient range of cellular responses to provide one or more cells exhibiting a desired response. Thus, the invention provides an interaction library large enough so that at least one of its members will have a structure that gives it affinity for some molecule, protein, or other factor whose activity is necessary for completion of a signaling pathway.

In one aspect, a peptide library of the invention is fully randomized, with no sequence preferences or constants at any position. In another aspect, the library is biased, that is, some positions within the sequence are either held constant, or are selected from a limited number of possibilities. For example, in one aspect, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc. For example, individual residues may be fixed in the random peptide sequence of the insert to create a structural bias. In an alternative aspect, the random libraries can be biased to a particular secondary structure by including an appropriate number of residues (beyond the glycine linkers) that prefer the particular secondary structure.

In one aspect, the bias is towards peptides that interact with known classes of molecules. For example, it is known that much of intracellular signaling is carried out via short regions of polypeptides interacting with other polypeptides through small peptide domains. For instance, a short region from the HIV-1 envelope cytoplasmic domain has been previously shown to block the action of cellular calmodulin. Regions of the Fas cytoplasmic domain, which shows homology to the mastoparan toxin from wasps, can be limited to a short peptide region with death-inducing apoptotic or G protein inducing functions. Thus, a number of molecules or protein domains are suitable as starting points for the generation of biased randomized peptides. A large number of small molecule domains are known, that confer a common function, structure or affinity. In addition, areas of weak amino acid homology may have strong structural homology. Exemplary molecules, domains, and/or corresponding consensus sequences used in the invention (e.g., incorporated into fusion proteins of the invention) include SH-2 domains, SH-3 domains, Pleckstrin, death domains, protease cleavage/recognition sites, enzyme inhibitors, enzyme substrates, Traf, etc. Similarly, there are a number of known nucleic acid binding proteins containing domains suitable for use in the invention, e.g., leucine zipper consensus sequences.

In alternative aspects, the invention provides ranges of random peptides from about 4 to about 50 residues in length, from about 5 to about 30 residues, or, from about 10 to about 20 residues in length. Random peptides can be fused to the fluorescent polypeptides of the invention in a variety of positions to form fusion polypeptides. The fusion polypeptide can include additional components, including, but not limited to, fusion partners and linkers.

In one aspect, a “fusion partner” of a fusion protein of the invention (comprising a sequence of the invention) is associated with a random peptide that confers upon all members of the library in that class a common function or ability. Fusion partners can be heterologous (i.e. not native to the host cell), or synthetic (not native to any cell). Suitable fusion partners include, but are not limited to: (a) presentation structures, which provide the peptides in a conformationally restricted or stable form; (b) targeting sequences, which allow the localization of the peptide into a subcellular or extracellular compartment; (c) rescue sequences as defined below, which allow the purification or isolation of either the peptides or the nucleic acids encoding them; (d) stability sequences, which confer stability or protection from degradation to the peptide or the nucleic acid encoding it, for example resistance to proteolytic degradation; (e) linker sequences, which conformationally decouple the random peptide elements from the fluorescent polypeptide itself, which keep the peptide from interfering with fluorescent protein folding; or (f), any combination of (a), (b), (c), (d) and (e) as well as linker sequences as needed. See, e.g., U.S. Pat. No. 6,180,343.

In one aspect, the fusion partner of a fusion protein of the invention (comprising a sequence of the invention) is a presentation structure. Presentation structure means a sequence, which, when fused to peptides, causes the peptides to assume a conformationally restricted form. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmacophore models and pharmaceuticals and will also likely lead to higher affinity interactions of the peptide with the target protein. In one aspect, presentation structures maximize accessibility to the peptide by presenting it on an exterior surface such as a loop, and also cause further conformational constraints in a peptide. Accordingly, suitable presentation structures comprise dimerization sequences, minibody structures, loops on beta turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc. In one aspect, the presentation structure is a coiled-coil structure, allowing the presentation of the randomized peptide on an exterior loop. See, for example, Myszka et al., Biochem. 33 (1994), 2362-2373. Using this system investigators have isolated peptides capable of high affinity interaction with the appropriate target.

In one aspect, the presentation structure is a minibody structure. A minibody is essentially composed of a minimal antibody complementarity region. The minibody presentation structure generally provides two randomizing regions that in the folded protein are presented along a single face of the tertiary structure. See, e.g., Bianchi et al., J. Mol. Biol. 236(2) (1994), 649-59.

In another aspect, the presentation structure is a sequence that contains generally two cysteine residues, such that a disulfide bond may be formed, resulting in a conformationally constrained sequence. This aspect can be used ex vivo, for example when secretory targeting sequences are used. Generally, any number of random sequences, with or without spacer or linking sequences, may be flanked with cysteine residues. In other aspects, effective presentation structures may be generated by the random regions themselves. For example, the random regions may be “doped” with cysteine residues that, under the appropriate redox conditions, may result in highly crosslinked structured conformations, similar to a presentation structure. Similarly, the randomization regions may be controlled to contain a certain number of residues to confer beta-sheet or alpha-helical structures.

In one aspect, the presentation structure is a dimerization sequence, including self-binding peptides. A dimerization sequence allows the non-covalent association of two peptide sequences, which can be the same or different, with sufficient affinity to remain associated under normal physiological conditions. These sequences may be used in several ways. In one aspect, one terminus of the random peptide is joined to a first dimerization sequence and the other terminus is joined to a second dimerization sequence, which can be the same or different from the first sequence. This allows the formation of a loop upon association of the dimerizing sequences. Alternatively, the use of these sequences effectively allows small libraries of random peptides to become large libraries if two peptides per cell are generated which then dimerize, to form an effective library. It also allows the formation of longer random peptides, if needed, or more structurally complex random peptide molecules. In one aspect, the dimers may be homo- or heterodimers. In another aspect, dimerization sequences may be a single sequence that self-aggregates, or two different sequences that associate.

In one aspect, the fusion partner of a fusion protein of the invention (comprising a sequence of the invention) is a targeting sequence and the fusion protein of the invention is used to target the movement and location of proteins in a cell. For example, RAF1 when localized to the mitochondrial membrane can inhibit the anti-apoptotic effect of BCL-2. Membrane bound Sos induces Ras mediated signaling in T-lymphocytes. These mechanisms are thought to rely on the principle of limiting the search space for ligands, that is to say, the localization of a protein to the plasma membrane limits the search for its ligand to that limited dimensional space near the membrane as opposed to the three dimensional space of the cytoplasm. Alternatively, the concentration of a protein can also be simply increased by nature of the localization. Shuttling the proteins into the nucleus confines them to a smaller space thereby increasing concentration. Finally, the ligand or target may simply be localized to a specific compartment, and inhibitors must be localized appropriately.

The invention provides targeting sequences comprising fluorescent proteins of the invention capable of causing binding of the expression product to a predetermined molecule or class of molecules while retaining bioactivity of the expression product, (for example by using enzyme inhibitor or substrate sequences to target a class of relevant enzymes); sequences signaling selective degradation, of itself or co-bound proteins; and signal sequences capable of constitutively localizing the peptides to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane; and b) extracellular locations via a secretory signal. In one aspect, localization can be to either subcellular locations or to the outside of the cell via secretion.

In one aspect, the fusion partner comprises a fluorescent protein of the invention and a rescue sequence. A rescue sequence is a sequence that may be used to purify or isolate either the peptide or the nucleic acid encoding it. Thus, for example, peptide rescue sequences include purification sequences for use with Ni affinity columns and epitope tags for detection, immunoprecipitation or FACS (fluorescence-activated cell sorting). In another aspect, the rescue sequence may be a unique oligonucleotide sequence that serves as a probe target site to allow the quick and easy isolation of the retroviral construct, via PCR, related techniques, or hybridization.

In one aspect, the fusion partner comprises a fluorescent protein of the invention and a stability sequence to confer stability to the peptide or the nucleic acid encoding it. Thus, for example, peptides may be stabilized by the incorporation of glycines after the initiation methionine (MG or MGGO), for protection of the peptide to ubiquitination as per Varshavsky's N-End Rule, thus conferring long half-life in the cytoplasm.

In one aspect, the fusion partner comprises a fluorescent protein of the invention and a linker or tethering sequence. Linker sequences between various targeting sequences (for example, membrane targeting sequences) and the other components of the constructs (such as the randomized peptides) may be desirable to allow the peptides to interact with potential targets unhindered. The peptide is connected to a fluorescent protein of the invention via linkers. While one aspect of the invention can provide the direct linkage of the peptide to the fluorescent polypeptide, or of the peptide and any fusion partners to the fluorescent polypeptide, another aspect of the invention provides linkers at one or both ends of the peptide. Therefore, when attached either to the N- or C-terminus, one linker may be used. When the peptide is inserted in an internal position, the invention provides at least one or two linker, one at each terminus of the peptide. Linkers are generally preferred in order to conformationally decouple any insertion sequence (i.e. the peptide) from the fluorescent polypeptide structure itself, to minimize local distortions in the fluorescent polypeptide structure that can either destabilize folding intermediates or allow access to the protein's buried tripeptide fluorophore, which decreases (or eliminates) fluorescence due to exposure to exogenous collisional fluorescence quenchers (see Phillips, Curr. Opin. Structural Biology 7 (1997), 821).

The fusion partners may be placed anywhere (i.e. N-terminal, C-terminal, internal) in the structure as the biology and activity permits. In addition, it is also possible to fuse one or more of these fusion partners to fluorescent proteins of the invention. Thus, for example, the fluorescent polypeptide may contain a targeting sequence (either N-terminally, C-terminally, or internally, as described below) at one location, and a rescue sequence in the same place or a different place on the molecule. Thus, any combination of fusion partners and peptides and fluorescent proteins may be made.

The invention further provides fusion (hybrid) nucleic acids comprising a nucleic acid of the invention and nucleic acids encoding polypeptides and fusion proteins of the invention. As will be appreciated by those in the art, due to the degeneracy of the genetic code, an extremely large number of nucleic acids may be made, all of which encode the fusion proteins of the present invention. Thus, having identified a particular amino acid sequence, skilled artisans could make any number of different nucleic acids, by simply modifying the sequence of one or more codons in a way that does not change the amino acid sequence of the fusion protein.

The invention provides a variety of expression vectors comprising nucleic acids of the invention, including those encoding a fusion protein. The expression vectors may be either self-replicating extra chromosomal vectors or vectors which integrate into a host genome. Generally, these expression vectors include transcriptional and translational regulatory nucleic acid operably linked to the nucleic acid encoding the fusion protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site.

Transcriptional and translational regulatory sequences used in the expression cassettes and vectors of the invention include, but are not limited to, promoter sequences, ribosomal binding sites, transcriptional start and stop sequences, translational start and stop sequences, and enhancer or activator sequences. In one aspect, the regulatory sequences include a promoter and transcriptional start and stop sequences. Promoter sequences encode either constitutive or inducible promoters. The promoters may be either naturally occurring promoters or hybrid promoters. Hybrid promoters, which combine elements of more than one promoter, are also known in the art, and are useful in the present invention. In one aspect, the promoters are strong promoters, allowing high expression in cells, particularly mammalian cells, such as the CMV promoter, particularly in combination with a Tet regulatory element.

In addition, the expression vector may comprise additional elements. In one exemplification, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences that flank the expression construct. The integrating vector may be directed to a specific locus in the host cell by selecting the appropriate homologous sequence for inclusion in the vector. Constructs for integrating vectors are well known in the art.

In one aspect, the nucleic acids or vectors of the invention are introduced into the cells for screening, thus, the nucleic acids enter the cells in a manner suitable for subsequent expression of the nucleic acid. The method of introduction is largely dictated by the targeted cell type. Exemplary methods include CaPO₄ precipitation, liposome fusion, lipofection (e.g., LIPOFECTIN™), electroporation, viral infection, etc. The candidate nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.). As many pharmaceutically important screens require human or model mammalian cell targets, retroviral vectors capable of transfecting such targets are preferred.

The fusion proteins of the present invention can be produced by culturing a host cell transformed with an expression vector comprising a nucleic acid encoding a fusion protein (including a sequence of the invention), under the appropriate conditions to induce or cause expression of the fusion protein. The conditions appropriate for fusion protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some aspects, the timing of the harvest is important. For example, the baculoviral systems used in insect cell expression are lytic viruses, and thus harvest time selection can be crucial for product yield. Host cells used to practice the invention include yeast, bacteria, Archaebacteria, fungi, and insect and animal cells, including mammalian cells, Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, immortalized mammalian myeloid and lymphoid cell lines, Jurkat cells, mast cells and other endocrine and exocrine cells, and neuronal cells.

In one aspect, the fusion proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for the fusion protein into mRNA. A promoter will have a transcription initiating region, which is usually placed Oproximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific posttranslational cleavage and polyadenylation. Examples of transcription terminator and polyadenylation signals include those derived form SV40.

Expression vectors of the invention may also include a selectable marker gene to allow for the selection of bacterial strains that have been transformed, e.g., genes that render the bacteria resistant to drugs such as ampicillin, chloramphenicol, erythromycin, kanamycin, neomycin and tetracycline. Selectable markers can also include biosynthetic genes, such as those in the histidine, tryptophan and leucine biosynthetic pathways.

### Industrial and Medical Uses

The invention provides many industrial uses and medical applications for the fluorescent polypeptides of the invention, including their use as reporters. Methods of using fluorescent polypeptides in industrial applications are well known in the art. See, e.g., U.S. Pat. No. 6,027,881, describing the use of the GFP mutants and their expression in prokaryotic and eukaryotic cells.

Retroviral Vectors

In one aspect, the fluorescent polypeptides of the invention can be used to trace retroviral vectors. Retroviral vectors can be useful to modify eukaryotic cells because of the high efficiency with which the retroviral vectors transduce target cells and integrate into the target cell genome. Additionally, the retroviruses harboring the retroviral vector are capable of infecting cells from a wide variety of tissues. Preparation of retroviral vectors and their uses are described in many publications including U.S. Pat. No. 4,405,712, Gilboa (1986), Biotechniques 4:504-512, Mann, et al. (1983), Cell 33:153-159, Cone and Mulligan (1984), Proc. Natl. Acad. Sci. USA 81:6349-6353, Eglitis, M. A, et al. (1988) Biotechniques 6:608-614, Miller, A. D. et al. (1989) Biotechniques 7:981-990.

Detection of Nucleic Acids and Polypeptides

The nucleic acids and proteins of the invention can be detected, confirmed and quantified by any of a number of means well known to those of skill in the art. General methods for detecting both nucleic acids and corresponding proteins include analytic biochemical methods such as spectrophotometry, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunosorbent assays (ELISAs), immunofluorescent assays, and the like. The detection of nucleic acids proceeds by well known methods such as Southern analysis, northern analysis, gel electrophoresis, PCR, radiolabeling, scintillation counting, and affinity chromatography.

Fluorescence Assays

The fluorescent proteins of the invention can be detected using fluorescence assays. When a fluorophore such as protein that is capable of fluorescing is exposed to a light of appropriate wavelength, it will absorb and store light and then release the stored light energy. The range of wavelengths that a fluorophore is capable of absorbing is the excitation spectrum and the range of wavelengths of light that a fluorophore is capable of emitting is the emission or fluorescence spectrum. The excitation and fluorescence spectra for a given fluorophore usually differ and may be readily measured using known instruments and methods. For example, scintillation counters and photometers (e.g. luminometers), photographic film, and solid state devices such as charge coupled devices, may be used to detect and measure the emission of light.

The fluorescent polypeptides of the present invention can be used in standard assays involving a fluorescent marker. For example, ligand-ligator (e.g., receptor-ligand) binding pairs that can be modified with fluorescent proteins of the invention without disrupting the ability of each to bind to the other can form the basis of an assay encompassed by the present invention. These and other assays are known in the art and their use with the fluorescent polypeptides of the present invention will become obvious to one skilled in the art in light of the teachings disclosed herein. Examples of such assays include competitive assays wherein labeled and unlabeled ligands competitively bind to a ligator, noncompetitive assay where a ligand is captured by a ligator and either measured directly or “sandwiched” with a secondary ligator that is labeled. Still other types of assays include immunoassays, single-step homogeneous assays, multiple-step heterogeneous assays, and enzyme assays.

The fluorescent polypeptides of the invention can be combined with fluorescent microscopy using known techniques (see, e.g., Stauber (1995) Virol. 213:439-454) or with fluorescence activated cell sorting (FACS) to detect and optionally purify or clone cells that express specific recombinant constructs. For a brief overview of the FACS and its uses, see: Herzenberg (1976) Sci. Amer. 234, 108; see also FLOW CYTOMETRY AND SORTING, eds. Melamad, Mullaney and Mendelsohn, John Wiley and Sons, Inc., New York, 1979). Briefly, fluorescence activated cell sorters take a suspension of cells and pass them single file into the light path of a laser placed near a detector. The laser usually has a set wavelength. The detector measures the fluorescent emission intensity of each cell as it passes through the instrument and generates a histogram plot of cell number versus fluorescent intensity. Gates or limits can be placed on the histogram thus identifying a particular population of cells. In one aspect, the cell sorter is set up to select cells having the highest probe intensity, usually a small fraction of the cells in the culture, and to separate these selected cells away from all the other cells. The level of intensity at which the sorter is set and the fraction of cells that is selected, depend on the condition of the parent culture and the criteria of the isolation.

A skilled artisan can design a number of fluorescence-based assays using the fluorescent polypeptides of the invention. For example, translocation of proteins fused to the polypeptides of the invention can be visualized. The translocation of intracellular proteins to a specific organelle, can be visualized by fusing the protein of interest to one fluorescent protein, e.g. fluorescent proteins of the invention, and labeling the organelle with another fluorescent protein which emits light of a different wavelength. Translocation can then be detected as a spectral shift of the fluorescent proteins in the specific organelle. See, e.g., U.S. Pat. No. 6,172,188.

The fluorescent polypeptides of the invention can also be used as a secretion marker. By fusion of the fluorescent polypeptides to a signal peptide or a peptide to be secreted, secretion may be followed on-line in living cells. A precondition for that is that the maturation of a detectable number of novel fluorescent protein molecules occurs faster than the secretion.

In another aspect, the fluorescent polypeptides of the invention can be used as genetic reporter or protein tag in transgenic animals (e.g., fish, mice, goats, rabbits, etc.). Due to the strong fluorescence of the fluorescent polypeptides, they are suitable as tags for proteins and gene expression. In one aspect, the fluorescent polypeptides can be used to produce transgenic animals such as fish, mice, goats, rabbits and the like.

In one aspect, the fluorescent polypeptides of the invention can be used as a marker for changes in cell morphology. Expression of the fluorescent polypeptides in cells allows easy detection of changes in cell morphology, e.g. blabbing, caused by cytotoxic agents or apoptosis. Such morphological changes are difficult to visualize in intact cells without the use of fluorescent probes.

Gene Therapy

The nucleic acids, vectors and fluorescent proteins of the invention are used in gene therapy. Gene therapy in general is the correction of genetic defects by insertion of exogenous cellular genes that encode a desired function into cells that lack that function, such that the expression of an exogenous gene corrects a genetic defect or causes the destruction of cells that are genetically defective. Methods of gene therapy are well known in the art, see, for example, Lu (1994) Human Gene Therapy 5:203; Smith (1992) J. Hematotherapy 1:155; Cassel (1993) Exp. Hematol. 21-:585 (1993); Larrick, J. W. and Burck, K. L., GENE THERAPY: APPLICATION OF MOLECULAR BIOLOGY, Elsevier Science Publishing Co., Inc., New York, N.Y. (1991) and Kreigler, M. GENE TRANSFER AND EXPRESSION: A LABORATORY MANUAL, W. H. Freeman and Company, New York (1990). See also U.S. Pat. No. 6,027,881.

An exemplary method provides (a) obtaining from a patient a viable sample of cells; (b) inserting into these cells a nucleic acid segment encoding a desired gene product; (c) identifying and isolating cells and cell lines that express the gene product; (d) re-introducing cells that express the gene product; (e) removing from the patient an aliquot of tissue including cells resulting from step c and their progeny; and (f) determining the quantity of the cells resulting from step c and their progeny, in said aliquot. The introduction into cells in step (c) of a polycistronic vector that encodes fluorescent polypeptide of the invention in addition to the desired gene allows for the quick identification of viable cells that contain and express the desired gene.

In one aspect, a nucleic acid of the invention is inserted into selected tissue cells in situ, for example into cancerous or diseased cells, by contacting the target cells in situ with retroviral vectors that encode the gene product in question. Here, it is important to quickly and reliably assess which and what proportion of cells have been transfected. Co-expression of the fluorescent proteins of the invention permits a quick assessment of proportion of cells that are transfected, and levels of expression

Diagnostics

The fluorescent proteins of the invention are used in diagnostic testing. A gene encoding a fluorescent polypeptide, when placed under the control of promoters induced by various agents, can serve as an indicator for these agents. Established cell lines or cells and tissues from transgenic animals carrying fluorescent proteins of the invention expressed under the desired promoter will become fluorescent in the presence of the inducing agent. The transgenic animals can be transgenic animals of the invention.

Viral promoters which are transactivated by the corresponding virus, promoters of heat shock genes which are induced by various cellular stresses as well as promoters which are sensitive to organismal responses, e.g. inflammation, can be used in combination with the fluorescent proteins of the invention in diagnostics.

The effect of selected culture conditions and components (salt concentrations, pH, temperature, trans-acting regulatory substances, hormones, cell-cell contacts, ligands of cell surface and internal receptors) can be assessed by incubating cells in which sequences encoding fluorescent proteins of the invention are operably linked to nucleic acids (especially regulatory elements such as promoters) derived from a selected gene, and detecting the expression and location of fluorescence. See, e.g., U.S. Pat. No. 6,027,881.

Toxicology

The fluorescent proteins of the invention are used in toxicology methodologies. Assessment of the mutagenic potential of any compound is a prerequisite for its use. Until recently, the Ames assay in Salmonella and tests based on chromosomal aberrations or sister chromatid exchanges in cultured mammalian cells were the main tools in toxicology. However, both assays are of limited sensitivity and specificity and do not allow studies on mutation induction in various organs or tissues of the intact organism. The introduction of transgenic mice with a mutational target in a shuttle vector has made possible the detection of induced mutations in different tissues in vivo. The assay involves DNA isolation from tissues of exposed mice, packaging of the target DNA into bacteriophage lambda particles and subsequent infection of E. coli. The mutational target in this assay is either the lacZ or lad genes and quantitation of blue vs. white plaques on the bacterial lawn allows for mutagenic assessment.

Use of the fluorescent proteins of the invention simplifies both the tissue culture and transgenic mouse procedures. Expression of fluorescent proteins of the invention under the control of a repressor, which in turn is driven by the promoter of a constitutively expressed gene, is a method for evaluating the mutagenic potential of an agent. The presence of fluorescent cells, following exposure of a cell line, tissue or whole animal carrying the fluorescent protein detection construct, will reflect the mutagenicity of the compound in question. Fluorescent proteins of the invention expressed under the control of the target DNA, the repressor gene, will only be synthesized when the repressor is inactivated or turned off or the repressor recognition sequences are mutated. Direct visualization of the detector cell line or tissue biopsy can qualitatively assess the mutagenicity of the agent, while FACS of the dissociated cells can provide for quantitative analysis.

Drug Screening

The fluorescent proteins of the invention are also used in drug detection system. These methods expedite and reduce the cost of some current drug screening procedures. A dual color screening system (DCSS), in which a fluorescent protein is placed under the promoter of a target gene and the fluorescent protein is expressed from a constitutive promoter, provides rapid analysis of agents that specifically affect the target gene. Established cell lines with the DCSS could be screened with hundreds of compounds in few hours. The desired drug will only influence the expression of fluorescent protein. Non-specific or cytotoxic effects can be detected by a second marker. The advantages of this system are that no exogenous substances are required for fluorescent protein detection, the assay can be used with single cells, cell populations, or cell extracts, and that the same detection technology and instrumentation is used for very rapid and non-destructive detection.

DCSS is used to search for antiviral agents that specifically block viral transcription without affecting cellular transcription. In the case of HIV, appropriate cell lines expressing a fluorescent protein of the invention under the HIV LTR and a fluorescent protein of the invention under a cellular constitutive promoter can be used to identify compounds that selectively inhibit HIV transcription. Reduction of only the green but not the cyan fluorescent signal will indicate drug specificity for the HIV promoter. Similar approaches could also be designed for other viruses.

DCSS is also used to search for antiparasitic agents. Established cell lines or transgenic nematodes or even parasitic extracts where expression of a fluorescent protein of the invention depends on parasite-specific trans-splicing sequences while a second fluorescent protein of the invention is under the control of host-specific cis splicing elements provides rapid screen of selective antiparasitic drugs.

Cancer Applications

In one aspect, the fluorescent polypeptides of the invention can be used in imaging of cancer invasion and metastasis. Thus, the use of fluorescent proteins to fluorescently tag tumor cells allows investigators to open the “black box” of metastasis in order to visualize the behavior of tumor cells in living tissues. Analysis of cells leaving the primary tumor indicates that highly metastatic cells are able to polarize more effectively towards blood vessels while poorly metastatic cells fragment more often when interacting with blood. In addition, there appear to be greater numbers of host immune system cells interacting with metastatic tumors. After arresting in target organs such as the lungs or liver, most tumor cells become dormant or apoptosis. A small fraction of the arrested cells form metastases. In some target organs, migration of tumor cells may enhance the ability to form metastases. Cancer cell lines can be stably transfected with the fluorescent polypeptides of the invention in order to track metastases in fresh tissue at ultra-high resolution. This can be further used for innovative drug discovery and mechanism studies and serve as a bridge linking preclinical and clinical research and drug development. See, e.g., Hoffman, Invest New Drugs 1999; 17(4):343-59, and Condeelis et al., Eur J. Cancer. 2000 October; 36(16):2172-3.

Screening Methodologies and “on-Line” Monitoring Devices

In practicing the methods of the invention, a variety of apparatus and methodologies can be used to in conjunction with the polypeptides and nucleic acids of the invention, e.g., to screen polypeptides for fluorescent activity, to screen compounds as potential quenchers of fluorescent activity, for antibodies that bind to a polypeptide of the invention, for nucleic acids that hybridize to a nucleic acid of the invention, to screen for cells expressing a polypeptide of the invention and the like.

Capillary Arrays

Capillary arrays, such as the GIGAMATRIX™, Diversa Corporation, San Diego, Calif., can be used to in the methods of the invention. Nucleic acids or polypeptides of the invention can be immobilized to or applied to an array, including capillary arrays. Arrays can be used to screen for or monitor libraries of compositions (e.g., small molecules, antibodies, nucleic acids, etc.) for their ability to bind to or modulate the activity of a nucleic acid or a polypeptide of the invention. Capillary arrays provide another system for holding and screening samples. For example, a sample screening apparatus can include a plurality of capillaries formed into an array of adjacent capillaries, wherein each capillary comprises at least one wall defining a lumen for retaining a sample. The apparatus can further include interstitial material disposed between adjacent capillaries in the array, and one or more reference indicia formed within of the interstitial material. A capillary for screening a sample, wherein the capillary is adapted for being bound in an array of capillaries, can include a first wall defining a lumen for retaining the sample, and a second wall formed of a filtering material, for filtering excitation energy provided to the lumen to excite the sample.

A polypeptide or nucleic acid, e.g., a ligand, can be introduced into a first component into at least a portion of a capillary of a capillary array. Each capillary of the capillary array can comprise at least one wall defining a lumen for retaining the first component. An air bubble can be introduced into the capillary behind the first component. A second component can be introduced into the capillary, wherein the second component is separated from the first component by the air bubble. A sample of interest can be introduced as a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method can further include removing the first liquid from the capillary tube, wherein the bound detectable particle is maintained within the capillary, and introducing a second liquid into the capillary tube.

The capillary array can include a plurality of individual capillaries comprising at least one outer wall defining a lumen. The outer wall of the capillary can be one or more walls fused together. Similarly, the wall can define a lumen that is cylindrical, square, hexagonal or any other geometric shape so long as the walls form a lumen for retention of a liquid or sample. The capillaries of the capillary array can be held together in close proximity to form a planar structure. The capillaries can be bound together, by being fused (e.g., where the capillaries are made of glass), glued, bonded, or clamped side-by-side. The capillary array can be formed of any number of individual capillaries, for example, a range from 100 to 4,000,000 capillaries. A capillary array can form a micro titer plate having about 100,000 or more individual capillaries bound together.

Arrays, or “Biochips”

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. For example, in one aspect of the invention, a monitored parameter is transcript expression of a fluorescent polypeptide gene. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays” can also be used to simultaneously quantify a plurality of proteins. The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts.

In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

Antibodies and Antibody-Based Screening Methods

The invention provides isolated or recombinant antibodies that specifically bind to a fluorescent polypeptide of the invention. These antibodies can be used to isolate, identify or quantify the fluorescent polypeptides of the invention or related polypeptides. These antibodies can be used to isolate other polypeptides within the scope the invention or other related fluorescent polypeptides.

The antibodies can be used in immunoprecipitation, staining (e.g., FACS), immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention. Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

Polypeptides or peptides can be used to generate antibodies that bind specifically to the polypeptides of the invention. The resulting antibodies may be used in immunoaffinity chromatography procedures to isolate or purify the polypeptide or to determine whether the polypeptide is present in a biological sample. In such procedures, a protein preparation, such as an extract, or a biological sample is contacted with an antibody capable of specifically binding to one of the polypeptides of the invention.

In immunoaffinity procedures, the antibody is attached to a solid support, such as a bead or other column matrix. The protein preparation is placed in contact with the antibody under conditions in which the antibody specifically binds to one of the polypeptides of the invention. After a wash to remove non-specifically bound proteins, the specifically bound polypeptides are eluted.

The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays, and Western Blots.

Polyclonal antibodies generated against the polypeptides of the invention can be obtained by direct injection of the polypeptides into an animal or by administering the polypeptides to a non-human animal. The antibody so obtained will then bind the polypeptide itself. In this manner, even a sequence encoding only a fragment of the polypeptide can be used to generate antibodies that may bind to the whole native polypeptide. Such antibodies can then be used to isolate the polypeptide from cells expressing that polypeptide.

For preparation of monoclonal antibodies, any technique that provides antibodies produced by continuous cell line cultures can be used. Examples include the hybridoma technique, the trioma technique, the human B-cell hybridoma technique, and the EBV-hybridoma technique (see, e.g., Cole (1985) in Monoclonal Antibodies and Cancer Therapy, Alan R. Liss, Inc., pp. 77-96).

Techniques described for the production of single chain antibodies (see, e.g., U.S. Pat. No. 4,946,778) can be adapted to produce single chain antibodies to the polypeptides of the invention. Alternatively, transgenic mice may be used to express humanized antibodies to these polypeptides or fragments thereof.

Antibodies generated against the polypeptides of the invention may be used in screening for similar polypeptides from other organisms and samples. In such techniques, polypeptides from the organism are contacted with the antibody and those polypeptides that specifically bind the antibody are detected. Any of the procedures described above may be used to detect antibody binding.

### Kits

The invention provides kits comprising the compositions, e.g., nucleic acids, expression cassettes, vectors, cells, polypeptides (e.g., fluorescent polypeptides) and/or antibodies of the invention. The kits also can contain instructional material teaching the methodologies and industrial uses of the invention, as described herein.

### Measuring Metabolic Parameters

The methods of the invention provide whole cell evolution, or whole cell engineering, of a cell to develop a new cell strain having a new phenotype by modifying the genetic composition of the cell, where the genetic composition is modified by addition to the cell of a nucleic acid. To detect the new phenotype, at least one metabolic parameter of a modified cell is monitored in the cell in a “real time” or “on-line” time frame. In one aspect, a plurality of cells, such as a cell culture, is monitored in “real time” or “on-line.” In one aspect, a plurality of metabolic parameters is monitored in “real time” or “on-line.” Metabolic parameters can be monitored using the fluorescent polypeptides of the invention.

Metabolic flux analysis (MFA) is based on a known biochemistry framework. A linearly independent metabolic matrix is constructed based on the law of mass conservation and on the pseudo-steady state hypothesis (PSSH) on the intracellular metabolites. In practicing the methods of the invention, metabolic networks are established, including the:
identity of all pathway substrates, products and intermediary metabolites
identity of all the chemical reactions interconverting the pathway
metabolites, the stoichiometry of the pathway reactions,
identity of all the enzymes catalyzing the reactions, the enzyme reaction kinetics,
the regulatory interactions between pathway components, e.g. allosteric interactions, enzyme-enzyme interactions etc,
intracellular compartmentalization of enzymes or any other supramolecular organization of the enzymes, and,
the presence of any concentration gradients of metabolites, enzymes or effector molecules or diffusion barriers to their movement.

Once the metabolic network for a given strain is built, mathematic presentation by matrix notion can be introduced to estimate the intracellular metabolic fluxes if the on-line metabolome data is available. Metabolic phenotype relies on the changes of the whole metabolic network within a cell. Metabolic phenotype relies on the change of pathway utilization with respect to environmental conditions, genetic regulation, developmental state and the genotype, etc. In one aspect of the methods of the invention, after the on-line MFA calculation, the dynamic behavior of the cells, their phenotype and other properties are analyzed by investigating the pathway utilization. For example, if the glucose supply is increased and the oxygen decreased during the yeast fermentation, the utilization of respiratory pathways will be reduced and/or stopped, and the utilization of the fermentative pathways will dominate. Control of physiological state of cell cultures will become possible after the pathway analysis. The methods of the invention can help determine how to manipulate the fermentation by determining how to change the substrate supply, temperature, use of inducers, etc. to control the physiological state of cells to move along desirable direction. In practicing the methods of the invention, the MFA results can also be compared with transcriptome and proteome data to design experiments and protocols for metabolic engineering or gene shuffling, etc.

In practicing the methods of the invention, any modified or new phenotype can be conferred and detected, including new or improved characteristics in the cell. Any aspect of metabolism or growth can be monitored.

Monitoring Expression of an mRNA Transcript

In one aspect of the invention, the engineered phenotype comprises increasing or decreasing the expression of an mRNA transcript or generating new transcripts in a cell. This increased or decreased expression can be traced by use of a fluorescent polypeptide of the invention. mRNA transcripts, or messages, also can be detected and quantified by any method known in the art, including, e.g., Northern blots, quantitative amplification reactions, hybridization to arrays, and the like. Quantitative amplification reactions include, e.g., quantitative PCR, including, e.g., quantitative reverse transcription polymerase chain reaction, or RT-PCR; quantitative real time RT-PCR, or “real-time kinetic RT-PCR” (see, e.g., Kreuzer (2001) Br. J. Haematol. 114:313-318; Xia (2001) Transplantation 72:907-914).

In one aspect of the invention, the engineered phenotype is generated by knocking out expression of a homologous gene. The gene's coding sequence or one or more transcriptional control elements can be knocked out, e.g., promoters, enhancers. Thus, the expression of a transcript can be completely ablated or only decreased.

In one aspect of the invention, the engineered phenotype comprises increasing the expression of a homologous gene. This can be effected by knocking out of a negative control element, including a transcriptional regulatory element acting in cis- or trans-, or, mutagenizing a positive control element. One or more, or, all the transcripts of a cell can be measured by hybridization of a sample comprising transcripts of the cell, or, nucleic acids representative of or complementary to transcripts of a cell, by hybridization to immobilized nucleic acids on an array.

Monitoring Expression of a Polypeptides, Peptides and Amino Acids

In one aspect of the invention, the engineered phenotype comprises increasing or decreasing the expression of a polypeptide or generating new polypeptides in a cell. This increased or decreased expression can be traced by use of a fluorescent polypeptide of the invention. Polypeptides, peptides and amino acids also can be detected and quantified by any method known in the art, including, e.g., nuclear magnetic resonance (NMR), spectrophotometry, radiography (protein radiolabeling), electrophoresis, capillary electrophoresis, high performance liquid chromatography (HPLC), thin layer chromatography (TLC), hyperdiffusion chromatography, various immunological methods, e.g. immunoprecipitation, immunodiffusion, immuno-electrophoresis, radioimmunoassays (RIAs), enzyme-linked immunosorbent assays (ELISAs), immuno-fluorescent assays, gel electrophoresis (e.g., SDS-PAGE), staining with antibodies, fluorescent activated cell sorter (FACS), pyrolysis mass spectrometry, Fourier-Transform Infrared Spectrometry, Raman spectrometry, GC-MS, and LC-Electrospray and cap-LC-tandem-electrospray mass spectrometries, and the like. Novel bioactivities can also be screened using methods, or variations thereof, described in U.S. Pat. No. 6,057,103. Furthermore, as discussed below in detail, one or more, or, all the polypeptides of a cell can be measured using a protein array.

The invention will be further described with reference to the following examples; however, it is to be understood that the invention is not limited to such examples.

### EXAMPLES

### Example 1

### Expression Screening of cDNA Libraries from Eukaryotic Marine Sources

Expression screening of cDNA libraries from eukaryotic marine sources on the flow cytometer was performed. Sample collections were performed in diverse marine environments. Organisms previously identified to exhibit fluorescence were initially targeted. When possible, samples were collected using UV and/or blue light illumination (at night) to target sampling of specific areas within the organism exhibiting fluorescence. These samples presumably have an enriched level of expression of the fluorescing molecule and thus, would increase the likelihood of screening success. The UV/blue illumination technique was extended to other uncharacterized organisms to identifying novel sources of fluorescence. These samples were frozen in liquid nitrogen at the site of collection and sent packed in dry ice.

RNA was extracted from these samples and cDNA libraries synthesized. The cDNA was cloned into both lambda ZapExpress vectors as well as an expression vector containing an origin of replication (e.g., REPori, polyoma ori, etc.) designed for replication in mammalian cells. These libraries were screened in both prokaryotic and eukaryotic hosts.

The lambda ZapExpress libraries were excised and infected into E. coli hosts and screened for expression of fluorescent proteins using flow cytometry. The E. coli libraries were screened at several excitation (UV, 488, 568, 647 nm) and emission (400-700 nm) wavelengths and positive clones sorted. In some cases, multiple rounds of enrichment sorting were performed to identify a positive clone. The marine organism, Anenomia sulcata, was collected and used as a positive control for this entire protocol. The fluorescing tips of the Anenomia sulcata were collected and a cDNA library was synthesized and cloned it into lambda ZAPEXPRESS™. This library was expressed in E. coli. It exhibited a positive fluorescent colony at a rate of 1 in 700 using a plate-based system.

Use of a eukaryotic host can be important because of the possibility of enhanced expression due to similar codon usages, post-translational processing events, etc. The cDNA libraries in mammalian expression vectors were transfected into appropriate mammalian cells (e.g. CHO—P, COS, etc.) and screened 48 hours later for expression of fluorescent proteins on a flow cytometer. The screens were performed at several excitation and emission wavelengths on the flow cytometer. These libraries were also screened using sequenced-based methods (i.e., biopanning) using degenerate primers derived from the growing family of fluorescent proteins.

Optimal transfection conditions will be determined using the cycle 3 GFP from Invitrogen (San Diego, Calif.). If necessary, there were extra rounds of enrichment before isolating single positive clones. In one case, cells were be analyzed on a flow cytometer 48 hours after transfection and the fluorescent cells were be bulk sorted for enrichment. DNA was recovered from these cells using a Hirt's procedure, transformed into E. coli for amplification, and recovered by mini-prep from the E. coli. The DNA were transfected back into the mammalian cells and the positive cells singly sorted by FACS 48 hours later. Recovery of the gene can be accomplished using PCR with primers generated against the vector sequences.

Alternatively, a sequence-based approach to discovery of novel fluorescent proteins can be used. Using degenerate primers generated against conserved regions in known fluorescent proteins, lambda cDNA libraries can be screened for novel fluorescent proteins by biopanning using standard protocols. Again, the cDNA library from Anenomia sulcata can be screened as a positive control.

### Example 2

### Isolation of Exemplary Polypeptides of the Invention

Marine specimens were collected from Costa Rican and Bermudan waters with the aid of an underwater UV or blue light. Those samples that fluoresced when illuminated by the lights were collected, immediately frozen in liquid nitrogen, and subsequently stored at −80 C until further processing.

Total RNA was extracted from the frozen samples using a modified protocol from Chomezynzki and Sacchi (1987). In brief, the tissue sample was homogenized in guanidinium buffer using a Polytron and proteins/DNA separated from the RNA using phenol/chloroform. Subsequently, total RNA was selectively precipitated, washed, and resuspended in H₂0, Samples were enriched for mRNA by selection on an oligo (dT) cellulose column. Single stranded and double stranded cDNA were synthesized using the SMART cDNA synthesis kit from Clontech. The cDNAs were subsequently used as templates in PCR-based reactions for recovery of novel genes encoding for fluorescent proteins.

To generate primers for exemplary samples 1659 and 1663, the fluorescent proteins from these samples were extracted using traditional protein purification methods. In brief, the samples were homogenized using a mortar and pestle with a small amount of phosphate buffer. The homogenate was sonicated, diluted, and clarified by centrifugation. The fluorescent protein was precipitated from the supernatant by gradual isopropanol precipitation. The pellet containing the fluorescent protein was dissolved in phosphate buffer. Gel filtration, ion-exchange, and iso-electric focusing chromatography were used to purify the fluorescent protein for N′ terminal protein sequencing. Degenerate primers were generated from the N-terminal protein sequence and used as 5′ primers. Degenerate 3′ primers were generated from conserved sequences in known fluorescent proteins found in the public database. Using these 5′ and 3′ primers, PCR reactions were performed using the cDNA templates and specific DNA fragments were amplified. These fragments were sequenced and new primers generated at the 5′ end that corresponded exactly to the amplified sequences. To recover full length genes, the 3′ primer used for the cDNA synthesis reaction was used in another PCR reaction with the new 5′ gene-specific primer. Specific fragments were recovered and sequenced and new gene-specific primers were generated against the 3′ end of the coding sequence of the gene. The full coding sequence of the genes were amplified and cloned into an E. coli expression vector. The ligated vectors were introduced into BL21(DE3) cells and plated on agar plates. The colonies were scraped and run through the FACS where cells expressing a high level of fluorescence were isolated. The DNA was recovered using standard mini-prep DNA isolation procedures and the vector insert was sequenced.

For sample 1659, cells exhibiting a lower level of fluorescence were also chosen, resulting in the discovery of a novel fluorescent clone that had only 73-75% identity to the highly fluorescent clone. An additional step was necessary for the clones discovered from sample 1663. In this case, the N terminal protein sequence did not contain the expected methionine site. It was therefore necessary to recover the full 5′ end by cloning the cDNA into a TOPO vector and amplifying a fragment by PCR using a 5′ vector specific primer and a 3′ gene specific primer. This fragment was sequenced and a 5′ gene-specific primer was generated and used together with the 3′ gene-specific primer to amplify the full coding sequence of the gene.

Clones from sample 1658 were found exclusively using degenerate 5′ and 3′ primers generated against conserved sequences from the database. A specific DNA fragment was recovered and sequenced. Using a protocol similar to that described above, the full coding sequence was recovered by 2 separate PCR reactions using the either the 5′ or the 3′ primers employed during the synthesis of the cDNA together with the appropriate gene-specific primers generated from the first recovered fragment. The final full length coding sequence of the gene was recovered using a 5′ and 3′ gene-specific primer.

### Example 3

### Measuring Excitation and Emission Spectra

The excitation and emission spectra were measured using purified cyan and the green fluorescent proteins of the invention on a Perkin Elmer LS50B. Quantum yield and extinction coefficient measurements were determined following similar protocols as described in Matz et al. (Matz M. V., Fradkov, A. F., Labas Y. A., Savitsky A. P., Zaraisky A. G., Markelov M. L., and Lukyanov S. A., 1999, Fluorescent proteins from nonbioluminescent Anthozoa species. Nature Biotechnology, 17: 969-973). Specifically, Matz et al. determined concentrations of the proteins as described by Gill et al. (Gill, S. C. & Hippel, P. H., 1989, Calculation of protein extinction coefficients from amino acid sequence data. Anal. Biochem. 182:319-326), using the average extinction coefficients of tryptophan, tyrosine, and cysteine. Matz et al. calculated the extinction coefficients at 280 nm for proteins, using the model by Mach et al. (Mach, H., Middaugh, C. R. & Lewis, R. V., 1992, Statistical determination of the average values of the extinction coefficients of tryptophan and tyrosine in native proteins. Anal. Biochem. 200, 74-80). These values were then used to determine the concentrations of proteins and thereby the molar extinction coefficients in the visible band. Quantum yields were determined relative to wild-type GFP (Clontech). A Perkin-Elmer LS50B spectrometer (Beaconsfield, UK) was used for quantitative measurements. All samples were excited at 470 nm, at absorbance 0.02, and excitation and emission slits were 5 nm. The spectra were corrected for photomultiplier response and monochromator transmittance, transformed to a wave number and integrated.

A number of embodiments of the invention have been described. Nevertheless, it will be understood that various modifications may be made without departing from the spirit and scope of the invention. Accordingly, other embodiments are within the scope of the following claims.

What is claimed is:

1. An isolated or recombinant nucleic acid comprising a nucleic acid sequence having at least 75% sequence identity to SEQ ID NO:17 over a region of at least about 100 residues,
wherein the nucleic acid encodes a fluorescent polypeptide and the sequence identities are determined by analysis with a sequence comparison algorithm or by a visual inspection.

2. The isolated or recombinant nucleic acid of claim 1, wherein the nucleic acid comprises a nucleic acid sequence having at least 75% sequence identity to SEQ ID NO:17 over a region of at least about 200 residues.

3. The isolated or recombinant nucleic acid of claim 1, wherein the nucleic acid comprises a nucleic acid sequence having at least 75% sequence identity to SEQ ID NO:17 over a region of at least about 300 residues.

4. The isolated or recombinant nucleic acid of claim 1, wherein the nucleic acid comprises a nucleic acid sequence having at least 75% sequence identity to SEQ ID NO:17 over a region of at least about 400 residues.

5. The isolated or recombinant nucleic acid of claim 1, wherein the nucleic acid comprises a nucleic acid sequence having at least 75% sequence identity to SEQ ID NO:17 over a region of at least about 500 residues.

6. The isolated or recombinant nucleic acid of claim 1, wherein the nucleic acid comprises a nucleic acid sequence having at least 75% sequence identity to SEQ ID NO:17 over a region of at least about 600 residues.

7. The isolated or recombinant nucleic acid of claim 1, wherein the nucleic acid comprises a nucleic acid sequence having at least 80%, at least 85%, at least 90%, at least 95%, or at least 100% sequence identity to SEQ ID NO:17 over a region of at least about 100 residues.

8. The isolated or recombinant nucleic acid of claim 1, wherein the fluorescent polypeptide comprises a green fluorescent protein.

9. The isolated or recombinant nucleic acid of claim 1, wherein the fluorescent polypeptide comprises a cyan fluorescent protein.

10. The isolated or recombinant nucleic acid of claim 1, wherein a fluorescent activity comprises emission between about 500 nm (green) and 507 nm (green).

11. The isolated or recombinant nucleic acid of claim 1, wherein a fluorescent activity comprises emission between about 490 nm (cyan) and 491 nm (cyan).

12. The isolated or recombinant nucleic acid of claim 1, wherein the polypeptide comprises fluorescent activity after excitation at 485 nm (for green).

13. The isolated or recombinant nucleic acid of claim 1, wherein the polypeptide comprises fluorescent activity after excitation at 460 nm (for cyan).

14. The isolated or recombinant nucleic acid of claim 1, wherein the polypeptide retains a fluorescent activity under conditions comprising about pH 3.0.

15. The isolated or recombinant nucleic acid of claim 14, wherein the polypeptide retains a fluorescent activity under conditions comprising about pH 3.5.

16. The isolated or recombinant nucleic acid of claim 14, wherein the polypeptide retains a fluorescent activity under conditions comprising about pH 4.0.

17. The isolated or recombinant nucleic acid of claim 1, wherein the fluorescence is thermostable.

18. The isolated or recombinant nucleic acid of claim 17, wherein the polypeptide retains a fluorescent activity under conditions comprising a temperature range of between about 30° C. to about 90° C.

19. The isolated or recombinant nucleic acid of claim 1, wherein the fluorescence is thermotolerant.

20. The isolated or recombinant nucleic acid of claim 19, wherein the polypeptide retains a fluorescent activity under conditions comprising a temperature range of between about 30° C. to about 100° C.

21. The isolated or recombinant nucleic acid of claim 1, wherein the polypeptide retains a fluorescent activity under conditions comprising treatment for a period up to about 50 hours with 6M guanidine HCL, 8M urea or 1% SDS.

22. The isolated or recombinant nucleic acid of claim 1, wherein the polypeptide retains a fluorescent activity under conditions comprising treatment for a period up to about 50 hours with trypsin, chymotrypsin, papain, subtilisin, thermolisin, or pancreatin under conditions comprising a concentration range of up to about 1 mg/ml.

23. An isolated or recombinant nucleic acid, wherein the nucleic acid comprises a sequence that hybridizes under stringent conditions to a sequence comprising
a nucleic acid sequence as set forth in SEQ ID NO:17, wherein the nucleic acid encodes a fluorescent polypeptide.

24. The isolated or recombinant nucleic acid of claim 23, wherein the nucleic acid is at least about 100, 200, 300, 400, 500, or 600 residues in length or the full length of the gene or transcript.

25. The isolated or recombinant nucleic acid of claim 23, wherein the stringent conditions include a wash step comprising a wash in 0.2×SSC at a temperature of about 65° C. for about 15 minutes.

26. A nucleic acid probe for identifying a nucleic acid encoding a fluorescent polypeptide, wherein the probe comprises at least 10 consecutive bases of a sequence comprising a sequence as set forth in SEQ ID NO:17, wherein the probe identifies the nucleic acid by binding or hybridization.

27. The nucleic acid probe of claim 26, wherein the probe comprises an oligonucleotide comprising at least about 10 to 50, about 20 to 60, about 30 to 70, about 40 to 80, or about 60 to 100 consecutive bases of a sequence comprising a sequence as set forth in SEQ ID NO:17.

28. A nucleic acid probe for identifying a nucleic acid encoding a fluorescent polypeptide, wherein the probe comprises a nucleic acid sequence comprising a nucleic acid sequence having at least 75% sequence identity to SEQ ID NO:17 over a region of at least about 100 residues wherein the sequence identities are determined by analysis with a sequence comparison algorithm or by visual inspection.

29. The nucleic acid probe of claim 28, wherein the probe comprises an oligonucleotide comprising at least about 10 to 50, about 20 to 60, about 30 to 70, about 40 to 80, or about 60 to 100 consecutive bases of a nucleic acid sequence selected from the group consisting of a sequence as set forth in SEQ ID NO:17, or a subsequence thereof.

30. The nucleic acid probe of claim 28, wherein the probe comprises a nucleic acid sequence having at least 90%, at least 95%, at least 98%, or at least 100% sequence identity to a nucleic acid sequence comprising a sequence as set forth in SEQ ID NO:17.

31. An amplification primer sequence pair for amplifying a nucleic acid encoding a polypeptide with a fluorescent activity, wherein the primer pair is capable of amplifying a nucleic acid comprising a sequence as set forth in SEQ ID NO:17, or a subsequence thereof.

32. The nucleic acid probe of claim 31, wherein each member of the amplification primer sequence pair comprises an oligonucleotide comprising at least about 10 to 50 consecutive bases of the sequence.

33. A method of amplifying a nucleic acid encoding a fluorescent polypeptide comprising amplification of a template nucleic acid with an amplification primer sequence pair capable of amplifying a nucleic acid sequence comprising a sequence as set forth in SEQ ID NO:17, or a subsequence thereof.

34. An expression cassette comprising a nucleic acid comprising
(i) a nucleic acid comprising
a nucleic acid sequence having at least 75% sequence identity to SEQ ID NO:17 over a region of at least about 100 residues,
wherein the sequence identities are determined by analysis with a sequence comparison algorithm or by visual inspection; or,
(ii) a nucleic acid that hybridizes under stringent conditions to a nucleic acid comprising a sequence as set forth in SEQ ID NO:17, or a subsequence thereof.

35. A vector comprising a nucleic acid comprising
(i) a nucleic acid comprising
a nucleic acid sequence having at least 75% sequence identity to SEQ ID NO:17 over a region of at least about 100 residues,
wherein the sequence identities are determined by analysis with a sequence comparison algorithm or by visual inspection; or,
(ii) a nucleic acid that hybridizes under stringent conditions to a nucleic acid comprising a sequence as set forth in SEQ ID NO:17, or a subsequence thereof.

36. A transformed cell comprising
(i) a nucleic acid comprising
a nucleic acid sequence having at least 75% sequence identity to SEQ ID NO:17 over a region of at least about 100 residues,
wherein the sequence identities are determined by analysis with a sequence comparison algorithm or by visual inspection; or,
(ii) a nucleic acid that hybridizes under stringent conditions to a nucleic acid comprising a sequence as set forth in SEQ ID NO:17, or a subsequence thereof.

37. The transformed cell of claim 36, wherein the cell is a bacterial cell, a mammalian cell, a fungal cell, a yeast cell, an insect cell or a plant cell.

38. An antisense oligonucleotide comprising a nucleic acid sequence complementary to or capable of hybridizing under stringent conditions to
(i) a nucleic acid comprising
a nucleic acid sequence having at least 75% sequence identity to SEQ ID NO:17 over a region of at least about 100 residues,
wherein the sequence identities are determined by analysis with a sequence comparison algorithm or by visual inspection; or,
(ii) a nucleic acid that hybridizes under stringent conditions to a nucleic acid comprising a sequence as set forth in SEQ ID NO:17, or a subsequence thereof.

39. The antisense oligonucleotide of claim 38, wherein the antisense oligonucleotide is between about 10 to 50, about 20 to 60, about 30 to 70, about 40 to 80, or about 60 to 100 bases in length.

40. An isolated or recombinant polypeptide comprising
(a) a polypeptide sequence comprising
an amino acid sequence having at least 65% sequence identity to SEQ ID NO:18 over a region of at least about 100 residues,
wherein the sequence identities are determined by analysis with a sequence comparison algorithm or by visual inspection; and,
(b) a polypeptide encoded by a nucleic acid comprising
(i) a nucleic acid comprising
a nucleic acid sequence having at least 75% sequence identity to SEQ ID NO:17 over a region of at least about 100 residues,
wherein the sequence identities are determined by analysis with a sequence comparison algorithm or by visual inspection; or,
(ii) a nucleic acid that hybridizes under stringent conditions to a nucleic acid comprising a sequence as set forth in SEQ ID NO:17, or a subsequence thereof.

41. The isolated or recombinant polypeptide of claim 40, wherein the polypeptide comprises a fluorescent activity.

42. The isolated or recombinant polypeptide of claim 41, wherein the polypeptide comprises an amino acid sequence having at least 65% sequence identity to SEQ ID NO:18 over a region of at least about 200 or at least about 228 residues.

43. The isolated or recombinant polypeptide of claim 40, wherein the polypeptide comprises an amino acid sequence having at least 70%, at least 75%, at least 80%, at least 85%, at least 90%, at least 95%, at least 98%, or at least 100% sequence identity to SEQ ID NO:18 over a region of at least about 100 residues.

44. The isolated or recombinant polypeptide of claim 42, wherein the polypeptide comprises an amino acid sequence as set forth in SEQ ID NO:18, or a subsequence thereof.

45. An isolated or recombinant polypeptide comprising the polypeptide as set forth in claim 42 and a heterologous signal sequence.

46. The isolated or recombinant polypeptide of claim 42, wherein the fluorescent activity comprises emission at 500 nm (green).

47. The isolated or recombinant polypeptide of claim 42, wherein the fluorescent activity comprises emission at 490 nm (cyan).

48. The isolated or recombinant polypeptide of claim 42, wherein the polypeptide comprises fluorescent activity after excitation at 485 nm (for green).

49. The isolated or recombinant polypeptide of claim 42, wherein the fluorescent activity comprises fluorescent activity after excitation at 460 nm (for cyan).

50. A protein preparation comprising a polypeptide as set forth in claim 42, wherein the protein preparation comprises a liquid, a solid or a gel.

51. A homodimer comprising a polypeptide of the invention as set forth in claim 42.

52. A heterodimer comprising a polypeptide as set forth in claim 42 and a second domain.

53. The heterodimer of claim 52, wherein the second domain is a polypeptide and the heterodimer is a fusion protein.

54. The heterodimer of claim 52 wherein the second domain is an epitope.

55. The heterodimer of claim 52, wherein the second domain is a tag or a signal sequence.

56. An immobilized fluorescent polypeptide, wherein the polypeptide comprises a sequence as set forth in claim 40.

57. The immobilized polypeptide of claim 56, wherein the polypeptide is immobilized on a cell, a metal, a resin, a polymer, a ceramic, a glass, a microelectrode, a graphitic particle, a bead, a gel, a plate, an array or a capillary tube.

58. An array comprising an immobilized polypeptide as set forth in claim 42.

59. An isolated or recombinant antibody that specifically binds to a polypeptide as set forth in SEQ ID NO: 18 or to a polypeptide encoded by a nucleic acid as set forth in claim 1.

60. A method of producing a recombinant polypeptide comprising the steps of:
(a) providing a nucleic acid operably linked to a promoter; wherein the nucleic acid comprises a sequence having at least 75% sequence identity to SEQ ID NO:17 over a region of at least about 100 residues; and
(b) expressing the nucleic acid of step (a) under conditions that allow expression of the polypeptide, thereby producing a recombinant polypeptide.

61. The method of claim 60 further comprising transforming a host cell with the nucleic acid of step (a) followed by expressing the nucleic acid of step (a), thereby producing a recombinant polypeptide in a transformed cell.

62. A method for identifying a polypeptide having a fluorescent activity comprising the following steps:
(a) providing a polypeptide as set forth in claim 40 or a polypeptide encoded by a nucleic acid sequence having at least 75% sequence identity to SEQ ID NO:17 over a region of at least about 100 residues;
(b) providing an excitation source; and
(c) subjecting the polypeptide or a fragment or variant thereof of step (a) to an excitation energy provided by the excitation source of step (b) and detecting an emitted light by the polypeptide of step (a) thereby identifying a polypeptide having a fluorescent activity.

63. The method of claim 62, wherein the excitation occurs at a wavelength comprising the range from about 380 nm to about 510 nm or 490 nm to about 510 nm.

64. A method for isolating or recovering a nucleic acid encoding a polypeptide with a fluorescent activity from an environmental sample comprising the steps of:
(a) providing an amplification primer sequence pair for amplifying a nucleic acid encoding a polypeptide with a fluorescent activity, wherein the primer pair is capable of amplifying SEQ ID NO:17, or a subsequence thereof;
(b) isolating a nucleic acid from the environmental sample or treating the environmental sample such that nucleic acid in the sample is accessible for hybridization to the amplification primer pair; and,
(c) combining the nucleic acid of step (b) with the amplification primer pair of step (a) and amplifying nucleic acid from the environmental sample, thereby isolating or recovering a nucleic acid encoding a fluorescent polypeptide from an environmental sample.

65. The method of claim 64, wherein each member of the amplification primer sequence pair comprises an oligonucleotide comprising at least about 10 to 50 consecutive bases of a sequence as set forth in SEQ ID NO:17, or a subsequence thereof.

66. A method for isolating or recovering a nucleic acid encoding a polypeptide with a fluorescent activity from an environmental sample comprising the steps of:
(a) providing a polynucleotide probe comprising a sequence or a subsequence comprising:
(i) a nucleic acid comprising
a nucleic acid sequence having at least 75% sequence identity to SEQ ID NO:17 over a region of at least about 100 residues, or
(ii) a nucleic acid that hybridizes under stringent conditions to a nucleic acid comprising a sequence as set forth in SEQ ID NO:17, or a subsequence thereof
(b) isolating a nucleic acid from the environmental sample or treating the environmental sample such that nucleic acid in the sample is accessible for hybridization to a polynucleotide probe of step (a);
(c) combining the isolated nucleic acid or the treated environmental sample of step (b) with the polynucleotide probe of step (a); and
(d) isolating a nucleic acid that specifically hybridizes with the polynucleotide probe of step (a), thereby isolating or recovering a nucleic acid encoding a polypeptide with a fluorescent activity from an environmental sample.

67. The method of claim 66, wherein the environmental sample comprises a water sample, a liquid sample, a soil sample, an air sample or a biological sample.

68. The method of claim 66, wherein the biological sample is derived from a bacterial cell, a protozoan cell, an insect cell, a yeast cell, a plant cell, a fungal cell or a mammalian cell.

69. A method of generating a variant of a nucleic acid encoding a fluorescent protein comprising the steps of:
(a) providing a template nucleic acid comprising:
(i) a nucleic acid comprising
a nucleic acid sequence having at least 75% sequence identity to SEQ ID NO:17 over a region of at least about 100 residues, or,
(ii) a nucleic acid that hybridizes under stringent conditions to a nucleic acid comprising a sequence as set forth in SEQ ID NO:17, or a subsequence thereof.

70. The method of claim 69, further comprising expressing the variant nucleic acid to generate a variant fluorescent polypeptide.

71. The method of claim 69, wherein the modifications, additions or deletions are introduced by a method selected from the group consisting of error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM™), synthetic ligation reassembly (SLR) and a combination thereof.

72. The method of claim 69, wherein the modifications, additions or deletions are introduced by a method selected from the group consisting of recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

73. A method for modifying codons in a nucleic acid encoding a fluorescent polypeptide to increase its expression in a host cell, the method comprising
(a) providing a nucleic acid encoding a fluorescent polypeptide comprising a sequence selected from the group consisting of:
(i) a nucleic acid comprising
a nucleic acid sequence having at least 75% sequence identity to SEQ ID NO:17 over a region of at least about 100 residues,
wherein the sequence identities are determined by analysis with a sequence comparison algorithm or by visual inspection; or,
(ii) a nucleic acid that hybridizes under stringent conditions to a nucleic acid comprising a sequence as set forth in SEQ ID NO:17, or a subsequence thereof, and
(b) modifying, deleting or adding one or more nucleotides in the template sequence, or a combination thereof, to generate a variant of the template nucleic acid (b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

74. The method of claim 73, wherein the host cell is a bacterial cell, a fungal cell, an insect cell, a yeast cell, a plant cell or a mammalian cell.

75. A method for producing a nucleic acid with a fluorescent tag comprising of following steps:
(a) providing a first fluorescent polypeptide wherein the polypeptide comprises an amino acid sequence as set forth in claim 48, or, is encoded by a nucleic acid sequence having at least 75% sequence identity to SEQ ID NO:17 over a region of at least about 100 residues; and
(b) providing a nucleic acid; and
(c) contacting the polypeptide of step (a) and the nucleic acid of step (b) under conditions wherein the fluorescent polypeptide can covalently bind with the nucleic acid, thereby producing a nucleic acid with a fluorescent tag.

76. A method for using a polypeptide as a fluorescent marker comprising the following steps:
(a) providing a fluorescent polypeptide wherein the polypeptide comprises an amino acid sequence as set forth in claim 48, or, is encoded by a nucleic acid sequence having at least 75% sequence identity to SEQ ID NO:17 over a region of at least about 100 residues;
(b) providing an excitation source emitting light at the absorption wavelength of the fluorescent polypeptide; and
(c) detecting a fluorescent activity of the compound of step (a) at the emission wavelength of the fluorescent polypeptide.

77. An isolated or recombinant nucleic acid comprising a sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:17.

78. An isolated or recombinant nucleic acid comprising a sequence as set forth in SEQ ID NO:17.

79. An isolated or recombinant polypeptide comprising a sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:18.

80. An isolated or recombinant polypeptide having a sequence as set forth in SEQ ID NO:18.

81. An isolated or recombinant nucleic acid having a sequence comprising any combination of segments whose overhangs as described in FIG. 15 can anneal to each other.


SEQUENCE LISTING (198 sequences)

SEQ ID NO: 1 - DNA, 684 bp, Unknown
  Obtained from an environmental sample
atgagtcattccaagagtgtgatcaaggatgaaatgttcatcaagattcatctggaaggaacgttcaatgggcataagtttgaaatagaaggcgaaggacacgggaagccttatgcaggcaccaatttcgttaagcttgtggttaccaggggtggacctttgccatttggttggcacattttgtcgccacaatttcagtatggaaacaagacgtttgtcagctaccctagagacatacccgattatataaagcagtcatttcctgagggctttacatgggaacggatcatgaccttcgaagacggtggcgtgtgttgtatcaccagtgatatcagtttgaaaagcaacaactgtttcttcaacgacatcaagttcactggcatgaactttcctccaaatggatctgttgtgcagaagaagacgataggctgggaacccagcactgagcgtttgtatctgcgtgacggggtgctgacaggagacattgataagacactgaagctcagcggaggtggtcattacacatgcgcctttaaaactatttacaggtcgaagaagaacttgacgctgcctgattgcctttactatgttgacaccaaacttgatataaggaagttcgacgaaaattacatcaacgttgagcaggatgaaattgctactgcacgccaccatgggcttaaataa

SEQ ID NO: 2 - protein, 227 aa, Unknown
  Obtained from an environmental sample
MSHSKSVIKDEMFIKIHLEGTFNGHKFEIEGEGHGKPYAGTNFVKLVVTRGGPLPFGWHILSPQFQYGNKTFVSYPRDIPDYIKQSFPEGFTWERIMTFEDGGVCCITSDISLKSNNCFFNDIKFTGMNFPPNGSVVQKKTIGWEPSTERLYLRDGVLTGDIDKTLKLSGGGHYTCAFKTIYRSKKNLTLPDCLYYVDTKLDIRKFDENYINVEQDEIATARHHGLK

SEQ ID NO: 3 - DNA, 684 bp, Unknown
  Obtained from an environmental sample
atgagtcattccaagagtgtgatcaaggatgaaatgttcatcaagattcatctggaaggaacgttcaatgggcacaagtttgaaatagaaggcgaaggacacgggaagccttatgcaggcaccaatttcgttaagcttgtggttaccaagggtggacctttgccatttggttggcacattttgtcgccacaatttcagtatggaaacaagacgtttgtcagctaccctagagacatacccgattatataaagcagtcatttcctgagggctttacatgggtacggatcatgacctttgaagacggtggcgtgtgttgtatcaccagtgatatcagtttgaaaagcaacaactgtttcttcaacgacatcaagttcactggcatgaactttcctccaaatggacctgttgtgcagaagaagacgataggctgggaacccagcactgagcgtttgtatctgcgtgacggggtgctgacaggagacattgataagacactgaagctcagcggaggtggtcattacacatgcgcctttaaaactatttacaggtcgaagaagaacttgacgctgcctgattgcttttactatgttgacaccaaacttgatataaggaagttcgacgaaaattacatcaacgttgagcaggatgaaattgctactgcacgccaccatgggcttaaataa

SEQ ID NO: 4 - protein, 227 aa, Unknown
  Obtained from an environmental sample
MSHSKSVIKDEMFIKIHLEGTFNGHKFEIEGEGHGKPYAGTNFVKLVVTKGGPLPFGWHILSPQFQYGNKTFVSYPRDIPDYIKQSFPEGFTWVRIMTFEDGGVCCITSDISLKSNNCFFNDIKFTGMNFPPNGPVVQKKTIGWEPSTERLYLRDGVLTGDIDKTLKLSGGGHYTCAFKTIYRSKKNLTLPDCFYYVDTKLDIRKFDENYINVEQDEIATARHHGLK

SEQ ID NO: 5 - DNA, 684 bp, Unknown
  Obtained from an environmental sample
atgagtcattctaagagtgtgatcaaggatgaaatgttcatcaagattcatctggaaggaacgttcaatgggcacaagtttgaaatagaaggcgaaggacacgggaagccttatgcaggcaccaatttcgttaagcttgtggttaccaagggtggacctttgccatttggttggcacattttgtcgccacaatttcagtatggaaacaagacgtttgtcagctaccctagagacatacccgattatataaagcagtcatttcctgagggctttacatgggaacggatcatgacctttgaagacggtggcgtgtgttgtatcaccagtgatatcagtttgaaaagcaacaactgtttcttcaacgacatcaagttcactggcatgaactttcctccaaatggacctgttgtgcagaagaagacgataggctgggaacccagcactgagcgtttgtatctgcgtgacggggtgctgacaggagacattgataagacactgaagctcagcggaggtggtcattacacatgcgcctttaaaactatttacaggtcgaagaagaacttgacgctgcctgattgcttttactatgttgacaccaaacttgatataaggaagttcgacgaaaattacatcaacgttgagcaggatgaaattgctactgcacgccaccatgggcttaaataa

SEQ ID NO: 6 - protein, 227 aa, Unknown
  Obtained from an environmental sample
MSHSKSVIKDEMFIKIHLEGTFNGHKFEIEGEGHGKPYAGTNFVKLVVTKGGPLPFGWHILSPQFQYGNKTFVSYPRDIPDYIKQSFPEGFTWERIMTFEDGGVCCITSDISLKSNNCFFNDIKFTGMNFPPNGPVVQKKTIGWEPSTERLYLRDGVLTGDIDKTLKLSGGGHYTCAFKTIYRSKKNLTLPDCFYYVDTKLDIRKFDENYINVEQDEIATARHHGLK

SEQ ID NO: 7 - DNA, 684 bp, Unknown
  Obtained from an environmental sample
atgagtcattccaagagtgtgatcaaggacgaaatgttcatcaagattcatctggaaggaacgttcaatgggcacaagtttgaaatagaaggcgagggaaacgggaagccttatgcaggcaccaatttcgttaagcttgtggttaccaagggtgggcctcttccatttggttggcacattttgtcgccacaattacaatacggaaacaagtcgtttgtcagctaccctgcagacatacctgattatataaagctgtcatttcctgagggctttacatgggaaaggatcatgacctttgaagacggtggcgtgtgttgtatcaccagtgatatcagtatgaaaagcaacaactgtttcttctacgacatcaagttcactggcatgaactttcctccaaatggacctgttgtgcagaagaagaccacaggctgggaacccagtactgagcgtttgtatctgcgtgacggggtgctgacaggagacattcataagacactgaagctcagcggaggtggtcattacacatgcgtctttaaaactatttacaggtcgaagaagaacttgacgctgcctgattgcttttactatgttgacaccaaacttgatataaggaagttcgacgaaaattacatcaacgttgagcaggatgaaattgctactgcacgccaccatgggcttaaataa

SEQ ID NO: 8 - protein, 227 aa, Unknown
  Obtained from an environmental sample
MSHSKSVIKDEMFIKIHLEGTFNGHKFEIEGEGNGKPYAGTNFVKLVVTKGGPLPFGWHILSPQLQYGNKSFVSYPADIPDYIKLSFPEGFTWERIMTFEDGGVCCITSDISMKSNNCFFYDIKFTGMNFPPNGPVVQKKTTGWEPSTERLYLRDGVLTGDIHKTLKLSGGGHYTCVFKTIYRSKKNLTLPDCFYYVDTKLDIRKFDENYINVEQDEIATARHHGLK

SEQ ID NO: 9 - DNA, 687 bp, Unknown
  Obtained from an environmental sample
atgaagggggtgaaggaagtaatgaagatcagtctggagatggactgcactgttaacggcgacaaatttaagatcactggggatggaacaggagaaccttacgaaggaacacagactttacatcttacagagaaggaaggcaagcctctgacgttttctttcgatgtattgacaccagcatttcagtatggaaaccgtacattcaccaaatacccaggcaatataccagactttttcaagcagaccgtttctggtggcgggtatacctgggagcgaaaaatgacttatgaagacgggggcataagtaacgtccgaagcgacatcagtgtgaaaggtgactctttctactataagattcacttcactggcgagtttcctcctcatggtccagtgatgcagaggaagacagtaaaatgggagccatccactgaagtaatgtatgttgacgacaagagtgacggtgtgctgaagggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagtgtgctgtagctcgctattctctgctgcctgagaagaacaagtca

SEQ ID NO: 10 - protein, 229 aa, Unknown
  Obtained from an environmental sample
MKGVKEVMKISLEMDCTVNGDKFKITGDGTGEPYEGTQTLHLTEKEGKPLTFSFDVLTPAFQYGNRTFTKYPGNIPDFFKQTVSGGGYTWERKMTYEDGGISNVRSDISVKGDSFYYKIHFTGEFPPHGPVMQRKTVKWEPSTEVMYVDDKSDGVLKGDVNMALLLKDGRHLRVDFNTSYIPKKKVENMPDYHFIDHRIEILGNPEDKPVKLYECAVARYSLLPEKNKS

SEQ ID NO: 11 - DNA, 684 bp, Unknown
  Obtained from an environmental sample
atgaagggggtgaaggaagtcatgaagatcagtctggagatggactgcactgttaacggcgacaaatttaagatcactggggatggaacaggagaaccttacgaaggaacacagactttacatcttacagagaaggaaggcaagcctctgacgttttctttcgatgtattgacaccagcatttcagtatggaaaccgtacattcaccaaatacccaggcaatataccagactttttcaagcagaccgtttctggtggcgggtatacctgggagcgaaaaatgacttatgaagacgggggcataagtaacgtccgaagcgacatcagtgtgaaaggtgactctttctactataagattcacttcactggcgagtttcctcctcatggtccagtgatgcagaggaagacagtaaaatgggagccatccactgaagtaatgtatgtggacgataagagtggtggtgagctgaagggagatgtcaacatggctctgttgcttaaagatggccgccatttgagagttgacttcaacacttcttacatacccaagaagaaggtcgagaatatgcctgactaccattttatagaccaccgcattgagattctgggcaacccagaagacaagccggtcaagctgtacgagtgtgctgtagctcgctattctctgctgcctgagaagaacaag

SEQ ID NO: 12 - protein, 228 aa, Unknown
  Obtained from an environmental sample
MKGVKEVMKISLEMDCTVNGDKFKITGDGTGEPYEGTQTLHLTEKEGKPLTFSFDVLTPAFQYGNRTFTKYPGNIPDFFKQTVSGGGYTWERKMTYEDGGISNVRSDISVKGDSFYYKIHFTGEFPPHGPVMQRKTVKWEPSTEVMYVDDKSGGELKGDVNMALLLKDGRHLRVDFNTSYIPKKKVENMPDYHFIDHRIEILGNPEDKPVKLYECAVARYSLLPEKNK

SEQ ID NO: 13 - DNA, 675 bp, Unknown
  Obtained from an environmental sample
gtgaaggaagtaatgaagatcagtctggagatggactgcactgttaacggcgacaaatttaagatcactggggatggaacaggagaaccttacgaaggaacacagactttacatcttacagagaaggaaggcaagcctctgacgttttctttcgatgtattgacaccagcatttcagtatggcaaccgtacattcaccaaatacccaggcaatataccagactttttcaagcagaccgtttctggtggcgggtatacctgggagcgaaaaatgacttatgaagacgggggcataagtaacgtccgaagcgacatcagtgtgaaaggtgactctttctactataagattcacttcactggcgaatttccttctcacggtccagtgatgcagaagaagacggtaaaatgggagccatccactgaagtaatgtatgtggacgataagagtgatggtgtgctgaagggagatgtcaacatggctctgttgcttaaagatggccgccatttgcgagtggacttcaacacttcttacatacccaagaagaaggtcgagaatatgcctgactaccattttatagaccaccgcattgagattctgggcaacccagatgacaatccggtcaagctgtacgagtgtgctgtagctcgctgttctctgctgcctgagaagaacaag

SEQ ID NO: 14 - protein, 225 aa, Unknown
  Obtained from an environmental sample
MKEVMKISLEMDCTVNGDKFKITGDGTGEPYEGTQTLHLTEKEGKPLTFSFDVLTPAFQYGNRTFTKYPGNIPDFFKQTVSGGGYTWERKMTYEDGGISNVRSDISVKGDSFYYKIHFTGEFPSHGPVMQKKTVKWEPSTEVMYVDDKSDGVLKGDVNMALLLKDGRHLRVDFNTSYIPKKKVENMPDYHFIDHRIEILGNPDDNPVKLYECAVARCSLLPEKNK

SEQ ID NO: 15 - DNA, 693 bp, Unknown
  Obtained from an environmental sample
atgaagggggtgaaggaagtgatgaagatccaggtgaagatgaacatcactgttaacggcgacaaatttgtgatcactggggatggaacaggcgaaccttacgacgggacacagattttaaatcttacagtggaaggaggcaagcctctgacattttctttcgatatattgacaccagtatttatgtatggcaacagagcattcaccaaatacccagagagtatcccagactttttcaagcagaccgtttctggtggcgggtatacttggaaacgaaagatgatttatgatcacgaggctgagggcgtgagtaccgttgacggggacatcagtgtgaatggagactgtttcatctataagattacgtttgacggcacatttcgtgaagatggtgcagtgatgcagaagatgacggaaaaatgggaaccatccactgaagtgatgtacaaggacgataaaaatgatgatgtgctgaagggagatgtcaaccatgctcttttgcttaaagatggccgccatgtgcgagttgatttcaatacctcttacaaagccaagtcaaagatcgagaatatgcctggttaccattttgtagaccaccgcattgagataatagggcgatcatcgcaagacacgaaggtcaagctgttcgagaacgctgtcgctcgctgttctctgctgcctgagaagaaccag

SEQ ID NO: 16 - protein, 231 aa, Unknown
  Obtained from an environmental sample
MKGVKEVMKIQVKMNITVNGDKFVITGDGTGEPYDGTQILNLTVEGGKPLTFSFDILTPVFMYGNRAFTKYPESIPDFFKQTVSGGGYTWKRKMIYDHEAEGVSTVDGDISVNGDCFIYKITFDGTFREDGAVMQKMTEKWEPSTEVMYKDDKNDDVLKGDVNHALLLKDGRHVRVDFNTSYKAKSKIENMPGYHFVDHRIEIIGRSSQDTKVKLFENAVARCSLLPEKNQ

SEQ ID NO: 17 - DNA, 687 bp, Unknown
  Obtained from an environmental sample
atgaagggggtgaaggaagtaatgaagatcagtctggagatggactgcactgttaacggcgacaaatttaagatcactggggatggaacaggagaaccttacgaaggaacacagactttacatcttacagagaaggaaggcaagcctctgacgttttctttcgatgtattgacaccagcatttcagtatggaaaccgtacattcaccaaatacccaggcaatataccagactttttcaagcagaccgtttctggtggcgggtatacctgggagcgaaaaatgacttatgaagacgggggcataagtaacgtccgaagcgacatcagtgtgaaaggtgactctttctactataagattcacttcactggcgagtttcctcctcatggtccagtgatgcagaggaagacagtaaaatgggagccatccactgaagtaatgtatgttgacgacaagagtgacggtgtgctgaagggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagtgtgctgtagctcgctattctctgctgcctgagaagaacaagtaa

SEQ ID NO: 18 - protein, 228 aa, Unknown
  Obtained from an environmental sample
MKGVKEVMKISLEMDCTVNGDKFKITGDGTGEPYEGTQTLHLTEKEGKPLTFSFDVLTPAFQYGNRTFTKYPGNIPDFFKQTVSGGGYTWERKMTYEDGGISNVRSDISVKGDSFYYKIHFTGEFPPHGPVMQRKTVKWEPSTEVMYVDDKSDGVLKGDVNMALLLKDGRHLRVDFNTSYIPKKKVENMPDYHFIDHRIEILGNPEDKPVKLYECAVARYSLLPEKNK

SEQ ID NO: 19 - DNA, 762 bp, Unknown
  Obtained from an environmental sample
atgaagggggtgaaggaagtaatgaagatcagtctggagatggactgcactgttaacggcgacaaatttaagatcactggggatggaacaggagaaccttacgaaggaacacagactttacatcttacagagaaggaaggcaagcctctgacgttttctttcgatgtattgacaccagcatttcagtatggaaaccgtacattcaccaaatacccaggcaatataccagactttttcaagcagaccgtttctggtggcgggtatacctgggagcgaaaaatgacttatgaagacgggggcataagtaacgtccgaagcgacatcagtgtgaaaggtgactctttctactataagattcacttcactggcgagtttcctcctcatggtccagtgatgcagaggaagacagtaaaatgggagccatccactgaagtaatgtatgttgacgacaagagtgacggtgtgctgaagggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagtgtgctgtagctcgctattctctgctgcctgagaagaacaagtcaaagggcaattcgaagcttgaaggtaagcctatccctaaccctctcctcggtctcgattctacgcgtaccggttaa

SEQ ID NO: 20 - protein, 253 aa, Unknown
  Obtained from an environmental sample
MKGVKEVMKISLEMDCTVNGDKFKITGDGTGEPYEGTQTLHLTEKEGKPLTFSFDVLTPAFQYGNRTFTKYPGNIPDFFKQTVSGGGYTWERKMTYEDGGISNVRSDISVKGDSFYYKIHFTGEFPPHGPVMQRKTVKWEPSTEVMYVDDKSDGVLKGDVNMALLLKDGRHLRVDFNTSYIPKKKVENMPDYHFIDHRIEILGNPEDKPVKLYECAVARYSLLPEKNKSKGNSKLEGKPIPNPLLGLDSTRTG

SEQ ID NO: 21 - DNA, 786 bp, Unknown
  Obtained from an environmental sample
gtgatggcgatttccgctctaaagaacgtcatcatcatcgtaatcatatactcctgcagcactagtgctgattcgtcgaactcttactctggatcctccttcgcgaatgggattgcagaggaaatgatgactgacctgcatttagagggtgctgttaacgggcaccactttacaattaaaggcgaaggaggaggctacccttacgagggagtgcagtttatgagcctcgaggtagtcaatggtgcccctcttccgttctcttttgatatcttgacaccggcattcatgtatggcaacagagtgttcaccaagtatccaaaagagataccacactatttcaagcagacgtttcctgaagggtatcactgggaaagaagcattccctttcaagatcaggcctcgtgcacggtaaccagccacataaggatgaaagaggaagaggagcggcattttcttcttaacgtcaaattttactgtgtgaattttccccccaatggtccagtcatgcagaggaggatacggggatgggagccatccactgagaacatttatccgcgtgatgaatttctagagggccatgatgacatgactcttcgggttgaaggaggtggctattaccgagctgaattcagaagttcttacaaaggaaagcactcaatcaacatgccagactttcacttcatagaccaccgcattgagattatggagcatgacgaagactacaaccatgttaagctgcgtgaagtagcccatgctcgttactctccgctgccttctgtgcactaa

SEQ ID NO: 22 - protein, 261 aa, Unknown
  Obtained from an environmental sample
VMAISALKNVIIIVIIYSCSTSADSSNSYSGSSFANGIAEEMMTDLHLEGAVNGHHFTIKGEGGGYPYEGVQFMSLEVVNGAPLPFSFDILTPAFMYGNRVFTKYPKEIPHYFKQTFPEGYHWERSIPFQDQASCTVTSHIRMKEEEERHFLLNVKFYCVNFPPNGPVMQRRIRGWEPSTENIYPRDEFLEGHDDMTLRVEGGGYYRAEFRSSYKGKHSINMPDFHFIDHRIEIMEHDEDYNHVKLREVAHARYSPLPSVH

SEQ ID NO: 23 - DNA, 786 bp, Unknown
  Obtained from an environmental sample
gtgatggcgatttccgctctaaagaacgtcatcatcatcgtaatcatatactcctgcagcactagtgctgattcgtcgaactcttactctggatcctccttcgcgaatgggattgcagaggaaatgatgactgacctgcatttagagggtgctgttaacgggcaccactttacaattaaaggcgaaggaggaggctacccttacgagggagtgcagtttatgagcctcgaggtagtcaatggtgcccctcttccgttctcttttgatatcttgacaccggcattcatgtatggcaacagagtgttcaccaagtatccaaaagagataccagactatttcaagcagacgtttcctgaagggtatcactgggaaagaagcattccctttcaagatcaggcctcgtgcacggtaaccagccacataaggatgaaagaggaagaggagcggcattttcttcttaacgtcaaattttactgtgtgaattttccccccaatggtccagtcatgcagaggaggatacggggatgggagccatccactgagaacatttatccgcgtgatgaatttctagagggccatgatgacatgactcttcgggttgaaggaggtggctattaccgagctgaattcagaagttcttacaaaggaaagcactcaatcaacatgccagactttcacttcatagaccaccgcattgagattatggagcatgacgaagactacaaccatgttaagctgcgtgaagtagcccatgctcgttactctccgctgccttctgtgcactaa

SEQ ID NO: 24 - protein, 261 aa, Unknown
  Obtained from an environmental sample
VMAISALKNVIIIVIIYSCSTSADSSNSYSGSSFANGIAEEMMTDLHLEGAVNGHHFTIKGEGGGYPYEGVQFMSLEVVNGAPLPFSFDILTPAFMYGNRVFTKYPKEIPDYFKQTFPEGYHWERSIPFQDQASCTVTSHIRMKEEEERHFLLNVKFYCVNFPPNGPVMQRRIRGWEPSTENIYPRDEFLEGHDDMTLRVEGGGYYRAEFRSSYKGKHSINMPDFHFIDHRIEIMEHDEDYNHVKLREVAHARYSPLPSVH

SEQ ID NO: 25 - DNA, 783 bp, Unknown
  Obtained from an environmental sample
atggcgatttccgctctaaagaacgtcatcatcatcgtaatcatatactcccgcagcactagtgctgattcgtcgaactcttactctggatcctccttcgcgaatgggattgcagaggaaatgatgactgacctgcatttagagggtgctgttaacgggcaccactttacaattaaaggcgaaggaggaggctacccttacgagggagtgcagtttatgagcctcgaggtagtcaatggtgcccctcttccgttctcttttgatatcttgacaccggcattcatgtatggcaacagagtgttcaccaagtatccaaaagagataccagactatttcaagcagacgtttcctgaagggtatcactgggaaagaagcattccctttcaagatcaggcctcgtgcacggtaaccagccacataaggatgaaagaggaagaggagcggcattttcttcttaacgtcaaattttactgtgtgaattttccccccaatggtccagtcatgcagaggaggatacggggatgggagccatccactgagaacatttatccgcgtgatgaatttctagagggccatgatgacatgactcttcgggttgaaggaggtggctattaccgagctgaattcagaagttcttacaaaggaaagcactcaatcaacatgccagactttcacttcatagaccaccgcattgagattatggagcatgacgaagactacaaccatgttaagctgcgtgaagtagcctatgctcgttactctccgctgccttctgtgcactaa

SEQ ID NO: 26 - protein, 260 aa, Unknown
  Obtained from an environmental sample
MAISALKNVIIIVIIYSRSTSADSSNSYSGSSFANGIAEEMMTDLHLEGAVNGHHFTIKGEGGGYPYEGVQFMSLEVVNGAPLPFSFDILTPAFMYGNRVFTKYPKEIPDYFKQTFPEGYHWERSIPFQDQASCTVTSHIRMKEEEERHFLLNVKFYCVNFPPNGPVMQRRIRGWEPSTENIYPRDEFLEGHDDMTLRVEGGGYYRAEFRSSYKGKHSINMPDFHFIDHRIEIMEHDEDYNHVKLREVAYARYSPLPSVH

SEQ ID NO: 27 - DNA, 684 bp, Artificial Sequence
  Synthetically generated
atgagtcattccaagagtgtgatcaaggacgaaatgttcatcaagattcatctggaaggcacttttaacggccacaaatttgagatcgaaggggagggaaacggaaaaccttacgcaggaacaaattttgtaaaacttgtagtgacgaaaggcgggcctctgccgtttggttggcatatattgtcaccacaattacagtatggaaacaagtcattcgtcagctacccagccgatataccagactatatcaagctgtcctttcctgagggctttacctgggagcgaataatgacttttgaggacgggggcgtatgttgcatcacaagcgacatcagtatgaaaagtaacaactgtttcttctatgacattaagttcactggcatgaactttcctcctaatggtccagtggtgcagaaaaagacaacaggatgggagccatccactgaacgattgtatcttcgcgacggtgtgctgacgggagatatccacaagactctgaaacttagcggtggcggccattacacatgtgtctttaaaactatttacagatccaagaagaacctcacgcttccggattgcttctattatgtagacaccaaacttgatattcggaagttcgacgaaaattacatcaacgtcgagcaggacgagattgctacagctcgccatcatgggctgaagtag

SEQ ID NO: 28 - protein, 227 aa, Artificial Sequence; identical to SEQ ID NO: 8
  Synthetically generated

SEQ ID NO: 29 - DNA, 687 bp, Artificial Sequence
  Synthetically generated
atgaagggggtgaaggaagtaatgaagatcagtctggagatggactgcactgttaacggcgacaaatttaagatcactggggatggaacaggagaaccttacgaaggaacacagactttacatcttacagagaaggaaggcaagcctctgacgttttctttcgatgtattgacaccagcatttcagtatggaaaccgtacattcaccaaatacccaggcaatataccagactttttcaagcagaccgtttctggtggcgggtatacctgggagcgaaaaatgacttatgaggacgggggcataagtaacgtccgaagcgacatcagtgtgaaaggtgactctttctactataagattcacttcactggcgagtttcctcctcatggtccagtgatgcagagaaagacagtaaaatgggagccatccactgaagtaatgtatgttgacgacaagagtgacggtgtgctgaagggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagtgtgctgtagctcgctattctctgctgcctgagaagaacaagtag

SEQ ID NO: 30 - protein, 228 aa, Artificial Sequence; identical to SEQ ID NO: 18
  Synthetically generated

SEQ ID NO: 31 - DNA, 786 bp, Artificial Sequence
  Synthetically generated
atgatggcgatttccgctctaaagaacgtcatcatcatcgtaatcatatactcctgcagcactagtgctgattcgtcgaactcttactctggatcctccttcgcgaatgggattgcggaagaaatgatgaccgatctgcatctggagggcgctgttaacggccaccactttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaagcattccttttcaagaccaggcctcatgtaccgtcacaagccacatcaggatgaaagaggaagaggagcggcatttcctccttaacgttaaattctattgcgtgaattttcctcctaatggtccagtgatgcagaggaggatacgaggatgggagccatccactgaaaacatttatcctcgcgacgaatttctggagggacatgacgacatgactctgcgggttgaaggtggcggctattacagagctgaatttagaagttcttacaaaggcaagcactcgatcaacatgccggatttccattttatagaccaccgcattgagattatggagcatgacgaggactacaaccatgtcaagctgcgcgaggttgctcatgctcgctattctccgctgccttcggtgcactag

SEQ ID NO: 32 - protein, 261 aa, Artificial Sequence
  Synthetically generated
MMAISALKNVIIIVIIYSCSTSADSSNSYSGSSFANGIAEEMMTDLHLEGAVNGHHFTIKGEGGGYPYEGVQFMSLEVVNGAPLPFSFDILTPAFMYGNRVFTKYPKEIPDYFKQTFPEGYHWERSIPFQDQASCTVTSHIRMKEEEERHFLLNVKFYCVNFPPNGPVMQRRIRGWEPSTENIYPRDEFLEGHDDMTLRVEGGGYYRAEFRSSYKGKHSINMPDFHFIDHRIEIMEHDEDYNHVKLREVAHARYSPLPSVH

SEQ ID NO: 33 - DNA, 729 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggaacacagactttacatcttacagagaaggaaggcaagcctctgccgttttctttcgatatattgacaccagcatttatgtatggaaaccgtgtattcaccaaatacccaaaagagataccagactatttcaagcagacctttcctgaaggctatcactgggagcgaaaaatgacttatgaggacgggggcataagtaacgtccgaagcgacatcagtgtgaaaggtgactctttctactataagattcacttcactggcgagtttcctcctcatggtccagtgatgcagagaaagacagtaaaatgggagccatccactgaacgattgtatcttcgcgacggtgtgctgacgggagatgtcaacatggctctgttgcttaaagatggcggccattacacatgtgtctttaaaactatttacagatccaagaagaaggtcgagaatatgcctgactaccattttatagaccaccgcattgagattatggagcatgacgaggactacaaccatgtcaagctgcgcgagtgtgctgtagctcgctattctctgctgcctgagaagaacaagggtaagcctatccctaaccctctcctcggactcgattctacgcgtaccggttag

SEQ ID NO: 34 - protein, 242 aa, Artificial Sequence
  Synthetically generated
MKGVKEVMKISLEMDCTVNGDKFTIKGEGGGYPYEGTQTLHLTEKEGKPLPFSFDILTPAFMYGNRVFTKYPKEIPDYFKQTFPEGYHWERKMTYEDGGISNVRSDISVKGDSFYYKIHFTGEFPPHGPVMQRKTVKWEPSTERLYLRDGVLTGDVNMALLLKDGGHYTCVFKTIYRSKKKVENMPDYHFIDHRIEIMEHDEDYNHVKLRECAVARYSLLPEKNKGKPIPNPLLGLDSTRTG

SEQ ID NO: 35 - DNA, 741 bp, Artificial Sequence
  Synthetically generated
atgagtcattccaagagtgtgatcaaggacgaaatgttcatcaagattcatctggaaggcacttttaacggccacaaatttgagatcgaaggggagggaaacggaaaaccttacgcaggagtacagtttatgtctcttgaagtggtgaatggcgcgcctctgccgtttggttggcatatattgtcaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaacgattgtatcttcgcgacggtgtgctgacgggacatgacgacatgactctgcgggttgaaggtggcggccattacacatgtgtctttaaaactatttacagatccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccgggttagctcgagggggg

SEQ ID NO: 36 - protein, 247 aa, Artificial Sequence
  Synthetically generated
MSHSKSVIKDEMFIKIHLEGTFNGHKFEIEGEGNGKPYAGVQFMSLEVVNGAPLPFGWHILSPAFMYGNRVFTKYPKEIPDYFKQTFPEGYHWERIMTFEDGGVCCITSDISVKGDSFFYDIKFTGMNFPPHGPVMQRKTVKWEPSTERLYLRDGVLTGHDDMTLRVEGGGHYTCVFKTIYRSKKKVENMPDYHFIDHRIEILGNPEDKPVKLYEIATARHHGLKGKPIPNPLLGLDSTRTGLARGG

SEQ ID NO: 37 - DNA, 720 bp, Artificial Sequence
  Synthetically generated
atgaagggggtgaaggaagtaatgaagatcagtctggagatggagggcgctgttaacggccaccactttgagatcgaaggggagggaaacggaaaaccttacgcaggaacacagactttacatcttacagagaaggaaggcaagcctctgacgttttctttcgatgtattgacaccagcatttatgtatggaaaccgtgtattcaccaaatacccaaaagagataccagactatttcaagcagacctttcctgaaggctatcactgggagcgaagcattccttttcaagaccaggcctcatgtaccgtcacaagccacatcaggatgaaagaggaagaggagcggcatttctactataagattcacttcactggcgagtttcctcctcatggtccagtgatgcagagaaagacagtaaaatgggagccatccactgaacgattgtatcttcgcgacggtgtgctgacgggagatgtcaacatggctctgttgcttaaagatggcggctattacagagctgaatttagaagttcttacaaaggcaagcactcgatcaacatgccggatttccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 38 - protein, 239 aa, Artificial Sequence
  Synthetically generated
MKGVKEVMKISLEMEGAVNGHHFEIEGEGNGKPYAGTQTLHLTEKEGKPLTFSFDVLTPAFMYGNRVFTKYPKEIPDYFKQTFPEGYHWERSIPFQDQASCTVTSHIRMKEEEERHFYYKIHFTGEFPPHGPVMQRKTVKWEPSTERLYLRDGVLTGDVNMALLLKDGGYYRAEFRSSYKGKHSINMPDFHFIDHRIEILGNPEDKPVKLYEIATARHHGLKGKPIPNPLLGLDSTRTG

SEQ ID NO: 39 - DNA, 738 bp, Artificial Sequence
  Synthetically generated
atgagtcattccaagagtgtgatcaaggacgaaatgttcatcaagattcatctggaaggcacttttaacggccacaaatttgagatcgaaggggagggaaacggaaaaccttacgcaggagtacagtttatgtctcttgaagtggtgaatggcgcgcctctgccgtttggttggcatatattgtcaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaacgattgtatcttcgcgacggtgtgctgacgggacatgacgacatgactctgcgggttgaaggtggcggccattacacatgtgtctttaaaactatttacagatccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggtagctcgaggagg

SEQ ID NO: 40 - protein, 246 aa, Artificial Sequence
  Synthetically generated
MSHSKSVIKDEMFIKIHLEGTFNGHKFEIEGEGNGKPYAGVQFMSLEVVNGAPLPFGWHILSPAFMYGNRVFTKYPKEIPDYFKQTFPEGYHWERIMTFEDGGVCCITSDISVKGDSFFYDIKFTGMNFPPHGPVMQRKTVKWEPSTERLYLRDGVLTGHDDMTLRVEGGGHYTCVFKTIYRSKKKVENMPDYHFIDHRIEILGNPEDKPVKLYEIATARHHGLKGKPIPNPLLGLDSTRTGSSRR

SEQ ID NO: 41 - DNA, 726 bp, Artificial Sequence
  Synthetically generated
atgaagggggtgaaggaagtaatgaagatcagtctggagatggactgcactgttaacggcgacaaatttgagatcgaaggggagggaaacggaaaaccttacgcaggagtacagtttatgtctcttgaagtggtgaatggcgcgcctctgccgttttctttcgatatattgacaccacaattacagtatggaaacaagtcattcgtcagctacccaaaagagataccagactatttcaagcagacctttcctgaaggctatcactgggagcgaagcattccttttcaagaccaggcctcatgtaccgtcacaagcgacatcagtatgaaaagtaacaactgtttctactataagattcacttcactggcgagtttcctcctcatggtccagtgatgcagagaaagacagtaaaatgggagccatccactgaagtaatgtatgttgacgacaagagtgacggtgtgctgaagggacatgacgacatgactctgcgggttgaaggtggccgccatttgagagttgactttaacacttcttacatacccaagcactcgatcaacatgccggatttccattttatagaccaccgcattgatattcggaagttcgacgaaaattacatcaacgtcgagcaggacgagattgctacagctcgccatcatgggctgaagggtaagcctatccctaaccctctcctcggactcgattctacgcgtaccggttag

SEQ ID NO: 42 - protein, 241 aa, Artificial Sequence
  Synthetically generated
MKGVKEVMKISLEMDCTVNGDKFEIEGEGNGKPYAGVQFMSLEVVNGAPLPFSFDILTPQLQYGNKSFVSYPKEIPDYFKQTFPEGYHWERSIPFQDQASCTVTSDISMKSNNCFYYKIHFTGEFPPHGPVMQRKTVKWEPSTEVMYVDDKSDGVLKGHDDMTLRVEGGRHLRVDFNTSYIPKHSINMPDFHFIDHRIDIRKFDENYINVEQDEIATARHHGLKGKPIPNPLLGLDSTRTG

SEQ ID NO: 43 - DNA, 720 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggaacaaattttgtaaaacttgtagtgacgaaaggcgggcctctgacgttttctttcgatgtattgacaccagcatttatgtatggaaaccgtgtattcaccaaatacccaaaagagataccagactatttcaagcagacctttcctgaaggctatcactgggagcgaagcattccttttcaagaccaggcctcatgtaccgtcacaagcgacatcagtatgaaaagtaacaactgtttcttctatgacattaagttcactggcatgaactttcctcctcatggtccagtgatgcagagaaagacagtaaaatgggagccatccactgaaaacatttatcctcgcgacgaatttctggagggagatgtcaacatggctctgttgcttaaagatggccgccatttgagagttgactttaacacttcttacatacccaagcactcgatcaacatgccggatttccattttatagaccaccgcattgatattcggaagttcgacgaaaattacatcaacgtcgagcaggacgagattgctacagctcgccatcatgggctgaagggtaagcctatccctaaccctctcctcggactcgattctacgcgtaccggttag

SEQ ID NO: 44 - protein, 239 aa, Artificial Sequence
  Synthetically generated
MKGVKEVMKISLEMDCTVNGDKFTIKGEGGGYPYEGTNFVKLVVTKGGPLTFSFDVLTPAFMYGNRVFTKYPKEIPDYFKQTFPEGYHWERSIPFQDQASCTVTSDISMKSNNCFFYDIKFTGMNFPPHGPVMQRKTVKWEPSTENIYPRDEFLEGDVNMALLLKDGRHLRVDFNTSYIPKHSINMPDFHFIDHRIDIRKFDENYINVEQDEIATARHHGLKGKPIPNPLLGLDSTRTG

SEQ ID NO: 45 - DNA, 738 bp, Artificial Sequence
  Synthetically generated
atgagtcattccaagagtgtgatcaaggacgaaatgttcatcaagattcatctggaaggcacttttaacggccacaaatttgagatcgaaggggagggaaacggaaaaccttacgcaggaacacagactttacatcttacagagaaggaaggcaagcctctgccgttttctttcgatatattgacaccacaattacagtatggaaacaagtcattcgtcagctacccagccgatataccagactatatcaagctgtcctttcctgagggctttacctgggagcgaagcattccttttcaagaccaggcctcatgtaccgtcacaagcgacatcagtatgaaaagtaacaactgtttctactataagattcacttcactggcgagtttcctcctcatggtccagtgatgcagagaaagacagtaaaatgggagccatccactgaagtaatgtatgttgacgacaagagtgacggtgtgctgaagggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 46 - protein, 245 aa, Artificial Sequence
  Synthetically generated
MSHSKSVIKDEMFIKIHLEGTFNGHKFEIEGEGNGKPYAGTQTLHLTEKEGKPLPFSFDILTPQLQYGNKSFVSYPADIPDYIKLSFPEGFTWERSIPFQDQASCTVTSDISMKSNNCFYYKIHFTGEFPPHGPVMQRKTVKWEPSTEVMYVDDKSDGVLKGDVNMALLLKDGRHLRVDFNTSYIPKKKVENMPDYHFIDHRIEILGNPEDKPVKLYEIATARHHGLKGKPIPNPLLGLDSTRTG

SEQ ID NO: 47 - DNA, 603 bp, Artificial Sequence
  Synthetically generated
atggcgcgccttctgccgttttctttcgatatattgacaccagcatttatgtatggaaaccgtgtattcaccaaatacccaaaagagataccagactatttcaagcagacctttcctgaaggctatcactgggagcgaaaaatgacttatgaggacgggggcataagtaacgtccgaagccacatcaggatgaaagaggaagaggagcggcatttcttctatgacattaagttcactggcatgaactttcctcctcatggtccagtgatgcagagaaagacagtaaaatgggagccatccactgaagtaatgtatgttgacgacaagagtgacggtgtgctgaagggacatgacgacatgactctgcgggttgaaggtggccgccatttgagagttgactttaacacttcttacatacccaagaagaacctcacgcttccggattgcttctattatgtagacaccaaacttgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 48 - protein, 200 aa, Artificial Sequence
  Synthetically generated
MARLLPFSFDILTPAFMYGNRVFTKYPKEIPDYFKQTFPEGYHWERKMTYEDGGISNVRSHIRMKEEEERHFFYDIKFTGMNFPPHGPVMQRKTVKWEPSTEVMYVDDKSDGVLKGHDDMTLRVEGGRHLRVDFNTSYIPKKNLTLPDCFYYVDTKLEIMEHDEDYNHVKLREIATARHHGLKGKPIPNPLLGLDSTRTG

SEQ ID NO: 49 - DNA, 828 bp, Artificial Sequence
  Synthetically generated
atgatggcgatttccgctctaaagaacgtcatcatcatcgtaatcatatactcctgcagcactagtgctgattcgtcgaactcttactctggatcctccttcgcgaatgggattgcggaagaaatgatgaccgatctgcatctggactgcactgttaacggcgacaaatttgagatcgaaggggagggaaacggaaaaccttacgcaggagtacagtttatgtctcttgaagtggtgaatggcgcgcctctgccgttttctttcgatatattgacaccacaattacagtatggaaacaagtcattcgtcagctacccaaaagagataccagactatttcaagcagacctttcctgaaggctatcactgggagcgaagcattccttttcaagaccaggcctcatgtaccgtcacaagcgacatcagtgtgaaaggtgactctttcttctatgacattaagttcactggcatgaactttcctcctcatggtccagtgatgcagagaaagacagtaaaatgggagccatccactgaagtaatgtatgttgacgacaagagtgacggtgtgctgaagggacatgacgacatgactctgcgggttgaaggtggccgccatttgagagttgactttaacacttcttacatacccaagcactcgatcaacatgccggatttccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 50 - protein, 275 aa, Artificial Sequence
  Synthetically generated
MMAISALKNVIIIVIIYSCSTSADSSNSYSGSSFANGIAEEMMTDLHLDCTVNGDKFEIEGEGNGKPYAGVQFMSLEVVNGAPLPFSFDILTPQLQYGNKSFVSYPKEIPDYFKQTFPEGYHWERSIPFQDQASCTVTSDISVKGDSFFYDIKFTGMNFPPHGPVMQRKTVKWEPSTEVMYVDDKSDGVLKGHDDMTLRVEGGRHLRVDFNTSYIPKHSINMPDFHFIDHRIEIMEHDEDYNHVKLREIATARHHGLKGKPIPNPLLGLDSTRTG

SEQ ID NO: 51 - DNA, 717 bp, Artificial Sequence
  Synthetically generated
atgaagggggtgaaggaagtaatgaagatcagtctggagatggactgcactgttaacggcgacaaatttgagatcgaaggggagggaaacggaaaaccttacgcaggagtacagtttatgtctcttgaagtggtgaatggcgcgcctctgacgttttctttcgatgtattgacaccagcatttcagtatggaaaccgtacattcaccaaatacccagccgatataccagactatatcaagctgtcctttcctgagggctttacctgggagcgaagcattccttttcaagaccaggcctcatgtaccgtcacaagcgacatcagtgtgaaaggtgactctttcttctatgacattaagttcactggcatgaactttcctcctaatggtccagtgatgcagaggaggatacgaggatgggagccatccactgaaaacatttatcctcgcgacgaatttctggagggacatgacgacatgactctgcgggttgaaggtggcggctattacagagctgaatttagaagttcttacaaaggcaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 52 - protein, 238 aa, Artificial Sequence
  Synthetically generated
MKGVKEVMKISLEMDCTVNGDKFEIEGEGNGKPYAGVQFMSLEVVNGAPLTFSFDVLTPAFQYGNRTFTKYPADIPDYIKLSFPEGFTWERSIPFQDQASCTVTSDISVKGDSFFYDIKFTGMNFPPNGPVMQRRIRGWEPSTENIYPRDEFLEGHDDMTLRVEGGGYYRAEFRSSYKGKKKVENMPDYHFIDHRIEILGNPEDKPVKLYEIATARHHGLKGKPIPNPLLGLDSTRTG

SEQ ID NO: 53 - DNA, 714 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaagtaatgtatgttgacgacaagagtgacggtgtgctgaagggagatgtcaacatggctctgttgcttaaagatggcggccattacacatgtgtctttaaaactatttacagatccaagaagaaggtcgagaatatgcctgactaccattttatagaccaccgcattgagattatggagcatgacgaggactacaaccatgtcaagctgcgcgagattgctacagctcgccatcatgggctgaagggtaagcctatccctaaccctctcctcggactcgattga

SEQ ID NO: 54 - protein, 237 aa, Artificial Sequence
  Synthetically generated
MKGVKEVMKISLEMDCTVNGDKFTIKGEGGGYPYEGVQFMSLEVVNGAPLPFSFDILTPAFMYGNRVFTKYPKEIPDYFKQTFPEGYHWERIMTFEDGGVCCITSDISVKGDSFFYDIKFTGMNFPPHGPVMQRKTVKWEPSTEVMYVDDKSDGVLKGDVNMALLLKDGGHYTCVFKTIYRSKKKVENMPDYHFIDHRIEIMEHDEDYNHVKLREIATARHHGLKGKPIPNPLLGLD

SEQ ID NO: 55 - DNA, 711 bp, Artificial Sequence
  Synthetically generated
atgaagggggtgaaggaagtaatgaagatcagtctggagatggactgcactgttagcggcgacaaatttgagatcgaaggggagggaaacggaaaaccttacgcaggaacaaattttgtaaaacttgtagtgacgaaaggcgggcctctgccgtttggttggcatatattgtcaccagcatttatgtatggaaaccgtgtattcaccaaatacccaaaagagataccagactatttcaagcagacctttcctgaaggctatcactgggagcgaagcattccttttcaagaccaggcctcatgtaccgtcacaagcgacatcagtgtgaaaggtgactctttctactataagattcacttcactggcgagtttcctcctcatggtccagtgatgcagagaaagacagtaaaatgggagccatccactgaacgattgtatcttcgcgacggtgtgctgacgggacatgacgacatgactctgcgggttgaaggtggccgccatttgagagttgactttaacacttcttacatacccaagcactcgatcaacatgccggatttccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 56 - protein, 236 aa, Artificial Sequence
  Synthetically generated
MKGVKEVMKISLEMDCTVSGDKFEIEGEGNGKPYAGTNFVKLVVTKGGPLPFGWHILSPAFMYGNRVFTKYPKEIPDYFKQTFPEGYHWERSIPFQDQASCTVTSDISVKGDSFYYKIHFTGEFPPHGPVMQRKTVKWEPSTERLYLRDGVLTGHDDMTLRVEGGRHLRVDFNTSYIPKHSINMPDFHFIDHRIEILGNPEDKPVKLYEIATARHHGLKGKPIPNPLLGLDSTRTG

SEQ ID NO: 57 - DNA, 735 bp, Artificial Sequence
  Synthetically generated
atgagtcattccaagagtgtgatcaaggacgaaatgttcatcaagattcatctggaaggcacttttaacggccacaaatttacgatcaaaggggaaggaggaggatacccttacgaaggaacaaattttgtaaaacttgtagtgacgaaaggcgggcctctgccgttttctttcgatatattgacaccagcatttcagtatggaaaccgtacattcaccaaatacccagccgatataccagactatatcaagctgtcctttcctgagggctttacctgggagcgaagcattccttttcaagaccaggcctcatgtaccgtcacaagccacatcaggatgaaagaggaagaggagcggcatttctactataagattcacttcactggcgagtttcctcctaatggtccagtgatgcagaggaggatacgaggatgggagccatccactgaacgattgtatcttcgcgacggtgtgctgacgggacatgacgacatgactctgcgggttgaaggtggccgccatttgagagttgactttaacacttcttacatacccaagcactcgatcaacatgccggatttccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 58 - protein, 244 aa, Artificial Sequence
  Synthetically generated
MSHSKSVIKDEMFIKIHLEGTFNGHKFTIKGEGGGYPYEGTNFVKLVVTKGGPLPFSFDILTPAFQYGNRTFTKYPADIPDYIKLSFPEGFTWERSIPFQDQASCTVTSHIRMKEEEERHFYYKIHFTGEFPPNGPVMQRRIRGWEPSTERLYLRDGVLTGHDDMTLRVEGGRHLRVDFNTSYIPKHSINMPDFHFIDHRIEIMEHDEDYNHVKLREIATARHHGLKGKPIPNPLLGLDSTRTG

SEQ ID NO: 59 - DNA, 720 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaggcaatataccagactttttcaagcagaccgtttctggtggcgggtatacctgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaacgattgtatcttcgcgacggtgtgctgacgggacatgacgacatgactctgcgggttgaaggtggcggccattacacatgtgtctttaaaactatttacagatccaagcactcgatcaacatgccggatttccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 60 - protein, 239 aa, Artificial Sequence
  Synthetically generated
MKGVKEVMKISLEMDCTVNGDKFTIKGEGGGYPYEGVQFMSLEVVNGAPLPFSFDILTPAFMYGNRVFTKYPGNIPDFFKQTVSGGGYTWERIMTFEDGGVCCITSDISVKGDSFFYDIKFTGMNFPPHGPVMQRKTVKWEPSTERLYLRDGVLTGHDDMTLRVEGGGHYTCVFKTIYRSKHSINMPDFHFIDHRIEIMEHDEDYNHVKLREIATARHHGLKGKPIPNPLLGLDSTRTG

SEQ ID NO: 61 - DNA, 720 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttctactataagattcacttcactggcgagtttcctcctcatggtccagtgatgcagagaaagacagtaaaatgggagccatccactgaagtaatgtatgttgacgacaagagtgacggtgtgctgaagggagatgtcaacatggctctgttgcttaaagatggcggtcattacacatgtgtctttaaaactatttacagatccaagcactcgatcaacatgccggatttccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 62 - protein, 239 aa, Artificial Sequence
  Synthetically generated
MKGVKEVMKISLEMDCTVNGDKFTIKGEGGGYPYEGVQFMSLEVVNGAPLPFSFDILTPAFMYGNRVFTKYPKEIPDYFKQTFPEGYHWERIMTFEDGGVCCITSDISVKGDSFYYKIHFTGEFPPHGPVMQRKTVKWEPSTEVMYVDDKSDGVLKGDVNMALLLKDGGHYTCVFKTIYRSKHSINMPDFHFIDHRIEILGNPEDKPVKLYEIATARHHGLKGKPIPNPLLGLDSTRTG

SEQ ID NO: 63 - DNA, 516 bp, Artificial Sequence
  Synthetically generated
atgagtcattccaagagtgtgatcaaggacgaaatgttcatcaagattcatctggaaggcacttttaacggccacaaatttacgatcaaaggggaaggaggaggatacccttacgaaggagtacagtttatgtctcttgaagtggtgaatggcgcgcctctgacgttttctttcgatgtattgacaccacaattacagtatggaaacaagtcattcgtcagctacccaaaagagataccagactatttcaagcagacctttcctgaaggctatcactgggagcgaataatgacttttgaggacgggggcgtatgttgcatcacaagcgacatcagtgtgaaaggtgactctttctactataagattcacttcactggcgagtttcctcctcatggtccagtgatgcagagaaagacagtaaaatgggagccatccactgaaaacatttatcctcgcgacgaatttctggagggagatgtcaacatggctctgttgcttaaagaggccgccatttga

SEQ ID NO: 64 - protein, 171 aa, Artificial Sequence
  Synthetically generated
MSHSKSVIKDEMFIKIHLEGTFNGHKFTIKGEGGGYPYEGVQFMSLEVVNGAPLTFSFDVLTPQLQYGNKSFVSYPKEIPDYFKQTFPEGYHWERIMTFEDGGVCCITSDISVKGDSFYYKIHFTGEFPPHGPVMQRKTVKWEPSTENIYPRDEFLEGDVNMALLLKEAAI

SEQ ID NO: 65 - DNA, 714 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttctactataagattcacttcactggcgagtttcctcctcatggtccagtgatgcagagaaagacagtaaaatgggagccatccactgaaaacatttatcctcgcgacgaatttctggagggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 66 - protein, 237 aa, Artificial Sequence
  Synthetically generated
MKGVKEVMKISLEMDCTVNGDKFTIKGEGGGYPYEGVQFMSLEVVNGAPLPFSFDILTPAFMYGNRVFTKYPKEIPDYFKQTFPEGYHWERIMTFEDGGVCCITSDISVKGDSFYYKIHFTGEFPPHGPVMQRKTVKWEPSTENIYPRDEFLEGDVNMALLLKDGRHLRVDFNTSYIPKKKVENMPDYHFIDHRIEILGNPEDKPVKLYEIATARHHGLKGKPIPNPLLGLDSTRTG

SEQ ID NO: 67 - DNA, 639 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgtttggttggcatatattgtcaccacaattacagtatggaaacaagtcattcgtcagctacccaggcaatataccagactttttcaagcagaccgtttctggtggcgggtatacctactataagattcacttcactggcgagtttcctcctaatggtccagtgatgcagaggaggatacgaggatgggagccatccactgaacgattgtatcttcgcgacggtgtgctgacgggagatatccacaagactctgaaacttagcggtggccgccatttgagagttgactttaacacttcttacatacccaagcactcgatcaacatgccggatttccattttatagaccaccgcattgatattcggaagttcgacgaaaattacatcaacgtcgagcaggacgagattgctacagctcgccatcatgggctgaagggtaagcctatccctaaccctctcctcggactcgattctacgcgtaccggttag

SEQ ID NO: 68 - protein, 212 aa, Artificial Sequence
  Synthetically generated
MKGVKEVMKISLEMDCTVNGDKFTIKGEGGGYPYEGVQFMSLEVVNGAPLPFGWHILSPQLQYGNKSFVSYPGNIPDFFKQTVSGGGYTYYKIHFTGEFPPNGPVMQRRIRGWEPSTERLYLRDGVLTGDIHKTLKLSGGRHLRVDFNTSYIPKHSINMPDFHFIDHRIDIRKFDENYINVEQDEIATARHHGLKGKPIPNPLLGLDSTRTG

SEQ ID NO: 69 - DNA, 741 bp, Artificial Sequence
  Synthetically generated
atgagtcattccaagagtgtgatcaaggacgaaatgttcatcaagattcatctggaaggcacttttaacggccacaaatttgagatcgaaggggagggaaacggaaaaccttacgcaggaacaaattttgtaaaacttgtagtgacgaaaggcgggcctctgacgttttctttcgatgtattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaagtaatgtatgttgacgacaagagtgacggtgtgctgaagggagatgtcaacatggctctgttgcttaaagatggcggctattacagagctgaatttagaagttcttacaaaggcaagaagaaggtcgagaatatgcctgactaccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 70 - protein, 246 aa, Artificial Sequence
  Synthetically generated
MSHSKSVIKDEMFIKIHLEGTFNGHKFEIEGEGNGKPYAGTNFVKLVVTKGGPLTFSFDVLTPAFMYGNRVFTKYPKEIPDYFKQTFPEGYHWERIMTFEDGGVCCITSDISVKGDSFFYDIKFTGMNFPPHGPVMQRKTVKWEPSTEVMYVDDKSDGVLKGDVNMALLLKDGGYYRAEFRSSYKGKKKVENMPDYHFIDHRIEIMEHDEDYNHVKLREIATARHHGLKGKPIPNPLLGLDSTRTG

SEQ ID NO: 71 - DNA, 462 bp, Artificial Sequence
  Synthetically generated
atgatgaccgatctgcatctggactgcactgttaacggcgacaaatttacgatcaaaggggaaggaggaggatacccttacgaaggagtacagtttatgtctcttgaagtggtgaatggcgcgcctctgccgttttctttcgatatattgacaccacaattacagtatggaaacaagtcattcgtcagctacccaaaagagataccagactatttcaagcagacctttcctgaaggctatcactgggagcgaataatgacttttgaggacgggggcgtatgttgcatcacaagcgacatcagtgtgaaaggtgactctttctactataagattcacttcactggcgagtttcctcctcatggtccagtgatgcagagaaagacagtaaaatgggagccatccactgaagtaatgtatgttgacgacaagagtgacggtgtgcgaagggacatgacgacatga

SEQ ID NO: 72 - protein, 153 aa, Artificial Sequence
  Synthetically generated
MMTDLHLDCTVNGDKFTIKGEGGGYPYEGVQFMSLEVVNGAPLPFSFDILTPQLQYGNKSFVSYPKEIPDYFKQTFPEGYHWERIMTFEDGGVCCITSDISVKGDSFYYKIHFTGEFPPHGPVMQRKTVKWEPSTEVMYVDDKSDGVRRDMTT

SEQ ID NO: 73 - DNA, 726 bp, Artificial Sequence
  Synthetically generated
atgaagggggtgaaggaagtaatgaagatcagtctggagatggagggcgctgttaacggccaccactttgagatcgaaggggagggaaacggaaaaccttacgcaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagccacatcaggatgaaagaggaagaggagcggcatttctactataagattcacttcactggcgagtttcctcctcatggtccagtgatgcagagaaagacagtaaaatgggagccatccactgaaaacatttatcctcgcgacgaatttctggagggagatgtcaacatggctctgttgcttaaagatggccgccatttgagagttgactttaacacttcttacatacccaagaagaaggtcgagaatatgcctgactaccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 74 - protein, 241 aa, Artificial Sequence
  Synthetically generated
MKGVKEVMKISLEMEGAVNGHHFEIEGEGNGKPYAGVQFMSLEVVNGAPLPFSFDILTPAFMYGNRVFTKYPKEIPDYFKQTFPEGYHWERIMTFEDGGVCCITSHIRMKEEEERHFYYKIHFTGEFPPHGPVMQRKTVKWEPSTENIYPRDEFLEGDVNMALLLKDGRHLRVDFNTSYIPKKKVENMPDYHFIDHRIEIMEHDEDYNHVKLREIATARHHGLKGKPIPNPLLGLDSTRTG

SEQ ID NO: 75 - DNA, 492 bp, Artificial Sequence
  Synthetically generated
atgatgaccgatctgcatctggagggcgctgttaacggccaccactttacgatcaaaggggaaggaggaggatacccttacgaaggaacacagactttacatcttacagagaaggaaggcaagcctctgccgtttggttggcatatattgtcaccacaattacagtatggaaacaagtcattcgtcagctacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaaaacatttatcctcgcgacgaatttctggagggacatgacgacatgactctgcgggtgaagtggccgccatttgagagttga

SEQ ID NO: 76 - protein, 163 aa, Artificial Sequence
  Synthetically generated
MMTDLHLEGAVNGHHFTIKGEGGGYPYEGTQTLHLTEKEGKPLPFGWHILSPQLQYGNKSFVSYPKEIPDYFKQTFPEGYHWERIMTFEDGGVCCITSDISVKGDSFFYDIKFTGMNFPPHGPVMQRKTVKWEPSTENIYPRDEFLEGHDDMTLRVKWPPFES

SEQ ID NO: 77 - DNA, 717 bp, Artificial Sequence
  Synthetically generated
atgaagggggtgaaggaagtaatgaagatcagtctggagatggagggcgctgttaacggccaccactttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaacgattgtatcttcgcgacggtgtgctgacgggacatgacgacatgactctgcgggttgaaggtggcggccattacacatgtgtctttaaaactatttacagatccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 78 - protein, 238 aa, Artificial Sequence
  Synthetically generated
MKGVKEVMKISLEMEGAVNGHHFTIKGEGGGYPYEGVQFMSLEVVNGAPLPFSFDILTPAFMYGNRVFTKYPKEIPDYFKQTFPEGYHWERIMTFEDGGVCCITSDISVKGDSFFYDIKFTGMNFPPHGPVMQRKTVKWEPSTERLYLRDGVLTGHDDMTLRVEGGGHYTCVFKTIYRSKKKVENMPDYHFIDHRIEILGNPEDKPVKLYEIATARHHGLKGKPIPNPLLGLDSTRTG

SEQ ID NO: 79 - DNA, 726 bp, Artificial Sequence
  Synthetically generated
atgaagggggtgaaggaagtaatgaagatcagtctggagatggagggcgctgttaacggccaccactttacgatcaaaggggaaggaggaggatacccttacgaaggaacaaattttgtaaaacttgtagtgacgaaaggcgggcctctgccgttttctttcgatatattgacaccacaattacagtatggaaacaagtcattcgtcagctacccaaaagagataccagactatttcaagcagacctttcctgaaggctatcactgggagcgaaaaatgacttatgaggacgggggcataagtaacgtccgaagccacatcaggatgaaagaggaagaggagcggcatttcttctatgacattaagttcactggcatgaactttcctcctcatggtccagtgatgcagagaaagacagtaaaatgggagccatccactgaaaacatttatcctcgcgacgaatttctggagggacatgacgacatgactctgcgggttgaaggtggcggccattacacatgtgtctttaaaactatttacagatccaagcactcgatcaacatgccggatttccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 80 - protein, 241 aa, Artificial Sequence
  Synthetically generated
MKGVKEVMKISLEMEGAVNGHHFTIKGEGGGYPYEGTNFVKLVVTKGGPLPFSFDILTPQLQYGNKSFVSYPKEIPDYFKQTFPEGYHWERKMTYEDGGISNVRSHIRMKEEEERHFFYDIKFTGMNFPPHGPVMQRKTVKWEPSTENIYPRDEFLEGHDDMTLRVEGGGHYTCVFKTIYRSKHSINMPDFHFIDHRIEIMEHDEDYNHVKLREIATARHHGLKGKPIPNPLLGLDSTRTG

SEQ ID NO: 81 - DNA, 726 bp, Artificial Sequence
  Synthetically generated
atgagtcattccaagagtgtgatcaaggacgaaatgttcatcaagattcatctggaaggcacttttaacggccacaaatttgagatcgaaggggagggaaacggaaaaccttacgcaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctaatggtccagtgatgcagaggaggatacgaggatgggagccatccactgaaaacatttatcctcgcgacgaatttctggagggacatgacgacatgactctgcgggttgaaggtggcggccattacacatgtgtctttaaaactatttacagatccaagcactcgatcaacatgccggatttccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 82 - protein, 241 aa, Artificial Sequence
  Synthetically generated
MSHSKSVIKDEMFIKIHLEGTFNGHKFEIEGEGNGKPYAGVQFMSLEVVNGAPLPFSFDILTPAFMYGNRVFTKYPKEIPDYFKQTFPEGYHWERIMTFEDGGVCCITSDISVKGDSFFYDIKFTGMNFPPNGPVMQRRIRGWEPSTENIYPRDEFLEGHDDMTLRVEGGGHYTCVFKTIYRSKHSINMPDFHFIDHRIEILGNPEDKPVKLYEIATARHHGLKGKPIPNPLLGLDSTRTG

SEQ ID NO: 83 - DNA, 717 bp, Artificial Sequence
  Synthetically generated
atgaagggggtgaaggaagtaatgaagatcagtctggagatggactgcactgttaacggcgacaaatttgagatcgaaggggagggaaacggaaaaccttacgcaggaacaaattttgtaaaacttgtagtgacgaaaggcgggcctctgacgttttctttcgatgtattgacaccacaattacagtatggaaacaagtcattcgtcagctacccaaaagagataccagactatttcaagcagacctttcctgaaggctatcactgggagcgaataatgacttttgaggacgggggcgtatgttgcatcacaagcgacatcagtgtgaaaggtgactctttctactataagattcacttcactggcgagtttcctcctcatggtccagtgatgcagagaaagacagtaaaatgggagccatccactgaaaacatttatcctcgcgacgaatttctggagggacatgacgacatgactctgcgggttgaaggtggcggctattacagagctgaatttagaagttcttacaaaggcaagaagaacctcacgcttccggattgcttctattatgtagacaccaaacttgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 84 - protein, 238 aa, Artificial Sequence
  Synthetically generated
MKGVKEVMKISLEMDCTVNGDKFEIEGEGNGKPYAGTNFVKLVVTKGGPLTFSFDVLTPQLQYGNKSFVSYPKEIPDYFKQTFPEGYHWERIMTFEDGGVCCITSDISVKGDSFYYKIHFTGEFPPHGPVMQRKTVKWEPSTENIYPRDEFLEGHDDMTLRVEGGGYYRAEFRSSYKGKKNLTLPDCFYYVDTKLEIMEHDEDYNHVKLREIATARHHGLKGKPIPNPLLGLDSTRTG

SEQ ID NO: 85 - DNA, 546 bp, Artificial Sequence
  Synthetically generated
ttgacaccagcatttatgtatggaaaccgtgtattcaccaaatacccagccgatataccagactatatcaagctgtcctttcctgagggctttacctgggagcgaagcattccttttcaagaccaggcctcatgtaccgtcacaagcgacatcagtatgaaaagtaacaactgtttctactataagattcacttcactggcgagtttcctcctaatggtccagtgatgcagaggaggatacgaggatgggagccatccactgaacgattgtatcttcgcgacggtgtgctgacgggagatatccacaagactctgaaacttagcggtggcggctattacagagctgaatttagaagttcttacaaaggcaagcactcgatcaacatgccggatttccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 86 - protein, 181 aa, Artificial Sequence
  Synthetically generated
MTPAFMYGNRVFTKYPADIPDYIKLSFPEGFTWERSIPFQDQASCTVTSDISMKSNNCFYYKIHFTGEFPPNGPVMQRRIRGWEPSTERLYLRDGVLTGDIHKTLKLSGGGYYRAEFRSSYKGKHSINMPDFHFIDHRIEILGNPEDKPVKLYEIATARHHGLKGKPIPNPLLGLDSTRTG

SEQ ID NO: 87 - DNA, 717 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccagccgatataccagactatatcaagctgtcctttcctgagggctttacctgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaaaacatttatcctcgcgacgaatttctggagggagatgtcaacatggctctgttgcttaaagatggcggccattacacatgtgtctttaaaactatttacagatccaagcactcgatcaacatgccggatttccattttatagaccaccgcattgatattcggaagttcgacgaaaattacatcaacgtcgagcaggacgagattgctacagctcgccatcatgggctgaagggtaagcctatccctaaccctctcctcggactcgattctacgcgtaccggttag

SEQ ID NO: 88 - protein, 238 aa, Artificial Sequence
  Synthetically generated
MKGVKEVMKISLEMDCTVNGDKFTIKGEGGGYPYEGVQFMSLEVVNGAPLPFSFDILTPAFMYGNRVFTKYPADIPDYIKLSFPEGFTWERIMTFEDGGVCCITSDISVKGDSFFYDIKFTGMNFPPHGPVMQRKTVKWEPSTENIYPRDEFLEGDVNMALLLKDGGHYTCVFKTIYRSKHSINMPDFHFIDHRIDIRKFDENYINVEQDEIATARHHGLKGKPIPNPLLGLDSTRTG

SEQ ID NO: 89 - DNA, 732 bp, Artificial Sequence
  Synthetically generated
atgagtcattccaagagtgtgatcaaggacgaaatgttcatcaagattcatctggaaggcacttttaacggccacaaatttacgatcaaaggggaaggaggaggatacccttacgaaggagtacagtttatgtctcttgaagtggtgaatggcgcgcctctgccgttttctttcgatatattgacaccagcatttcagtatggaaaccgtacattcaccaaatacccaaaagagataccagactatttcaagcagacctttcctgaaggctatcactgggagcgaaaaatgacttatgaggacgggggcataagtaacgtccgaagcgacatcagtgtgaaaggtgactctttcttctatgacattaagttcactggcatgaactttcctcctcatggtccagtgatgcagagaaagacagtaaaatgggagccatccactgaaaacatttatcctcgcgacgaatttctggagggagatgtcaacatggctctgttgcttaaagatggccgccatttgagagttgactttaacacttcttacatacccaagaagaaggtcgagaatatgcctgactaccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 90 - protein, 243 aa, Artificial Sequence
  Synthetically generated
MSHSKSVIKDEMFIKIHLEGTFNGHKFTIKGEGGGYPYEGVQFMSLEVVNGAPLPFSFDILTPAFQYGNRTFTKYPKEIPDYFKQTFPEGYHWERKMTYEDGGISNVRSDISVKGDSFFYDIKFTGMNFPPHGPVMQRKTVKWEPSTENIYPRDEFLEGDVNMALLLKDGRHLRVDFNTSYIPKKKVENMPDYHFIDHRIEIMEHDEDYNHVKLREIATARHHGLKGKPIPNPLLGLDSTRTG

SEQ ID NO: 91 - DNA, 723 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccacaattacagtatggaaacaagtcattcgtcagctacccagccgatataccagactatatcaagctgtcctttcctgagggctttacctgggagcgaataatgacttttgaggacgggggcgtatgttgcatcacaagcgacatcagtgtgaaaggtgactctttctactataagattcacttcactggcgagtttcctcctcatggtccagtgatgcagagaaagacagtaaaatgggagccatccactgaagtaatgtatgttgacgacaagagtgacggtgtgctgaagggagatgtcaacatggctctgttgcttaaagatggcggccattacacatgtgtctttaaaactatttacagatccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 92 - protein, 240 aa, Artificial Sequence
  Synthetically generated
MKGVKEVMKISLEMDCTVNGDKFTIKGEGGGYPYEGVQFMSLEVVNGAPLPFSFDILTPQLQYGNKSFVSYPADIPDYIKLSFPEGFTWERIMTFEDGGVCCITSDISVKGDSFYYKIHFTGEFPPHGPVMQRKTVKWEPSTEVMYVDDKSDGVLKGDVNMALLLKDGGHYTCVFKTIYRSKKKVENMPDYHFIDHRIEILGNPEDKPVKLYEIATARHHGLKGKPIPNPLLGLDSTRTG

SEQ ID NO: 93 - DNA, 732 bp, Artificial Sequence
  Synthetically generated
atgaagggggtgaaggaagtaatgaagatcagtctggagatggagggcgctgttaacggccaccactttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaaaaatgacttatgaggacgggggcataagtaacgtccgaagccacatcaggatgaaagaggaagaggagcggcatttctactataagattcacttcactggcgagtttcctcctcatggtccagtgatgcagagaaagacagtaaaatgggagccatccactgaagtaatgtatgttgacgacaagagtgacggtgtgctgaagggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 94 - protein, 243 aa, Artificial Sequence
  Synthetically generated
MKGVKEVMKISLEMEGAVNGHHFTIKGEGGGYPYEGVQFMSLEVVNGAPLPFSFDILTPAFMYGNRVFTKYPKEIPDYFKQTFPEGYHWERKMTYEDGGISNVRSHIRMKEEEERHFYYKIHFTGEFPPHGPVMQRKTVKWEPSTEVMYVDDKSDGVLKGDVNMALLLKDGRHLRVDFNTSYIPKKKVENMPDYHFIDHRIEILGNPEDKPVKLYEIATARHHGLKGKPIPNPLLGLDSTRTG

SEQ ID NO: 95 - DNA, 744 bp, Artificial Sequence
  Synthetically generated
atgagtcattccaagagtgtgatcaaggacgaaatgttcatcaagattcatctggaaggcacttttaacggccacaaatttgagatcgaaggggagggaaacggaaaaccttacgcaggagtacagtttatgtctcttgaagtggtgaatggcgcgcctctgacgttttctttcgatgtattgacaccagcatttcagtatggaaaccgtacattcaccaaatacccaaaagagataccagactatttcaagcagacctttcctgaaggctatcactgggagcgaataatgacttttgaggacgggggcgtatgttgcatcacaagcgacatcagtatgaaaagtaacaactgtttctactataagattcacttcactggcgagtttcctcctcatggtccagtgatgcagagaaagacagtaaaatgggagccatccactgaaaacatttatcctcgcgacgaatttctggagggagatgtcaacatggctctgttgcttaaagatggccgccatttgagagttgactttaacacttcttacatacccaagaagaaggtcgagaatatgcctgactaccattttatagaccaccgcattgagattatggagcatgacgaggactacaaccatgtcaagctgcgcgagtgtgctgtagctcgctattctctgctgcctgagaagaacaagggtaagcctatccctaaccctctcctcggactcgattctacgcgtaccggttag

SEQ ID NO: 96 - protein, 247 aa, Artificial Sequence
  Synthetically generated
MSHSKSVIKDEMFIKIHLEGTFNGHKFEIEGEGNGKPYAGVQFMSLEVVNGAPLTFSFDVLTPAFQYGNRTFTKYPKEIPDYFKQTFPEGYHWERIMTFEDGGVCCITSDISMKSNNCFYYKIHFTGEFPPHGPVMQRKTVKWEPSTENIYPRDEFLEGDVNMALLLKDGRHLRVDFNTSYIPKKKVENMPDYHFIDHRIEIMEHDEDYNHVKLRECAVARYSLLPEKNKGKPIPNPLLGLDSTRTG

SEQ ID NO: 97 - DNA, 558 bp, Artificial Sequence
  Synthetically generated
atggaaaccgtgtattcaccaaatacccaggcaatataccagactttttcaagcagaccgtttctggggcgggtataccgggagcgaaaaatgacttatgaggacgggggcataagtaacgtccgaagccacatcaggatgaaagaggaagaggagcggcatttctactataagattcacttcactggcgagtttcctcctcatggtccagtgatgcagagaaagacagtaaaatgggagccatccactgaagtaatgtatgttgacgacaagagtgacggtgtgctgaagggacatgacgacatgactctgcgggttgaaggtggcggctattacagagctgaatttagaagttcttacaaaggcaagaagaaggtcgagaatatgcctgactaccattttatagaccaccgcattgagattctgggcaacccagaagacaagccggtcaagctgtacgagtgtgctgtagctcgctattctctgctgcctgagaagaacaagggtaagcctatccctaaccctctcctcggactcgattctacgcgtaccggttag

SEQ ID NO: 98 - protein, 185 aa, Artificial Sequence
  Synthetically generated
METVYSPNTQAIYQTFSSRPFLGRVYRERKMTYEDGGISNVRSHIRMKEEEERHFYYKIHFTGEFPPHGPVMQRKTVKWEPSTEVMYVDDKSDGVLKGHDDMTLRVEGGGYYRAEFRSSYKGKKKVENMPDYHFIDHRIEILGNPEDKPVKLYECAVARYSLLPEKNKGKPIPNPLLGLDSTRTG

SEQ ID NO: 99 - DNA, 720 bp, Artificial Sequence
  Synthetically generated
gtgaaggaagtaatgaagatcagtctggagatggactgcactgttaacggcgacaaatttgagatcgaaggggagggaaacggaaaaccttacgcaggaacaaattttgtaaaacttgtagtgacgaaaggcgggcctctgacgttttctttcgatgtattgacaccacaattacagtatggaaacaagtcattcgtcagctacccagccgatataccagactatatcaagctgtcctttcctgagggctttacctgggagcgaagcattccttttcaagaccaggcctcatgtaccgtcacaagcgacatcagtgtgaaaggtgactctttctactataagattcacttcactggcgagtttcctcctcatggtccagtgatgcagagaaagacagtaaaatgggagccatccactgaacgattgtatcttcgcgacggtgtgctgacgggacatgacgacatgactctgcgggttgaaggtggccgccatttgagagttgactttaacacttcttacatacccaagaagaacctcacgcttccggattgcttctattatgtagacaccaaacttgatattcggaagttcgacgaaaattacatcaacgtcgagcaggacgagtgtgctgtagctcgctattctctgctgcctgagaagaacaagggtaagcctatccctaaccctctcctcggactcgattctacgcgtaccggttag

SEQ ID NO: 100 - protein, 239 aa, Artificial Sequence
  Synthetically generated
MKEVMKISLEMDCTVNGDKFEIEGEGNGKPYAGTNFVKLVVTKGGPLTFSFDVLTPQLQYGNKSFVSYPADIPDYIKLSFPEGFTWERSIPFQDQASCTVTSDISVKGDSFYYKIHFTGEFPPHGPVMQRKTVKWEPSTERLYLRDGVLTGHDDMTLRVEGGRHLRVDFNTSYIPKKNLTLPDCFYYVDTKLDIRKFDENYINVEQDECAVARYSLLPEKNKGKPIPNPLLGLDSTRTG

SEQ ID NO: 101 - DNA, 714 bp, Artificial Sequence
  Synthetically generated
atgaagggggtgaaggaagtaatgaagatcagtctggagatggagggcgctgttaacggccaccactttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaaaacatttatcctcgcgacgaatttctggagggagatgtcaacatggctctgttgcttaaagatggcggctattacagagctgaatttagaagttcttacaaaggcaagcactcgatcaacatgccggatttccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 102 - protein, 237 aa, Artificial Sequence
  Synthetically generated
MKGVKEVMKISLEMEGAVNGHHFTIKGEGGGYPYEGVQFMSLEVVNGAPLPFSFDILTPAFMYGNRVFTKYPKEIPDYFKQTFPEGYHWERIMTFEDGGVCCITSDISVKGDSFFYDIKFTGMNFPPHGPVMQRKTVKWEPSTENIYPRDEFLEGDVNMALLLKDGGYYRAEFRSSYKGKHSINMPDFHFIDHRIEILGNPEDKPVKLYEIATARHHGLKGKPIPNPLLGLDSTRTG

SEQ ID NO: 103 - DNA, 717 bp, Artificial Sequence
  Synthetically generated
atgaagggggtgaaggaagtaatgaagatcagtctggagatggagggcgctgttaacggccaccactttgagatcgaaggggagggaaacggaaaaccttacgcaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaaaacatttatcctcgcgacgaatttctggagggagatgtcaacatggctctgttgcttaaagatggcggccattacacatgtgtctttaaaactatttacagatccaagcactcgatcaacatgccggatttccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 104 - protein, 238 aa, Artificial Sequence
  Synthetically generated
MKGVKEVMKISLEMEGAVNGHHFEIEGEGNGKPYAGVQFMSLEVVNGAPLPFSFDILTPAFMYGNRVFTKYPKEIPDYFKQTFPEGYHWERIMTFEDGGVCCITSDISVKGDSFFYDIKFTGMNFPPHGPVMQRKTVKWEPSTENIYPRDEFLEGDVNMALLLKDGGHYTCVFKTIYRSKHSINMPDFHFIDHRIEIMEHDEDYNHVKLREIATARHHGLKGKPIPNPLLGLDSTRTG

SEQ ID NO: 105 - DNA, 723 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggaacacagactttacatcttacagagaaggaaggcaagcctctgccgttttctttcgatatattgacaccacaattacagtatggaaacaagtcattcgtcagctacccagccgatataccagactatatcaagctgtcctttcctgagggctttacctgggagcgaagcattccttttcaagaccaggcctcatgtaccgtcacaagccacatcaggatgaaagaggaagaggagcggcatttctactataagattcacttcactggcgagtttcctcctaatggtccagtgatgcagaggaggatacgaggatgggagccatccactgaaaacatttatcctcgcgacgaatttctggagggagatatccacaagactctgaaacttagcggtggccgccatttgagagttgactttaacacttcttacatacccaagcactcgatcaacatgccggatttccattttatagaccaccgcattgatattcggaagttcgacgaaaattacatcaacgtcgagcaggacgagattgctacagctcgccatcatgggctgaagggtaagcctatccctaaccctctcctcggactcgattctacgcgtaccggttag

SEQ ID NO: 106 - protein, 240 aa, Artificial Sequence
  Synthetically generated
MKGVKEVMKISLEMDCTVNGDKFTIKGEGGGYPYEGTQTLHLTEKEGKPLPFSFDILTPQLQYGNKSFVSYPADIPDYIKLSFPEGFTWERSIPFQDQASCTVTSHIRMKEEEERHFYYKIHFTGEFPPNGPVMQRRIRGWEPSTENIYPRDEFLEGDIHKTLKLSGGRHLRVDFNTSYIPKHSINMPDFHFIDHRIDIRKFDENYINVEQDEIATARHHGLKGKPIPNPLLGLDSTRTG

SEQ ID NO: 107 - DNA, 720 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggaacacagactttacatcttacagagaaggaaggcaagcctctgacgttttctttcgatgtattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagccacatcaggatgaaagaggaagaggagcggcatttctactataagattcacttcactggcgagtttcctcctaatggtccagtgatgcagaggaggatacgaggatgggagccatccactgaaaacatttatcctcgcgacgaatttctggagggacatgacgacatgactctgcgggttgaaggtggcggctattacagagctgaatttagaagttcttacaaaggcaagcactcgatcaacatgccggatttccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 108 - protein, 239 aa, Artificial Sequence
  Synthetically generated
MKGVKEVMKISLEMDCTVNGDKFTIKGEGGGYPYEGTQTLHLTEKEGKPLTFSFDVLTPAFMYGNRVFTKYPKEIPDYFKQTFPEGYHWERIMTFEDGGVCCITSHIRMKEEEERHFYYKIHFTGEFPPNGPVMQRRIRGWEPSTENIYPRDEFLEGHDDMTLRVEGGGYYRAEFRSSYKGKHSINMPDFHFIDHRIEILGNPEDKPVKLYEIATARHHGLKGKPIPNPLLGLDSTRTG

SEQ ID NO: 109 - DNA, 747 bp, Artificial Sequence
  Synthetically generated
atgagtcattccaagagtgtgatcaaggacgaaatgttcatcaagattcatctggaaggcacttttaacggccacaaatttacgatcaaaggggaaggaggaggatacccttacgaaggagtacagtttatgtctcttgaagtggtgaatggcgcgcctctgccgtttggttggcatatattgtcaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctaatggtccagtgatgcagaggaggatacgaggatgggagccatccactgaagtaatgtatgttgacgacaagagtgacggtgtgctgaagggacatgacgacatgactctgcgggttgaaggtggcggccattacacatgtgtctttaaaactatttacagatccaagcactcgatcaacatgccggatttccattttatagaccaccgcattgagattctgggcaacccagaagacaagccggtcaagctgtacgagtgtgctgtagctcgctattctctgctgcctgagaagaacaagggtaagcctatccctaaccctctcctcggactcgattctacgcgtaccggttag

SEQ ID NO: 110 - protein, 248 aa, Artificial Sequence
  Synthetically generated
MSHSKSVIKDEMFIKIHLEGTFNGHKFTIKGEGGGYPYEGVQFMSLEVVNGAPLPFGWHILSPAFMYGNRVFTKYPKEIPDYFKQTFPEGYHWERIMTFEDGGVCCITSDISVKGDSFFYDIKFTGMNFPPNGPVMQRRIRGWEPSTEVMYVDDKSDGVLKGHDDMTLRVEGGGHYTCVFKTIYRSKHSINMPDFHFIDHRIEILGNPEDKPVKLYECAVARYSLLPEKNKGKPIPNPLLGLDSTRTG

SEQ ID NO: 111 - DNA, 561 bp, Artificial Sequence
  Synthetically generated
ttgacaccacaattacagtatggaaacaagtcattcgtcagctacccagccgatataccagactatatcaagctgtcctttcctgagggctttacctgggagcgaataatgacttttgaggacgggggcgtatgttgcatcacaagcgacatcagtgtgaaaggtgactctttctactataagattcacttcactggcgagtttcctcctaatggtccagtgatgcagaggaggatacgaggatgggagccatccactgaaaacatttatcctcgcgacgaatttctggagggacatgacgacatgactctgcgggttgaaggtggcggccattacacatgtgtctttaaaactatttacagatccaagaagaaggtcgagaatatgcctgactaccattttatagaccaccgcattgagattatggagcatgacgaggactacaaccatgtcaagctgcgcgagtgtgctgtagctcgctattctctgctgcctgagaagaacaagggtaagcctatccctaaccctctcctcggactcgattctacgcgtaccggttag

SEQ ID NO: 112 - protein, 186 aa, Artificial Sequence
  Synthetically generated
MTPQLQYGNKSFVSYPADIPDYIKLSFPEGFTWERIMTFEDGGVCCITSDISVKGDSFYYKIHFTGEFPPNGPVMQRRIRGWEPSTENIYPRDEFLEGHDDMTLRVEGGGHYTCVFKTIYRSKKKVENMPDYHFIDHRIEIMEHDEDYNHVKLRECAVARYSLLPEKNKGKPIPNPLLGLDSTRTG

SEQ ID NO: 113 - DNA, 720 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgtttggttggcatatattgtcaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtatgaaaagtaacaactgtttcttctatgacattaagttcactggcatgaactttcctcctaatggtccagtgatgcagaggaggatacgaggatgggagccatccactgaacgattgtatcttcgcgacggtgtgctgacgggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 114 - protein, 239 aa, Artificial Sequence
  Synthetically generated
MKGVKEVMKISLEMDCTVNGDKFTIKGEGGGYPYEGVQFMSLEVVNGAPLPFGWHILSPAFMYGNRVFTKYPKEIPDYFKQTFPEGYHWERIMTFEDGGVCCITSDISMKSNNCFFYDIKFTGMNFPPNGPVMQRRIRGWEPSTERLYLRDGVLTGDVNMALLLKDGRHLRVDFNTSYIPKKKVENMPDYHFIDHRIEILGNPEDKPVKLYEIATARHHGLKGKPIPNPLLGLDSTRTG

SEQ ID NO: 115 - DNA, 723 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgacgttttctttcgatgtattgacaccagcatttcagtatggaaaccgtacattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctaatggtccagtgatgcagaggaggatacgaggatgggagccatccactgaacgattgtatcttcgcgacggtgtgctgacgggagatgtcaacatggctctgttgcttaaagatggcggccattacacatgtgtctttaaaactatttacagatccaagaagaaggtcgagaatatgcctgactaccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggtagctcg

SEQ ID NO: 116 - protein, 241 aa, Artificial Sequence
  Synthetically generated
MKGVKEVMKISLEMDCTVNGDKFTIKGEGGGYPYEGVQFMSLEVVNGAPLTFSFDVLTPAFQYGNRTFTKYPKEIPDYFKQTFPEGYHWERIMTFEDGGVCCITSDISVKGDSFFYDIKFTGMNFPPNGPVMQRRIRGWEPSTERLYLRDGVLTGDVNMALLLKDGGHYTCVFKTIYRSKKKVENMPDYHFIDHRIEIMEHDEDYNHVKLREIATARHHGLKGKPIPNPLLGLDSTRTGSS

SEQ ID NO: 117 - DNA, 717 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgacgttttctttcgatgtattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttctactataagattcacttcactggcgagtttcctcctcatggtccagtgatgcagagaaagacagtaaaatgggagccatccactgaacgattgtatcttcgcgacggtgtgctgacgggacatgacgacatgactctgcgggttgaaggtggcggccattacacatgtgtctttaaaactatttacagatccaagaagaaggtcgagaatatgcctgactaccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 118 - protein, 238 aa, Artificial Sequence
  Synthetically generated
MKGVKEVMKISLEMDCTVNGDKFTIKGEGGGYPYEGVQFMSLEVVNGAPLTFSFDVLTPAFMYGNRVFTKYPKEIPDYFKQTFPEGYHWERIMTFEDGGVCCITSDISVKGDSFYYKIHFTGEFPPHGPVMQRKTVKWEPSTERLYLRDGVLTGHDDMTLRVEGGGHYTCVFKTIYRSKKKVENMPDYHFIDHRIEIMEHDEDYNHVKLREIATARHHGLKGKPIPNPLLGLDSTRTG

SEQ ID NO: 119 - DNA, 723 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtatgaaaagtaacaactgtttcttctatgacattaagttcactggcatgaactttcctcctaatggtccagtgatgcagaggaggatacgaggatgggagccatccactgaaaacatttatcctcgcgacgaatttctggagggagatgtcaacatggctctgttgcttaaagatggcggctattacagagctgaatttagaagttcttacaaaggcaagaagaaggtcgagaatatgcctgactaccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 120 - protein, 240 aa, Artificial Sequence
  Synthetically generated
MKGVKEVMKISLEMDCTVNGDKFTIKGEGGGYPYEGVQFMSLEVVNGAPLPFSFDILTPAFMYGNRVFTKYPKEIPDYFKQTFPEGYHWERIMTFEDGGVCCITSDISMKSNNCFFYDIKFTGMNFPPNGPVMQRRIRGWEPSTENIYPRDEFLEGDVNMALLLKDGGYYRAEFRSSYKGKKKVENMPDYHFIDHRIEIMEHDEDYNHVKLREIATARHHGLKGKPIPNPLLGLDSTRTG

SEQ ID NO: 121 - DNA, 639 bp, Artificial Sequence
  Synthetically generated
atgatgaccgatctgcatctggactgcactgttaacggcgacaaatttacgatcaaaggggaaggaggaggatacccttacgaaggaacaaattttgtaaaacttgtagtgacgaaaggcgggcctctgccgtttggttggcatatattgtcaccagcatttatgtatggaaaccgtgtattcaccaaatacccagccgatataccagactatatcaagctgtcctttcctgagggctttacctgggagcgaagcattccttttcaagaccaggcctcatgtaccgtcacaagcgacatcagtgtgaaaggtgactctttcttctatgacattaagttcactggcatgaactttcctcctaatggtccagtgatgcagaggaggatacgaggatgggagccatccactgaacgattgtatcttcgcgacggtgtgctgacgggacatgacgacatgactctgcgggttgaaggtggcggccattacacatgtgtctttaaaactatttacagatccaagcactcgatcaacatgccggatttccattttatagaccaccgcattgatattcggaagttcgacgaaaattacatcaacgtcagcaggacgagattgctacagctcgccatcatgggctga

SEQ ID NO: 122 - protein, 212 aa, Artificial Sequence
  Synthetically generated
MMTDLHLDCTVNGDKFTIKGEGGGYPYEGTNFVKLVVTKGGPLPFGWHILSPAFMYGNRVFTKYPADIPDYIKLSFPEGFTWERSIPFQDQASCTVTSDISVKGDSFFYDIKFTGMNFPPNGPVMQRRIRGWEPSTERLYLRDGVLTGHDDMTLRVEGGGHYTCVFKTIYRSKHSINMPDFHFIDHRIDIRKFDENYINVSRTRLLQLAIMG

SEQ ID NO: 123 - DNA, 714 bp, Artificial Sequence
  Synthetically generated
atgaagggggtgaaggaagtaatgaagatcagtctggagatggagggcgctgttaacggccaccactttacgatcaaaggggaaggaggaggatacccttacgaaggagtacagtttatgtctcttgaagtggtgaatggcgcgcctctgacgttttctttcgatgtattgacaccagcatttatgtatggaaaccgtgtattcaccaaatacccaaaagagataccagactatttcaagcagacctttcctgaaggctatcactgggagcgaaaaatgacttatgaggacgggggcataagtaacgtccgaagcgacatcagtatgaaaagtaacaactgtttctactataagattcacttcactggcgagtttcctcctcatggtccagtgatgcagagaaagacagtaaaatgggagccatccactgaaaacatttatcctcgcgacgaatttctggagggagatgtcaacatggctctgttgcttaaagatggcggccattacacatgtgtctttaaaactatttacagatccaagcactcgatcaacatgccggatttccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 124 - protein, 237 aa, Artificial Sequence
  Synthetically generated
MKGVKEVMKISLEMEGAVNGHHFTIKGEGGGYPYEGVQFMSLEVVNGAPLTFSFDVLTPAFMYGNRVFTKYPKEIPDYFKQTFPEGYHWERKMTYEDGGISNVRSDISMKSNNCFYYKIHFTGEFPPHGPVMQRKTVKWEPSTENIYPRDEFLEGDVNMALLLKDGGHYTCVFKTIYRSKHSINMPDFHFIDHRIEILGNPEDKPVKLYEIATARHHGLKGKPIPNPLLGLDSTRTG

SEQ ID NO: 125 - DNA, 714 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgtttggttggcatatattgtcaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaaaacatttatcctcgcgacgaatttctggagggagatgtcaacatggctctgttgcttaaagatggcggccattacacatgtgtctttaaaactatttacagatccaagcactcgatcaacatgccggatttccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 126 - protein, 237 aa, Artificial Sequence
  Synthetically generated
MKGVKEVMKISLEMDCTVNGDKFTIKGEGGGYPYEGVQFMSLEVVNGAPLPFGWHILSPAFMYGNRVFTKYPKEIPDYFKQTFPEGYHWERIMTFEDGGVCCITSDISVKGDSFFYDIKFTGMNFPPHGPVMQRKTVKWEPSTENIYPRDEFLEGDVNMALLLKDGGHYTCVFKTIYRSKHSINMPDFHFIDHRIEILGNPEDKPVKLYEIATARHHGLKGKPIPNPLLGLDSTRTG

SEQ ID NO: 127 - DNA, 741 bp, Artificial Sequence
  Synthetically generated
atgagtcattccaagagtgtgatcaaggacgaaatgttcatcaagattcatctggaaggcacttttaacggccacaaatttacgatcaaaggggaaggaggaggatacccttacgaaggagtacagtttatgtctcttgaagtggtgaatggcgcgcctctgacgttttctttcgatgtattgacaccagcatttcagtatggaaaccgtacattcaccaaatacccaaaagagataccagactatttcaagcagacctttcctgaaggctatcactgggagcgaataatgacttttgaggacgggggcgtatgttgcatcacaagcgacatcagtatgaaaagtaacaactgtttcttctatgacattaagttcactggcatgaactttcctcctcatggtccagtgatgcagagaaagacagtaaaatgggagccatccactgaagtaatgtatgttgacgacaagagtgacggtgtgctgaagggagatgtcaacatggctctgttgcttaaagatggccgccatttgagagttgactttaacacttcttacatacccaagcactcgatcaacatgccggatttccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 128 - protein, 246 aa, Artificial Sequence
  Synthetically generated
MSHSKSVIKDEMFIKIHLEGTFNGHKFTIKGEGGGYPYEGVQFMSLEVVNGAPLTFSFDVLTPAFQYGNRTFTKYPKEIPDYFKQTFPEGYHWERIMTFEDGGVCCITSDISMKSNNCFFYDIKFTGMNFPPHGPVMQRKTVKWEPSTEVMYVDDKSDGVLKGDVNMALLLKDGRHLRVDFNTSYIPKHSINMPDFHFIDHRIEIMEHDEDYNHVKLREIATARHHGLKGKPIPNPLLGLDSTRTG

SEQ ID NO: 129 - DNA, 723 bp, Artificial Sequence
  Synthetically generated
atgaagggggtgaaggaagtaatgaagatcagtctggagatggagggcgctgttaacggccaccactttacgatcaaaggggaaggaggaggatacccttacgaaggaacacagactttacatcttacagagaaggaaggcaagcctctgccgtttggttggcatatattgtcaccacaattacagtatggaaacaagtcattcgtcagctacccaaaagagataccagactatttcaagcagacctttcctgaaggctatcactgggagcgaaaaatgacttatgaggacgggggcataagtaacgtccgaagccacatcaggatgaaagaggaagaggagcggcatttctactataagattcacttcactggcgagtttcctcctcatggtccagtgatgcagagaaagacagtaaaatgggagccatccactgaacgattgtatcttcgcgacggtgtgctgacgggacatgacgacatgactctgcgggttgaaggtggccgccatttgagagttgactttaacacttcttacatacccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 130 - protein, 240 aa, Artificial Sequence
  Synthetically generated
MKGVKEVMKISLEMEGAVNGHHFTIKGEGGGYPYEGTQTLHLTEKEGKPLPFGWHILSPQLQYGNKSFVSYPKEIPDYFKQTFPEGYHWERKMTYEDGGISNVRSHIRMKEEEERHFYYKIHFTGEFPPHGPVMQRKTVKWEPSTERLYLRDGVLTGHDDMTLRVEGGRHLRVDFNTSYIPKKKVENMPDYHFIDHRIEILGNPEDKPVKLYEIATARHHGLKGKPIPNPLLGLDSTRTG

SEQ ID NO: 131 - DNA, 717 bp, Artificial Sequence
  Synthetically generated
  misc_feature (6)..(6): N is A, G, C, or T
  misc_feature (32)..(32): N is A, G, C, or T
atgaanggggtgaaggaagtaatgaagatcantctggagatggagggcgctgttaacggccaccactttacgatcaaaggggaaggaggaggatacccttacgaaggagtacagtttatgtctcttgaagtggtgaatggcgcgcctctgccgtttggttggcatatattgtcaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttctactataagattcacttcactggcgagtttcctcctcatggtccagtgatgcagagaaagacagtaaaatgggagccatccactgaaaacatttatcctcgcgacgaatttctggagggagatgtcaacatggctctgttgcttaaagatggcggctattacagagctgaatttagaagttcttacaaaggcaagaagaaggtcgagaatatgcctgactaccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 132 - protein, 238 aa, Artificial Sequence
  Synthetically generated
  UNSURE (2)..(2): Xaa is Lys or Asp
  UNSURE (11)..(11): Xaa is Ile, Asp, Ser, or Thr
MXGVKEVMKIXLEMEGAVNGHHFTIKGEGGGYPYEGVQFMSLEVVNGAPLPFGWHILSPAFMYGNRVFTKYPKEIPDYFKQTFPEGYHWERIMTFEDGGVCCITSDISVKGDSFYYKIHFTGEFPPHGPVMQRKTVKWEPSTENIYPRDEFLEGDVNMALLLKDGGYYRAEFRSSYKGKKKVENMPDYHFIDHRIEIMEHDEDYNHVKLREIATARHHGLKGKPIPNPLLGLDSTRTG

SEQ ID NO: 133 - DNA, 732 bp, Artificial Sequence
  Synthetically generated
atgagtcattccaagagtgtgatcaaggacgaaatgttcatcaagattcatctggaaggcacttttaacggccacaaatttacgatcaaaggggaaggaggaggatacccttacgaaggagtacagtttatgtctcttgaagtggtgaatggcgcgcctctgccgttttctttcgatatattgacaccagcatttcagtatggaaaccgtacattcaccaaatacccaaaagagataccagactatttcaagcagacctttcctgaaggctatcactgggagcgaataatgacttttgaggacgggggcgtatgttgcatcacaagcgacatcagtgtgaaaggtgactctttctactataagattcacttcactggcgagtttcctcctaatggtccagtgatgcagaggaggatacgaggatgggagccatccactgaagtaatgtatgttgacgacaagagtgacggtgtgctgaagggacatgacgacatgactctgcgggttgaaggtggccgccatttgagagttgactttaacacttcttacatacccaagcactcgatcaacatgccggatttccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 134 - protein, 243 aa, Artificial Sequence
  Synthetically generated
MSHSKSVIKDEMFIKIHLEGTFNGHKFTIKGEGGGYPYEGVQFMSLEVVNGAPLPFSFDILTPAFQYGNRTFTKYPKEIPDYFKQTFPEGYHWERIMTFEDGGVCCITSDISVKGDSFYYKIHFTGEFPPNGPVMQRRIRGWEPSTEVMYVDDKSDGVLKGHDDMTLRVEGGRHLRVDFNTSYIPKHSINMPDFHFIDHRIEILGNPEDKPVKLYEIATARHHGLKGKPIPNPLLGLDSTRTG

SEQ ID NO: 135 - DNA, 717 bp, Artificial Sequence
  Synthetically generated
atgaagggggtgaaggaagtaatgaagatcagtctggagatggagggcgctgttaacggccaccactttgagatcgaaggggagggaaacggaaaaccttacgcaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaaaacatttatcctcgcgacgaatttctggagggagatgtcaacatggctctgttgcttaaagatggcggccattacacatgtgtctttaaaactatttacagatccaagcactcgatcaacatgccggatttccattttatagaccaccgcattgagattatggagcatgacgaggactacaaccatgtcaagctgcgcgagattgctacagctcgccatcatgggctgaagggtaagcaaatccctaaccctctcctcggactcgattctacgggtaccggttag

SEQ ID NO: 136 - protein, 238 aa, Artificial Sequence
  Synthetically generated
MKGVKEVMKISLEMEGAVNGHHFEIEGEGNGKPYAGVQFMSLEVVNGAPLPFSFDILTPAFMYGNRVFTKYPKEIPDYFKQTFPEGYHWERIMTFEDGGVCCITSDISVKGDSFFYDIKFTGMNFPPHGPVMQRKTVKWEPSTENIYPRDEFLEGDVNMALLLKDGGHYTCVFKTIYRSKHSINMPDFHFIDHRIEIMEHDEDYNHVKLREIATARHHGLKGKQIPNPLLGLDSTGTG

SEQ ID NO: 137 - DNA, 738 bp, Artificial Sequence
  Synthetically generated
atgagtcattccaagagtgtgatcaaggacgaaatgttcatcaagattcatctggaaggcacttttaacggccacaaatttacgatcaaaggggaaggaggaggatacccttacgaaggagtacagtttatgtctcttgaagtggtgaatggcgcgcctctgacgttttctttcgatgtattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaacgattgtatcttcgcgacggtgtgctgacgggacatgacgacatgactctgcgggttgaaggtggccgccatttgagagttgactttaacacttcttacatacccaagcactcgatcaacatgccggatttccattttatagaccaccgcattgagattctgggcaacccagaagacaagccggtcaagctgtacgagtgtgctgtagctcgctattctctgctgcctgagaagaacaagggtaagcctatccctaaccctctcctcggactcgattctacgcgtaccggttag

SEQ ID NO: 138 - protein, 245 aa, Artificial Sequence
  Synthetically generated
MSHSKSVIKDEMFIKIHLEGTFNGHKFTIKGEGGGYPYEGVQFMSLEVVNGAPLTFSFDVLTPAFMYGNRVFTKYPKEIPDYFKQTFPEGYHWERIMTFEDGGVCCITSDISVKGDSFFYDIKFTGMNFPPHGPVMQRKTVKWEPSTERLYLRDGVLTGHDDMTLRVEGGRHLRVDFNTSYIPKHSINMPDFHFIDHRIEILGNPEDKPVKLYECAVARYSLLPEKNKGKPIPNPLLGLDSTRTG

SEQ ID NO: 139 - DNA, 729 bp, Artificial Sequence
  Synthetically generated
atgagtcattccaagagtgtgatcaaggacgaaatgttcatcaagattcatctggaaggcacttttaacggccacaaatttacgatcaaaggggaaggaggaggatacccttacgaaggagtacagtttatgtctcttgaagtggtgaatggcgcgcctctgacgttttctttcgatgtattgacaccagcatttatgtatggaaaccgtgtattcaccaaatacccaaaagggataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctaatggtccagtgatgcagaggaggatactaggatgggagccatccactgaacgattgtatcttcgcgacggtgtgctgacgggacatgacgacatgactctgcgggttgaaggtggcggccattacacatgtgtctttaaaactatttacagatccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 140 - protein, 242 aa, Artificial Sequence
  Synthetically generated
MSHSKSVIKDEMFIKIHLEGTFNGHKFTIKGEGGGYPYEGVQFMSLEVVNGAPLTFSFDVLTPAFMYGNRVFTKYPKGIPDYFKQTFPEGYHWERIMTFEDGGVCCITSDISVKGDSFFYDIKFTGMNFPPNGPVMQRRILGWEPSTERLYLRDGVLTGHDDMTLRVEGGGHYTCVFKTIYRSKKKVENMPDYHFIDHRIEILGNPEDKPVKLYEIATARHHGLKGKPIPNPLLGLDSTRTG

SEQ ID NO: 141 - DNA, 726 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccacaattacagtatggaaacaagtcattcgtcagctacccaaaagagataccagactatttcaagcagacctttcctgaaggctatcactgggagcgaataatgacttttgaggacgggggcgtatgttgcatcacaagccacatcaggatgaaagaggaagaggagcggcatttcttctatgacattaagttcactggcatgaactttcctcctcatggtccagtgatgcagagaaagacagtaaaatgggagccatccactgaaaacatttatcctcgcgacgaatttctggagggacatgacgacatgactctgcgggttgaaggtggccgccatttgagagttgactttaacacttcttacatacccaagcactcgatcaacatgccggatttccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 142 - protein, 241 aa, Artificial Sequence
  Synthetically generated
MKGVKEVMKISLEMDCTVNGDKFTIKGEGGGYPYEGVQFMSLEVVNGAPLPFSFDILTPQLQYGNKSFVSYPKEIPDYFKQTFPEGYHWERIMTFEDGGVCCITSHIRMKEEEERHFFYDIKFTGMNFPPHGPVMQRKTVKWEPSTENIYPRDEFLEGHDDMTLRVEGGRHLRVDFNTSYIPKHSINMPDFHFIDHRIEIMEHDEDYNHVKLREIATARHHGLKGKPIPNPLLGLDSTRTG

SEQ ID NO: 143 - DNA, 732 bp, Artificial Sequence
  Synthetically generated
atgaagggggtgaaggaagtaatgaagatcagtctggagatggactgcactgttaacggcgacaaatttgagatcgaaggggagggaaacggaaaaccttacgcaggagtacagtttatgtctcttgaagtggtgaatggcgcgcctctgccgttttctttcgatatattgacaccacaattacagtatggaaacaagtcattcgtcagctacccagccgatataccagactatatcaagctgtcctttcctgagggctttacctgggagcgaagcattccttttcaagaccaggcctcatgtaccgtcacaagccacatcaggatgaaagaggaagaggagcggcatttctactataagattcacttcactggcgagtttcctcctcatggtccagtgatgcagagaaagacagtaaaatgggagccatccactgaacgattgtatcttcgcgacggtgtgctgacgggagatgtcaacatggctctgttgcttaaagatggccgccatttgagagttgactttaacacttcttacatacccaagcactcgatcaacatgccggatttccattttatagaccaccgcattgagattctgggcaacccagaagacaagccggtcaagctgtacgagtgtgctgtagctcgctattctctgctgcctgagaagaacaagggtaagcctatccctaaccctctcctcggactcgattctacgcgtaccggttag

SEQ ID NO: 144 - protein, 243 aa, Artificial Sequence
  Synthetically generated
MKGVKEVMKISLEMDCTVNGDKFEIEGEGNGKPYAGVQFMSLEVVNGAPLPFSFDILTPQLQYGNKSFVSYPADIPDYIKLSFPEGFTWERSIPFQDQASCTVTSHIRMKEEEERHFYYKIHFTGEFPPHGPVMQRKTVKWEPSTERLYLRDGVLTGDVNMALLLKDGRHLRVDFNTSYIPKHSINMPDFHFIDHRIEILGNPEDKPVKLYECAVARYSLLPEKNKGKPIPNPLLGLDSTRTG

SEQ ID NO: 145 - DNA, 717 bp, Artificial Sequence
  Synthetically generated
atgaagggggtgaaggaagtaatgaagatcagtctggagatggactgcactgttaacggcgacaaatttgagatcgaaggggagggaaacggaaaaccttacgcaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaacgattgtatcttcgcgacggtgtgctgacgggagatgtcaacatggctctgttgcttaaagatggcggccattacacatgtgtctttaaaactatttacagatccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 146 - protein, 238 aa, Artificial Sequence
  Synthetically generated
MKGVKEVMKISLEMDCTVNGDKFEIEGEGNGKPYAGVQFMSLEVVNGAPLPFSFDILTPAFMYGNRVFTKYPKEIPDYFKQTFPEGYHWERIMTFEDGGVCCITSDISVKGDSFFYDIKFTGMNFPPHGPVMQRKTVKWEPSTERLYLRDGVLTGDVNMALLLKDGGHYTCVFKTIYRSKKKVENMPDYHFIDHRIEILGNPEDKPVKLYEIATARHHGLKGKPIPNPLLGLDSTRTG

SEQ ID NO: 147 - DNA, 513 bp, Artificial Sequence
  Synthetically generated
ttgagatcgaaggggagggaaacggaaaaccttacgcaggaacacagactttacatcttacagagaaggaaggcaagcctctgccgtttggttggcatatattgtcaccacaattacagtatggaaacaagtcattcgtcagctacccaggcaatataccagactttttcaagcagaccgtttctggtggcgggtatacccactgaagtaatgtatgttgacgacaagagtgacggtgtgctgaagggacatgacgacatgactctgcgggttgaaggtggccgccatttgagagttgactttaacacttcttacatacccaagcactcgatcaacatgccggatttccattttatagaccaccgcattgatattcggaagttcgacgaaaattacatcaacgtcgagcaggacgagtgtgctgtagctcgctattctctgctgcctgagaagaacaagggtaagcctatccctaaccctctcctcggactcgattctacgcgtaccggttag

SEQ ID NO: 148 - protein, 170 aa, Artificial Sequence
  Synthetically generated
MRSKGRETENLTQEHRLYILQRRKASLCRLVGIYCHHNYSMETSHSSATQAIYQTFSSRPFLVAGIPTEVMYVDDKSDGVLKGHDDMTLRVEGGRHLRVDFNTSYIPKHSINMPDFHFIDHRIDIRKFDENYINVEQDECAVARYSLLPEKNKGKPIPNPLLGLDSTRTG

SEQ ID NO: 149 - DNA, 690 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctattactgggagcgaaaaatgacttatgaggacgggggcataagtaacgtccgaagcgacatcagtgtgaaaggtgactctttctactataagattcacttcactggcgagtttcctcctcatggtccagtgatgcagagaaagacagtaaaatgggagccatccactgaaaacatttatcctcgcgacgaatttctggagggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcgga

SEQ ID NO: 150 - protein, 230 aa, Artificial Sequence
  Synthetically generated
MKGVKEVMKISLEMDCTVNGDKFTIKGEGGGYPYEGVQFMSLEVVNGAPLPFSFDILTPAFMYGNRVFTKYPKEIPDYFKQTFPEGYYWERKMTYEDGGISNVRSDISVKGDSFYYKIHFTGEFPPHGPVMQRKTVKWEPSTENIYPRDEFLEGDVNMALLLKDGRHLRVDFNTSYIPKKKVENMPDYHFIDHRIEILGNPEDKPVKLYEIATARHHGLKGKPIPNPLLG

SEQ ID NO: 151 - DNA, 393 bp, Artificial Sequence
  Synthetically generated
atggaaaccgtacattcaccaaatacccaggcaatataccagactttttcaagcagaccgtttctggtggcgggtatacccactgaagtaatgtatgttgacgacaagagtgacggtgtgctgaagggagatgtcaacatggctctgttgcttaaagatggccgccatttgagagttgactttaacacttcttacatacccaagcactcgatcaacatgccggatttccattttatagaccaccgcattgagattatggagcatgacgaggactacaaccatgtcaagctgcgcgagtgtgctgtagctcgctattctctgctgcctgagaagaacaagggtaagcctatccctaaccctctcctcggactcgattctacgcgtaccggttag

SEQ ID NO: 152 - protein, 130 aa, Artificial Sequence
  Synthetically generated
METVHSPNTQAIYQTFSSRPFLVAGIPTEVMYVDDKSDGVLKGDVNMALLLKDGRHLRVDFNTSYIPKHSINMPDFHFIDHRIEIMEHDEDYNHVKLRECAVARYSLLPEKNKGKPIPNPLLGLDSTRTG

SEQ ID NO: 153 - DNA, 750 bp, Artificial Sequence
  Synthetically generated
atgagtcattccaagagtgtgatcaaggacgaaatgttcatcaagattcatctggaaggcacttttaacggccacaaatttacgatcaaaggggaaggaggaggatacccttacgaaggagtacagtttatgtctcttgaagtggtgaatggcgcgcctctgacgttttctttcgatgtattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagccacatcaggatgaaagaggaagaggagcggcatttcttctatgacattaagttcactggcatgaactttcctcctcatggtccagtgatgcagagaaagacagtaaaatgggagccatccactgaagtaatgtatgttgacgacaagagtgacggtgtgctgaagggagatgtcaacatggctctgttgcttaaagatggcggctattacagagctgaatttagaagttcttacaaaggcaagaagaaggtcgagaatatgcctgactaccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 154 - protein, 249 aa, Artificial Sequence
  Synthetically generated
MSHSKSVIKDEMFIKIHLEGTFNGHKFTIKGEGGGYPYEGVQFMSLEVVNGAPLTFSFDVLTPAFMYGNRVFTKYPKEIPDYFKQTFPEGYHWERIMTFEDGGVCCITSHIRMKEEEERHFFYDIKFTGMNFPPHGPVMQRKTVKWEPSTEVMYVDDKSDGVLKGDVNMALLLKDGGYYRAEFRSSYKGKKKVENMPDYHFIDHRIEIMEHDEDYNHVKLREIATARHHGLKGKPIPNPLLGLDSTRTG

SEQ ID NO: 155 - DNA, 720 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtatgaaaagtaacaactgtttcttctatgacattaagttcactggcatgaactttcctcctcatggtccagtgatgcagagaaagacagtaaaatgggagccatccactgaacgattgtatcttcgcgacggtgtgctgacgggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 156 - protein, 239 aa, Artificial Sequence
  Synthetically generated
MKGVKEVMKISLEMDCTVNGDKFTIKGEGGGYPYEGVQFMSLEVVNGAPLPFSFDILTPAFMYGNRVFTKYPKEIPDYFKQTFPEGYHWERIMTFEDGGVCCITSDISMKSNNCFFYDIKFTGMNFPPHGPVMQRKTVKWEPSTERLYLRDGVLTGDVNMALLLKDGRHLRVDFNTSYIPKKKVENMPDYHFIDHRIEILGNPEDKPVKLYEIATARHHGLKGKPIPNPLLGLDSTRTG

SEQ ID NO: 157 - DNA, 738 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaaaaatgacttatgaggacgggggcataagtaacgtccgaagcgacatcagtgtgaaaggtgactctttcttctatgacattaagttcactggcatgaactttcctcctaatggtccagtgatgcagaggaggatacgaggatgggagccatccactgaagtaatgtatgttgacgacaagagtgacggtgtgctgaagggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagtgtgctgtagctcgctattctctgctgcctgagaagaacaagggtaagcctatccctaaccctctcctcggactcgattctacgcgtaccggttag

SEQ ID NO: 158 - protein, 245 aa, Artificial Sequence
  Synthetically generated
MKGVKEVMKISLEMDCTVNGDKFTIKGEGGGYPYEGVQFMSLEVVNGAPLPFSFDILTPAFMYGNRVFTKYPKEIPDYFKQTFPEGYHWERKMTYEDGGISNVRSDISVKGDSFFYDIKFTGMNFPPNGPVMQRRIRGWEPSTEVMYVDDKSDGVLKGDVNMALLLKDGRHLRVDFNTSYIPKKKVENMPDYHFIDHRIEILGNPEDKPVKLYECAVARYSLLPEKNKGKPIPNPLLGLDSTRTG

SEQ ID NO: 159 - DNA, 588 bp, Artificial Sequence
  Synthetically generated
gtgacgaaaggcgggcctctgacgttttctttcgatgtattgacaccagcatttcagtatggaaaccgtacattcaccaaatacccaaaagagataccagactatttcaagcagacctttcctgaaggctatcactgggagcgaagcattccttttcaagaccaggcctcatgtaccgtcacaagcgacatcagtgtgaaaggtgactctttcttctatgacattaagttcactggcatgaactttcctcctcatggtccagtgatgcagagaaagacagtaaaatgggagccatccactgaacgattgtatcttcgcgacggtgtgctgacgggagatatccacaagactctgaaacttagcggtggcggccattacacatgtgtctttaaaactatttacagatccaagcactcgatcaacatgccggatttccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttactcg

SEQ ID NO: 160 - protein, 196 aa, Artificial Sequence
  Synthetically generated
MTKGGPLTFSFDVLTPAFQYGNRTFTKYPKEIPDYFKQTFPEGYHWERSIPFQDQASCTVTSDISVKGDSFFYDIKFTGMNFPPHGPVMQRKTVKWEPSTERLYLRDGVLTGDIHKTLKLSGGGHYTCVFKTIYRSKHSINMPDFHFIDHRIEILGNPEDKPVKLYEIATARHHGLKGKPIPNPLLGLDSTRTGYS

SEQ ID NO: 161 - DNA, 738 bp, Artificial Sequence
  Synthetically generated
atgaagggggtgaaggaagtaatgaagatcagtctggagatggactgcactgttaacggcgacaaatttgagatcgaaggggagggaaacggaaaaccttacgcaggaacacagactttacatcttacagagaaggaaggcaagcctctgccgttttctttcgatatattgacaccagcatttatgtatggaaaccgtgtattcaccaaatacccaaaagagataccagactatttcaagcagacctttcctgaaggctatcactgggagcgaaaaatgacttatgaggacgggggcataagtaacgtccgaagccacatcaggatgaaagaggaagaggagcggcatttctactataagattcacttcactggcgagtttcctcctcatggtccagtgatgcagagaaagacagtaaaatgggagccatccactgaaaacatttatcctcgcgacgaatttctggagggacatgacgacatgactctgcgggttgaaggtggcggctattacagagctgaatttagaagttcttacaaaggcaagaagaaggtcgagaatatgcctgactaccattttatagaccaccgcattgagattatggagcatgacgaggactacaaccatgtcaagctgcgcgagtgtgctgtagctcgctattctctgctgcctgagaagaacaagggtaagcctatccctaaccctctcctcggactcgattctacgcgtaccggttag

SEQ ID NO: 162 - protein, 245 aa, Artificial Sequence
  Synthetically generated
MKGVKEVMKISLEMDCTVNGDKFEIEGEGNGKPYAGTQTLHLTEKEGKPLPFSFDILTPAFMYGNRVFTKYPKEIPDYFKQTFPEGYHWERKMTYEDGGISNVRSHIRMKEEEERHFYYKIHFTGEFPPHGPVMQRKTVKWEPSTENIYPRDEFLEGHDDMTLRVEGGGYYRAEFRSSYKGKKKVENMPDYHFIDHRIEIMEHDEDYNHVKLRECAVARYSLLPEKNKGKPIPNPLLGLDSTRTG

SEQ ID NO: 163 - DNA, 603 bp, Artificial Sequence
  Synthetically generated
gtgacgaaaggcgggcctctgccgttttctttcgatatattgacaccacaattacagtatggaaacaagtcattcgtcagctacccaaaagagataccagactatttcaagcagacctttcctgaaggctatcactgggagcgaataatgacttttgaggacgggggcgtatgttgcatcacaagcgacatcagtatgaaaagtaacaactgtttcttctatgacattaagttcactggcatgaactttcctcctaatggtccagtgatgcagaggaggatacgaggatgggagccatccactgaacgattgtatcttcgcgacggtgtgctgacgggagatgtcaacatggctctgttgcttaaagatggcggctattacagagctgaatttagaagttcttacaaaggcaagaagaacctcacgcttccggattgcttctattatgtagacaccaaacttgagattctgggcaacccagaagacaagccggtcaagctgtacgagtgtgctgtagctcgctattctctgctgcctgagaagaacaagggtaagcctatccctaaccctctcctcggactcgattctacgcgtaccggttag

SEQ ID NO: 164 - protein, 200 aa, Artificial Sequence
  Synthetically generated
MTKGGPLPFSFDILTPQLQYGNKSFVSYPKEIPDYFKQTFPEGYHWERIMTFEDGGVCCITSDISMKSNNCFFYDIKFTGMNFPPNGPVMQRRIRGWEPSTERLYLRDGVLTGDVNMALLLKDGGYYRAEFRSSYKGKKNLTLPDCFYYVDTKLEILGNPEDKPVKLYECAVARYSLLPEKNKGKPIPNPLLGLDSTRTG

SEQ ID NO: 165 - DNA, 663 bp, Artificial Sequence
  Synthetically generated
atgaagggggtgaaggaagtaatgaagatcagtctggagatggactgcactgttaacggcgacaaatttgagatcgaaggggagggaaacggaaaaccttacgcaggaacacagactttacatcttacagagaaggaaggcaagcctctgccgtttggttggcatatattgtcaccagcatttatgtatggaaaccgtgtattcaccaaatacccaaaagagataccagactatttcaagcagacctttcctgaaggctatcactgggagcgaagcattccttttcaagaccaggcctcatgtaccgtcacaagcgacatcagtatgaaaagtaacaactgtttcttctatgacattaagttcactggcatgaactttcctcctcatggtccagtgatgcagagaaagacagtaaaatgggagccatccactgaaaacatttatcctcgcgacgaatttctggagggagatgtcaacatggctctgttgcttaaagatggcggccattacacatgtgtctttaaaactatttacagatccaagcactcgatcaacatgccggatttccattttatagaccaccgcattgatattcggaagttcgacgaaaattacatcaacgcgagcaggacgagattgctacagctcgccatcatgggctga

SEQ ID NO: 166 - protein, 220 aa, Artificial Sequence
  Synthetically generated
MKGVKEVMKISLEMDCTVNGDKFEIEGEGNGKPYAGTQTLHLTEKEGKPLPFGWHILSPAFMYGNRVFTKYPKEIPDYFKQTFPEGYHWERSIPFQDQASCTVTSDISMKSNNCFFYDIKFTGMNFPPHGPVMQRKTVKWEPSTENIYPRDEFLEGDVNMALLLKDGGHYTCVFKTIYRSKHSINMPDFHFIDHRIDIRKFDENYINASRTRLLQLAIMG

SEQ ID NO: 167 - DNA, 726 bp, Artificial Sequence
  Synthetically generated
atgaagggggtgaaggaagtaatgaagatcagtctggagatggagggcgctgttaacggccaccactttacgatcaaaggggaaggaggaggatacccttacgaaggagtacagtttatgtctcttgaagtggtgaatggcgcgcctctgccgttttctttcgatatattgacaccagcatttcagtatggaaaccgtacattcaccaaatacccaaaagagataccagactatttcaagcagacctttcctgaaggctatcactgggagcgaataatgacttttgaggacgggggcgtatgttgcatcacaagccacatcaggatgaaagaggaagaggagcggcatttctactataagattcacttcactggcgagtttcctcctcatggtccagtgatgcagagaaagacagtaaaatgggagccatccactgaaaacatttatcctcgcgacgaatttctggagggagatgtcaacatggctctgttgcttaaagatggcggccattacacatgtgtctttaaaactatttacagatccaagaagaaggtcgagaatatgcctgactaccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 168 - protein, 241 aa, Artificial Sequence
  Synthetically generated
MKGVKEVMKISLEMEGAVNGHHFTIKGEGGGYPYEGVQFMSLEVVNGAPLPFSFDILTPAFQYGNRTFTKYPKEIPDYFKQTFPEGYHWERIMTFEDGGVCCITSHIRMKEEEERHFYYKIHFTGEFPPHGPVMQRKTVKWEPSTENIYPRDEFLEGDVNMALLLKDGGHYTCVFKTIYRSKKKVENMPDYHFIDHRIEIMEHDEDYNHVKLREIATARHHGLKGKPIPNPLLGLDSTRTG

SEQ ID NO: 169 - DNA, 624 bp, Artificial Sequence
  Synthetically generated
atggagggcgctgttaacggccaccactttgagatcgaaggggagggaaacggaaaaccttacgcaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaaaacatttatcctcgcgacgaatttctggagggagatgtcaacatggctctgttgcttaaagatggcggccattacacatgtgtctttaaaactatttacagatccaagcactcgatcaacatgccggatttccattttatagaccaccgcattgagattatggagcatgacgaggactacaaccatgtcaagctgcgcgagattgctacagctcgccatcatgggctgaag

SEQ ID NO: 170 - protein, 208 aa, Artificial Sequence
  Synthetically generated
MEGAVNGHHFEIEGEGNGKPYAGVQFMSLEVVNGAPLPFSFDILTPAFMYGNRVFTKYPKEIPDYFKQTFPEGYHWERIMTFEDGGVCCITSDISVKGDSFFYDIKFTGMNFPPHGPVMQRKTVKWEPSTENIYPRDEFLEGDVNMALLLKDGGHYTCVFKTIYRSKHSINMPDFHFIDHRIEIMEHDEDYNHVKLREIATARHHGLK

SEQ ID NO: 171 - DNA, 702 bp, Artificial Sequence
  Synthetically generated
atgatgaccgatctgcatctggactgcactgttaacggcgacaaatttacgatcaaaggggaaggaggaggatacccttacgaaggaacaaattttgtaaaacttgtagtgacgaaaggcgggcctctgccgtttggttggcatatattgtcaccacaattacagtatggaaacaagtcattcgtcagctacccagccgatataccagactatatcaagctgtcctttcctgagggctttacctgggagcgaaaaatgacttatgaggacgggggcataagtaacgtccgaagccacatcaggatgaaagaggaagaggagcggcatttctactataagattcacttcactggcgagtttcctcctcatggtccagtgatgcagagaaagacagtaaaatgggagccatccactgaaaacatttatcctcgcgacgaatttctggagggacatgacgacatgactctgcgggttgaaggtggcggccattacacatgtgtctttaaaactatttacagatccaagaagaacctcacgcttccggattgcttctattatgtagacaccaaacttgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 172 - protein, 233 aa, Artificial Sequence
  Synthetically generated
MMTDLHLDCTVNGDKFTIKGEGGGYPYEGTNFVKLVVTKGGPLPFGWHILSPQLQYGNKSFVSYPADIPDYIKLSFPEGFTWERKMTYEDGGISNVRSHIRMKEEEERHFYYKIHFTGEFPPHGPVMQRKTVKWEPSTENIYPRDEFLEGHDDMTLRVEGGGHYTCVFKTIYRSKKNLTLPDCFYYVDTKLEILGNPEDKPVKLYEIATARHHGLKGKPIPNPLLGLDSTRTG

SEQ ID NO: 173 - DNA, 729 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaaaaatgacttatgaggacgggggcataagtaacgtccgaagcgacatcagtgtgaaaggtgactctttctactataagattcacttcactggcgagtttcctcctaatggtccagtgatgcagaggaggatacgaggatgggagccatccactgaaaacatttatcctcgcgacgaatttctggagggacatgacgacatgactctgcgggttgaaggtggccgccatttgagagttgactttaacacttcttacatacccaagaagaaggtcgagaatatgcctgactaccattttatagaccaccgcattgagattatggagcatgacgaggactacaaccatgtcaagctgcgcgagtgtgctgtagctcgctattctctgctgcctgagaagaacaagggtaagcctatccctaaccctctcctcggactcgattctacgcgtaccggttag

SEQ ID NO: 174 - protein, 242 aa, Artificial Sequence
  Synthetically generated
MKGVKEVMKISLEMDCTVNGDKFTIKGEGGGYPYEGVQFMSLEVVNGAPLPFSFDILTPAFMYGNRVFTKYPKEIPDYFKQTFPEGYHWERKMTYEDGGISNVRSDISVKGDSFYYKIHFTGEFPPNGPVMQRRIRGWEPSTENIYPRDEFLEGHDDMTLRVEGGRHLRVDFNTSYIPKKKVENMPDYHFIDHRIEIMEHDEDYNHVKLRECAVARYSLLPEKNKGKPIPNPLLGLDSTRTG

SEQ ID NO: 175 - DNA, 663 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggaacacagactttacatcttacagagaaggaaggcaagcctctgccgtttggttggcatatattgtcaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttctactataagattcacttcactggcgagtttcctcctcatggtccagtgatgcagagaaagacagtaaaatgggagccatccactgaaaacatttatcctcgcgacgaatttctggagggagatgtcaacatggctctgttgcttaaagatggcggccattacacatgtgtctttaaaactatttacagatccaagaagaaggtcgagaatatgcctgactaccattttatagaccaccgcattgagattatggagcatgacgaggactacaaccatgtcaagctgcgcgagattgctacagctcgccatcatgggctgtag

SEQ ID NO: 176 - protein, 220 aa, Artificial Sequence
  Synthetically generated
MKGVKEVMKISLEMDCTVNGDKFTIKGEGGGYPYEGTQTLHLTEKEGKPLPFGWHILSPAFMYGNRVFTKYPKEIPDYFKQTFPEGYHWERIMTFEDGGVCCITSDISVKGDSFYYKIHFTGEFPPHGPVMQRKTVKWEPSTENIYPRDEFLEGDVNMALLLKDGGHYTCVFKTIYRSKKKVENMPDYHFIDHRIEIMEHDEDYNHVKLREIATARHHGL

SEQ ID NO: 177 - DNA, 726 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgtttggttggcatatattgtcaccagcatttatgtatggaaaccgtgtattcaccaaatacccaaaagagataccagactatttcaagcagacctttcctgaaggctatcactgggagcgaaaaatgacttatgaggacgggggcataagtaacgtccgaagcgacatcagtgtgaaaggtgactctttctactataagattcacttcactggcgagtttcctcctcatggtccagtgatgcagagaaagacagtaaaatgggagccatccactgaagtaatgtatgttgacgacaagagtgacggtgtgctgaagggagatgtcaacatggctctgttgcttaaagatggcggccattacacatgtgtctttaaaactatttacagatccaagaagaaggtcgagaatatgcctgactaccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 178 - protein, 241 aa, Artificial Sequence
  Synthetically generated
MKGVKEVMKISLEMDCTVNGDKFTIKGEGGGYPYEGVQFMSLEVVNGAPLPFGWHILSPAFMYGNRVFTKYPKEIPDYFKQTFPEGYHWERKMTYEDGGISNVRSDISVKGDSFYYKIHFTGEFPPHGPVMQRKTVKWEPSTEVMYVDDKSDGVLKGDVNMALLLKDGGHYTCVFKTIYRSKKKVENMPDYHFIDHRIEIMEHDEDYNHVKLREIATARHHGLKGKPIPNPLLGLDSTRTG

SEQ ID NO: 179 - DNA, 825 bp, Artificial Sequence
  Synthetically generated
atgatggcgatttccgctctaaagaacgtcatcatcatcgtaatcatatactcctgcagcactagtgctgattcgtcgaactcttactctggatcctccttcgcgaatgggattgcggaagaaatgatgaccgatctgcatctggactgcactgttaacggcgacaaatttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctaatggtccagtgatgcagaggaggatacgaggatgggagccatccactgaacgattgtatcttcgcgacggtgtgctgacgggacatgacgacatgactctgcgggttgaaggtggccgccatttgagagttgactttaacacttcttacatacccaagaagaacctcacgcttccggattgcttctattatgtagacaccaaacttgatattcggaagttcgacgaaaattacatcaacgtcgagcaggacgagattgctacagctcgccatcatgggctgaagggtaagcctatccctaaccctctcctcggactcgattctacgcgtaccggtagctcg

SEQ ID NO: 180 - protein, 275 aa, Artificial Sequence
  Synthetically generated
MMAISALKNVIIIVIIYSCSTSADSSNSYSGSSFANGIAEEMMTDLHLDCTVNGDKFTIKGEGGGYPYEGVQFMSLEVVNGAPLPFSFDILTPAFMYGNRVFTKYPKEIPDYFKQTFPEGYHWERIMTFEDGGVCCITSDISVKGDSFFYDIKFTGMNFPPNGPVMQRRIRGWEPSTERLYLRDGVLTGHDDMTLRVEGGRHLRVDFNTSYIPKKNLTLPDCFYYVDTKLDIRKFDENYINVEQDEIATARHHGLKGKPIPNPLLGLDSTRTGSS

SEQ ID NO: 181 - DNA, 750 bp, Artificial Sequence
  Synthetically generated
atgagtcattccaagagtgtgatcaaggacgaaatgttcatcaagattcatctggaaggcacttttaacggccacaaatttacgatcaaaggggaaggaggaggatacccttacgaaggagtacagtttatgtctcttgaagtggtgaatggcgcgcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagccacatcaggatgaaagaggaagaggagcggcatttcttctatgacattaagttcactggcatgaactttcctcctcatggtccagtgatgcagagaaagacagtaaaatgggagccatccactgaacgattgtatcttcgcgacggtgtgctgacgggacatgacgacatgactctgcgggttgaaggtggccgccatttgagagttgactttaacacttcttacatacccaagcactcgatcaacatgccggatttccattttatagaccaccgcattgagattatggagcatgacgaggactacaaccatgtcaagctgcgcgagtgtgctgtagctcgctattctctgctgcctgagaagaacaagggtaagcctatccctaaccctctcctcggactcgattctacgcgtaccggttag

SEQ ID NO: 182 - protein, 249 aa, Artificial Sequence
  Synthetically generated
MSHSKSVIKDEMFIKIHLEGTFNGHKFTIKGEGGGYPYEGVQFMSLEVVNGAPLPFSFDILTPAFMYGNRVFTKYPKEIPDYFKQTFPEGYHWERIMTFEDGGVCCITSHIRMKEEEERHFFYDIKFTGMNFPPHGPVMQRKTVKWEPSTERLYLRDGVLTGHDDMTLRVEGGRHLRVDFNTSYIPKHSINMPDFHFIDHRIEIMEHDEDYNHVKLRECAVARYSLLPEKNKGKPIPNPLLGLDSTRTG

SEQ ID NO: 183 - DNA, 726 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggaacaaattttgtaaaacttgtagtgacgaaaggcgggcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttcttctatgacattaagttcactggcatgaactttcctcctcatggtccagtgatgcagagaaagacagtaaaatgggagccatccactgaagtaatgtatgttgacgacaagagtgacggtgtgctgaagggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 184 - protein, 241 aa, Artificial Sequence
  Synthetically generated
MKGVKEVMKISLEMDCTVNGDKFTIKGEGGGYPYEGTNFVKLVVTKGGPLPFSFDILTPAFMYGNRVFTKYPKEIPDYFKQTFPEGYHWERIMTFEDGGVCCITSDISVKGDSFFYDIKFTGMNFPPHGPVMQRKTVKWEPSTEVMYVDDKSDGVLKGDVNMALLLKDGRHLRVDFNTSYIPKKKVENMPDYHFIDHRIEILGNPEDKPVKLYEIATARHHGLKGKPIPNPLLGLDSTRTG

SEQ ID NO: 185 - DNA, 726 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggaacacagactttacatcttacagagaaggaaggcaagcctctgacgttttctttcgatgtattgacaccacaattacagtatggaaacaagtcattcgtcagctacccaaaagagataccagactatttcaagcagacctttcctgaaggctatcactgggagcgaagcattccttttcaagaccaggcctcatgtaccgtcacaagccacatcaggatgaaagaggaagaggagcggcatttcttctatgacattaagttcactggcatgaactttcctcctcatggtccagtgatgcagagaaagacagtaaaatgggagccatccactgaaaacatttatcctcgcgacgaatttctggagggacatgacgacatgactctgcgggttgaaggtggccgccatttgagagttgactttaacacttcttacatacccaagaagaaggtcgagaatatgcctgactaccattttatagaccaccgcattgagattctgggcaacccagaagacaagccggtcaagctgtacgagattgctacagctcgccatcatgggctgaagggtaagcctatccctaacactctcctcggactcgattctacgcgtaccggttag

SEQ ID NO: 186 - protein, 241 aa, Artificial Sequence
  Synthetically generated
MKGVKEVMKISLEMDCTVNGDKFTIKGEGGGYPYEGTQTLHLTEKEGKPLTFSFDVLTPQLQYGNKSFVSYPKEIPDYFKQTFPEGYHWERSIPFQDQASCTVTSHIRMKEEEERHFFYDIKFTGMNFPPHGPVMQRKTVKWEPSTENIYPRDEFLEGHDDMTLRVEGGRHLRVDFNTSYIPKKKVENMPDYHFIDHRIEILGNPEDKPVKLYEIATARHHGLKGKPIPNTLLGLDSTRTG

SEQ ID NO: 187 - DNA, 714 bp, Artificial Sequence
  Synthetically generated
atgaagggggtgaaggaagtaatgaagatcagtctggagatggagggcgctgttaacggccaccactttacgatcaaaggggaaggaggaggatacccttacgaaggaacacagactttacatcttacagagaaggaaggcaagcctctgccgttttctttcgatatattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttctactataagattcacttcactggcgagtttcctcctcatggtccagtgatgcagagaaagacagtaaaatgggagccatccactgaaaacatttatcctcgcgacgaatttctggagggagatgtcaacatggctctgttgcttaaagatggccgccatttgagagttgactttaacacttcttacatacccaagaagaaggtcgagaatatgcctgactaccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 188 - protein, 237 aa, Artificial Sequence
  Synthetically generated
MKGVKEVMKISLEMEGAVNGHHFTIKGEGGGYPYEGTQTLHLTEKEGKPLPFSFDILTPAFMYGNRVFTKYPKEIPDYFKQTFPEGYHWERIMTFEDGGVCCITSDISVKGDSFYYKIHFTGEFPPHGPVMQRKTVKWEPSTENIYPRDEFLEGDVNMALLLKDGRHLRVDFNTSYIPKKKVENMPDYHFIDHRIEILGNPEDKPVKLYEIATARHHGLKGKPIPNPLLGLDSTRTG

SEQ ID NO: 189 - DNA, 720 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgacgttttctttcgatgtattgacaccagcatttatgtatggaaaccgtgtattcaccaaatacccaaaagagataccagactatttcaagcagacctttcctgaaggctatcactgggagcgaataatgacttttgaggacgggggcgtatgttgcatcacaagcgacatcagtgtgaaaggtgactctttctactataagattcacttcactggcgagtttcctcctcatggtccagtgatgcagagaaagacagtaaaatgggagccatccactgaagtaatgtatgttgacgacaagagtgacggtgtgctgaagggagatgtcaacatggctctgttgcttaaagatggcggccattacacatgtgtctttaaaactatttacagatccaagcactcgatcaacatgccggatttccattttatagaccaccgcattgagattctgggcaacccagaagacaagccggtcaagctgtacgagattgctacagctcgccatcatgggctgaagggtaagcctatccctaaccctctcctcggactcgattctacgcgtaccggttag

SEQ ID NO: 190 - protein, 239 aa, Artificial Sequence
  Synthetically generated
MKGVKEVMKISLEMDCTVNGDKFTIKGEGGGYPYEGVQFMSLEVVNGAPLTFSFDVLTPAFMYGNRVFTKYPKEIPDYFKQTFPEGYHWERIMTFEDGGVCCITSDISVKGDSFYYKIHFTGEFPPHGPVMQRKTVKWEPSTEVMYVDDKSDGVLKGDVNMALLLKDGGHYTCVFKTIYRSKHSINMPDFHFIDHRIEILGNPEDKPVKLYEIATARHHGLKGKPIPNPLLGLDSTRTG

SEQ ID NO: 191 - DNA, 408 bp, Artificial Sequence
  Synthetically generated
atgaaagaggaagaggagcggcatttctactataagattcacttcactggcgagtttcctcctcatggtccagtgatgcagagaaagacagtaaaatgggagccatccactgaagtaatgtatgttgacgacaagagtgacggtgtgctgaagggagatgtcaacatggctctgttgcttaaagatggcggccattacacatgtgtctttaaaactatttacagatccaagcactcgatcaacatgccggatttccattttatagaccaccgcattgagattatggagcatgacgaggactacaaccatgtcaagctgcgcgagattgctacagctcgccatcatgggctgaagggtaagcctatccctaaccctctcctcggactcgattctacgcgtaccggttag

SEQ ID NO: 192 - protein, 135 aa, Artificial Sequence
  Synthetically generated
MKEEEERHFYYKIHFTGEFPPHGPVMQRKTVKWEPSTEVMYVDDKSDGVLKGDVNMALLLKDGGHYTCVFKTIYRSKHSINMPDFHFIDHRIEIMEHDEDYNHVKLREIATARHHGLKGKPIPNPLLGLDSTRTG

SEQ ID NO: 193 - DNA, 327 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggagtacagtttatgtctcttgaagtggtgaatggcgcgcctctgccgttttctttcgatatattgacaccagcatttcagtatggaaaccgtacattcaccaaataccagccgatataccagactatatcaagctgtcctttcctgagggctttacctgggagcgaagcattccttttcaagaccaggcctcatgtaccgtcacaagccacatcaggatga

SEQ ID NO: 194 - protein, 108 aa, Artificial Sequence
  Synthetically generated
MKGVKEVMKISLEMDCTVNGDKFTIKGEGGGYPYEGVQFMSLEVVNGAPLPFSFDILTPAFQYGNRTFTKYQPIYQTISSCPFLRALPGSEAFLFKTRPHVPSQATSG

SEQ ID NO: 195 - DNA, 327 bp, Artificial Sequence
  Synthetically generated
atgaagggggtgaaggaagtaatgaagatcagtctggagatggactgcactgttaacggcgacaaatttacgatcaaaggggaaggaggaggatacccttacgaaggaacacagactttacatcttacagagaaggaaggcaagcctctgacgttttctttcgatgtattgacaccacaattacagtatggaaacaagtcattcgtcagctacccagccgatataccagactatatcaagctgtccttcctgagggctttacctgggagcgaagcattccttttcaagaccaggcctcatgtaccgtcacaagcgacatcagtatga

SEQ ID NO: 196 - protein, 108 aa, Artificial Sequence
  Synthetically generated
MKGVKEVMKISLEMDCTVNGDKFTIKGEGGGYPYEGTQTLHLTEKEGKPLTFSFDVLTPQLQYGNKSFVSYPADIPDYIKLSFLRALPGSEAFLFKTRPHVPSQATSV

SEQ ID NO: 197 - DNA, 408 bp, Artificial Sequence
  Synthetically generated
atgaaaagtaacaactgtttctactataagattcacttcactggcgagtttcctcctcatggtccagtgatgcagagaaagacagtaaaatgggagccatccactgaacgattgtatcttcgcgacggtgtgctgacgggacatgacgacatgactctgcgggttgaaggtggccgccatttgagagttgactttaacacttcttacatacccaagaagaaggtcgagaatatgcctgactaccattttatagaccaccgcattgagattatggagcatgacgaggactacaaccatgtcaagctgcgcgagtgtgctgtagctcgctattctctgctgcctgagaagaacaagggtaagcctatccctaaccctctcctcggactcgattctacgcgtaccggttag

SEQ ID NO: 198 - protein, 135 aa, Artificial Sequence
  Synthetically generated
MKSNNCFYYKIHFTGEFPPHGPVMQRKTVKWEPSTERLYLRDGVLTGHDDMTLRVEGGRHLRVDFNTSYIPKKKVENMPDYHFIDHRIEIMEHDEDYNHVKLRECAVARYSLLPEKNKGKPIPNPLLGLDSTRTG